(19) United States
(12) Patent Application Publication
(10) Pub. No.: US 2026/0297615 A1
(43) Pub. Date: Oct. 1, 2026

(54) LIVER-SPECIFIC REGULATORY NUCLEIC ACID SEQUENCES

(71) Applicant: ASKBIO INC., Research Triangle Park, NC (US)

(72) Inventors: Lester SUAREZ, Research Triangle Park, NC (US); Juan Manuel IGLESIAS GONZALEZ, Research Triangle Park, NC (US); Flavia SCIALPI, Research Triangle Park, NC (US)

(73) Assignee: ASKBIO INC., Research Triangle Park, NC (US)

(21) Appl. No.: 19/476,522

(22) PCT Filed: Apr. 19, 2024
(86) PCT No.: PCT/US2024/025395

§ 371 Date: Oct. 17, 2025

Related U.S. Application Data
(60) Provisional application No. 63/497,354, filed on Apr. 20, 2023.

(51) Int. Cl. C12N 15/86; A61K 38/37; A61K 48/00; A61P 7/04

(52) U.S. Cl. CPC C12N 15/86; A61K 38/37; A61K 48/0058; A61P 7/04; C12N 2750/14143; C12N 2830/008

(57) ABSTRACT

The present invention relates to regulatory nucleic acid sequences, in particular short liver-specific cis-regulatory elements, cis-regulatory modules, promoters and other such nucleic acid sequences, that are short and capable of enhancing liver-specific expression of genes. The invention also relates to expression constructs, vectors and cells comprising such short liver-specific regulatory nucleic acid sequences, and to methods of their use. The liver-specific regulatory nucleic acid sequences are of particular utility for gene therapy applications, but also find utility in other areas such as bioprocessing and biotechnology.

### CROSS-REFERENCE TO RELATED APPLICATIONS

This application claims benefit under 35 U.S.C. § 119(e) of U.S. Provisional Application No. 63/497,354 filed Apr. 20, 2023, the contents of which are incorporated herein by reference in their entirety.

### SEQUENCE LISTING

The instant application contains a Sequence Listing which has been submitted in XML format via Patent Center and is hereby incorporated by reference in its entirety. Said XML copy, created on Apr. 19, 2024, is named 046192-000106WOPT_SL.xml and is 246,627 bytes in size.

### FIELD OF THE INVENTION

The present invention relates to regulatory nucleic acid sequences, in particular short liver-specific cis-regulatory elements, cis-regulatory modules, promoters and other such nucleic acid sequences, that are short and capable of enhancing liver-specific expression of genes. The invention also relates to expression constructs, vectors and cells comprising such short liver-specific regulatory nucleic acid sequences, and to methods of their use. The liver-specific regulatory nucleic acid sequences are of particular utility for gene therapy applications, but also find utility in other areas such as bioprocessing and biotechnology.

### BACKGROUND OF THE INVENTION

The following discussion is provided to aid the reader in understanding the disclosure and does not constitute any admission as to the contents or relevance of the prior art.

In many areas, including gene therapy, it is desirable to provide regulatory nucleic acid sequences that are capable of driving expression of a gene to produce a protein or nucleic acid expression product within a desired cell, tissue or organ.

Expression in the liver is of particular interest as it is involved in a wide range of essential functions in the body, including the synthesis of many proteins involved in metabolism, haemostasis, and protection against infection. Given that many diseases are linked to disruption of gene expression in the liver, there is a significant interest in developing gene therapy strategies that allow expression of a transgene in the liver to produce a therapeutic expression product. Examples of diseases of the liver associated with abnormal expression of genes include haemophilia (including haemophilia A or B), familial hypercholesterolemia, ornithine transcarbamylase deficiency, α-antitrypsin deficiency, hepatitis virus infection, non-viral hepatitis, liver cancer, and various other liver diseases (such as non-alcoholic fatty liver disease (NAFLD), and alcohol-related liver disease (ARLD).

A significant challenge in using gene therapy to treat liver diseases is the ability to provide liver-specific (also known as hepato-specific) therapeutic gene expression. It is known to target mammalian hepatocytes by injecting DNA or viral vectors into the liver parenchyma, hepatic artery or portal vein. Adenoviral vectors have also been reported to primarily target the liver in mice. However, they also infect other tissues, in particular lung and skeletal muscle, leading to “off-target” effects. Some forms of adeno-associated viral vectors (AAV) or lentiviral vectors preferentially transduce hepatocytes, but off-target effects do again arise.

It is therefore desirable to provide systems to regulate gene expression in a liver-specific manner. Ideally, such systems are highly-specific to the liver (thereby avoiding or minimising off-target expression in non-target tissues) and are also powerful, i.e. they drive high expression levels in the liver. The use of cis-acting regulatory elements has been proposed to provide both specificity and activity. Typically, this concerns cis-regulatory enhancer sequences, i.e. nucleic acid sequences that act in cis to increase the activity of a promoter. Enhancers are typically active regardless of their orientation, and they can act over distances of up to several kilobases away from the promoter in some cases, though they typically also act when much closer to the promoter.

Various enhancer sequences for liver-specific expression of genes have been described in the literature. WO95/011308 and WO01/098482 describe a gene therapy vector comprising a hepatocyte-specific apolipoprotein E-Hepatocyte Control Region enhancer linked to a promoter and a transgene. Other liver-specific constructs have also been proposed in the literature, e.g. with the AAT promoter and the albumin or hepatitis B enhancers, or the alcohol dehydrogenase 6 (ADH6) basal promoter linked to two tandem copies of the apolipoprotein E enhancer element.

A further challenge in using gene therapy to treat liver diseases is the limited effective packaging capacity (payload) of vectors, in particular recombinant adeno-associated virus (rAAV) vectors. rAAV vectors are advantageous in gene therapy as they mediate stable transgene expression without inducing significant inflammatory toxicity. This is an important limitation for gene therapy of diseases where the length of the coding sequence approaches the payload limit such as Duchenne muscular dystrophy, haemophilia A and cystic fibrosis.

One way to mitigate this challenge is to provide liver-specific regulatory sequences of short length while maintaining the desired specificity and strength of expression in order to minimise the proportion of a gene therapy vector taken up by regulatory sequences.

There remains a need in the art for regulatory nucleic acids of short length which are able to drive liver-specific gene expression. In particular, there is a need for short liver-specific regulatory sequences (e.g. cis-regulatory elements and minimal or proximal promoter elements), and for short liver-specific cis-regulatory modules and promoters comprising such elements, which can be incorporated in expression constructs and vectors for liver-specific expression of a desired gene (e.g. a therapeutic transgene in a gene therapy context). Such short liver-specific regulatory sequences would be of particular interest in rAAV gene therapy where the length of the coding sequence approaches the payload limit.

### SUMMARY OF THE INVENTION

In a first aspect of the present invention, there is provided a synthetic liver-specific promoter comprising, consisting of, or consisting essentially of one of the combinations of cis-regulatory elements (CREs), or a functional variants thereof, operably linked to a promoter element, or functional variant thereof, as set out in Table A:

TABLE A
Combinations of one or more CREs in
combination with specific promoter elements

| | | | |
|---|---|---|---|
| CRE0051 | CRE0042 | CRE0059 | 5′UTR |
| CRE0051 | CRE0058 | CRE0070 | |
| CRE0051 | CRE0058 | CRE0071 | |
| CRE0042 | CRE0099 | | |
| CRE0042 | CRE0073 | | |
| CRE0051 | CRE0099 | | |
| CRE0051 | CRE0073 | | |
| CRE0051 | CRE0058 | CRE0054 | |
| CRE0094 | CRE0052 | | |
| CRE0056 | CRE0094 | CRE0052 | |
| CRE0056 | CRE0094 | CRE0059 | |
| CRE0048 | CRE0056 | CRE0054 | |
| CRE0048 | CRE0056 | CRE0052 | |
| CRE0048 | CRE0056 | CRE0059 | |

The CREs may be present in the recited order or may not present in any order. They may also be contiguous or non-contiguous (i.e. they can be positioned immediately adjacent to one another or they can be separated by a spacer or other sequences). The CREs are preferably present in the recited order, and are preferably adjacent to one another. The promoter element typically lies downstream of the CREs, and it is typically adjacent to the proximal CRE. The promoter element can be contiguous with the adjacent CRE, or it can be separated by a spacer. The sequences of the CREs are set out in Table 1 of Example 1. The sequences of the promoter elements are set out in Table 2 of Example 1. Table A sets out combinations of one or more CREs in combination with specific promoter elements that have been found to provide high level of liver-specific expression. In some preferred embodiments, the various elements of the synthetic promoters (CREs, promoter elements and UTRs) are operably linked.

The elements CRE0051, CRE0042, CRE0058, CRE0048, CRE0094 and CRE0056 are CREs. They function in combination with a promoter element to modulate, typically enhance, liver-specific transcription from the promoter in which they are comprised. Their sequences and functional variants thereof are discussed further below.

The elements CRE0059, CRE0070, CRE0071, CRE0054, CRE0073, CRE0099 and CRE0052 are minimal or proximal promoters. They function in combination with the CREs to provide liver-specific transcription from the promoter in which they are comprised. Their sequences and functional variants thereof are discussed further below.

In some embodiments, the synthetic liver-specific promoter comprises, consists of, or consists essentially of a combination of one or more CREs (or functional variants of any thereof) operably linked to a promoter element (or a functional variant of any thereof) selected from the group consisting of:
CRE0051, CRE0042, CRE0059 and 5′UTR; CRE0051, CRE0058 and CRE0070;
CRE0051, CRE0058 and CRE0071; CRE0042 and CRE0099; CRE0042 and CRE0073; CRE0051 and CRE0099; CRE0051 and CRE0073; CRE0051, CRE0058 and CRE0054; CRE0094 and CRE0052; CRE0056, CRE0094 and CRE0052; CRE0056, CRE0094 and CRE0059; CRE0048, CRE0056 and CRE0054; CRE0048, CRE0056 and CRE0052; and CRE0048, CRE0056 and CRE0059.

In a preferred embodiment, the synthetic liver-specific promoter comprises a combination of CRE0051 and CRE0042 or a functional variant of any thereof operably linked to CRE0059 and a 5′UTR. In one embodiment, the 5′UTR may comprise or consist of SEQ ID NO: 13, or a functional variant thereof. In another embodiment, the 5′UTR may comprise or consist of SEQ ID NO: 119, or a functional variant thereof.

In yet another preferred embodiment, the synthetic liver-specific promoter comprises a combination of CRE0042 or a functional variant thereof operably linked CRE0073 or a functional variant thereof.

In some embodiments of the present invention, the synthetic liver-specific promoter comprises, consists of, or consists essentially of a promoter selected from the group consisting of: SP0412_v2, SP0382, SP0383, SP0471, SP0472, SP0473, SP0474, SP0475, SP0476, SP0477, SP0478, SP0479, SP0480 and SP0481, or a functional variant of any thereof. Suitably the functional variant of any of said promoters comprises a sequence that is at least 70% identical to a reference synthetic liver-specific promoter, more preferably at least 80%, 90%, 91%, 92%, 93%, 94%, 95%, 96%, 97%, 98% or 99% identical to the reference synthetic liver-specific promoter. The sequences and SEQ ID NOs corresponding to these promoters are set out in Example 1.

In some embodiments of the present invention, the synthetic liver-specific promoter comprises, consists of, or consists essentially of any one of SEQ ID NO: 22-35. Suitably, the functional variant of any of said promoters comprises, consists of, or consists essentially of a sequence which is at least 70% identical SEQ ID NO: 22-35, more preferably at least 80%, 90%, 95%, 96%, 97%, 98% or 99% identical to SEQ ID NO: 22-35.

In some embodiments of the present invention, the synthetic liver-specific promoter comprises a promoter selected from the group consisting of:
SP0382, SP0383, SP0471, SP0473, SP0475, and SP0479 or a functional variant of any thereof. Suitably the functional variant of any of said promoters comprises a sequence that is at least 70% identical to a reference synthetic liver-specific promoter, more preferably at least 80%, 90%, 91%, 92%, 93%, 94%, 95%, 96%, 97%, 98% or 99% identical to the reference synthetic liver-specific promoter.

In some embodiments of the present invention, the synthetic liver-specific promoter comprises, consists of, consists essentially of a promoter selected from the group consisting of:
SP0477, SP0478, SP0479, SP0480 and SP0481 or a functional variant of any thereof.

Suitably the functional variant of any of said promoters comprises, consists of or consists essentially of a sequence that is at least 70% identical to the reference synthetic liver-specific promoter, more preferably at least 80%, 90%, 91%, 92%, 93%, 94%, 95%, 96%, 97%, 98% or 99% identical to the reference synthetic liver-specific promoter.

In some embodiments of the present invention the synthetic liver-specific promoter has a length of 350 or fewer nucleotides, preferably 340 or fewer nucleotides, more preferably 330 or fewer nucleotides, most preferably 320 or fewer nucleotides. In some embodiments of the present invention the synthetic liver-specific promoter has a length of 310 or fewer nucleotides, preferably 300 or fewer nucleotides, more preferably 290 or fewer nucleotides, most preferably 280 or fewer nucleotides. In some embodiments of the present invention the synthetic liver-specific promoter has a length of 270 or fewer nucleotides, preferably 280 or fewer nucleotides, more preferably 260 or fewer nucleotides, most preferably 250 or fewer nucleotides. In some embodiments of the present invention the synthetic liver-specific promoter has a length of 240 or fewer nucleotides, preferably 230 or fewer nucleotides, more preferably 220 or fewer nucleotides, most preferably 210 or fewer nucleotides. In some embodiments of the present invention the synthetic liver-specific promoter has a length of 200 or fewer nucleotides, preferably 190 or fewer nucleotides, more preferably 180 or fewer nucleotides, most preferably 170 or fewer nucleotides.

The synthetic liver-specific promoter may further comprise a UTR. The UTR may be a 5′UTR. In some embodiments, the 5′UTR may comprise, consist of, or consist essentially of SEQ ID NO: 13, or a functional variant thereof. In some embodiments, the 5′UTR may comprise, consist of, or consist essentially of SEQ ID NO: 119, or a functional variant thereof.

In some embodiments, it is particularly preferred that the synthetic liver-specific promoter comprises, consists of, or consists essentially of SP0412_v2. SP0412_v2 may be particularly preferred as it comprises the 5′ UTR of SEQ ID NO: 13. The 5′ UTR of SEQ ID NO: 13 may, for example, enhance expression compared to the liver-specific promoter which is not operably linked the additional regulatory sequence such as a UTR. Generally, it is preferred that the additional regulatory sequence does not substantively reduce the specificity of the liver-specific promoter.

In some embodiments, the synthetic liver-specific promoter may further comprise an intron. In some embodiments, the intron may be HBB2 intron (SEQ ID NO: 40) or a functional variant thereof, UBC intron (SEQ ID NO: 41), or a functional variant thereof, CMV-IE intron (SEQ ID NO: 42), or a functional variant thereof or other intron. Addition of the intron may increase the activity of the synthetic promoter. Functional variant of a reference intron may be a variant which when substituted in a reference promoter substantially retains its activity.

The sequences of the introns are set out in Table 7 of Example 1. In some embodiments, the intron comprises, consists of, or consists essentially of SEQ ID NO: 40, 41 or 42, or a functional variant thereof.

In some embodiments, the synthetic liver-specific promoter comprises, consists of, or consists essentially of any one of SEQ ID NO: 22-35, or a functional variant thereof, and HBB2 intron, the CMV-IE intron or the UBC intron, or a functional variant thereof. In some embodiments, the synthetic liver-specific promoter comprises, consists of, or consists essentially of any one of SEQ ID NO: 22-35, or a functional variant thereof, and any one of SEQ ID NO: 40, 41 or 42, or a functional variant thereof.

In some embodiments, the synthetic liver-specific promoters may further comprise an exon and splice donor sequence. The exon and splice donor sequence may assist the function of the intron, such as the HBB2 intron. In some embodiments, the synthetic liver-specific promoter comprises, consists of, or consists essentially of any one of SEQ ID NO: 22-35, or a functional variant thereof, an exon and splice donor, or a functional variant thereof and an intron, or a functional variant thereof. In some embodiments, the synthetic liver-specific promoter comprises, consists of, or consists essentially of any one of SEQ ID NO: 22-35, or a functional variant thereof, and an exon and splice donor and the HBB2 intron. The sequence of exon and splice donors is shown in Table 8 of Example 1. In some embodiments, the synthetic liver-specific promoter comprises, consists of, or consists essentially of any one of SEQ ID NO: 22-35, or a functional variant thereof, any one of SEQ ID NO: 43 and SEQ ID NO: 44, or a functional variant thereof and SEQ ID NO: 40.

The intron, when present in the synthetic liver-specific promoter, can be contiguous with the promoter element. Alternatively, the intron can be non-contiguous. For example, the promoter element and the intron may be separated by a spacer. Alternatively, the promoter element and the intron may be separated by exon and splice donor sequence such as exon and splice donor_CRE0059 (SEQ ID NO: 43) or exon and splice donor_CRE0052.1 (SEQ ID NO: 44). This embodiment is particularly preferred when the intron is the HBB2 intron as the HBB2 intron according to SEQ ID NO: 40 lacks a splice donor. In these embodiments, the splice donor is provided by a separate exon and splice donor sequence.

In a further aspect, the present invention provides a synthetic liver-specific promoter comprising, consisting of, or consisting essentially of one of the following promoter elements: CRE0070, CRE0071, CRE0099 and CRE0054 or a functional variant of any thereof. These are either proximal or minimal promoters. Each of these promoter elements has been found to provide high levels of activity when combined with one or more liver-specific CREs (e.g. as discussed above). Their sequences and functional variants thereof are discussed further below.

There is also provided in another aspect of the invention a promoter element comprising any one of CRE0070, CRE0071, CRE0099 and CRE0054, or a functional variant of any thereof, optionally wherein the promoter element has a length of 400 or fewer nucleotides, preferably 350 or fewer nucleotides, more preferably 300 or fewer nucleotides, preferably 250 or fewer nucleotides, preferably 200 or fewer nucleotides, preferably 150 or fewer nucleotides, preferably 100 or fewer nucleotides. The invention also provides a promoter element consisting of CRE0070, CRE0071, CRE0099 and CRE0054, or a functional variant of any thereof.

In a further aspect of the invention, there is provided a liver-specific CRE comprising, consisting of, or consisting essentially of CRE0094, or a functional variant of any thereof. Suitably the functional variant of any of said CREs comprises a sequence that is at least 70% identical to a reference synthetic liver-specific CRE, more preferably at least 80%, 90%, 95%, 96%, 97%, 98% or 99% identical to the reference synthetic liver-specific CRE. The sequence and SEQ ID NO corresponding to this CREs are set out in Example 1.

In another aspect of the invention, there is provided a liver-specific CRE selected from the group consisting of: CRE0051, CRE0042, CRE0058, CRE0048, CRE0094 and CRE0056, or a functional variant of any thereof, wherein the functional variant of any of said CREs comprises a sequence that is at least 70% identical to a reference synthetic liver-specific CRE, more preferably at least 80%, 90%, 95%, 96%, 97%, 98% or 99% identical to the reference synthetic liver-specific CRE.

In a further aspect of the invention, there is provided a synthetic liver-specific promoter comprising CRE0094, or a functional variant of any thereof. In some embodiments, the CRE is CRE0094 or a functional variant thereof. The CRE can be operably linked to any suitable promoter element. Exemplary, but non-limiting, promoter elements include CRE0052, CRE0059, CRE0070, CRE0071, CRE0054, CRE0099 and CRE0073, or a functional variant of any thereof. In some preferred embodiments, CRE0094 is operably linked to promoter element CRE0052 or CRE0059.

In a further aspect, there is provided a synthetic liver-specific cis-regulatory module (CRM) or synthetic liver-specific promoter comprising one or more CRE selected from the group consisting of: CRE0051, CRE0042, CRE0058, CRE0048, CRE0094 and CRE0056, or a functional variants of thereof, or a combination of said CREs.

In some embodiments the CRM comprises, consists of, or consists essentially of one of the following combinations of CREs, or functional variants thereof:
CRE0051 and CRE0042;
CRE0051 and CRE0058;
CRE0056 and CRE0094; and
CRE0048 and CRE0056.

In some preferred embodiments, there is provided a synthetic liver-specific cis-regulatory module (CRM) comprising CRE0056 operably linked to CRE0094 or CRE0048.

In particularly preferred embodiments, the synthetic liver-specific CRM of the present invention comprises, consists of, or consists essentially of a combination of CREs, or functional variants thereof, selected from the group consisting of:
CRE0048 and CRE0056; and
CRE0056 and CRE0094.

In any of the combinations of CREs, or functional variants thereof, disclosed herein, the recited CREs may be present in any order. In some preferred embodiments, the CREs are present in the recited order (i.e. in an upstream to downstream order, with reference to their position with respect to an operably linked promoter element or gene). The CREs may be contiguous or non-contiguous (i.e. they can be positioned immediately adjacent to one another or they can be separated by a spacer or other sequence). In any of the combinations of CREs, or functional variants thereof, disclosed herein, some or all of the recited CREs may suitably be positioned adjacent to one other in the CRM (i.e. without any intervening CREs or other regulatory elements). In some embodiments it is preferred that some or all of the CREs are contiguous. In some preferred embodiments, the CREs, or functional variants thereof, are provided in the recited order and are adjacent to one another. For example, synthetic liver-specific CRM may comprise CRE0048 immediately upstream of CRE0056, and so forth.

CRMs comprising the abovementioned combinations CREs have been found to provide significant liver-specific enhancer activity when combined with a suitable promoter element. Particularly high levels of activity have been observed when the CREs are present in the recited order and adjacent to one another. Thus, these represent some preferred CRE “motifs”, which typically correlate to high levels of liver-specific promoter activity.

In some embodiments of the present invention, the synthetic liver-specific CRM comprises a CRM selected from the group consisting of:
CRM_SP0412_v2, CRM_SP0382 and SP0383, CRM_SP0471, CRM_SP0473, CRM_SP0475, CRM_SP0477 and SP0478, CRM_SP0479 and CRM_SP0480 and SP0481, or a functional variant of any thereof. Suitably the functional variant of any of said CRMs comprises a sequence that is at least 70% identical to a reference synthetic liver-specific CRM, more preferably at least 80%, 90%, 91%, 92%, 93%, 94%, 95%, 96%, 97%, 98% or 99% identical to the reference synthetic liver-specific CRM. The sequences and SEQ ID NOs corresponding to these CRMs are set out in Example 1.

In some embodiments of the present invention, the synthetic liver-specific CRM comprises a CRM selected from the group consisting of: CRM_SP0477 and SP0478, CRM_SP0479, and CRM_SP0480 and SP0481, or a functional variant of any thereof. Suitably the functional variant of any of said CRMs comprises a sequence that is at least 70% identical to a reference synthetic liver-specific CRM, more preferably at least 80%, 90%, 91%, 92%, 93%, 94%, 95%, 96%, 97%, 98% or 99% identical to the reference synthetic liver-specific CRM. The sequences and SEQ ID NOs corresponding to these CRMs are set out in Example 1.

In a further aspect of the present invention, there is provided a synthetic liver-specific promoter comprising:
a) a CRM according to the previous aspect, preferably CRM comprising CRE0056 operably linked to CRE0094 or CRE0048, operably linked to a promoter element (preferably a minimal promoter or liver-specific proximal promoter); or
b) at least one of the following CREs or functional variants thereof:
   CRE0051 or a functional variant thereof;
   CRE0058 or a functional variant thereof;
   CRE0042 or a functional variant thereof;
   CRE0056 or a functional variant thereof; and
   CRE0048 or a functional variant thereof;
   operably linked to a promoter element selected from CRE0070, or a functional variant thereof, CRE0071, or a functional variant thereof, CRE0099, or a functional variant thereof, or CRE0054, or a functional variant thereof.

Suitable promoter elements for use in the synthetic liver-specific promoter of group a) are discussed herein. By way of non-limiting example, the promoter element can be selected from CRE0052, CRE0059, CRE0099 and CRE0054.

In some embodiments, the synthetic liver-specific promoter of b) comprises at least two of the recited cis-regulatory elements, or functional variants thereof, operably linked to a promoter element selected from CRE0070 or a functional variant thereof, CRE0071 or a functional variant, CRE0099 or a functional variant thereof, or a CRE0054, or a functional variant thereof.

In some embodiments, the synthetic liver-specific promoter comprises one of the individual CREs, or functional variants thereof, or combinations of CREs, or functional variants thereof, as set out in Table B operably linked to a promoter element selected from CRE0070, or a functional variant thereof, CRE0071, or a functional variant thereof, CRE0099, or a functional variant thereof, or CRE0054, or a functional variant thereof:

TABLE B

| | |
|---|---|
| CRE0051 | CRE0058 |
| CRE0042 | |
| CRE0051 | |
| CRE0048 | CRE0056 |

Again, the CREs are preferably present in the recited order, and are preferably adjacent to one another. They may also be contiguous. The promoter element lies downstream of the CREs, and it is typically adjacent to the proximal CRE. The promoter element can be contiguous with the adjacent CRE, or it can be separated by a spacer.

Table B sets out various individual CREs, or combinations of CREs, selected from CRE0051, CRE0058, CRE0042, CRE0056 and CRE0048 (or a functional variant thereof) that can suitably be provided operably linked to with promoter elements CRE0070, CRE0071, CRE0099 or CRE0054 (or functional variants thereof) in accordance with some embodiments of the present invention.

In a further aspect of the invention, there is provided an expression cassette comprising a synthetic liver-specific promoter of the present invention operably linked to a sequence encoding an expression product, suitably a gene, e.g. a transgene. Suitably, in some embodiments, the sequence encoding the expression product is codon optimised. In some embodiments, the expression product is a blood-clotting protein. In some preferred embodiments, the expression product is FVIII protein. In some particularly preferred embodiments, the transgene in a codon optimised FVIII gene. In some particularly preferred embodiments, the transgene in a CpG-depleted FVIII gene. In some particularly preferred embodiments, the transgene in a CpG-depleted and codon optimised FVIII gene.

In a further aspect of the invention, there is provided an expression cassette comprising a promoter element of the present invention operably linked to a sequence encoding an expression product, suitably a gene, e.g. a transgene. Suitably, in some embodiments, the sequence encoding the expression product is codon optimised. In some embodiments, the expression product is a blood-clotting protein. In some preferred embodiments, the expression product is FVIII protein. In some particularly preferred embodiments, the transgene in a codon optimised FVIII gene. In some particularly preferred embodiments, the transgene in a CpG-depleted FVIII gene. In some particularly preferred embodiments, the transgene in a CpG-depleted and codon optimised FVIII gene.

In a further aspect of the invention, there is provided an expression cassette comprising a liver-specific CRE, wherein the liver-specific CRE is selected from the group consisting of: CRE0051, CRE0042, CRE0058, CRE0048, CRE0094 and CRE0056, or a functional variant of any thereof operably linked to a sequence encoding an expression product, optionally wherein the sequence encoding the expression product is codon optimised. In some embodiments, the expression product is a blood-clotting protein. In some preferred embodiments, the expression product is FVIII protein. In some particularly preferred embodiments, the transgene in a codon optimised FVIII gene. In some particularly preferred embodiments, the transgene in a CpG-depleted FVIII gene. In some particularly preferred embodiments, the transgene in a CpG-depleted and codon optimised FVIII gene. Suitably, in some embodiments the functional variant of any of said CREs comprises a sequence that is at least 70% identical to a reference synthetic liver-specific CRE, more preferably at least 80%, 90%, 95%, 96%, 97%, 98% or 99% identical to the reference synthetic liver-specific CRE. Suitably, in some embodiments the liver-specific CRE comprises CRE0094 or a functional variant thereof. Suitably, in some embodiments the liver-specific CRE is CRE0094 or a functional variant thereof.

According to a further aspect of the present invention, there is provided an expression cassette comprising a synthetic liver-specific CRM of the present invention, operably linked to a sequence encoding an expression product, optionally wherein the sequence encoding the expression product is codon optimised. In some embodiments, the expression product is a blood-clotting protein. In some preferred embodiments, the expression product is FVIII protein. In some particularly preferred embodiments, the transgene in a codon optimised FVIII gene. In some particularly preferred embodiments, the transgene in a CpG-depleted FVIII gene. In some particularly preferred embodiments, the transgene in a CpG-depleted and codon optimised FVIII gene.

In a further aspect, there is provided a vector comprising a synthetic liver-specific CRM, a synthetic liver-specific promoter, or an expression cassette according to the present invention. In some embodiments the vector is an expression vector. In some embodiments the vector is a viral vector. In some embodiments the vector is a gene therapy vector, suitably an AAV vector, an adenoviral vector, a retroviral vector or a lentiviral vector. AAV vectors are of particular interest.

In a further aspect, there is provided a virion (viral particle) comprising a vector, suitably a viral vector, according to the present invention.

In a further aspect, there is provided a pharmaceutical composition comprising a synthetic liver-specific CRM, synthetic liver-specific promoter, expression cassette, vector or virion according to the present invention.

In a further aspect, there is provided a synthetic liver-specific regulatory CRM, synthetic liver-specific promoter, expression cassette, vector, virion or pharmaceutical composition according to the present invention for use in therapy, i.e. the prevention or treatment of a medical condition or disease. Suitably the condition or disease is associated with aberrant gene expression, optionally aberrant gene expression in the liver. Suitably the use is for gene therapy, preferably for use in treatment of a disease involving aberrant gene expression. In some embodiments, the disease is Pompe disease. Suitably the gene therapy involves expression of a therapeutic expression product in the liver. In some preferred embodiments, the synthetic liver-specific regulatory CRM, synthetic liver-specific promoter, expression cassette, vector, virion or pharmaceutical composition according to the present invention for use in therapy of haemophilia A.

In one aspect, there is provided a synthetic liver-specific regulatory CRM of any embodiments as described herein for use in therapy. Suitably, in some embodiments the use is for gene therapy, preferably for use in treatment of a disease involving aberrant gene expression.

In another aspect, there is provided a synthetic liver-specific promoter of any embodiments as described herein for use in therapy. Suitably, in some embodiments the use is for gene therapy, preferably for use in treatment of a disease involving aberrant gene expression.

In another aspect, there is provided an expression cassette of any embodiments as described herein for use in therapy. Suitably, in some embodiments the use is for gene therapy, preferably for use in treatment of a disease involving aberrant gene expression.

In another aspect, there is provided a vector of any embodiments as described herein for use in therapy. Suitably, in some embodiments the use is for gene therapy, preferably for use in treatment of a disease involving aberrant gene expression.

In another aspect, there is provided a virion of any embodiments as described herein for use in therapy. Suitably, in some embodiments the use is for gene therapy, preferably for use in treatment of a disease involving aberrant gene expression.

In another aspect, there is provided a pharmaceutical composition of any embodiments as described herein for use in therapy. Suitably, in some embodiments the use is for gene therapy, preferably for use in treatment of a disease involving aberrant gene expression.

In a further aspect, there is provided a cell comprising a synthetic liver-specific CRM, synthetic liver-specific promoter, expression cassette, vector, or virion as described herein. In some embodiments the cell is a eukaryotic cell, optionally a mammalian cell, optionally a human cell. Suitably the cell can be a liver cell, optionally wherein the cell is a human liver cell. The synthetic liver-specific CRM, synthetic liver-specific promoter or expression cassette can be in a vector or can be in the genome of the cell.

In a further aspect, there is provided a synthetic liver-specific CRM, synthetic liver-specific promoter, expression cassette, vector, virion or pharmaceutical composition as described herein for use in the manufacture of a pharmaceutical composition for the treatment of a medical condition or disease as discussed herein. In some embodiments, the disease is Pompe disease. In some preferred embodiments, the synthetic liver-specific CRM, synthetic liver-specific promoter, expression cassette, vector, virion or pharmaceutical composition as described herein are for use in the manufacture of a pharmaceutical composition for the treatment of haemophilia A.

In a further aspect, there is provided a method for producing an expression product, the method comprising providing a synthetic liver-specific expression cassette of the present invention in a liver cell and expressing the gene present in the synthetic liver-specific expression cassette. The method can be in vitro or ex vivo, or it can be in vivo. In some embodiments the method is a bioprocessing method. In some preferred embodiments, the expression product is Factor VIII protein.

In a further aspect, there is provided a method of expressing a therapeutic transgene in a liver cell, the method comprising introducing into the liver cell a synthetic liver-specific expression cassette, vector or virion as described herein. In some preferred embodiments, the therapeutic transgene is the Factor VIII gene.

In a further aspect, there is provided a method of therapy of a subject, preferably a human, in need thereof, the method comprising:
administering to the subject an expression cassette, vector, virion or pharmaceutical composition as described herein, which comprises a sequence encoding a therapeutic product operably linked to a promoter according to the present invention; and
expressing a therapeutic amount of the therapeutic product in the liver of said subject.

In one aspect there is provided a method of therapy of a subject, preferably a human, in need thereof, the method comprising:
administering to the subject an expression cassette of any one of the embodiments described herein; and
expressing a therapeutic amount of the therapeutic product in the liver of said subject.

In one aspect there is provided a method of therapy of a subject, preferably a human, in need thereof, the method comprising:
administering to the subject a vector of any one of the embodiments described herein; and
expressing a therapeutic amount of the therapeutic product in the liver of said subject.

In one aspect there is provided a method of therapy of a subject, preferably a human, in need thereof, the method comprising:
administering to the subject a virion of any one of the embodiments described herein; and
expressing a therapeutic amount of the therapeutic product in the liver of said subject.

In one aspect there is provided a method of therapy of a subject, preferably a human, in need thereof, the method comprising:
administering to the subject a pharmaceutical composition of any one of the embodiments described herein; and
expressing a therapeutic amount of the therapeutic product in the liver of said subject.

In some preferred embodiments, the therapeutic product is Factor VIII.

In some embodiments the method comprises:
introducing into the liver of the subject an expression cassette, vector, virion or pharmaceutical composition as described herein, which comprises a gene encoding a therapeutic product; and
expressing a therapeutic amount of the therapeutic product in the liver of said subject.

In some preferred embodiments, the therapeutic product is Factor VIII.

Suitably the method comprises administering a vector, virion or pharmaceutical composition as described herein to the subject. In some preferred embodiments the vector is a viral gene therapy vector, preferably an AAV vector.

### BRIEF DESCRIPTION OF THE FIGURES

FIG. 1: Circulating human FVIII at weeks 2 (day 14) and 4 (day 28). 2 novel promoters (SP0472 and SP0412+UTR (SP0412_v2)) and a state-of-the-art benchmark promoter (HLP) were operably linked to a sequence encoding FVIII and packaged within an AAV8 capsid. Shown are circulating hFVIII levels following administration of 1×10¹⁰ vg/mouse of AAV8-FVIII.

FIG. 2: Vector copies normalized per diploid genome in liver at week 4 after the administration of 1×10¹⁰ vg/mouse of AAV8-FVIII. Closed circles represent individual mice. Bars and lines represent mean and SD, respectively.

FIG. 3: Circulating hFVIII levels normalized per VCN at week 4 after the administration of 1×10¹⁰ vg/mouse of AAV8-FVIII.

FIG. 4A-4B: Circulating human FVIII at weeks 2 (day 14) and 4 (day 28). Novel promoter SP0472, state-of-the-art benchmark promoter TTR, and prior art promoters SP0412 and SP0246 were operably linked to a sequences encoding codon optimised FVIII and packaged within an AAV8 capsid. Shown are circulating hFVIII levels following administration of with 5e9 vg/mouse (FIG. 4A) and 1.68e9 vg/mouse vg/mouse (FIG. 4B) of AAV8-FVIII. Vector copy numbers and circulating levels of FVIII normalised per VCN are also shown.

FIG. 5: FVIII mRNA expression levels in the indicated organs at terminal sacrifice 28 days after administration. Shown are the fold change relative to vector-derived FVIII expression in the liver in each animal. N=5 mice per group (except those groups with low quality RNA samples) and their values are represented as geometric mean±SD.

FIG. 6: Bar graph of experimental results indicating circulating hFVIII levels in mice that have been administered the plasmids containing the indicated FVIII nucleic acids, by hydrodynamic tail vein injection. Results are shown as hFVIII levels (% of normal) in mice that received a successful hydrodynamic injection. Values are represented as Mean±standard deviation. Values below the limit of quantification of 1.56% of normal hFVIII are shown as 1.56 for illustration purposes. The vertical dotted line separates experimental round 1 and 2 on the graph. * p<0.05 vs QQ00 Round 2, one-way ANOVA.

### DETAILED DESCRIPTION OF EMBODIMENTS OF THE INVENTION AND EXAMPLES

### CREs and Functional Variants Thereof:

Disclosed herein are various CREs that can be used in the construction of liver-specific promoters. These CREs are generally derived from genomic promoter and enhancer sequences, but they are used herein in contexts quite different from their native genomic environment. Generally, the CREs constitute small parts of much larger genomic regulatory domains, which control expression of the genes with which they are normally associated. It has been surprisingly found that these CREs, many of which are very small, can be isolated form their normal environment and retain liver-specific regulatory activity when used to construct various synthetic promoters. This is surprising because the removal of a regulatory sequence from the complex and “three dimensional” natural context in the genome often results in a significant loss of activity, so there is no reason to expect a given CRE to retain the levels of activity observed once removed from their natural environment. Many combinations of these CREs have been tested and found to be highly effective at enhancing liver-specific promoter activity when combined with minimal and proximal promoters. It should be noted that the sequences of the CREs of the present invention can be altered without causing a substantial loss of activity. Thus, functional variants of the CREs discussed below can be prepared by modifying the sequence of the CREs, provided that modifications which are significantly detrimental to activity of the CRE are avoided. In view of the information provided in the present disclosure, modification of CREs to provide functional variants is straightforward. Moreover, the present disclosure provides methodologies for simply assessing the functionality of any given CRE variant. Functional variants for CREs are discussed below.

The relatively small size of certain CREs according to the present invention is advantageous because it allows for the CREs, more specifically promoters containing them, to be provided in vectors while taking up the minimal amount of the payload of the vector. This is particularly important when a CRE is used in a vector with limited capacity, such as an AAV-based vector.

The CREs of the present invention comprise certain liver-specific transcription factor binding sites (TFBS). It is generally desired that in functional variants of the CREs these liver-specific TFBS remain functional. In some cases, it may be preferred that all TFBS (whether liver-specific or otherwise) remain functional. The skilled person is well aware that TFBS sequences can vary yet retain functionality. In view of this, the sequence for a TFBS is typically illustrated by a consensus sequence from which some degree of variation is typically present. Further information about the variation that occurs in a TFBS can be illustrated using a positional weight matrix (PWM), which represents the frequency with which a given nucleotide is typically found at a given location in the consensus sequence. Details of transcription factor (TF) consensus sequences and associated positional weight matrices can be found in, for example, the Jaspar or Transfac databases jaspar.genereg.net/and gene-regulation.com/pub/databases.html). This information allows the skilled person to modify the sequence in any given TFBS of a CRE in a manner which retains, and in some cases even increases, CRE functionality. CREs can be scanned against all PWM from JASPAR database to identify/analyse all TFBS. The skilled person can of course find additional guidance in the literature, and, moreover, routine experimentation can be used to confirm TF binding to a putative TFBS in any variant CRE. Moreover, it is routine for the skilled person to analyse the CRE recited herein to identify the liver-specific TFBS present in the CRE, and this allows the skilled person to provide variants of a CRE that retain all liver-specific TFBS. It will be apparent that significant sequence modification in a CRE, even within TFBS in a CRE, can be made while retaining function.

By way of example, if we consider the TFBS for HNF1 found in CRE0051 as set out below. The TFBS for HNF1 in CRE0051 has the sequence GTTAATTTTTAAA (SEQ ID NO: 101). In view of this the skilled person has ample guidance on how the TFBS for HNF1 can be modified, while maintaining ability to bind the desired TF; the Jaspar system will, for example, score a putative TFBS based on its similarity to a given PWM. Furthermore, CREs can be scanned against all PWM from JASPAR database to identify/analyse all TFBS. The skilled person can of course find additional guidance in the literature, and, moreover, routine experimentation can be used to confirm TF binding to a putative TFBS in any variant CRE. While HNF1 has been discussed in this example, the skilled person can do the same for the other TFs and TFBS mentioned herein. It will be apparent that significant sequence modification in a CRE, even within TFBS in a CRE, can be made while retaining function.

Functional variants of CRE0051, CRE0042, CRE0058, CRE0048, CRE0094 and CRE0056 are regulatory elements with sequences which vary from the reference element, but which substantially retain their activity as liver-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments the functional variant of CRE0051 TFBS for the same liver-specific TF as CRE0051. The liver-specific TFBS present in CRE0051, listed in the order in which they are present, are: HNF1, HNF4, HNF3, HNF1 and HNF3. The functional variant of CRE0051 thus preferably comprises all of these TFBS. Preferably, they are present in the same order that they are present in CRE0051, i.e. in the order HNF1, HNF4, HNF3, HNF1 then HNF3. When the cis-regulatory element is associated with a promoter and gene, this order is preferably considered in an upstream to downstream direction (i.e. in the direction from distal from the transcription start site (TSS) to proximal to the TSS). Spacer sequences may be provided between adjacent TFBS. In some embodiments the TFBS may suitably overlap, provided they remain functional, i.e. overlapping sequences are both able to bind their respective TFs.

In some embodiments the functional variant of CRE0051 comprises the following TFBS sequences: GTTAATTTTTAAA (HNF1), GTGGCCCTTGG (HNF4), TGTTTGC (HNF3), TGGTTAATAATCTCA (HNF1) then ACAAACA (HNF3), sequences complementary thereto, or functional variants of these TFBS sequences that maintain the ability to bind to their respective TF (see Table 9 for TFBS SEQ ID NOs). These may be present in the same order as CRE0051, i.e. the order in which they are set out above. It is well-known in the art that there is sequence variability associated with TFBS, and that for a given TFBS there is typically a consensus sequence, from which some degree of deviation is typically present.

In some embodiments the functional variant of CRE0042 comprises TFBS for the same liver-specific TF as CRE0042. The liver-specific TFBS present in CRE0042, listed in the order in which they are present, are: HNF3, C/EBP, HNF4, and C/EBP. The functional variant of CRE0042 thus preferably comprises all of these TFBS. Preferably, they are present in the same order that they are present in CRE0042, i.e. in the order HNF3, C/EBP, HNF4, then C/EBP. When the cis-regulatory element is associated with a promoter and gene, this order is preferably considered in an upstream to downstream direction (i.e. in the direction from distal from the transcription start site (TSS) to proximal to the TSS). Spacer sequences may be provided between adjacent TFBS. In some embodiments the TFBS may suitably overlap, provided they remain functional, i.e. overlapping sequences are both able to bind their respective TFs.

In some embodiments the functional variant of CRE0042 comprises the following TFBS sequences: GTTCAAACATG (HNF3), CTAATACTCTG (C/EBP), TGCAAGGGTCAT (HNF4), then TTACTCAACA (C/EBP), sequences complementary thereto, or functional variants of these TFBS sequences that maintain the ability to bind to their respective TF (see Table 10 for TFBS SEQ ID NOs). These may be present in the same order as CRE0042, i.e. the order in which they are set out above. It is well-known in the art that there is sequence variability associated with TFBS, and that for a given TFBS there is typically a consensus sequence, from which some degree of deviation is typically present.

In some embodiments the functional variant of CRE0058 comprises TFBS for the same liver-specific TF as CRE0058. The liver-specific TFBS present in CRE0058, listed in the order in which they are present, are: HNF-4 and c/EBP. The functional variant of CRE0058 thus preferably comprises all of these TFBS. Preferably, they are present in the same order that they are present in CRE0058, i.e. in the order HNF4 then c/EBP. When the cis-regulatory element is associated with a promoter and gene, this order is preferably considered in an upstream to downstream direction (i.e. in the direction from distal from the transcription start site (TSS) to proximal to the TSS). Spacer sequences may be provided between adjacent TFBS. In some embodiments the TFBS may suitably overlap, provided they remain functional, i.e. overlapping sequences are both able to bind their respective TFs.

In some embodiments the functional variant of CRE0058 comprises the following TFBS sequence: CGCCCTTTGGACC (HNF4) and GACCTTTTGCAATCCTGG (c/EBP) sequences complementary thereto, or functional variants of these TFBS sequences that maintain the ability to bind to their respective TF (see Table 11 for TFBS SEQ ID NO). These may be present in the same order as CRE0058, i.e. the order in which they are set out above. It is well-known in the art that there is sequence variability associated with TFBS, and that for a given TFBS there is typically a consensus sequence, from which some degree of deviation is typically present.

In some embodiments the functional variant of CRE0056 comprises TFBS for the same liver-specific TF as CRE0056. The liver-specific TFBS present in CRE0056, listed in the order in which they are present, are: HNF4, HNF3 and HNF3b. The functional variant of CRE0056 thus preferably comprises all of these TFBS. Preferably, they are present in the same order that they are present in CRE0056, i.e. in the order HNF1, HNF4, HNF3, then HNF3b. When the cis-regulatory element is associated with a promoter and gene, this order is preferably considered in an upstream to downstream direction (i.e. in the direction from distal from the transcription start site (TSS) to proximal to the TSS). Spacer sequences may be provided between adjacent TFBS. In some embodiments the TFBS may suitably overlap, provided they remain functional, i.e. overlapping sequences are both able to bind their respective TFs.

In some embodiments the functional variant of CRE0056 comprises the following TFBS sequences: ACTGAACCCTTGACCCCTGCCCT (HNF4), CTGTTTGCCCACTCTATTTGCCC (HNF3), then TGCCCACTCTATTTGCCCAGCC (HNF3b), sequences complementary thereto, or functional variants of these TFBS sequences that maintain the ability to bind to their respective TF (see Table 12 for TFBS SEQ ID NOs). These may be present in the same order as CRE0056, i.e. the order in which they are set out above. It is well-known in the art that there is sequence variability associated with TFBS, and that for a given TFBS there is typically a consensus sequence, from which some degree of deviation is typically present.

In some embodiments the functional variant of CRE0094 comprises TFBS for the same liver-specific TF as CRE0094. The liver-specific TFBS present in CRE0094, listed in the order in which they are present, are: HNF4A, FOXA1 and FOXA1. The functional variant of CRE0094 thus preferably comprises all of these TFBS. Preferably, they are present in the same order that they are present in CRE0094, i.e. in the order HNF4A, FOXA1 then FOXA1. When the cis-regulatory element is associated with a promoter and gene, this order is preferably considered in an upstream to downstream direction (i.e. in the direction from distal from the transcription start site (TSS) to proximal to the TSS). Spacer sequences may be provided between adjacent TFBS. In some embodiments the TFBS may suitably overlap, provided they remain functional, i.e. overlapping sequences are both able to bind their respective TFs.

In some embodiments the functional variant of CRE0094 comprises the following TFBS sequences: AAGTCCAAAGGTAGA (HNF4A), GAGTCAACATGA (FOXA1), and CAGTCAACATTT (FOXA1), sequences complementary thereto, or functional variants of these TFBS sequences that maintain the ability to bind to their respective TF (see Table 13 for TFBS SEQ ID NOs). These may be present in the same order as CRE0094, i.e. the order in which they are set out above. It is well-known in the art that there is sequence variability associated with TFBS, and that for a given TFBS there is typically a consensus sequence, from which some degree of deviation is typically present.

Bioinformatic analysis of CRE0048 revealed that it does not apparently contain any known liver-specific TFBS. Nonetheless, the present inventors have determined that CRE0048 nonetheless contributes to liver-specific activity of promoters. Without wishing to be bound by theory, this may be through cooperative interaction with other CREs that do contain liver-specific TFBS to enhance their activity.

In some embodiments, a functional variant of CRE0051, CRE0042, CRE0058, CRE0048, CRE0094 and CRE0056 can be viewed as a CRE which, when substituted in place of the reference CRE in a CRM or promoter, substantially retains its activity. For example, a promoter which comprises a functional variant of CRE0051, CRE0042, CRE0058, CRE0048, CRE0094 and CRE0056 substituted in place of the reference promoter preferably retains 80% of its activity, more preferably 90% of its activity, more preferably 95% of its activity, and yet more preferably 100% of its activity (compared to the reference promoter comprising the reference CRE). For example, considering promoter SP0481 as an example, CRE0048 in SP0481 can be replaced with a functional variant of CRE0048, and the promoter substantially retains its activity. Retention of activity can be assessed by comparing expression of a suitable reporter under the control of the reference promoter with an otherwise identical promoter comprising the substituted CRE under equivalent conditions. Suitable assays for assessing liver-specific promoter activity are disclosed herein, e.g. in Example 2, Example 3 and Example 4.

In some embodiments, a functional variant of CRE0051, CRE0042, CRE0058, CRE0048, CRE0094 or CRE0056 suitably comprises a sequence that is at least 70% identical to the sequence of the reference CRE, more preferably at least 80%, 90%, 95%, 96%, 97%, 98% or 99% identical to the sequence of the reference CRE (i.e. any one of SEQ ID NOs: 1 to 6), or functional variants thereof, fall within the scope of the invention. Additionally or alternatively, a functional variant of CRE0051, CRE0042, CRE0058, CRE0048, CRE0094 or CRE0056 suitably comprises a sequence which hybridises under stringent conditions to a nucleic acid comprising the reference CRE sequence.

In some embodiments of the invention the CRE consists of any one of SEQ ID NOs: 1-6, or functional variants thereof.

It will be noted that the CRE or functional variant thereof can be provided on either strand of a double stranded polynucleotide and can be provided in either orientation. As such, complementary and reverse complementary sequences of SEQ ID NOs: 1 to 6, or functional variants thereof, fall within the scope of the invention. Single stranded nucleic acids comprising a sequence according to any one of SEQ ID NOs: 1 to 6, or a functional variant, thereof also fall within the scope of the invention.

In some preferred embodiments, there is provided a CRE comprising, consisting of, or consisting essentially of CRE0051, CRE0042, CRE0058, CRE0048, CRE0094 or CRE0056 or a functional variant thereof which has a length of 200 or fewer nucleotides, 150 or fewer nucleotides, 125 or fewer nucleotides, or 100 or fewer nucleotides, 75 or fewer nucleotides, 50 or fewer nucleotides, or 30 or fewer nucleotides.

### Promoter Elements and Functional Variants Thereof:

Various promoter elements are disclosed herein that can be used in the construction of synthetic liver-specific promoters. These promoter elements are either minimal promoters or liver-specific proximal promoters. While the CREs and CRMs of the present invention can be used in combination with a range of suitable minimal promoters or liver-specific proximal promoters, some proximal promoters have been found to act synergistically with the CREs or CRMs to contribute significantly to activity of the liver-specific promoter. In addition, some liver-specific proximal promoters as disclosed herein have been found to have high levels of activity even in the absence of additional CRE or CRM sequences.

Functional variants of a promoter element include sequences which vary from the reference promoter element, but which substantially retain their activity as liver-specific promoter elements. It will be appreciated by the skilled person that it is possible to vary the sequence of a promoter element while retaining its ability to recruit RNA polymerase II, and, where relevant, bind to liver-specific transcription factors (TFs) to enhance expression. A functional variant of a promoter element can comprise substitutions, deletions and/or insertions compared to a reference promoter element, provided they do not render the promoter element non-functional.

In some embodiments, a functional variant of a promoter element can be viewed as a promoter element which, when substituted in place of a reference promoter element in a promoter, substantially retains its activity. For example, a liver-specific promoter which comprises a functional variant of a given promoter element preferably retains at least 80% of its activity, more preferably at least 90% of its activity, more preferably at least 95% of its activity, and yet more preferably 100% of its activity (compared to the reference promoter comprising the unmodified reference promoter element). Suitable assays for assessing liver-specific promoter activity are disclosed herein, e.g. in Example 2, Example 3 and Example 4.

Suitably, functional variants of a promoter element retain a significant level of sequence identity to a reference promoter element. Suitably functional variants comprise a sequence that is at least 70% identical to the reference promoter element, more preferably at least 80%, 90%, 95%, 96%, 97%, 98% or 99% identical to the reference promoter element. As discussed above, functional variants of CRE0059, CRE0070, CRE0071, CRE0099, CRE0073, CRE0054, CRE0052 substantially retain the ability of the reference promoter element to act as a liver-specific promoter element. For example, when a functional variant of any of the recited promoter elements is substituted into a liver-specific promoter comprising the reference promoter element, the modified liver-specific promoter retains at least 80% of its activity, more preferably at least 90% of its activity, more preferably at least 95% of its activity, and yet more preferably 100% of its activity. Suitably the functional variant of the promoter element comprises a sequence which has at least 70%, 80%, 90%, 95%, 96%, 97%, 98% or 99% identity to any one of SEQ ID NOs: 7-12, 36.

In the case of promoter elements that are proximal promoters, it is preferred that functional variants retain TFBS for the liver-specific TFs that bind to the reference promoter element (the above discussion regarding PWMs applies equally here). Preferably the TFBS are retained in the same order and at substantially the same location as in the reference promoter element. It is generally preferred that the sequence of the transcription start site (TSS) is substantially unaltered in a functional variant of a promoter element. In some cases a promoter element may comprise a 5′ untranslated region. It is preferred that such 5′ UTR is retained in a functional variant, but in some cases it can be removed or the sequence can be altered, provided suitable activity is retained.

Retention of activity can be assessed by comparing expression of a suitable reporter under the control of the reference promoter with an otherwise identical promoter comprising the substituted promoter element under equivalent conditions. Suitable assays for assessing liver-specific promoter activity are disclosed herein, e.g. in Example 2, Example 3 and Example 4.

Promoter elements used in the present invention can be natural (i.e. obtained or derived from a naturally occurring gene promoter) or can be synthetic (i.e. non-naturally occurring).

### Other Promoter Elements:

Non-limiting examples of other liver-specific proximal promoters that may be used in the present invention include, but are not limited to, the ApoA-I promoter, the ApoA-II promoter, the ApoA-IV promoter, the ApoB promoter, the ApoC-1 promoter, the ApoC-II promoter, the ApoC-III promoter, the ApoE promoter, the albumin promoter, the α-fetoprotein promoter, the phosphoenolpyruvate carboxykinase (PCK1) promoter, the phosphoenolpyruvate carboxykinase 2 (PCK2) promoter, the transthyretin (TTR) promoter, the α-antitrypsin (AAT or SERPINA1) promoter, the TK (thymidine kinase) promoter, the hemopexin promoter, the alcohol dehydrogenase 6 promoter, the cholesterol 7alpha-25 hydroxylase promoter, the factor IX promoter, the a-microglobulin promoter, the SV40 promoter, the CMV promoter, the Rous Sarcoma Virus-L TR promoter and the HBV promoter. Minimal promoters derived from these promoters can of course also be used.

### Synthetic Liver-Specific CRMs and Functional Variants Thereof:

Various synthetic liver-specific CRMs are disclosed herein that can be used in the constructions of synthetic liver-specific promoters. CRMs of the present invention can be used in combination with a wide range of suitable minimal promoters or liver-specific proximal promoters, as discussed above.

Functional variants of a CRM include sequences which vary from the reference CRM element, but which substantially retain activity as liver-specific CRMs. It will be appreciated by the skilled person that it is possible to vary the sequence of a CRM while retaining its ability to recruit suitable liver-specific transcription factors (TFs) and thereby enhance expression. A functional variant of a CRM can comprise substitutions, deletions and/or insertions compared to a reference CRM, provided they do not render the CRM substantially non-functional. Reference synthetic liver-specific CRMs include: CRM_SP0412_v2, CRM_SP0382, CRM_SP0383, CRM_SP0471, CRM_SP0473, CRM_SP0475, CRM_SP0477, CRM_SP0478, CRM_SP0479 and CRM_SP0480.

In some embodiments, a functional variant of a CRM can be viewed as a CRM which, when substituted in place of a reference CRM in a promoter, substantially retains its activity. For example, a liver-specific promoter which comprises a functional variant of a given CRM preferably retains at least 80% of its activity, more preferably at least 90% of its activity, more preferably at least 95% of its activity, and yet more preferably 100% of its activity (compared to the reference promoter comprising the unmodified CRM).

Suitably, functional variants of a CRM retain a significant level of sequence identity to a reference CRM. Suitably functional variants comprise a sequence that is at least 70% identical to the reference CRM, more preferably at least 80%, 90%, 95%, 96%, 97%, 98% or 99% identical to the reference CRM.

Retention of activity can be assessed by comparing expression of a suitable reporter under the control of the reference promoter with an otherwise identical promoter comprising the substituted CRM under equivalent conditions. Suitable assays for assessing liver-specific promoter activity are disclosed herein, e.g. in Example 2, Example 3 and Example 4.

Functional variants of a given CRM can, in some embodiments, comprise functional variants of one or more of the CREs present in the reference CRM. For example, functional variants of a given CRM can comprise functional variants of 1 or 2 of the CREs present in the reference CRM. Functional variants of CREs are discussed above.

Functional variants of a given CRM can, in some embodiments, comprise the same combination of CREs as a reference CRM, but the CREs can be present in a different order from the reference CRM. It is usually preferred that the CREs are present in the same order as the reference CRM (thus, the functional variant of a CRM suitably comprises the same permutation of the CREs as set out in a reference CRM).

Functional variants of a given CRM can, in some embodiments, comprise one or more additional CREs to those present in a reference CRM. Additional CREs can be provided upstream of the CREs present in the reference CRM, downstream of the CREs present in the reference CRM, and/or between the CREs present in the reference CRM. The additional CREs can be CREs disclosed herein, or they can be other CREs. Generally, it is preferred that a functional variant of a given CRM comprises the same CREs (or functional variants thereof) and does not comprise additional CREs.

Functional variants of a given CRM can comprise one or more additional regulatory elements compared to a reference CRM. For example, they may comprise an inducible or repressible element, a boundary control element, an insulator, a locus control region, a response element, a binding site, a segment of a terminal repeat, a responsive site, a stabilizing element, a de-stabilizing element, and a splicing element, etc., provided that they do not render the CRM substantially non-functional.

Functional variants of a given CRM can comprise additional spacers between adjacent CREs or, if one or more spacers are present in the reference CRM, said one or more spacers can be longer or shorter than in the reference CRM. Spacers present in the reference CRM can also be removed in the functional variant.

It will be apparent that the CRMs as disclosed herein, or functional variants thereof, can be combined with any suitable promoter elements in order to provide a synthetic liver-specific promoter according to the present invention.

In many instances, shorter promoter sequences are preferred, particularly for use in situations where a vector (e.g. a viral vector such as AAV) has limited capacity. Accordingly, in some embodiments the synthetic liver-specific CRM has length of 200, 150, 100, 75, 60, 50 or fewer nucleotides.

In some instances, CREs and/or CRMs with a low CpG dinucleotide content are preferred. A CpG dinucleotide represents a cytosine 5′ to a guanine, linked by a phosphodiester bond. Suitably, in some embodiments, it is preferred that the CREs or CRMs of the present invention have a low CpG dinucleotide content. By low CpG content it is meant that there are fewer CpG dinucleotides compared to a reference CRE or CRM. Suitably, in some embodiments, a low CpG dinucleotide content is less than 5%, less than 2%, less than 1%, less than 0.75%, less than 0.5%, less than 0.25%, less than 0.1% or less than 0.01% of the total dinucleotide content. Suitably, in some embodiments, the synthetic liver-specific CRE is CpG-depleted. Suitably, in some embodiments, the synthetic liver-specific CRM is CpG-depleted.

### Synthetic Liver-Specific Promoters and Functional Variants Thereof:

Various synthetic liver-specific promoters are disclosed herein.

A functional variant of a reference synthetic liver-specific promoter is a promoter which comprise a sequence which varies from the reference synthetic liver-specific promoter, but which substantially retains liver-specific promoter activity. It will be appreciated by the skilled person that it is possible to vary the sequence of a synthetic liver-specific promoter while retaining its ability to recruit suitable liver-specific transcription factors (TFs) and to recruit RNA polymerase II to provide liver-specific expression of an operably linked sequence (e.g. open reading frame). A functional variant of a synthetic liver-specific promoter can comprise substitutions, deletions and/or insertions compared to a reference promoter, provided such substitutions, deletions and/or insertions do not render the synthetic liver-specific promoter substantially non-functional compared to the reference promoter. Reference synthetic liver-specific promoters include: SP0412_v2, SP0382, SP0383, SP0471, SP0472, SP0473, SP0474, SP0475, SP0476, SP0477, SP0478, SP0479, SP0480 and SP0481. The sequences (SEQ ID NOs: 22 to 35) corresponding to these promoters are set out in Example 1.

Accordingly, in some embodiments, a functional variant of a synthetic liver-specific promoter can be viewed as a variant which substantially retains the liver-specific promoter activity of the reference promoter. For example, a functional variant of a synthetic liver-specific promoter preferably retains at least 70% of the activity of the reference promoter, more preferably at least 80% of its activity, more preferably at least 90% of its activity, more preferably at least 95% of its activity, and yet more preferably 100% of its activity.

Functional variants of a synthetic liver-specific promoter often retain a significant level of sequence similarity to a reference synthetic liver-specific promoter. In some embodiments, functional variants comprise a sequence that is at least 70% identical to the reference synthetic liver-specific promoter, more preferably at least 80%, 90%, 95%, 96%, 97%, 98% or 99% identical to the reference synthetic liver-specific promoter. Reference sequences for the abovementioned promoters are SEQ ID NOs: 22 to 35, as set out in Table 1.

Activity in a functional variant can be assessed by comparing expression of a suitable reporter under the control of the reference synthetic liver-specific promoter with the putative functional variant under equivalent conditions. Suitable assays for assessing liver-specific promoter activity are disclosed herein, e.g. in Example 2, Example 3 and Example 4.

Functional variants of a given synthetic liver-specific promoter can comprise functional variants of one or more CREs present in the reference synthetic liver-specific promoter. For example, functional variant of a given synthetic liver-specific promoter can comprise 1 or 2 of the CREs present in the reference synthetic liver-specific promoter. Functional variants of CREs are discussed above.

Functional variants of a given synthetic liver-specific promoter can comprise functional variants of the promoter element, or a different promoter element when compared to the reference synthetic liver-specific promoter.

Functional variants of a given synthetic liver-specific promoter can comprise the same CREs as a reference synthetic liver-specific promoter, but the CREs can be present in a different order from the reference synthetic liver-specific promoter.

Functional variants of a given synthetic liver-specific promoter can comprise one or more additional CREs to those present in a reference synthetic liver-specific promoter. Additional CREs can be provided upstream of the CREs present in the reference CRM, downstream of the CREs present in the reference synthetic liver-specific promoter, and/or between the CREs present in the reference synthetic liver-specific promoter. The additional CREs can be CREs disclosed herein, or they can be other CREs.

Functional variants of a given synthetic liver-specific promoter can comprise one or more additional regulatory elements compared to a reference synthetic liver-specific promoter. For example, they may comprise an inducible elements, an intronic element, a boundary control element, an insulator, a locus control region, a response element, a binding site, a segment of a terminal repeat, a responsive site, a stabilizing element, a de-stabilizing element, and a splicing element, etc., provided that they do not render the promoter substantially non-functional. Functional variants can also include a 5′ UTR sequence.

Functional variants of a given synthetic liver-specific promoter can comprise additional spacers between adjacent CREs and promoter elements or, if one or more spacer are present in the reference synthetic liver-specific promoter, said one or more spacers can be longer or shorter than in the reference synthetic liver-specific promoter. Spacers present in the reference liver-specific promoter may also be removed in the functional variant.

It will be apparent that synthetic liver-specific promoters of the present invention can comprise a CRM of the present invention and additional regulatory sequences. For example, they may comprise one or more additional CRMs, an inducible or repressible element, a boundary control element, an insulator, a locus control region, a response element, a binding site, a segment of a terminal repeat, a responsive site, a stabilizing element, a de-stabilizing element, and a splicing element, etc., provided that they do not render the promoter substantially non-functional.

Preferred synthetic liver-specific promoters of the present invention exhibit liver-specific promoter activity which is at least 15%, 20%, 25%, 30%, 40%, 50%, 60%, 70%, 80%, 90%, 100%, 125%, 150%, 175%, 200%, 250%, 300%, 350% or 400% of the activity of the TBG promoter. In some embodiments, the synthetic liver-specific promoters of the invention are suitable for promoting liver-specific transgene expression at a level at least 100% of the activity of the LP1 promoter, preferably 150%, 200%, 300% or 500% of the activity of the LP1 promoter. In some embodiments, the synthetic liver-specific promoters of the invention are suitable for promoting liver-specific transgene expression at a level at least 70%, 80%, 90%, or 100% of the activity of the HLP promoter, preferably 150%, 200%, 300% or 500% of the activity of the HLP promoter. In many cases higher levels of promoter activity is preferred, but this is not always the case; thus, in some cases more moderate levels of expression may be preferred. In some cases more moderate levels of expression may be preferred, e.g. to prevent toxicity or accumulation of protein. Activity of a given synthetic liver-specific promoter of the present invention compared to TBG can be assessed by comparing liver-specific expression of a reporter gene under control of the synthetic liver-specific promoter with expression of the same reporter under control of the TBG promoter, when the two promoters are provided in otherwise equivalent expression constructs and under equivalent conditions. The same applies for LP1 and HLP.

In some embodiments a synthetic liver-specific promoter of the invention is able to increase expression of a gene (e.g. a therapeutic gene or gene of interest) in the liver of a subject or in a liver cell by at least 20%, at least 40%, at least 60%, at least 80%, at least 100%, at least 200%, at least 300%, at least 500%, at least 1000% or more relative to a known liver-specific promoter, suitably the LP-1 promoter.

Preferred synthetic liver-specific promoters of the present invention exhibit activity in non-liver cells (e.g. HEK293 cells) which is 50% or less when compared to CMV-IE, preferably 25% or less than CMV-IE, more preferably 10% or less than CMV-IE, and in some cases 5% or less than CMV-IE, or 1% or less than CMV-IE. In some embodiments, a promoter is liver-specific if it is more active in liver than in non-liver cells or tissues.

In many instances, shorter promoter sequences are preferred, particularly for use in situations where a vector (e.g. a viral vector such as AAV) has limited capacity. Accordingly, in some embodiments the synthetic liver-specific promoter has length of 350, preferably 300, more preferably 250, most preferably 200 or fewer nucleotides.

Particularly preferred synthetic liver-specific promoters are those that are both short and which exhibit high levels of activity.

In some instances, promoter sequences with a low CpG dinucleotide content are preferred. A CpG dinucleotide represents a cytosine 5′ to a guanine, linked by a phosphodiester bond.

Suitably, in some embodiments, it is preferred that the synthetic liver-specific promoter of the present invention has a low CpG dinucleotide content. By low CpG content it is meant that there are fewer CpG dinucleotides compared to a reference promoter. Suitably, in some embodiments, a low CpG dinucleotide content is less than 5%, less than 2%, less than 1%, less than 0.75%, less than 0.5%, less than 0.25%, less than 0.1% or less than 0.01% of the total dinucleotide content. Suitably, in some embodiments, the synthetic liver-specific promoter is CpG-depleted. The CpG dinucleotide content for the abovementioned promoters (SEQ ID NOs: 22 to 35), are exemplified in Table 5. Suitably, in some embodiments, the synthetic liver-specific promoter of the present invention has less than 25, less than 24, less than 23, less than 22, less than 21, less than 20, less than 19, less than 18, less than 17, less than 16, less than 15, less than 14, less than 13, less than 12, less than 11, less than 10, less than 9, less than 8, less than 7, less than 6, less than 5, less than 4, less than 3, less than 2 or less than 1 CpG dinucleotides.

### Synthetic Liver-Specific Expression Cassettes:

The present invention also provides a synthetic liver-specific expression cassette comprising a synthetic liver-specific promoter of the present invention operably linked to a sequence encoding an expression product, suitably a gene (e.g. a transgene). The gene typically encodes a desired gene expression product such as a polypeptide (protein) or RNA. The gene may be a full-length cDNA or genomic DNA sequence, or any fragment, subunit or mutant thereof that has at least some desired biological activity.

Where the gene encodes a protein, it can be essentially any type of protein. By way of non-limiting example, the protein can be an enzyme, an antibody or antibody fragment (e.g. a monoclonal antibody), a viral protein (e.g. REP-CAP, REV, VSV-G, or RD114), a therapeutic protein (e.g. FVIII), or a toxic protein (e.g. Caspase 3, 8 or 9).

In some embodiments, the gene is human factor FVIII (FVIII). The native human factor VIII (FVIII) gene has been characterized (Gene ID: 2157; Ensembl: ENSG00000185010 MIM: 300841; AllianceGenome: HGNC: 3546; UniProtKB—P00451). GenBank Accession Nos. NM_000132.3 and NP_000123.1 provide examples of the nucleotide and amino acid sequences of wild-type native human FVIII. The Factor VIII gene produces alternatively spliced transcripts. Transcript variant 1 encodes the large glycoprotein (sometimes referred to as isoform a), which is synthesized as a single chain polypeptide of 2351 amino acids. A 19-amino acid signal peptide is cleaved by a protease shortly after synthesis so that circulating plasma factor VIII is a heterodimer. This circulates in plasma and associates with von Willebrand factor in a noncovalent complex. This is considered the canonical isoform. One example of the protein sequence of isoform a is shown in Table 15 as SEQ ID NO: 95. This protein undergoes multiple cleavage events. The other transcript variants encode a smaller protein, one example of which is isoform b, which consists primarily of the phospholipid binding domain of factor VIIIc. This binding domain is essential for coagulant activity. One example of the protein sequence of isoform b is shown in Table 15 as SEQ ID NO: 96.

In some preferred embodiments of the present invention, the gene encodes a therapeutic expression product, preferably a therapeutic polypeptide suitable for use in treating a disease or condition associated with aberrant gene expression, optionally in the liver. The therapeutic expression product can be a protein, e.g. a secretable protein such as, e.g., a clotting factor (e.g., factor IX or factor VIII), a cytokine, a growth factor, an antibody or nanobody, a chemokine, a plasma factor, insulin, erythropoietin, lipoprotein lipase, or a toxic protein. Alternatively, the therapeutic expression product may be RNA, such as an siRNA or miRNA. A non-exhaustive list of therapeutic expression products (and sequences encoding them) envisaged for use in the present invention includes: factor VIII, factor IX, factor VII, factor X, von Willebrand factor, erythropoietin (EPO), interferon-a, interferon-B, interferon-y, interleukin 1 (IL-1), interleukin 2 (IL-2), interleukin 3 (IL-3), interleukin 4 (IL-4), interleukin 5 (IL-5), interleukin 6 (IL-6), interleukin 7 (IL-7), interleukin 8 (IL-8), interleukin 9 (IL-9), interleukin 10 (IL-10), interleukin 11 (IL-11), interleukin 12 (IL-12), chemokine (C—X—C motif) ligand 5 (CXCL5), granulocyte-colony stimulating factor (G-CSF), granulocyte-macrophage colony stimulating factor (GM-CSF), macrophage colony stimulating factor (M-CSF), stem cell factor (SCF), keratinocyte growth factor (KGF), monocyte chemoattractant protein-1 (MCP-1), tumour necrosis factor (TNF), afamin (AFM), acid alfa glucosidase (GAA), a1-antitrypsin, a-galactosidase A, α-L-iduronidase, ATP7b, ornithine transcarbamylase, phenylalanine hydroxylase, lipoprotein lipase, aromatic amino acid decarboxylase (AADC), ATPase Sarcoplasmic/Endoplasmic Reticulum Ca2+ Transporting 2 (ATP2A2), cystic fibrosis transmembrane conductance regulator (CTFR), glutamic acid decarboxylase 65 kDa protein (GAD65), glutamic acid decarboxylase 67 kDa protein (GAD67), lipoprotein lipase (LPL), nerve growth factor (NGF), neurturin (NTN), porphobilinogen deaminase (PBGD), sarcoglycan alpha (SGCA), soluble fms-like tyrosine kinase-1 (sFLT-1), apoliproteins, low-density lipoprotein receptor (LDL-R), albumin, glucose-6-phosphatase, antibodies, nanobodies, aptamers, anti-viral dominant-negative proteins, and functional fragments, subunits or mutants thereof. Preferably the protein is a primate protein, more preferably a human protein. In some embodiments, the protein is alpha-glucosidase (GAA).

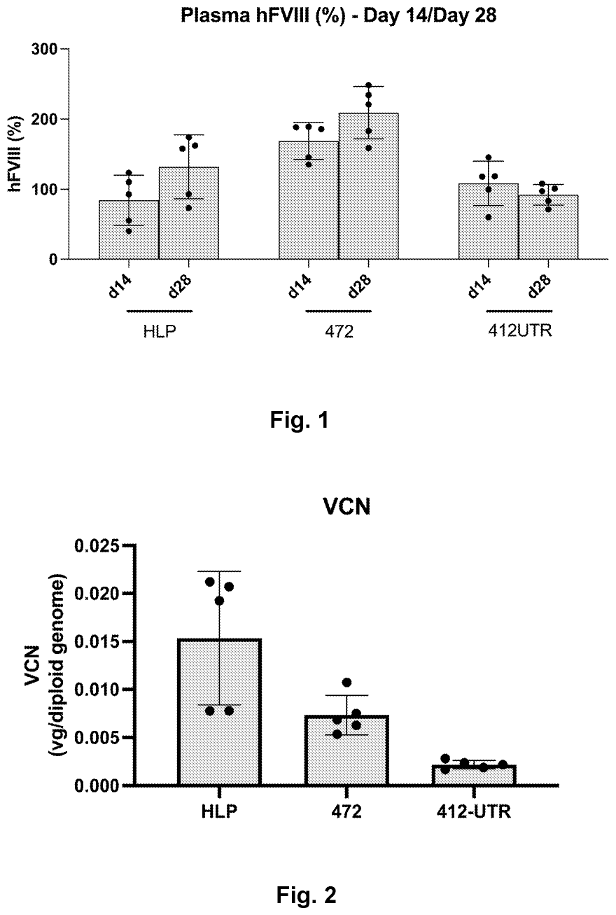

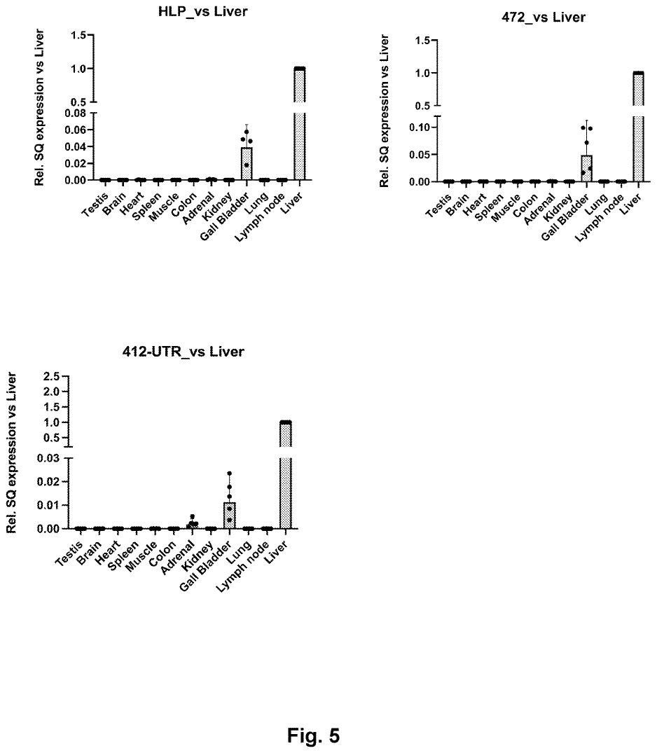

In some embodiments, the therapeutic expression product is human FVIII polypeptide. Suitably, in some embodiments, the human FVIII polypeptide is deleted for the B domain (also referred to as B domain deleted FVIII, also referred to as BDD FVIII or FVIIIAB or FVIIIdeltaB herein). The term “B domain deleted FVIII” encompasses for example, but without limitation, FVIII polypeptides wherein whole or a part of the B domain is deleted and FVIII mutants wherein the B domain is replaced by a linker. Non-limiting examples of B domain deleted FVIII are described in Ward et al. (2011) (see FIG. 1) and WO 2011/005968 (see pages 7, line 20-page 10, line 20 and FIGS. 5 and 8), which are specifically incorporated by reference herein.

In preferred embodiments, the FVIII polypeptide is B domain deleted and there is no further replacement of the domain.

In some embodiments, amino acid substitutions have been introduced at the two known FVIII APC cleavages sites, Arg355 and Arg581 (amino acid numbering refers to sequence that includes the signal peptide), generating a FVIII polypeptide that is resistant to APC cleavage. In some embodiments, the specific amino acid substitutions of Q for R at these sites (FVIII-R355Q/R581Q [FVIII-QQ]) are reflected in the FVIII polypeptide sequence of SEQ ID NO: 60. Consistent with APC having a significant in vivo role in FVIIIa regulation, the FVIII-QQ demonstrates superior hemostatic efficacy relative to wild-type FVIII in an APC-dependent manner. In some embodiments, functional variants of the human FVIII polypeptide described herein include those resulting from amino acid substitutions of the SEQ ID NO: 60 amino acid sequence. It is expected that different amino acids can be substituted at the position 355 and/or 581 positions to generate functional variants of the human FVIII polypeptides described herein. This includes substitution of Arginine to revert back to the wild type sequence at one or more of these sites. In one embodiment, the FVIII comprises a substitution at position 355 that is not with Gln (Q). In one embodiment, the amino acid at position 355 is substituted with Lys (K), Asp (D), Glu (E), or Asn (N). In one embodiment, the amino acid at position 355 is substituted with Asn (N).

In one embodiment, the FVIII comprises a substitution at position 581 that is not with Gln (Q). In one embodiment, the amino acid at position 581 is substituted with Lys (K), Asp (D), Glu (E), or Asn (N). In one embodiment, the amino acid at position 581 is substituted with Asn (N).

It has further been shown that the FVIII polypeptide that has only one of the R355Q or the R581Q substitutions reflected in SEQ ID NO: 60 also exhibit superior haemostatic efficacy. As such, functional variants of the human FVIII with a single one of the R355Q or the R581Q substitutions, or substituted with Lys (K), Asp (D), Glu (E), or Asn (N), are further envisioned.

In some embodiments, the first 19 amino acids of the human FVIII polypeptide (e.g., shown underlined in SEQ ID NO: 60) is an N-terminal secretory signal sequence (otherwise referred to as the signal sequence, signal peptide) with the amino acid sequence MQIELSTCFFLCLLRFCFS (SEQ ID NO: 80). In one embodiment, the FVIII polypeptide sequence does not contain the N-terminal signal sequence. In one embodiment, the FVIII polypeptide has a different secretory signal sequence. For example, one or more amino acids are modified (substituted, deleted, or inserted) to create a functional variant, or the entire sequence is replaced by a different amino acid that serve as a secretory sequence. In one embodiment, the FVIII polypeptide entirely lacks a signal sequence. In such embodiments, the codon-optimized nucleic acid will have the nucleotide sequence of one of SEQ ID NO: 61-78, further lacking the first 19 codons (the 5′ most 57 nucleotides) that encode the N-terminal signal sequence. In one embodiment, the human FVIII polypeptide further contains a heterologous signal sequence that promotes secretion from the liver, in place of the native signal sequence. In one embodiment, the heterologous secretory signal peptide is a signal peptide with the amino acid sequence set forth in Table 15, or a functional variant thereof. Non-limiting examples of heterologous signal peptides are disclosed herein, including, but not limited to, signal peptides comprising the amino acid sequence of any of SEQ ID NO: 81-94 or any signal sequence shown in Table 16. Nucleotide coding sequences for such signal peptides are shown in Table 16. In such embodiments, the codon optimized nucleic acid will lack the 5′ most 57 nucleotides that encode the native N-terminal signal sequence, and instead have a nucleotide sequence that encodes the heterologous signal sequence. Nucleotide sequences coding for such signal peptides are shown in Table 16 and further discussed herein.

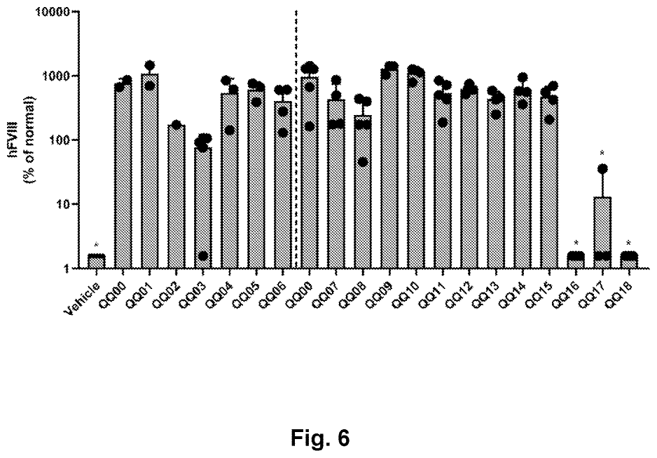

In some embodiments a sequence encoding an expression product is a codon optimized nucleic acid encoding human FVIII polypeptide. In some embodiments, the codon optimized nucleic acid comprises or consists of any one of the 18 specific identified codon optimized nucleic acids encoding human FVIII polypeptide are referred to herein as F8QQ1-F8QQ18, as shown in Table 15 (SEQ ID NO: 61-78). Surprisingly there were variations in activity among the 18 codon-optimized sequences (see, e.g., FIG. 6). In one embodiment, the codon optimized FVIII sequence has the nucleotide sequence set forth in one of SEQ ID NO: 61-78. In one embodiment, the codon-optimized FVIII sequence has the nucleotide sequence set forth in one of SEQ ID NOs 61, 62, 64, 65, 67-69, 71-75 or 78. In one embodiment, the codon-optimized FVIII sequence has the nucleotide sequence set forth in one of SEQ ID NOs 64, 65, 67, 72-75 or 78. In one embodiment, the codon-optimized FVIII sequence has the nucleotide sequence set forth in one of SEQ ID NOs 64, 65, 73, or 75. In one embodiment, the codon-optimized FVIII sequence has the nucleotide sequence set forth in one of SEQ ID NOs 64 or 65. Minor changes to the nucleotide sequence are not expected to appreciably alter the activity of the identified nucleic acids. Such sequence changes may be silent changes (not resulting in amino acid changes in the encoded protein) or alternatively may lead to amino acid substitutions and as such code for variants of the human FVIII polypeptide of SEQ ID NO: 60. Non-limiting examples of such polypeptide variants are described herein.

In some embodiments of the compositions and methods disclosed herein the codon optimized nucleic acid has a nucleotide sequence that is at least 90%, at least 91%, at least 92%, at least 93%, at least 94%, at least 95%, at least 96%, at least 97%, at least 98%, at least 99% or more similar to the sequence set forth in one of SEQ ID NO: 61-78.

In some embodiments of the compositions and methods described herein, the human FVIII polypeptide has the amino acid sequence set forth below (SEQ ID NO: 60), or is at least 90%, at least 91%, at least 92%, at least 93%, at least 94%, at least 95%, at least 96%, at least 97%, at least 98%, at least 99% or more identical thereto.

As described above, it may be desirable in some circumstances to codon-optimise the nucleic acid, suitably FVIII. Without wishing to be bound by theory, it is thought that codon-optimisation may improve gene expression and increase the translational efficiency of the gene. Suitably, in some embodiments, increased expression refers to at least 25% greater exogenous FVIII expression with the codon-optimized FVIII nucleic acid, as compared to the expression level resulting from the native FVIII nucleic acid sequence. In some embodiments, increased expression refers to at least 50% greater, at least 75% greater, at least 100% greater, at least 3-fold greater, at least 4-fold greater, at least 5-fold greater, at least 6-fold greater, at least 7-fold greater, at least 8-fold greater, at least 9-fold greater, at least 10-fold greater, at least 15-fold greater, at least 20-fold greater, at least 25-fold greater, at least 30-fold greater, at least 40-fold greater, at least 50-fold greater, at least 60-fold greater, at least 70-fold greater, at least 80-fold greater, at least 90-fold greater, at least 100-fold greater, at least 125-fold greater, at least 150-fold greater, at least 175-fold greater, at least 200-fold greater, at least 225-fold greater, or at least 250-fold greater exogenous FVIII expression with the codon-optimized nucleic acid encoding FVIII, as compared to the level of exogenous Factor VIII expression of the native FVIII encoding nucleic acid.

Suitably, the administration of the vector, virion or pharmaceutical composition containing the codon-optimized nucleic acid described herein may lead to increased expression of the FVIII polypeptide in a subject, as compared to the expression resulting from administration of an otherwise identical expression vector containing a non-codon optimized (native) nucleic acid encoding the same FVIII polypeptide. Such expression can be measured by the amount of the expressed polypeptide or by the activity of the polypeptide. In some embodiments, increased expression refers to at least 25% greater exogenous FVIII polypeptide or activity in the blood of an animal administered the codon-optimized FVIII nucleic acid, as compared to the level resulting from the native FVIII nucleic acid sequence. In some embodiments, increased expression refers to at least 50% greater, at least 75% greater, at least 100% greater, at least 3-fold greater, at least 4-fold greater, at least 5-fold greater, at least 6-fold greater, at least 7-fold greater, at least 8-fold greater, at least 9-fold greater, at least 10-fold greater, at least 15-fold greater, at least 20-fold greater, at least 25-fold greater, at least 30-fold greater, at least 40-fold greater, at least 50-fold greater, at least 60-fold greater, at least 70-fold greater, at least 80-fold greater, at least 90-fold greater, at least 100-fold greater, at least 125-fold greater, at least 150-fold greater, at least 175-fold greater, at least 200-fold greater, at least 225-fold greater, or at least 250-fold greater exogenous FVIII polypeptide or activity in the blood of an animal administered the codon-optimized nucleic acid encoding FVIII polypeptide, as compared to the level of exogenous Factor VIII polypeptide or activity in the blood of an animal administered the native FVIII encoding nucleic acid.

As discussed above, a low CpG content may be desirable. Suitably, in some embodiments, synthetic liver-specific expression cassette of the present invention has a low CpG dinucleotide content. By low CpG content it is meant that there are fewer CpG dinucleotides compared to a reference expression cassette. Suitably, in some embodiments, a low CpG dinucleotide content is less than 5%, less than 2%, less than 1%, less than 0.75%, less than 0.5%, less than 0.25%, less than 0.1% or less than 0.01% of the total dinucleotide content. Suitably, in some embodiments, the synthetic liver-specific expression cassette is CpG-depleted.

In some embodiments of the invention, the synthetic liver-specific expression cassette comprises a gene useful for gene editing, e.g. a gene encoding a site-specific nuclease, such as a meganuclease, zinc finger nuclease (ZFN), transcription activator-like effector-based nuclease (TALEN), or the clustered regularly interspaced short palindromic repeats system (CRISPR-Cas). Suitably the site-specific nuclease is adapted to edit a desired target genomic locus by making a cut (typically a site-specific double-strand break) which is then repaired via non-homologous end-joining (NHEJ) or homology dependent repair (HDR), resulting in a desired edit. The edit can be the partial or complete repair of a gene that is dysfunctional, or the knock-down or knock-out of a functional gene.

Suitably the synthetic liver-specific expression cassette comprises sequences providing or coding for one or more of, and preferably all of, a ribosomal binding site, a start codon, a stop codon, and a transcription termination sequence. Suitably the expression cassette comprises a nucleic acid encoding a posttranscriptional regulatory element. Suitably the expression cassette comprises a nucleic acid encoding a polyA element.

### Vectors and Viral Particles:

The present invention further provides a vector comprising a synthetic liver-specific CRM, synthetic liver-specific promoter, or expression cassette according to the present invention.

In some embodiments of the invention, the vector is a plasmid. Such a plasmid may include a variety of other functional nucleic acid sequences, such as one or more selectable markers, one or more origins of replication, multiple cloning sites and the like. In some embodiments of the invention, the vector is a viral vector.

In some embodiments of the invention, the vector is an expression vector for expression in eukaryotic cells. Examples of eukaryotic expression vectors include, but are not limited to, pW-LNEO, pSV2CAT, pOG44, pXTI and pSG available from Stratagene; pSVK3, pBPV, pMSG and pSVL available from Amersham Pharmacia Biotech; and pCMVDsRed2-express, pIRES2-DsRed2, pDsRed2-Mito, pCMV-EGFP available from Clontech. Many other vectors are well-known and commercially available. For mammalian cells adenoviral vectors, the pSV and the pCMV series of vectors are particularly well-known non-limiting examples. There are many well-known yeast expression vectors including, without limitation, yeast integrative plasmids (YIp) and yeast replicative plasmids (YRp). For plants the Ti plasmid of Agrobacterium is an exemplary expression vector, and plant viruses also provide suitable expression vectors, e.g. tobacco mosaic virus (TMV), potato virus X, and cowpea mosaic virus.

In some preferred embodiments, the vector is a gene therapy vector. Various gene therapy vectors are known in the art, and mention can be made of AAV vectors, adenoviral vectors, retroviral vectors and lentiviral vectors. Where the vector is a gene therapy vector the vector preferably comprises a nucleic acid sequence operably linked to the synthetic liver-specific promoter of the invention that encodes a therapeutic product, suitably a therapeutic protein. The therapeutic protein may be a secretable protein. Non-limiting examples of secretable proteins are discussed above, and exemplary secretable therapeutic proteins, include clotting factors, such as factor VIII or factor IX, insulin, erythropoietin, lipoprotein lipase, antibodies or nanobodies, growth factors, cytokines, chemokines, plasma factors, toxic proteins, etc. In some preferred embodiments, the therapeutic protein is FVIII.

In some embodiments of the invention, the vector is a viral vector, such as a retroviral, lentiviral, adenoviral, or adeno-associated viral (AAV) vector. In some preferred embodiments the vector is an AAV vector. In some preferred embodiments the AAV has a serotype suitable for liver transduction. In some embodiments, the AAV is selected from the group consisting of: AAV2, AAV5, AAV6, AAV7, AAV8, AAV9, or derivatives thereof. AAV vectors are preferably used as self-complementary, double-stranded AAV vectors (scAAV) in order to overcome one of the limiting steps in AAV transduction (i.e. single-stranded to double-stranded AAV conversion), although the use of single-stranded AAV vectors (ssAAV) is also encompassed herein. In some embodiments of the invention, the AAV vector is chimeric, meaning it comprises components from at least two AAV serotypes, such as the ITRs of an AAV2 and the capsid protein of an AAV5. In some embodiments, the AAV is AAV8, or derivatives thereof.

In some embodiments, the vector of the present invention has a low CpG dinucleotide content. By low CpG content it is meant that there are fewer CpG dinucleotides compared to a reference vector. Suitably, in some embodiments, a low CpG dinucleotide content is less than 5%, less than 2%, less than 1%, less than 0.75%, less than 0.5%, less than 0.25%, less than 0.1% or less than 0.01% of the total dinucleotide content. Suitably, in some embodiments, the vector is CpG-depleted.

The invention further provides recombinant virions (viral particles) comprising a vector as described above.

### Pharmaceutical Compositions:

The vectors or virions of the present invention may be formulated in a pharmaceutical composition with a pharmaceutically acceptable excipient, i.e., one or more pharmaceutically acceptable carrier substances and/or additives, e.g., buffers, carriers, excipients, stabilisers, etc. The pharmaceutical composition may be provided in the form of a kit.

Accordingly, a further aspect of the invention provides a pharmaceutical composition comprising an expression cassette, a vector or virion as described herein.

### Therapeutic and Other Methods and Uses:

The present invention also provides a synthetic liver-specific CRM, synthetic liver-specific promoter, UTR, expression cassette, vector, virion or pharmaceutical composition according to various aspects of the present invention for use in the treatment of a disease, preferably a disease associated with aberrant gene expression, optionally in the liver (e.g. a genetic liver disease). Various diseases associated with aberrant gene expression in the liver are discussed above, and these include but are not limited to haemophilia (including haemophilia A or B), familial hypercholesterolemia, ornithine transcarbamylase deficiency, phenylketonuria, ornithine transcarbamylase deficiency, glycogen storage disease (e.g. Pompe disease), a1-antitrypsin deficiency, hereditary hemochromatosis, tyrosinemia type 1, argininosuccinic aciduria, hepatitis virus infection, non-viral hepatitis, liver cancer, genetic cholestasis, Wilson's disease, and various other liver diseases (such as non-alcoholic fatty liver disease (NAFLD), alcohol-related liver disease (ARLD), and lysosomal storage disorders). Use for the treatment of haemophilia A or B represent preferred embodiments of the invention. Use for the treatment of haemophilia A represent particularly preferred embodiments of the invention. In some preferred embodiments, the disease is Pompe disease.

The present invention also provides a synthetic liver-specific CRM, synthetic liver-specific promoter, expression cassette, vector, or virion according to the various aspects of the present invention for use the manufacture of a pharmaceutical composition for treatment of any condition or disease mentioned herein. In some preferred embodiments, the synthetic liver-specific CRM, synthetic liver-specific promoter, expression cassette, vector, or virion according to the various aspects of the present invention for use the manufacture of pharmaceutical composition for treatment of haemophilia A.

The present invention further provides a cell comprising a synthetic liver-specific CRM, synthetic liver-specific promoter, expression cassette, vector, virion according to the various aspects of the invention. Suitably the cell is a eukaryotic cell. The eukaryotic cell can suitably be a fungal cell (e.g. yeast cell), an animal (metazoan) cell (e.g. a mammalian cell), or a plant cell. Alternatively, the cell may be a prokaryotic cell.

In some embodiments of the invention, the cell is ex vivo, e.g. in cell culture. In other embodiments of the invention the cell may be part of a tissue or multicellular organism.

In a preferred embodiment, the cell is a liver cell (hepatocyte), which may be ex vivo or in vivo. The liver cell may be a primary liver cell or a cell of a liver-derived cell line, e.g. an immortalised cell line. The cell may be present within a liver tissue environment (e.g. within a liver of an animal) or may be isolated from liver tissue, e.g. it may be in cell culture. Suitably the cell is a human cell.

The liver-specific CRM, synthetic liver-specific promoter, expression cassette, or vector, according to the invention may be inserted into the genome of the cell, or it may be episomal (e.g. present in an episomal vector).

In a further aspect the present invention provides a method for producing an expression product, the method comprising providing a synthetic liver-specific expression cassette according to the present invention (preferably in a vector as set out above) in a cell, preferably a liver cell, and expressing the gene present in the synthetic liver-specific expression cassette. The method suitably comprises maintaining said liver cell under suitable conditions for expression of the gene. In culture this may comprise incubating the cell, or tissue comprising the cell, under suitable culture conditions. The expression may of course be in vivo, e.g. in one or more cells in the liver of a subject. In some preferred embodiments, the expression product is FVIII protein.

Suitably the method comprises the step of introducing the synthetic liver-specific expression cassette into the liver cell. A wide range of methods of transfecting liver cells are well-known in the art. A preferred method of transfecting liver cells is transducing the cells with a viral vector comprising the synthetic liver-specific expression cassette, e.g. an AAV vector.

It will be evident to the skilled person that a synthetic liver-specific CRM, synthetic liver-specific promoter, expression cassette, vector or virion according to various aspects of the invention may be used for gene therapy. Accordingly, the use of such nucleic acid constructs in gene therapy forms part of the present invention.

The invention thus provides, in some embodiments, an expression cassettes, vectors or virion according to the present invention for use in gene therapy in a subject, preferably gene therapy through liver-specific expression of a therapeutic gene. The therapy may involve treatment of a disease through secretion of a therapeutic product from liver cells, suitably, a disease involving aberrant gene expression in the liver (for example, haemophilia A or B). In some preferred embodiments, the therapy involves treatment of haemophilia A. In some embodiments, the therapy involves treatment of Pompe disease.

The present invention also provides a method of expressing a therapeutic transgene in a liver cell, the method comprising introducing into the liver cell an expression cassette or vector according to the present invention. The liver cell can be in vivo or ex vivo. In some preferred embodiments, the therapeutic transgene is FVIII.

The present invention also provides a method of gene therapy of a subject, preferably a human, in need thereof, the method comprising:
administering to the subject (suitably introducing into the liver of the subject) a synthetic liver-specific expression cassette, vector, virion or pharmaceutical composition of the present invention, which comprises a gene encoding a therapeutic product.

In some preferred embodiments, the therapeutic product is FVIII protein.

The method suitably comprises expressing a therapeutic amount of the therapeutic product from the gene in the liver of said subject.

Genes encoding suitable therapeutic products are discussed above. However, specific mention may be made of therapeutic proteins, such as factor VIII and IX for the treatment of haemophilia.

The method suitably comprises administering a vector or virion according to the present invention to the subject. Suitably the vector is a viral gene therapy vector, for example an AAV vector.

In some embodiments, the method comprises administering the viral gene therapy vector systemically. Systemic administration may be enteral (e.g. oral, sublingual, and rectal) or parenteral (e.g. injection). Preferred routes of injection include intravenous, intramuscular, subcutaneous, intra-arterial, intra-articular, intrathecal, and intradermal injections.

In some embodiments, the viral gene therapy vector may be administered concurrently or sequentially with one or more additional therapeutic agents or with one or more saturating agents designed to prevent clearance of the vectors by the reticular endothelial system.

Where the vector is an AAV vector, the dosage of the vector may be from 1×10¹⁰ gc/kg to 1×10¹⁵ gc/kg or more, suitably from 1×10¹² gc/kg to 1×10¹⁴ gc/kg, suitably from 5×10¹² gc/kg to 5×10¹³ gc/kg.

In general, the subject in need thereof will be a mammal, and preferably primate, more preferably a human. Typically, the subject in need thereof will display symptoms characteristic of a disease. The method typically comprises ameliorating the symptoms displayed by the subject in need thereof, by expressing the therapeutic amount of the therapeutic product.

Gene therapy protocols for therapeutic gene expression in target cells in vitro and in vivo, are well-known in the art and will not be discussed in detail here. Briefly, they include intramuscular injection, interstitial injection, instillation in airways, application to endothelium, intra-hepatic parenchyme, and intravenous or intra-arterial administration (e.g. intra-hepatic artery, intra-hepatic vein) of plasmid DNA vectors (naked or in liposomes) or viral vectors. Various devices have been developed for enhancing the availability of DNA to the target cell. While a simple approach is to contact the target cell physically with catheters or implantable materials containing the relevant vector, more complex approaches can use jet injection devices and suchlike. Gene transfer into mammalian liver cells has been performed using both ex vivo and in vivo procedures. The ex vivo approach typically requires harvesting of the liver cells, in vitro transduction with suitable expression vectors, followed by reintroduction of the transduced hepatocytes in the liver. In vivo gene transfer has been achieved by injecting DNA or viral vectors into the liver parenchyma, hepatic artery, or portal vein.

According to some preferred embodiments, the methods set out above may be used for the treatment of a subject with haemophilia, e.g. haemophilia A or B. Accordingly, the invention provides a method of treating a subject with haemophilia A or B, the method comprising the steps of:
administering to the subject (suitably introducing into the liver of the subject) a synthetic liver-specific expression cassette, vector, virion or pharmaceutical composition of the present invention which comprises a gene encoding a suitable clotting factor (in particular, factor VIII in the case of haemophilia A or factor IX in the case of haemophilia B); and expressing a therapeutic amount of the clotting factor in the liver of said subject.

In some cases, the synthetic liver-specific expression cassette is provided in a gene therapy vector, suitably an AAV vector.

Preferably the method comprises expressing a suitable amount of the relevant clotting factor in the liver of the subject to alleviate or ameliorate the symptoms of haemophilia A or B. In some preferred embodiments, the method comprises expressing a suitable amount of factor IX in the liver of the subject to alleviate or ameliorate the symptoms of haemophilia B. In some preferred embodiments, the method comprises expressing a suitable amount of factor VIII in the liver of the subject to alleviate or ameliorate the symptoms of haemophilia A.

In a further aspect, there is provided a UTR. In some embodiments, the UTR is a 5′UTR. In some embodiments, the UTR comprises or consists of SEQ ID NO: 13 or a functional variant thereof. In some embodiments, the 5′UTR comprises or consists of SEQ ID NO: 13 or a functional variant thereof. Suitably the functional variant of any of said UTRs comprises a sequence that is at least 70% identical to the reference UTR sequence, more preferably at least 80%, 90%, 91%, 92%, 93%, 94%, 95%, 96%, 97%, 98% or 99% identical to the reference UTR sequence. In some embodiments, the UTR comprises or consists of SEQ ID NO: 119 or a functional variant thereof. In some embodiments, the 5′UTR comprises or consists of SEQ ID NO: 119 or a functional variant thereof. Suitably the functional variant of any of said UTRs comprises a sequence that is at least 70% identical to the reference UTR sequence, more preferably at least 80%, 90%, 91%, 92%, 93%, 94%, 95%, 96%, 97%, 98% or 99% identical to the reference UTR sequence.

In a further aspect, there is provided a synthetic liver-specific promoter comprising a UTR which comprises or consists of SEQ ID NO: 13 or a functional variant thereof.

In a further aspect, there is provided a synthetic liver-specific promoter comprising a UTR which comprises or consists of SEQ ID NO: 119 or a functional variant thereof.

### FVIII and Haemophilia A

Factor VIII, as it is found in nature, is central for coagulation activity and mutations in the FVIII gene result in haemophilia A, the most common form of haemophilia. Full-length FVIII is a large, 280-kDa protein primarily expressed in liver sinusoidal endothelial cells (LSECs), as well as extra-hepatic endothelial cells (Fahs, et al., Blood (2014) 123:3706-3713; Everett, et al., Blood (2014) 123:3697-3705). Native FVIII predominantly circulates as a heterodimer of a heavy chain and a light chain bound through noncovalent metal-dependent interactions (Lenting, et al., Blood (1998) 92:3983-3996). Native Factor VIII comprises several domains and is 2332 amino acids in length (mature without signal peptide). Generally, the domains are referred to as A1-A2-B-A3-C1-C2. The FVIII gene is translated into a single-peptide chain with the domain structure of Al-al-A2-a2-B-a3-A3-Cl-C2. Proteolytic cleavage of FVIII at R-1313 and/or R-1648 by the trans-Golgi protease furin results in heterodimer formation. The FVIII heavy chain (A1-a1-A2-a2-B) and light chain (a3-A3-Cl-C2) remain associated through non-covalent metal-ion-dependent interactions occurring between the Al and A3 domains. Initially, FVIII is in an inactive form bound to von Willebrand factor (vWF). FVIII is activated by cleavage by thrombin (Factor IIa) and release of the B domain. The activated form of FVIII (FVIIIa) separates from vWF and interacts with coagulation factor Factor IXa—leading to the formation of a blood clot via a coagulation cascade. During coagulation, FVIII single chain or heterodimer is activated to its heterotrimeric cofactor form by cleavage by thrombin at R-372, R-740, and R-1689. A2 remains associated with Al-al via non-covalent interactions. Inactivation of FVIIIa occurs via spontaneous A2 dissociation and/or proteolytic cleavage, primarily by activated protein C, at R-336 and R-562.

Specific changes in the amino acid sequence of native FVIII are known to be associated with enhanced activity (e.g., via protein resistance to proteolytic inactivation). It has been found that the FVIII B domain is dispensable for procoagulant activity. Consequently, FVIII constructs in which the B domain is deleted are typically used for gene transfer purposes since their smaller size is more easily incorporated into vectors. Furthermore, it has been shown that deletion of the B domain leads to a 17-fold increase in mRNA and primary translation product. FVIII wherein the B domain is deleted and replaced (e.g. by a short amino acid linker, such as a 14 a.a. linker) are currently used clinically for protein replacement therapy.

Similarly, the FVIII polypeptides encoded in gene therapy are typically engineered to be single chain polypeptides. Single-chain Factor VIII polypeptides have had the natural cleavage sites removed, and optionally have omitted, truncated B domains, or the B domains have been replaced with an alternative sequence. As such, they are not matured by cleavage (other than cleavage of a signal and/or leader peptide), and are active as a single chain. Non-limiting examples of single-chain Factor VIII polypeptides are described in Zollner et al. (Thromb Res, 134(1):125-31 (2014)) (see e.g. page 126, column 1, paragraph 2) and Donath et al. (Biochem J., 312(1):49-55 (1995)) (see e.g. page 50, column 1, paragraph 1), the contents of which are hereby incorporated by reference. Gene therapy using AAV vectors can only use shortened FVIII molecules such as a BDD-FVIII due to the limited packaging capacity of the AAV (4.7 Kb) and other vector systems (Lind, et al. (1995) Eur. J. Biochem., 232(1): 19-27). In some embodiments of the invention, the synthetic liver-specific promoter of the present invention is operably linked with the nucleic acid sequence that encodes single chain Factor VIII polypeptide as described herein. The B domain comprises 40% of the native protein (908 amino acids) and is not required for the protein procoagulant activity (Brinkhous, et al., Proc. Natl. Acad. Sci. (1985) 82:8752-8756). Functional variants of the human FVIII polypeptide described herein include various iterations of B domain deletions, optionally including replacement with a linker. The most common B-domain deleted (BDD) FVIII comprises 14 original amino acid residues (SFSQNPPVLKRHQR (SEQ ID NO: 79) as a linker (Lind, et al. (1995) Eur. J. Biochem., 232(1): 19-27). This BDD FVIII is typically referred to as BDD-SQ or hFVIII-SQ. Short peptide linkers (e.g., 25 or fewer amino acids, 20 or fewer amino acids, 15 or fewer amino acids, or 10 or fewer amino acids) substituted for the B-domain can also be used in FVIII polypeptide variants (Lind, et al. (1995) Eur. J. Biochem., 232(1): 19-27; Pittman, et al., Blood (1993) 81:2925-2935; Toole, et al., Proc. Natl. Acad. Sci. (1986) 83:5939-5942). In some variants, the peptide linker comprises a basic amino acid (e.g., Arg, His, or Lys) at position −1 and −4 to GluI649. This BDD FVIII form is commonly used to produce recombinant BDD-FVIII (~4.4 Kb) as well for gene therapy (Bemtorp, E., Semin. Hematol. (2001) 38(2 Suppl 4): 1-3; Gouw, et al., N. Engl. J. Med. (2013) 368:231-239; Xi, et al., J. Thromb. Haemost. (2013) 11:1655-1662; Recht, et al., Haemophilia (2009) 15:869-880; Sabatino, et al., Mol. Ther. (2011) 19:442-449; Scallan, et al., Blood (2003) 102:2031-2037). U.S. Pat. No. 8,816,054, incorporated by reference herein, also provides BDD FVIII molecules with linkers of different lengths and sequences. In some embodiments of the invention, the synthetic liver-specific promoter of the present invention is operably linked with the nucleic acid sequence that encodes single chain Factor VIII polypeptide as described herein.

A deletion of the entire B domain results in a 17-fold increase in mRNA and primary translation product; however, only a 30% increase occurs in the levels of secreted protein, which suggests that the rate of ER-Golgi transport is actually reduced (Pittman D D, et al. Blood. 1994; 84(12):4214-4225). Efficient FVIII secretion requires carbohydrate-facilitated transport by lectin mannose binding-1 (LMAN1) mediated by mannose residues of N-linked oligosaccharides post-translationally attached to the B domain. To build on the advantages of BDD-FVIII while aiding LMAN1-mediated transport, a short B domain sequence to the BDD-FVIII, optimally 226 amino acids and retaining 6 sites for N-linked glycosylation (N6) were added back. This resulted in a 10-fold increase in secretion in vitro from transfected COS-1 cells and a 5-fold increase in vivo following hydrodynamic hepatic gene delivery (Miao H Z, et al. Blood. 2004; 103(9):3412-3419; Ward et al, Blood 2011 Jan. 20; 117(3):798-807). Suitably, in some embodiments of the invention, the synthetic liver-specific promoter of the present invention is operably linked with the nucleic acid sequence that encodes Factor VIII-BDD polypeptide with added back 226 amino acid sequence of FVIII as described herein. It has been shown that the F309S modification and the addition of up to six glycosylation sites in the B domain can improve FVIII secretion by almost 20-fold (Pipe SW, Semin Thromb Hemost. 2004; 30: 227-237). The substitution of Phe (F) at position 309 with Ser(S) in the A1 domain reduces the affinity to the ER chaperone BiP and the dependence on adenosine triphosphate for secretion (Chen et al, Molecular Therapy, Volume 15, issue 10, P 1856-1862, October 2007). Suitably, in some embodiments of the invention, the synthetic liver-specific promoter of the present invention is operably linked with the nucleic acid sequence that encodes Factor VIII polypeptide comprising six glycosylation sites of B domain, and further comprising F309S modification as described herein.

Coagulation factor VIII (FVIII) is secreted as a heterodimer consisting of a heavy chain (HC) and a light chain (LC), which can be expressed independently and reassociate with recovery of biological activity. Because of the size limitation of adeno-associated virus (AAV) vectors, a strategy for delivering the HC and LC separately has been developed. However, the FVIII HC is secreted 10-100-fold less efficiently than the LC. The F309S mutation and enhanced B-domain glycosylations alone are not sufficient to improve FVIII HC secretion, which suggested a role of the FVIII LC in regulating HC secretion. In vitro, ligation of the LC to the HC and hydrodynamic injection of FVIII intein plasmids into hemophilia A mice significantly increased HC secretion. Furthermore, similar enhancement of HC secretion can also be observed when the LC is supplied in trans, which is probably due to the spontaneous association of the HC and the LC in the secretion pathway. Along the same line, a single-point mutation in the LC may also be able to disrupt FVIII folding and secretion. Point mutations from the human hemophilia A mutation database corroborates this hypothesis as point mutations for example, L1756V, G1760R, A1779P, S1888R, R1941Stop, and many other mutations are scattered almost evenly throughout the whole FVIII LC leading to less than 1% of FVIII antigen being detected in the patients. In summary, a proper LC is essential for efficient FVIII secretion (Chen et al, Molecular Therapy, Volume 15, issue 10, P1856-1862, October 2007). Suitably, in some embodiments of the invention, the synthetic liver-specific promoter of the present invention is operably linked with the nucleic acid sequence that encodes Factor VIII-HC polypeptide ligated with the LC polypeptide as described herein. In some embodiments of the present invention, two plasmids are co transfected to the animal or subject with hemophilia A, where one plasmid comprises a synthetic liver-specific promoter of the present invention operably linked with the nucleic acid sequence that encodes Factor VIII-HC polypeptide and the other plasmid comprises a synthetic liver-specific promoter of the present invention operably linked with the nucleic acid sequence that encodes Factor VIII-LC polypeptide as described herein.

In some of the embodiments of the invention, the synthetic liver-specific promoter of the present invention are operably linked with codon-optimized nucleic acid encoding a human Factor VIII (FVIII) polypeptide, where the encoded FVIII polypeptide lacks the B domain, and comprises an amino acid substitution of Glutamine for Arginine at position 355 (R355Q) and of Glutamine for Arginine at position 581 (R581Q) and is alternatively represented as codon optimized FVIII QQ sequences. In some examples, the codon optimized FVIII QQ nucleic acid sequences are set forth in any one of SEQ ID NOs 1, 2, 4, 5, 7-9, 11-15 or 18 as described Table 3, pages 134-146 of the published International Application PCT/US2023/019211 (publication number: WO2023205300) as incorporated by reference, or a nucleic acid having at least 90% sequence identity thereto. International Application PCT/US2023/019211 (publication number WO2023205300) are incorporated herein by reference in its entirety.

### Additional Matters

In some embodiments, the CRM or synthetic liver-specific promoter does not comprise both CRE0077 (or a functional variant thereof) and CRE0078 (or a functional variant thereof). In some embodiments of the invention, where a CRE, CRM or synthetic liver-specific promoter comprises either CRE0077 or CRE0078 (or a functional variant thereof), it does not comprise a further CRE selected from the group consisting of: CRE0077 (or a functional variant thereof) and CRE0078 (or a functional variant thereof). In some embodiments of the invention, the CRM or synthetic liver-specific promoter does not comprise more than one CRE selected from the group consisting of: CRE0077 (or a functional variant thereof) or CRE0078 (or a functional variant thereof).

In some embodiments of the invention, the CRE, CRM or synthetic liver-specific promoter does not comprise CRE0077 or a functional variant thereof or CRE0078 or a functional variant thereof.

CRE0077 (V1)-
(SEQ ID NO: 118)
AAGCAAATATTTGTGGTTATGGATTAACTCGAACTGTTTGCCCACTCTA
TTTGCCCTGTACC
CRE0078 (V2)-
(SEQ ID NO: 37)
GGCGCCCTTTGGACCTTTTGCAATCCTGGAGCAAACAGCAAACACTGTA
CC
In some embodiments of the invention, the CRM or
synthetic liver-specific promoter does not
comprise the sequence
(SEQ ID NO: 38)
GGACTTAGCCCCTGTTTGCTCCTCCGATAACTGGGGTGACCTTGGTTAA
TATTCACCA,
or
(SEQ ID NO: 39)
GCCCCTGTTTGCTCCTCCGATAACTGGGGTGACCTTGGTTAATATTCAC
CA,
or
a functional variant of either thereof. These
sequences are portions of the SEPRINA1 promoter.

In some embodiments of the invention, where the CRM or synthetic liver-specific promoter comprises either CRE0077 or CRE0078 (or functional variants of any thereof), it does not contain SEQ ID NO: 38 or SEQ ID NO: 39 (or functional variants of any thereof). Thus, in some embodiments, the CRM or synthetic liver-specific promoter contains not more than one of sequences V1, V2, SEQ ID NO: 38 and SEQ ID NO: 39.

In some embodiments of the invention, the CRM or synthetic liver-specific promoter comprises not more than two of the following elements: LVR_CRE0080_PROC, LVR_CRE0081_APOA1, LVR_CRE0061_APOB, LVR_CRE0082_APOC4, SEQ ID NO: 38 and SEQ ID NO: 39, or functional variants of any thereof. In some embodiments of the invention, the CRM or synthetic liver-specific promoter comprises not more than one of said elements, or functional variants of any thereof. In some embodiments of the invention, the CRM or synthetic liver-specific promoter does not comprise any of said elements, or functional variants of any thereof. LVR_CRE0080_PROC, and LVR_CRE0081_APOA1 are components of CRE0077, and LVR_CRE0061_APOB, LVR_CRE0082_APOC4 are components of CRE0078. The sequences of these elements are:

LVR_CRE0080_PROC-
(SEQ ID NO: 97)
AAGCAAATATTTGTGGTTATGGATTAACTCGAA.
LVR_CRE0081_APOA1-
(SEQ ID NO: 98)
CTGTTTGCCCACTCTATTTGCCC.
LVR_CRE0061_APOB-
(SEQ ID NO: 99)
GGCGCCCTTTGGACCTTTTGCAATCCTGG.
LVR_CRE0082_APOC4-
(SEQ ID NO: 100)
AGCAAACAGCAAACAC.

In some embodiments of the invention, the synthetic liver-specific promoter does not comprise the CRE0052 minimal promoter or a functional variant thereof.

In some embodiments of the invention, the CRM or synthetic liver-specific promoter does not comprise a sequence as disclosed in European patent application no 18207027.6.

The functional variants of any of the sequences in the disclaimers and embodiments discussed above can have, for example, a sequence having 60%, 70%, 80%, 90%, 95%, 96%, 97%, 98% or 99% identity to any of the reference sequences.

### Definitions and General Points

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Ausubel, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); U.S. Pat. No. 4,683,195; Nucleic Acid Hybridization (Harries and Higgins eds. 1984); Transcription and Translation (Hames and Higgins eds. 1984); Culture of Animal Cells (Freshney, Alan R. Liss, Inc., 1987); Immobilized Cells and Enzymes (IRL Press, 1986); Perbal, A Practical Guide to Molecular Cloning (1984); the series, Methods in Enzymology (Abelson and Simon, eds.—in-chief, Academic Press, Inc., New York), specifically, Vols. 154 and 155 (Wu et al. eds.) and Vol. 185, “Gene Expression Technology” (Goeddel, ed.); Gene Transfer Vectors For Mammalian Cells (Miller and Calos eds., 1987, Cold Spring Harbor Laboratory); Immunochemical Methods in Cell and Molecular Biology (Mayer and Walker, eds., Academic Press, London, 1987); Handbook of Experimental Immunology, Vols. I-IV (Weir and Blackwell, eds., 1986); and Manipulating the Mouse Embryo, (Cold Spring Harbor Laboratory Press, Cold Spring Harbor, N.Y., 1986).

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The term “cis-regulatory element” or “CRE”, is a term well-known to the skilled person, and means a nucleic acid sequence such as an enhancer, promoter, insulator, or silencer, that can regulate or modulate the transcription of a neighbouring gene (i.e. in cis). CREs are found in the vicinity of the genes that they regulate. CREs typically regulate gene transcription by binding to TFs, i.e. they include TFBS. A single TF may bind to many CREs, and hence control the expression of many genes (pleiotropy). CREs are usually, but not always, located upstream of the transcription start site (TSS) of the gene that they regulate. “Enhancers” are CREs that enhance (i.e. upregulate) the transcription of genes that they are operably associated with, and can be found upstream, downstream, and even within the introns of the gene that they regulate. Multiple enhancers can act in a coordinated fashion to regulate transcription of one gene. “Silencers” in this context relates to CREs that bind TFs called repressors, which act to prevent or downregulate transcription of a gene. The term “silencer” can also refer to a region in the 3′ untranslated region of messenger RNA, that binds proteins which suppress translation of that mRNA molecule, but this usage is distinct from its use in describing a CRE. Generally, the CREs of the present invention are liver-specific enhancer elements (often referred to as liver-specific CREs, or liver-specific CRE enhancers, or suchlike). In the present context, it is preferred that the CRE is located 1500 nucleotides or less from the transcription start site (TSS), more preferably 1000 nucleotides or less from the TSS, more preferably 500 nucleotides or less from the TSS, and suitably 250, 200, 150, 100, 80, 60 or 50 nucleotides or less from the TSS. CREs of the present invention are preferably comparatively short in length, preferably 200 nucleotides or less in length, for example they may be 175, 150, 90, 80, 70, 60, 50 or 40 nucleotides or less in length. The CREs of the present invention are typically provided in combination with an operably linked promoter element, which can be a minimal promoter or proximal promoter; the CREs of the present invention enhance the liver-specific activity of the promoter element.

The term “cis-regulatory module” or “CRM” means a functional regulatory nucleic acid module, which usually comprises two or more CREs; in the present invention the CREs are typically liver-specific enhancers and thus the CRM is a synthetic liver-specific regulatory nucleic acid. Thus, in the present application a CRM typically comprises a plurality of liver-specific CREs. Typically, the multiple CREs within the CRM act together (e.g. additively or synergistically) to enhance the transcription of a gene that a promoter comprising the CRM is operably associated with. There is considerable scope to shuffle (i.e. reorder), invert (i.e. reverse orientation), and alter spacing of CREs within a CRM. Accordingly, functional variants of CRMs of the present invention include, inter alia, variants of the referenced CRMs wherein CREs within them have been shuffled and/or inverted, and/or the spacing between CREs has been altered.

As used herein, the phrase “promoter” refers to a region of DNA that generally is located upstream of a nucleic acid sequence to be transcribed that is needed for transcription to occur, i.e. which initiates transcription. Promoters permit the proper activation or repression of transcription of a coding sequence under their control. A promoter typically contains specific sequences that are recognized and bound by plurality of TFs. TFs bind to the promoter sequences and result in the recruitment of RNA polymerase, an enzyme that synthesizes RNA from the coding region of the gene. Many diverse promoters are known in the art.

The term “synthetic promoter” as used herein relates to a promoter that does not occur in nature. In the present context it typically comprises a CRE and/or CRM of the present invention operably linked to a minimal (or core) promoter or liver-specific proximal promoter (promoter element). The CREs and/or CRMs of the present invention serve to enhance liver-specific transcription of a gene operably linked to the synthetic promoter. Parts of the synthetic promoter may be naturally occurring (e.g. the minimal promoter or one or more CREs in the promoter), but the synthetic promoter as a complete entity is not naturally occurring.

As used herein, “minimal promoter” (also known as the “core promoter”) refers to a short DNA segment which is inactive or largely inactive by itself, but can mediate transcription when combined with other transcription regulatory elements. Minimum promoter sequence can be derived from various different sources, including prokaryotic and eukaryotic genes. Examples of minimal promoters are discussed above, and include the dopamine beta-hydroxylase gene minimum promoter, cytomegalovirus (CMV) immediate early gene minimum promoter (CMV-MP), and the herpes thymidine kinase minimal promoter (MinTK). A minimal promoter typically comprises the transcription start site (TSS) and elements directly upstream, a binding site for RNA polymerase II, and general transcription factor binding sites (often a TATA box). A minimal promoter may also include some elements downstream of the TSS, but these typically have little functionality absent additional regulatory elements.

As used herein, “proximal promoter” relates to the minimal promoter plus the proximal sequence upstream of the gene that tends to contain primary regulatory elements. It often extends approximately 250 base pairs upstream of the TSS, and includes specific TFBS. A proximal promoter may also include one or more regulatory elements downstream of the TSS, for example a UTR or an intron. In the present case, the proximal promoter may suitably be a naturally occurring liver-specific proximal promoter that can be combined with one or more CREs or CRMs of the present invention. However, the proximal promoter can be synthetic.

As used herein, “promoter element” refers to either a minimal promoter or proximal promoter as defined above. In the context of the present invention a promoter element is typically combined with one or more CREs or one or more CRMs in order to provide a synthetic liver-specific promoter of the present invention.

A “functional variant” of a CRE, CRM, promoter element, synthetic promoter or other nucleic acid construct in the context of the present invention is a variant of a reference sequence that retains the ability to function in the same way as the reference sequence, e.g. as a liver-specific CRE, liver-specific CRM or liver-specific synthetic promoter. Alternative terms for such functional variants include “biological equivalents” or “equivalents”.

It will be appreciated that the ability of a given CRE to function as a liver-specific enhancer is determined principally by the ability of the sequence to bind the same liver-specific TFs that bind to the reference sequence. Accordingly, in most cases, a functional variant of a CRE or CRM will contain TFBS for the most or all of same TFs as the reference CRE or CRM. It is preferred, but not essential, that the TFBS of a functional variant are in the same relative positions (i.e. order and general position) as the reference CRE or CRM. It is also preferred, but not essential, that the TFBS of a functional variant are in the same orientation as the reference sequence (it will be noted that TFBS can in some cases be present in reverse orientation, e.g. as the reverse complement vis-à-vis the sequence in the reference sequence). It is also preferred, but not essential, that the TFBS of a functional variant are on the same strand as the reference sequence. Thus, in preferred embodiments, the functional variant comprises TFBS for the same TFs, in the same order, the same position, in the same orientation and on the same strand as the reference sequence. It will also be appreciated that the sequences lying between TFBS (referred to in some cases as spacer sequences, or suchlike) are of less consequence to the function of the CRE or CRM. Such sequences can typically be varied considerably, and their lengths can be altered. However, in preferred embodiments the spacing (i.e. the distance between adjacent TFBS) is substantially the same (e.g. it does not vary by more than 20%, preferably by not more than 10%, and more preferably it is approximately the same) in a functional variant as it is in the reference sequence. It will be apparent that in some cases a functional variant of a CRE can be present in the reverse orientation, e.g. it can be the reverse complement of a CRE as described above, or a variant thereof.

Levels of sequence identity between a functional variant and the reference sequence can also be an indicator or retained functionality. High levels of sequence identity in the TFBS of the CRE is of generally higher importance than sequence identity in the spacer sequences (where there is little or no requirement for any conservation of sequence). However, it will be appreciated that even within the TFBS, a considerable degree of sequence variation can be accommodated, given that the sequence of a functional TFBS does not need to exactly match the consensus sequence.

The ability of one or more TFs to bind to a TFBS in a given functional variant can determined by any relevant means known in the art, including, but not limited to, electromobility shift assays (EMSA), binding assays, chromatin immunoprecipitation (ChIP), and ChIP-sequencing (ChIP-seq). In a preferred embodiment the ability of one or more TFs to bind a given functional variant is determined by EMSA. Methods of performing EMSA are well-known in the art. Suitable approaches are described in Sambrook et al. cited above. Many relevant articles describing this procedure are available, e.g. Hellman and Fried, Nat Protoc. 2007; 2(8): 1849-1861.

“Liver-specific” or “liver-specific expression” refers to the ability of a cis-regulatory element, cis-regulatory module or promoter to enhance or drive expression of a gene in the liver (or in liver-derived cells) in a preferential or predominant manner as compared to other tissues (e.g. spleen, muscle, heart, lung, and brain). Expression of the gene can be in the form of mRNA or protein. In preferred embodiments, liver-specific expression is such that there is negligible expression in other (i.e. non-liver) tissues or cells, i.e. expression is highly liver-specific.

The ability of a CRE, CRM or synthetic promoter to function as a liver-specific CRE, CRM or synthetic promoter can be readily assessed by the skilled person. The skilled person can thus easily determine whether any variant of the specific CRE, CRM or synthetic promoter recited above remains functional (i.e. it is a functional variant as defined above). For example, any given CRM to be assessed can be operably linked to a minimal promoter (e.g. positioned upstream of CMV-MP) and the ability of the cis-regulatory element to drive liver-specific expression of a gene (typically a reporter gene) is measured. Alternatively, a variant of a CRE can be substituted into a synthetic liver-specific promoter in place of a reference CRE, and the effects on liver-specific expression driven by said modified promoter can be determined and compared to the unmodified form. Similarly, the ability of a CRM or promoter to drive liver-specific expression can be readily assessed by the skilled person (e.g. as described in the examples below). Expression levels of a gene driven by a variant of a reference promoter can be compared to the expression levels driven by the reference sequence. In some embodiments, where liver-specific expression levels driven by a variant promoter are at least 50%, at least 60%, at least 70%, at least 80%, at least 90%, or at least 100% of the expression levels driven by the reference promoter, it can be said that the variant remains functional. Suitable nucleic acid constructs and reporter assays to assess liver-specific expression enhancement can easily constructed, and the examples set out below give suitable methodologies (e.g. Example 2, Example 3 and Example 4).

Liver-specificity can be identified wherein the expression of a gene (e.g. a therapeutic or reporter gene) occurs preferentially or predominantly in liver-derived cells. Preferential or predominant expression can be defined, for example, where the level of expression is significantly greater in liver-derived cells than in other types of cells (i.e. non-liver-derived cells). For example, expression in liver-derived cells is suitably at least 5-fold higher than in non-liver cells, preferably at least 10-fold higher than in non-liver cells, and it may be 50-fold higher or more in some cases. For convenience, liver-specific expression can suitably be demonstrated via a comparison of expression levels in a hepatic cell line (e.g. liver-derived cell line such as Huh7 and/or HepG2 cells) or liver primary cells, compared with expression levels in a kidney-derived cell line (e.g. HEK-293), a cervical tissue-derived cell line (e.g. HeLa) and/or a lung-derived cell line (e.g. A549).

The synthetic liver-specific promoters of the present invention preferably exhibit reduced expression in non-liver-derived cells, suitably in HEK-293, HeLa, and/or A549 cells when compared to a non-tissue specific promoter such as CMV-IE. The synthetic liver-specific promoters of the present invention preferably have an activity of 50% or less than the CMV-IE promoter in non-liver-derived cells (suitably in HEK-293, HeLa, and/or A549 cells), suitably 25% or less, 20% or less, 15% or less, 10% or less, 5% or less or 1% or less. Generally, it is preferred that expression in non-liver-derived cells is minimized, but in some cases this may not be necessary. In some embodiments, the synthetic liver-specific promoters of the present invention are suitable for promoting gene expression at a level of at 50% or less than an LP1 promoter in non-liver-derived cells (e.g. HEK-293, HeLa, and/or A549 cells).

The synthetic liver-specific promoters of the present invention are preferably suitable for promoting expression in the liver of a subject, e.g. driving liver-specific expression of a transgene, preferably a therapeutic transgene. Preferred synthetic liver-specific promoters of the present invention are suitable for promoting liver-specific transgene expression and have an activity in liver cells which is at least 15%, 20%, 30%, 40%, 50%, 60%, 70%, 80%, 90%, 100%, 125%, 150%, 175%, 200%, 250%, 300%, 350% or 400% of the activity of the TBG promoter. In some embodiments, the synthetic liver-specific promoters of the invention are suitable for promoting liver-specific transgene expression at a level at least 100% of the activity of the LP1 promoter, preferably 150%, 200%, 300% or 500% of the activity of the LP1 promoter. Such liver-specific expression is suitably determined in liver-derived cells, e.g. in Huh7, and/or HepG2 cells or primary liver cells (suitably primary human hepatocytes).

Synthetic liver-specific promoters of the present invention may also be able to promote liver-specific expression of a gene at a level at least 150% compared to CMV-IE in liver-derived cells (e.g. Huh7 and/or HepG2 cells), preferably at least 200% CMV-IE promoter in liver-derived cells.

As used herein, the terms “SP0412 v2”, “SP0412v2”, “SP0412_v2”, “412 v2”, “SP0412+UTR”, “SP0412+UTR”, “412UTR”, “412-UTR”, and the like are synonymous terms and are used interchangeably herein and in the accompanying drawings.

The term “nucleic acid” as used herein typically refers to an oligomer or polymer (preferably a linear polymer) of any length composed essentially of nucleotides. A nucleotide unit commonly includes a heterocyclic base, a sugar group, and at least one, e.g. one, two, or three, phosphate groups, including modified or substituted phosphate groups. Heterocyclic bases may include inter alia purine and pyrimidine bases such as adenine (A), guanine (G), cytosine (C), thymine (T) and uracil (U) which are widespread in naturally-occurring nucleic acids, other naturally-occurring bases (e.g., xanthine, inosine, hypoxanthine) as well as chemically or biochemically modified (e.g., methylated), non-natural or derivatised bases. Sugar groups may include inter alia pentose (pentofuranose) groups such as preferably ribose and/or 2-deoxyribose common in naturally-occurring nucleic acids, or arabinose, 2-deoxyarabinose, threose or hexose sugar groups, as well as modified or substituted sugar groups. Nucleic acids as intended herein may include naturally occurring nucleotides, modified nucleotides or mixtures thereof. A modified nucleotide may include a modified heterocyclic base, a modified sugar moiety, a modified phosphate group or a combination thereof. Modifications of phosphate groups or sugars may be introduced to improve stability, resistance to enzymatic degradation, or some other useful property. The term “nucleic acid” further preferably encompasses DNA, RNA and DNA RNA hybrid molecules, specifically including hnRNA, pre-mRNA, mRNA, cDNA, genomic DNA, amplification products, oligonucleotides, and synthetic (e.g., chemically synthesised) DNA, RNA or DNA RNA hybrids. A nucleic acid can be naturally occurring, e.g., present in or isolated from nature; or can be non-naturally occurring, e.g., recombinant, i.e., produced by recombinant DNA technology, and/or partly or entirely, chemically or biochemically synthesised. A “nucleic acid” can be double-stranded, partly double stranded, or single-stranded. Where single-stranded, the nucleic acid can be the sense strand or the antisense strand. In addition, nucleic acid can be circular or linear.

The terms “identity” and “identical” and the like refer to the sequence similarity between two polymeric molecules, e.g., between two nucleic acid molecules, such as between two DNA molecules. Sequence alignments and determination of sequence identity can be done, e.g., using the Basic Local Alignment Search Tool (BLAST) originally described by Altschul et al. 1990 (J Mol Biol 215:403-10), such as the “Blast 2 sequences” algorithm described by Tatusova and Madden 1999 (FEMS Microbiol Lett 174:247-250).

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10.

The National Center for Biotechnology Information (NCBI) Basic Local Alignment Search Tool (BLAST™; Altschul et al. (1990)) is available from several sources, including the National Center for Biotechnology Information (Bethesda, MD), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn; Align Sequence Nucleotide BLAST) program may be employed using the default parameters. Nucleic acid sequences with even greater similarity to the reference sequences will show increasing percentage identity when assessed by this method. Typically, the percentage sequence identity is calculated over the entire length of the sequence.

For example, a global optimal alignment is suitably found by the Needleman-Wunsch algorithm with the following scoring parameters: Match score: +2, Mismatch score: −3; Gap penalties: gap open 5, gap extension 2. The percentage identity of the resulting optimal global alignment is suitably calculated by the ratio of the number of aligned bases to the total length of the alignment, where the alignment length includes both matches and mismatches, multiplied by 100.

The term “hybridising” means annealing to two at least partially complementary nucleotide sequences in a hybridization process. In order to allow hybridisation to occur complementary nucleic acid molecules are generally thermally or chemically denatured to melt a double strand into two single strands and/or to remove hairpins or other secondary structures from single-stranded nucleic acids. The stringency of hybridisation is influenced by conditions such as temperature, salt concentration and hybridisation buffer composition. Conventional hybridisation conditions are described in, for example, Sambrook (2001) Molecular Cloning: a laboratory manual, 3rd Edition Cold Spring Harbor Laboratory Press, CSH, New York, but the skilled craftsman will appreciate that numerous different hybridisation conditions can be designed in function of the known or the expected homology and/or length of the nucleic acid sequence. High stringency conditions for hybridisation include high temperature and/or low sodium/salt concentration (salts include sodium as for example in NaCl and Na-citrate) and/or the inclusion of formamide in the hybridisation buffer and/or lowering the concentration of compounds such as SDS (sodium dodecyl sulphate detergent) in the hybridisation buffer and/or exclusion of compounds such as dextran sulphate or polyethylene glycol (promoting molecular crowding) from the hybridisation buffer. By way of non-limiting example, representative salt and temperature conditions for stringent hybridization are: 1×SSC, 0.5% SDS at 65° C. The abbreviation SSC refers to a buffer used in nucleic acid hybridization solutions. One litre of a 20× (twenty times concentrate) stock SSC buffer solution (pH 7.0) contains 175.3 g sodium chloride and 88.2 g sodium citrate. A representative time period for achieving hybridisation is 12 hours.

The term “transcription factor binding site” (TFBS) is well known in the art. Disclosed herein are various specific TFBS sequences. It will be apparent to the skilled person that alternative TFBS sequences can be used, provided that they are bound by the intended TF. Consensus sequences for the various TFBS disclosed herein are known in the art, and the skilled person can readily use this information to determine alternative TFBS. Furthermore, the ability of a TF to bind to a given putative sequence can readily be determined experimentally by the skilled person (e.g. by EMSA and other approaches well known in the art and discussed herein).

The meaning of “consensus sequence” is well-known in the art. In the present application, the following notation is used for the consensus sequences, unless the context dictates otherwise. Considering the following exemplary DNA sequence:
A[CT]N{A}YR

A means that an A is always found in that position; [CT] stands for either C or T in that position; N stands for any base in that position; and {A} means any base except A is found in that position. Y represents any pyrimidine, and R indicates any purine.

“Synthetic” in the present application means a nucleic acid molecule that does not occur in nature. Synthetic nucleic acid expression constructs of the present invention are produced artificially, typically by recombinant technologies. Such synthetic nucleic acids may contain naturally occurring sequences (e.g. promoter, enhancer, intron, and other such regulatory sequences), but these are present in a non-naturally occurring context. For example, a synthetic gene (or portion of a gene) typically contains one or more nucleic acid sequences that are not contiguous in nature (chimeric sequences), and/or may encompass substitutions, insertions, and deletions and combinations thereof.

“Complementary” or “complementarity”, as used herein, refers to the Watson-Crick base-pairing of two nucleic acid sequences. For example, for the sequence 5′-AGT-3′ binds to the complementary sequence 3′-TCA-5′. Complementarity between two nucleic acid sequences may be “partial”, in which only some of the bases bind to their complement, or it may be complete as when every base in the sequence binds to its complementary base. The degree of complementarity between nucleic acid strands has significant effects on the efficiency and strength of hybridisation between nucleic acid strands.

“Transfection” in the present application refers broadly to any process of deliberately introducing nucleic acids into cells, and covers introduction of viral and non-viral vectors, and includes transformation, transduction and like terms and processes. Examples include, but are not limited to: transfection with viral vectors; transformation with plasmid vectors; electroporation (Fromm et al. (1986) Nature 319:791-3); lipofection (Feigner et al. (1987) Proc. Natl. Acad. Sci. USA 84:7413-7); microinjection (Mueller et al. (1978) Cell 15:579-85); Agrobacterium-mediated transfer (Fraley et al. (1983) Proc. Natl. Acad. Sci. USA 80:4803-7); direct DNA uptake; whiskers-mediated transformation; and microprojectile bombardment (Klein et al. (1987) Nature 327:70).

As used herein, the phrase “transgene” refers to an exogenous nucleic acid sequence. In one example, a transgene is a gene encoding an industrially or pharmaceutically useful compound, or a gene encoding a desirable trait. In yet another example, the transgene encodes an antisense nucleic acid sequence, wherein expression of the antisense nucleic acid sequence inhibits expression of a target nucleic acid sequence. The transgene preferably encodes a therapeutic product, e.g. a protein.

The term “vector” is well known in the art, and as used herein refers to a nucleic acid molecule, e.g. double-stranded DNA, which may have inserted into it a nucleic acid sequence according to the present invention. A vector is suitably used to transport an inserted nucleic acid molecule into a suitable host cell. A vector typically contains all of the necessary elements that permit transcribing the insert nucleic acid molecule, and, preferably, translating the transcript into a polypeptide. A vector typically contains all of the necessary elements such that, once the vector is in a host cell, the vector can replicate independently of, or coincidental with, the host chromosomal DNA; several copies of the vector and its inserted nucleic acid molecule may be generated. Vectors of the present invention can be episomal vectors (i.e., that do not integrate into the genome of a host cell), or can be vectors that integrate into the host cell genome. This definition includes both non-viral and viral vectors. Non-viral vectors include but are not limited to plasmid vectors (e.g. pMA-RQ, pUC vectors, bluescript vectors (pBS) and pBR322 or derivatives thereof that are devoid of bacterial sequences (minicircles)) transposons-based vectors (e.g. PiggyBac (PB) vectors or Sleeping Beauty (SB) vectors), etc. Larger vectors such as artificial chromosomes (bacteria (BAC), yeast (YAC), or human (HAC)) may be used to accommodate larger inserts. Viral vectors are derived from viruses and include but are not limited to retroviral, lentiviral, adeno-associated viral, adenoviral, herpes viral, hepatitis viral vectors or the like. Typically, but not necessarily, viral vectors are replication-deficient as they have lost the ability to propagate in a given cell since viral genes essential for replication have been eliminated from the viral vector. However, some viral vectors can also be adapted to replicate specifically in a given cell, such as e.g. a cancer cell, and are typically used to trigger the (cancer) cell-specific (onco) lysis. Virosomes are a non-limiting example of a vector that comprises both viral and non-viral elements, in particular they combine liposomes with an inactivated HIV or influenza virus (Yamada et al., 2003). Another example encompasses viral vectors mixed with cationic lipids.

The term “operably linked”, “operably connected” or equivalent expressions as used herein refer to the arrangement of various nucleic acid elements relative to each other such that the elements are functionally connected and are able to interact with each other in the manner intended. Such elements may include, without limitation, some or all of the following group: a promoter, a CRE (e.g. enhancer or other regulatory element), a promoter element, a polyadenylation sequence, one or more introns and/or exons, and a coding sequence of a gene of interest to be expressed. The nucleic acid sequence elements, when properly oriented or operably linked, act together to modulate the activity of one another, and ultimately may affect the level of expression of an expression product. By modulate is meant increasing, decreasing, or maintaining the level of activity of a particular element. The position of each element relative to other elements may be expressed in terms of the 5′ terminus and the 3′ terminus of each element or their position upstream or downstream of another element or position (such as a TSS or promoter element), and the distance between any particular elements may be referenced by the number of intervening nucleotides, or base pairs, between the elements. As understood by the skilled person, operably linked implies functional activity, and is not necessarily related to a natural positional link. Indeed, when used in nucleic acid expression cassettes, CREs will typically be located immediately upstream of the promoter element (although this is generally the case, it should definitely not be interpreted as a limitation or exclusion of positions within the nucleic acid expression cassette), but this needs not be the case in vivo, e.g., a regulatory element sequence naturally occurring downstream of a gene whose transcription it affects is able to function in the same way when located upstream of the promoter. Hence, according to a specific embodiment, the regulatory or enhancing effect of the regulatory element can be position-independent.

A “spacer sequence” or “spacer” as used herein is a nucleic acid sequence that separates two functional nucleic acid sequences (e.g. TFBS, CREs, CRMs, promoter element, etc.). It can have essentially any sequence, provided it does not prevent the functional nucleic acid sequence (e.g. cis-regulatory element) from functioning as desired (e.g. this could happen if it includes a silencer sequence, prevents binding of the desired transcription factor, or suchlike). Typically, it is non-functional, as in it is present only to space adjacent functional nucleic acid sequences from one another. In some embodiments, spacers may have a length of 75, 50, 40, 30, 30 or 10 nucleotides or fewer.

The term “pharmaceutically acceptable” as used herein is consistent with the art and means compatible with the other ingredients of the pharmaceutical composition and not deleterious to the recipient thereof.

“Therapeutically effective amount” and like phrases mean a dose or plasma concentration in a subject that provides the desired specific pharmacological effect, e.g. to express a therapeutic gene in the liver. A therapeutically effective amount may not always be effective in treating the conditions described herein, even though such dosage is deemed to be a therapeutically effective amount by those of skill in the art. The therapeutically effective amount may vary based on the route of administration and dosage form, the age and weight of the subject, and/or the disease or condition being treated.

The terms “treatment” or “treating” refer to reducing, ameliorating or eliminating one or more signs, symptoms, or effects of a disease or condition.

The “administration” of an agent to a subject includes any route of introducing or delivering to a subject the agent to perform its intended function. Administration can be carried out by any suitable route, including orally, intranasally, intraocularly, ophthalmically, parenterally (intravenously, intramuscularly, intraperitoneally, or subcutaneously), or topically. Administration includes self-administration and the administration by another.

The terms “individual,” “subject,” and “patient” are used interchangeably, and refer to any individual subject with a disease or condition in need of treatment. For the purposes of the present disclosure, the subject may be a primate, preferably a human, or another mammal, such as a dog, cat, horse, pig, goat, or bovine, and the like.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Some embodiments of the technology described herein can be defined according to any of the following numbered paragraphs:

1. A synthetic liver-specific promoter comprising a combination of one or more cis-regulatory elements (CREs) or functional variants of any thereof operably linked to a promoter element (or a functional variant of any thereof) selected from the group consisting of:
CRE0051, CRE0042, CRE0059 and 5′UTR;
CRE0051, CRE0058 and CRE0070;
CRE0051, CRE0058 and CRE0071;
CRE0042 and CRE0099;
CRE0042 and CRE0073;
CRE0051 and CRE0099;
CRE0051 and CRE0073;
CRE0051, CRE0058 and CRE0054;
CRE0094 and CRE0052;
CRE0056, CRE0094 and CRE0052;
CRE0056, CRE0094 and CRE0059;
CRE0048, CRE0056 and CRE0054;
CRE0048, CRE0056 and CRE0052; and
CRE0048, CRE0056 and CRE0059.

2. The synthetic liver-specific promoter of paragraph 1 which comprises a promoter selected from the group consisting of: SP0412_v2, SP0382, SP0383, SP0471, SP0472, SP0473, SP0474, SP0475, SP0476, SP0477, SP0478, SP0479, SP0480, and SP0481, or a functional variant of any thereof, wherein the functional variant of any of said promoters comprises a sequence that is at least 70% identical to a reference synthetic liver-specific promoter, more preferably at least 80%, 90%, 95%, 96%, 97%, 98% or 99% identical to the reference synthetic liver-specific promoter.

3. The synthetic liver-specific promoter of paragraph 1 or 2 which comprises a sequence selected from the group consisting of SEQ ID NO: 22 to SEQ ID NO: 35, or a functional variant of any thereof, wherein the functional variant of any of said promoters comprises a sequence that is at least 70% identical to a reference synthetic liver-specific promoter sequence, more preferably at least 80%, 90%, 95%, 96%, 97%, 98% or 99% identical to the reference synthetic liver-specific promoter sequence.

4. The synthetic liver-specific promoter of any preceding paragraph which has a length of 350 or fewer nucleotides, 340, 330, 320, 310, 300, 290, 280, 270, 260, 250, 240, 230, 220 or fewer nucleotides.

5. A synthetic liver-specific promoter comprising one of the following promoter elements: CRE0059, CRE0070, CRE0071, CRE0054, CRE0073, CRE0099 and CRE0052 or a functional variant thereof.

6. The synthetic liver-specific promoter according to paragraph 5 comprising one of the following promoter elements: CRE0070, CRE0071, CRE0099 and CRE0054 or a functional variant thereof.

7. The synthetic liver-specific promoter of paragraph 5 or 6 which has a length of 350 or fewer nucleotides, 340, 330, 320, 310, 300, 290, 280, 270, 260, 250, 240, 230, 220 or fewer nucleotides.

8. A promoter element comprising any one of CRE0059, CRE0070, CRE0071, CRE0054, CRE0073, CRE0099 and CRE0052 or a functional variant thereof.

9. The promoter element according to paragraph 8 comprising any one of CRE0070, CRE0071, CRE0099 and CRE0054 or a functional variant thereof.

10. The promoter element of paragraph 8 or 9 wherein the promoter element has a length of 200 or fewer nucleotides, 190, 180, 170, 160, 150, 140, 130, 120, 110, 100, 90, 80 or fewer nucleotides.

11. A liver-specific CRE selected from the group consisting of: CRE0051, CRE0042, CRE0058, CRE0048, CRE0094 and CRE0056, or a functional variant of any thereof, wherein the functional variant of any of said CREs comprises a sequence that is at least 70% identical to a reference synthetic liver-specific CRE, more preferably at least 80%, 90%, 95%, 96%, 97%, 98% or 99% identical to the reference synthetic liver-specific CRE.

12. The liver-specific CRE according to paragraph 11, wherein the CRE comprises CRE0094 or a functional variant thereof.

13. A synthetic liver-specific cis-regulatory module (CRM) or a synthetic liver-specific promoter comprising one or more of the liver-specific CREs according to paragraph 11, or functional variants thereof.

14. The synthetic liver-specific CRM according to paragraph 13 which comprises one of the following combinations of CREs, or functional variants thereof:
CRE0051 and CRE0042;
CRE0051 and CRE0058;
CRE0056 and CRE0094; and
CRE0048 and CRE0056.

15. The synthetic liver-specific CRM according to paragraph 14 which comprises one of the following combinations of CREs, or functional variants thereof:
CRE0048 and CRE0056; or
CRE0056 and CRE0094.

16. The synthetic liver-specific CRM according to paragraph 14 wherein the CRM is selected from the group consisting of: CRM_SP0412_v2, CRM_SP0382 and SP0383, CRM_SP0471, CRM_SP0473, CRM_SP0475, CRM_SP0477 and SP0478, CRM_SP0479 and CRM_SP0480 and SP0481, or a functional variant of any thereof, wherein the functional variant of any of said CRMs comprises a sequence that is at least 70% identical to a reference synthetic liver-specific CRM, more preferably at least 80%, 90%, 95%, 96%, 97%, 98% or 99% identical to the reference synthetic liver-specific CRM.

17. The synthetic liver-specific CRM according to paragraph 15 wherein the CRM is selected from the group consisting of: CRM_SP0477 and SP0478, CRM_SP0479, and CRM_SP0480 and SP0481, or a functional variant of any thereof, wherein the functional variant of any of said CRMs comprises a sequence that is at least 70% identical to a reference synthetic liver-specific CRM, more preferably at least 80%, 90%, 95%, 96%, 97%, 98% or 99% identical to the reference synthetic liver-specific CRM.

18. A synthetic liver-specific promoter comprising at least one liver-specific CRE according to paragraph 11 operably linked to a promoter element.

19. The synthetic liver-specific promoter according to paragraph 18 wherein the promoter element is selected from the group consisting of: CRE0054, CRE0099, CRE0070 and CRE0071, or a functional variant thereof.

20. An expression cassette comprising a synthetic liver-specific promoter according to any one of paragraphs 1-7, 18-19 or a promoter element according to any one of paragraphs 8-10, a CRE according to any one of paragraphs 11-12, or a CRM according to any one of paragraphs 13-17, operably linked to a sequence encoding an expression product, optionally wherein the sequence encoding the expression product is codon optimised.

21. A vector comprising a synthetic liver-specific promoter according to any one of paragraphs 1-7, 18-19, a promoter element according to any one of paragraphs 8-10, a CRE according to any one of paragraphs 11-12, or a CRM according to any one of paragraphs 13-17, or an expression cassette according to paragraph 20.

22. The vector of paragraph 21 which is a viral vector.

23. The vector of paragraph 22 wherein the viral vector is an AAV vector.

24. A virion comprising a vector according to any one of paragraphs 22-23.

25. A pharmaceutical composition comprising a synthetic liver-specific promoter according to any one of paragraphs 1-7, 18-19, a promoter element according to any one of paragraphs 8-10, a CRE according to any one of paragraphs 11-12, or a CRM according to any one of paragraphs 13-17, or an expression cassette according to paragraph 20, a vector according to any one of paragraphs 22-23, or a virion according to paragraph 24.

26. A synthetic liver-specific promoter according to any one of paragraphs 1-7, 18-19, a promoter element according to any one of paragraphs 8-10, a CRE according to any one of paragraphs 11-12, or a CRM according to any one of paragraphs 13-17, or an expression cassette according to paragraph 20, a vector according to any one of paragraphs 22-23, a virion according to paragraph 24, or a pharmaceutical composition according to paragraph 25 for use in therapy.

27. The composition for use according to paragraph 26 wherein the use is for gene therapy, suitably wherein the gene therapy involves expression of a therapeutic expression product in the liver.

28. The composition for use according to any one of paragraphs 26-27 wherein the use is for gene therapy for Haemophilia A or Haemophilia B.

29. An expression cassette according to paragraph 20, a vector according to any one of paragraphs 22-23, a virion according to paragraph 24, or a pharmaceutical composition according to paragraph 25 for use in gene therapy for Haemophilia A or Haemophilia B, wherein the expression product is Factor VIII or Factor IX.

30. The composition for use according to paragraph 29 wherein the use is for gene therapy for Haemophilia A, wherein the expression product is Factor VIII.

31. The composition for use according to paragraph 30, wherein the expression product is a codon optimised Factor VIII.

32. A cell comprising a synthetic liver-specific promoter according to any one of 1-7, 18-19, a promoter element according to any one of paragraphs 8-10, a CRE according to any one of paragraphs 11-12, or a CRM according to any one of paragraphs 13-17, or an expression cassette according to paragraph 20, a vector according to any one of paragraphs 22-23, or a virion according to paragraph 24.

33. The cell of paragraph 32 wherein the cell is a liver cell, optionally a human liver cell.

34. A synthetic liver-specific promoter according to any one of 1-7, 18-19, a promoter element according to any one of paragraphs 8-10, a CRE according to any one of paragraphs 11-12, or a CRM according to any one of paragraphs 13-17, or an expression cassette according to paragraph 20, a vector according to any one of paragraphs 22-23, a virion according to paragraph 24, or a pharmaceutical composition according to paragraph 25 for use in the manufacture of a pharmaceutical composition for the treatment of a medical condition or disease.

35. A method for producing an expression product, the method comprising providing a synthetic liver-specific expression cassette according to paragraph 20 in a liver cell and expressing the gene present in the synthetic liver-specific expression cassette.

36. A method of expressing a therapeutic transgene in a liver cell, the method comprising introducing into the liver cell a synthetic liver-specific expression cassette according to paragraph 20, a vector according to any one of paragraphs 21-23, or a virion according to paragraph 24.

37. A method of therapy of a subject, preferably a human, in need thereof, the method comprising:
administering to the subject an expression cassette according to paragraph 20, a vector according to any one of paragraphs 22-23, a virion according to paragraph 24, or a pharmaceutical composition according to paragraph 25, which comprises a sequence encoding a therapeutic product operably linked to the synthetic liver-specific promoter; and
expressing a therapeutic amount of the therapeutic product in the liver of said subject.

38. The method according to paragraph 37 wherein the therapeutic product is FVIII and the subject has Haemophilia A.

39. The method according to any one of paragraphs 37-38 comprising administering a vector according to any one of paragraphs 22-23, a virion according to paragraph 24, or a pharmaceutical composition according to paragraph 25.

The technology described herein is further illustrated by the following examples which in no way should be construed as being further limiting.

### EXAMPLES

### Example 1—Sequences

The following sequences are of relevance to the present disclosure:

TABLE 1
Cis-regulatory elements (CREs)

| Name (also | |
|---|---|
| known as) | Sequence |
| CRE0051 | aggttaatttttaaaaagcagtcaaaagtccaagtggcccttggcagcatttactctctctgtttgctctgg |
| (SEQ ID | ttaataatctcaggagcacaaacattcc |
| NO: 1) | |
| CRE0042 | ccctgttcaaacatgtcctaatactctgtctctgcaagggtcatcagtagttttccatcttactcaacatcct |
| (SEQ ID | cccagtg |
| NO: 2) | |
| CRE0058 | ggcccgggaggcgccctttggaccttttgcaatcctggcg |
| (SEQ ID | |
| NO: 3) | |
| CRE0048 | tgctctctgacaaagatacggtgggtcccactgatgaactgtgctgccacagtaaatgtagccactat |
| (SEQ ID | gcctatctccattctgaagatgtg |
| NO: 4) | |
| CRE0094 | cctgaagtccaaaggtagaggaaccgagtgtttaaaaattactgtggctgtggagtcaacatgatgta |
| (SEQ ID | aaaaaacaaacatttggataacaccaagaagccagatatggttgaaatgttgactggttgacaaaa |
| NO: 5) | ataatttgggttgcttaatggtgcacaaaggtaatgc |
| CRE0056 | ccgcccccactgaacccttgacccctgccctgcagcccccgcagcttgctgtttgcccactctatttgc |
| (SEQ ID | ccagccccag |
| NO: 6) | |

TABLE 2
Minimal/Proximal Promoters:

| Name | Sequence |
|---|---|
| CRE0059 | agtcatatgtttgctcactgaaggttactagttaacaggcatcccttaaacaggatataaaaggact |
| (SEQ ID NO: | tcagcaggactgctcgaaacatcccact |
| 7) | |
| CRE0070 | tctagagggtatataatgggggccactagtgccagcagcagcctgaccacatctcatcctcaatc |
| (SEQ ID NO: | accaccaagcctggaataactgca |
| 8) | |
| CRE0071 | tctagagggtatataatgggggccagccgctggcgtttccgttgcttggatcagtctaggtgcagct |
| (SEQ ID NO: | gccggat |
| 9) | |
| CRE0054 | agatcccagccagtggacttagcccctgtttgctcctccgataactggggtgaccttggttaatattc |
| (SEQ ID NO: | accagcagcctcccccgttgcccctctggatccactgcttaaatacggacgaggacagggccct |
| 10) | gtct |
| CRE0073 | gggcgactcagatcccagccagtggacttagcccctgtttgctcctccgataactggggtgacctt |
| (SEQ ID NO: | ggttaatattcaccagcagcctcccccgttgcccctctggatccactgcttaaatacggacgagga |
| 11) | cagggccctgtctcctcagcttcaggcaccaccactgacctgggacagtgaatc |
| CRE0052 | gggcatataaaacaggggcaaggcacagactcatagcagagcaatcaccaccaagcctgga |
| (SEQ ID NO: | ataactgcagccacc |
| 12) | |
| CRE0099 | cgggcgactcagatcccagccagtggacttagcccctgtttgctcctccgataactggggtgacct |
| (SEQ ID NO: | tggttaatattcaccagcagcctcccccgttgcccctctggatccactgcttaaatacggacga |
| 36) | |

TABLE 3
UTR

| Name | Sequence |
|---|---|
| 5′UTR | caggtggattcttgggcatttgcttaagcttggt |
| (SEQ ID NO: | accgccacc |
| 13) | |
| 5′ UTR (24 bp) | caggtggattcttgggcatttgct |
| (SEQ ID NO: | |
| 119) | |

TABLE 4
Cis-regulatory modules (CRMs):

| CRM NAME | SEQUENCE |
|---|---|
| CRM_SP0412_ | aggttaatttttaaaaagcagtcaaaagtccaagtggcccttggcagcatttactctctctgtttgctct |
| v2 | ggttaataatctcaggagcacaaacattccccctgttcaaacatgtcctaatactctgtctctgcaa |
| (SEQ ID NO: | gggtcatcagtagttttccatcttactcaacatcctcccagtg |
| 14) | |
| CRM_SP0382 | aggttaatttttaaaaagcagtcaaaagtccaagtggcccttggcagcatttactctctctgtttgctct |
| and SP0383 | ggttaataatctcaggagcacaaacattccggcccgggaggcgccctttggaccttttgcaatcct |
| (SEQ ID NO: | ggcg |
| 15) | |
| CRM_SP0471 | ccctgttcaaacatgtcctaatactctgtctctgcaagggtcatcagtagttttccatcttactcaacat |
| (SEQ ID NO: | cctcccagtgcgggcgactc |
| 16) | |
| CRM_SP0473 | aggttaatttttaaaaagcagtcaaaagtccaagtggcccttggcagcatttactctctctgtttgctct |
| (SEQ ID NO: | ggttaataatctcaggagcacaaacattcccgggcgactc |
| 17) | |
| CRM_SP0475 | aggttaatttttaaaaagcagtcaaaagtccaagtggcccttggcagcatttactctctctgtttgctct |
| (SEQ ID NO: | ggttaataatctcaggagcacaaacattcctgtaccggcccgggaggcgccctttggaccttttgc |
| 18) | aatcctggcg |
| CRM_SP0477 | ccgcccccactgaacccttgacccctgccctgcagcccccgcagcttgctgtttgcccactctattt |
| and SP0478 | gcccagccccagcctgaagtccaaaggtagaggaaccgagtgtttaaaaattactgtggctgtg |
| (SEQ ID NO: | gagtcaacatgatgtaaaaaaacaaacatttggataacaccaagaagccagatatggttgaaa |
| 19) | tgttgactggttgacaaaaataatttgggttgcttaatggtgcacaaaggtaatgc |
| CRM_SP0479 | tgctctctgacaaagatacggtgggtcccactgatgaactgtgctgccacagtaaatgtagccact |
| (SEQ ID NO: | atgcctatctccattctgaagatgtgccgcccccactgaacccttgacccctgccctgcagccccc |
| 20) | gcagcttgctgtttgcccactctatttgcccagccccaggggcgactc |
| CRM_SP0480 | Tgctctctgacaaagatacggtgggtcccactgatgaactgtgctgccacagtaaatgtagccac |
| and SP0481 | tatgcctatctccattctgaagatgtgccgcccccactgaacccttgacccctgccctgcagccccc |
| (SEQ ID NO: | gcagcttgctgtttgcccactctatttgcccagccccag |
| 21) | |

TABLE 5
Synthetic promoters:

| | | | CpG |
|---|---|---|---|
| NAME | SEQUENCE | Length | content |
| SP0412_v2 | aggttaatttttaaaaagcagtcaaaagtccaagtggcccttggcagcattta | 318 | 2 |
| (SEQ ID | ctctctctgtttgctctggttaataatctcaggagcacaaacattccccctgttca | | |
| NO: 22) | aacatgtcctaatactctgtctctgcaagggtcatcagtagttttccatcttactc | | |
| | aacatcctcccagtgagtcatatgtttgctcactgaaggttactagttaacagg | | |
| | catcccttaaacaggatataaaaggacttcagcaggactgctcgaaacatc | | |
| | ccactcaggtggattcttgggcatttgcttaagcttggtaccgccacc | | |
| SP0382 | aggttaatttttaaaaagcagtcaaaagtccaagtggcccttggcagcattta | 229 | 4 |
| (SEQ ID | ctctctctgtttgctctggttaataatctcaggagcacaaacattccggcccgg | | |
| NO: 23) | gaggcgccctttggaccttttgcaatcctggcgtctagagggtatataatggg | | |
| | ggccactagtgccagcagcagcctgaccacatctcatcctcaatcaccacc | | |
| | aagcctggaataactgca | | |
| SP0383 | aggttaatttttaaaaagcagtcaaaagtccaagtggcccttggcagcattta | 214 | 8 |
| (SEQ ID | ctctctctgtttgctctggttaataatctcaggagcacaaacattccggcccgg | | |
| NO: 24) | gaggcgccctttggaccttttgcaatcctggcgtctagagggtatataatggg | | |
| | ggccagccgctggcgtttccgttgcttggatcagtctaggtgcagctgccgg | | |
| | at | | |
| SP0471 | ccctgttcaaacatgtcctaatactctgtctctgcaagggtcatcagtagttttc | 226 | 6 |
| (SEQ ID | catcttactcaacatcctcccagtgcgggcgactcagatcccagccagtgg | | |
| NO: 25) | acttagcccctgtttgctcctccgataactggggtgaccttggttaatattcacc | | |
| | agcagcctcccccgttgcccctctggatccactgcttaaatacggacgagg | | |
| | acagggccctgtct | | |
| SP0472 | ccctgttcaaacatgtcctaatactctgtctctgcaagggtcatcagtagttttc | 268 | 7 |
| (SEQ ID | catcttactcaacatcctcccagtgcgggcgactcagatcccagccagtgg | | |
| NO: 26) | acttagcccctgtttgctcctccgataactggggtgaccttggttaatattcacc | | |
| | agcagcctcccccgttgcccctctggatccactgcttaaatacggacgagg | | |
| | acagggccctgtctcctcagcttcaggcaccaccactgacctgggacagtg | | |
| | aatcg | | |
| SP0473 | aggttaatttttaaaaagcagtcaaaagtccaagtggcccttggcagcattta | 246 | 6 |
| (SEQ ID | ctctctctgtttgctctggttaataatctcaggagcacaaacattcccgggcga | | |
| NO: 27) | ctcagatcccagccagtggacttagcccctgtttgctcctccgataactgggg | | |
| | tgaccttggttaatattcaccagcagcctcccccgttgcccctctggatccact | | |
| | gcttaaatacggacgaggacagggccctgtct | | |
| SP0474 | aggttaatttttaaaaagcagtcaaaagtccaagtggcccttggcagcattta | 288 | 7 |
| (SEQ ID | ctctctctgtttgctctggttaataatctcaggagcacaaacattcccgggcga | | |
| NO: 28) | ctcagatcccagccagtggacttagcccctgtttgctcctccgataactgggg | | |
| | tgaccttggttaatattcaccagcagcctcccccgttgcccctctggatccact | | |
| | gcttaaatacggacgaggacagggccctgtctcctcagcttcaggcaccac | | |
| | cactgacctgggacagtgaatcg | | |
| SP0475 | aggttaatttttaaaaagcagtcaaaagtccaagtggcccttggcagcattta | 290 | 9 |
| (SEQ ID | ctctctctgtttgctctggttaataatctcaggagcacaaacattcctgtaccgg | | |
| NO: 29) | cccgggaggcgccctttggaccttttgcaatcctggcgggcgactcagatcc | | |
| | cagccagtggacttagcccctgtttgctcctccgataactggggtgaccttgg | | |
| | ttaatattcaccagcagcctcccccgttgcccctctggatccactgcttaaata | | |
| | cggacgaggacagggccctgtct | | |
| SP0476 | cctgaagtccaaaggtagaggaaccgagtgtttaaaaattactgtggctgtg | 247 | 2 |
| (SEQ ID | gagtcaacatgatgtaaaaaaacaaacatttggataacaccaagaagcc | | |
| NO: 30) | agatatggttgaaatgttgactggttgacaaaaataatttgggttgcttaatggt | | |
| | gcacaaaggtaatgcgggcatataaaacaggggcaaggcacagactca | | |
| | tagcagagcaatcaccaccaagcctggaataactgcagccacc | | |
| SP0477 | ccgcccccactgaacccttgacccctgccctgcagcccccgcagcttgctgt | 326 | 4 |
| (SEQ ID | ttgcccactctatttgcccagccccagcctgaagtccaaaggtagaggaac | | |
| NO: 31) | cgagtgtttaaaaattactgtggctgtggagtcaacatgatgtaaaaaaaca | | |
| | aacatttggataacaccaagaagccagatatggttgaaatgttgactggttg | | |
| | acaaaaataatttgggttgcttaatggtgcacaaaggtaatgcgggcatata | | |
| | aaacaggggcaaggcacagactcatagcagagcaatcaccaccaagc | | |
| | ctggaataactgcagccacc | | |
| SP0478 | ccgcccccactgaacccttgacccctgccctgcagcccccgcagcttgctgt | 345 | 4 |
| (SEQ ID | ttgcccactctatttgcccagccccagcctgaagtccaaaggtagaggaac | | |
| NO: 32) | cgagtgtttaaaaattactgtggctgtggagtcaacatgatgtaaaaaaaca | | |
| | aacatttggataacaccaagaagccagatatggttgaaatgttgactggttg | | |
| | acaaaaataatttgggttgcttaatggtgcacaaaggtaatgcagtcatatgt | | |
| | ttgctcactgaaggttactagttaacaggcatcccttaaacaggatataaaa | | |
| | ggacttcagcaggactgctcgaaacatcccact | | |
| SP0479 | tgctctctgacaaagatacggtgggtcccactgatgaactgtgctgccacag | 316 | 8 |
| (SEQ ID | taaatgtagccactatgcctatctccattctgaagatgtgccgcccccactga | | |
| NO: 33) | acccttgacccctgccctgcagcccccgcagcttgctgtttgcccactctattt | | |
| | gcccagccccaggggcgactcagatcccagccagtggacttagcccctgt | | |
| | ttgctcctccgataactggggtgaccttggttaatattcaccagcagcctcccc | | |
| | cgttgcccctctggatccactgcttaaatacggacgaggacagggccctgtc | | |
| | t | | |
| SP0480 | tgctctctgacaaagatacggtgggtcccactgatgaactgtgctgccacag | 247 | 3 |
| (SEQ ID | taaatgtagccactatgcctatctccattctgaagatgtgccgcccccactga | | |
| NO: 34) | acccttgacccctgccctgcagcccccgcagcttgctgtttgcccactctattt | | |
| | gcccagccccaggggcatataaaacaggggcaaggcacagactcatag | | |
| | cagagcaatcaccaccaagcctggaataactgcagccacc | | |
| SP0481 | tgctctctgacaaagatacggtgggtcccactgatgaactgtgctgccacag | 266 | 4 |
| (SEQ ID | taaatgtagccactatgcctatctccattctgaagatgtgccgcccccactga | | |
| NO: 35) | acccttgacccctgccctgcagcccccgcagcttgctgtttgcccactctattt | | |
| | gcccagccccagagtcatatgtttgctcactgaaggttactagttaacaggc | | |
| | atcccttaaacaggatataaaaggacttcagcaggactgctcgaaacatcc | | |
| | cact | | |
| SP0412 | aggttaatttttaaaaagcagtcaaaagtccaagtggcccttggcagcattta | 277 | 1 |
| (SEQ ID | ctctctctgtttgctctggttaataatctcaggagcacaaacattccccctgttca | | |
| NO: 123) | aacatgtcctaatactctgtctctgcaagggtcatcagtagttttccatcttactc | | |
| | aacatcctcccagtgagtcatatgtttgctcactgaaggttactagttaacagg | | |
| | catcccttaaacaggatataaaaggacttcagcaggactgctcgaaacatc | | |
| | ccactca | | |
| SP0246 | aggttaatttttaaaaagcagtcaaaagtccaagtggcccttggcagcattta | 282 | 5 |
| (SEQ ID | ctctctctgtttgctctggttaataatctcaggagcacaaacattccggcccgg | | |
| NO: 124) | gaggcgccctttggaccttttgcaatcctggcgcactgaacccttgacccctg | | |
| | ccctgcagcccccgcagcttgctgtttgcccactctatttgcccagccccagg | | |
| | ggcatataaaacaggggcaaggcacagactcatagcagagcaatcacc | | |
| | accaagcctggaataactgca | | |

TABLE 6
Synthetic promoters and parts thereof

| Synthetic | | | | |
|---|---|---|---|---|
| promoter | Element | Element | Element | Element |
| SP0412_v2 | CRE0051 | CRE0042 | CRE0059 | 5′UTR |
| SP0382 | CRE0051 | CRE0058 | CRE0070 | |
| SP0383 | CRE0051 | CRE0058 | CRE0071 | |
| SP0471 | CRE0042 | CRE0099 | | |
| SP0472 | CRE0042 | CRE0073 | | |
| SP0473 | CRE0051 | CRE0099 | | |
| SP0474 | CRE0051 | CRE0073 | | |
| SP0475 | CRE0051 | CRE0058 | CRE0054 | |
| SP0476 | CRE0094 | CRE0052 | | |
| SP0477 | CRE0056 | CRE0094 | CRE0052 | |
| SP0478 | CRE0056 | CRE0094 | CRE0059 | |
| SP0479 | CRE0048 | CRE0056 | CRE0054 | |
| SP0480 | CRE0048 | CRE0056 | CRE0052 | |
| SP0481 | CRE0048 | CRE0056 | CRE0059 | |

TABLE 7
Intron

| | |
|---|---|
| HBB2 | tactagcagctacaatccagctaccattctgcttttattttatggttgggataaggctggattattctgagtcca |
| intron | agctaggcccttttgctaatcatgttcatacctcttatcttcctcccacagctcctgggcaacgtgctggtctgt |
| (SEQ ID | gtgctggcccatcactttggcaaagaatt |
| NO: 40) | |
| UBC | acttggtgagtagcgggctgctgggctggccggggctttcgtggccgccgggccgctcggtgggacgg |
| intron | aagcgtgtggagagaccgccaagggctgtagtctgggtccgcgagcaaggttgccctgaactgggggt |
| (SEQ ID | tggggggagcgcagcaaaatggcggctgttcccgagtcttgaatggaagacgcttgtgaggcgggctg |
| NO: 41) | tgaggtcgttgaaacaaggtggggggcatggtgggcggcaagaacccaaggtcttgaggccttcgcta |
| | atgcgggaaagctcttattcgggtgagatgggctggggcaccatctggggaccctgacgtgaagtttgtc |
| | actgactggagaactcggtttgtcgtctgttgcgggggcggcagttatgcggtgccgttgggcagtgcacc |
| | cgtacctttgggagcgcgcgccctcgtcgtgtcgtgacgtcacccgttctgttggcttataatgcagggtgg |
| | ggccacctgccggtaggtgtgcggtaggcttttctccgtcgcaggacgcagggttcgggcctagggtag |
| | gctctcctgaatcgacaggcgccggacctctggtgaggggagggataagtgaggcgtcagtttctttggt |
| | cggttttatgtacctatcttcttaagtagctgaagctccggttttgaactatgcgctcggggttggcgagtgtgtt |
| | ttgtgaagttttttaggcaccttttgaaatgtaatcatttgggtcaatatgtaattttcagtgttagactagtaaatt |
| | gtccgctaaattctggccgtttttggcttttttgttagac |
| CMV-IE | caagagtgacgtaagtaccgcctatagactctataggcacacccctttggctcttatgcatgaacggtgg |
| intron | agggcagtgtagtctgagcagtactcgttgctgccgcgcgcgccaccagacataatagctgacagacta |
| (SEQ ID | acagactgttcctttccatgggtcttttctgcag |
| NO: 42) | |

TABLE 8
Exon and splice donor

| | |
|---|---|
| Exon and splice | tccagcactgcctgcggtgaaggaaccagcagcctacatttcgctgcgtccaaagcat |
| donor_CRE0059 | tgcacgaaaaagagtttgggataggtaagatgct |
| (SEQ ID NO: 43) | |
| Exon and splice | atcttggtgtccgtgatcgcagacctcaggaagccttctacgtcctcttccccatctggttc |
| donor_CRE0052.1 | catcttcaggaagctgtgggcattaaactcctttgggtagctgtgattggagactggctc |
| (SEQ ID NO: 44) | aacctcgtctttaagtggtaag |

TABLE 9
TFBS in CRE0051

| CRE0051 | Aggttaatttttaaaaagcagtcaaaagtccaagtggcccttggcagcatttactctctc | | |
|---|---|---|---|
| | tgtttgctctggttaataatctcaggagcacaaacattcc (SEQ ID NO: 1) | | |
| | (TFBS shown in bold) | | |
| TF | Position | TFBS sequence | SEQ ID NO |
| HNF1 | 3..15 | GTTAATTTTTAAA | 101 |
| HNF4 | 34..44 | GTGGCCCTTGG | 102 |
| HNF3 | 61..67 | TGTTTGC | 103 |
| HNF1 | 70..84 | TGGTTAATAATCTCA | 104 |
| HNF3 | 90..96 | ACAAACA | 105 |

TABLE 10
TFBS in CRE0042

| CRE0042 | Ccctgttcaaacatgtcctaatactctgtctctgcaagggtcatcagtagttttccatc | | |
|---|---|---|---|
| | ttactcaacatcctcccagtg (SEQ ID NO: 2) (TFBS shown in bold) | | |
| TF | Position | TFBS sequence | SEQ ID NO |
| HNF-3 | 5..15 | GTTCAAACATG | 106 |
| C/EBP | 18..28 | CTAATACTCTG | 107 |
| HNF-4 | 33..44 | TGCAAGGGTCAT | 108 |
| C/EBP | 60..69 | TTACTCAACA | 109 |

TABLE 11
TFBS in CRE0058

| CRE0058 | GGCCCGGGAGGCGCCCTTTGGACCTTTTGCAATCCTG | | |
|---|---|---|---|
| | GCG (SEQ ID NO: 3) (TFBS shown in bold) | | |
| TF | Position | TFBS sequence | SEQ ID NO |
| HNF-4 | 12..24 | CGCCCTTTGGACC | 110 |
| c/EBP | 21..38 | GACCTTTTGCAATCC | 117 |
| | | TGG | |

TABLE 12
TFBS in CRE0056

| CRE0056 | Ccgcccccactgaacccttgacccctgccctgcagcccccgc | | |
|---|---|---|---|
| | agcttgctgtttgcccactctatttgcccagccccag (SEQ ID | | |
| | NO: 6) (TFBS shown in bold) | | |
| TF | Position | TFBS sequence | SEQ ID NO |
| HNF-4 | 9..31 | ACTGAACCCTTGACC | 111 |
| | | CCTGCCCT | |
| HNF-3 | 48..71 | CTGTTTGCCCACTCT | 112 |
| | | ATTTGCCC | |
| HNF-3b | 54..75 | TGCCCACTCTATTTG | 113 |
| | | CCCAGCC | |

TABLE 13
TFBS in CRE0094

| CRE0094 | Cctgaagtccaaaggtagaggaaccgagtgtttaaaaattactgt | | |
|---|---|---|---|
| | ggctgtggagtcaacatgatgtaaaaaaacaaacatttggataac | | |
| | accaagaagccagatatggttgaaatgttgactggttgacaaaaa | | |
| | taatttgggttgcttaatggtgcacaaaggtaatgc | | |
| | (SEQ ID NO: 5) (TFBS shown in bold) | | |
| TF | Position | TFBS sequence | SEQ ID NO |
| HNF4A | 5..19 | aagtccaaaggtaga | 114 |
| FOXA1 | 53..64 | gagtcaacatga | 115 |
| FOXA1 | 124..113 | cagtcaacattt | 116 |

Human FVIII Polypeptide
(SEQ ID NO: 60)
MQIELSTCFFLCLLRFCFSATRRYYLGAVELSWDYMQSDLGELPVDARFP
PRVPKSFPFNTSVVYKKTLFVEFTDHLFNIAKPRPPWMGLLGPTIQAEVY
DTVVITLKNMASHPVSLHAVGVSYWKASEGAEYDDQTSQREKEDDKVFPG
GSHTYVWQVLKENGPMASDPLCLTYSYLSHVDLVKDLNSGLIGALLVCRE
GSLAKEKTQTLHKFILLFAVFDEGKSWHSETKNSLMQDRDAASARAWPKM
HTVNGYVNRSLPGLIGCHRKSVYWHVIGMGTTPEVHSIFLEGHTFLVRNH
RQASLEISPITFLTAQTLLMDLGQFLLFCHISSHQHDGMEAYVKVDSCPE
EPQLQMKNNEEAEDYDDDLTDSEMDVVRFDDDNSPSFIQIRSVAKKHPKT
WVHYIAAEEEDWDYAPLVLAPDDRSYKSQYLNNGPQRIGRKYKKVRFMAY
TDETFKTREAIQHESGILGPLLYGEVGDTLLIIFKNQASRPYNIYPHGIT
DVRPLYSRRLPKGVKHLKDFPILPGEIFKYKWTVTVEDGPTKSDPRCLTR
YYSSFVNMERDLASGLIGPLLICYKESVDQQGNQIMSDKRNVILFSVFDE
NRSWYLTENIQRFLPNPAGVQLEDPEFQASNIMHSINGYVFDSLQLSVCL
HEVAYWYILSIGAQTDFLSVFFSGYTFKHKMVYEDTLTLFPFSGETVFMS
MENPGLWILGCHNSDFRNRGMTALLKVSSCDKNTGDYYEDSYEDISAYLL
SKNNAIEPRSFSQNPPVLKRHQREITRTTLQSDQEEIDYDDTISVEMKKE
DFDIYDEDENQSPRSFQKKTRHYFIAAVERLWDYGMSSSPHVLRNRAQSG
SVPQFKKVVFQEFTDGSFTQPLYRGELNEHLGLLGPYIRAEVEDNIMVTF
RNQASRPYSFYSSLISYEEDQRQGAEPRKNFVKPNETKTYFWKVQHHMAP
TKDEFDCKAWAYFSDVDLEKDVHSGLIGPLLVCHTNTLNPAHGRQVTVQE
FALFFTIFDETKSWYFTENMERNCRAPCNIQMEDPTFKENYRFHAINGYI
MDTLPGLVMAQDQRIRWYLLSMGSNENIHSIHFSGHVFTVRKKEEYKMAL
YNLYPGVFETVEMLPSKAGIWRVECLIGEHLHAGMSTLFLVYSNKCQTPL
GMASGHIRDFQITASGQYGQWAPKLARLHYSGSINAWSTKEPFSWIKVDL
LAPMIIHGIKTQGARQKFSSLYISQFIIMYSLDGKKWQTYRGNSTGTLMV
FFGNVDSSGIKHNIFNPPIIARYIRLHPTHYSIRSTLRMELMGCDLNSCS
MPLGMESKAISDAQITASSYFTNMFATWSPSKARLHLQGRSNAWRPQVNN
PKEWLQVDFQKTMKVTGVTTQGVKSLLTSMYVKEFLISSSQDGHQWTLFF
QNGKVKVFQGNQDSFTPVVNSLDPPLLTRYLRIHPQSWVHQIALRMEVLG
CEAQDLY

TABLE 15
FVIII sequences

F8QQ-00
atgcagattgagctgagcacctgcttcttcctgtgcctgctgaggttctgcttctctgccaccaggagatactacctgggggctgtgg
agctgagctgggactacatgcagtctgacctgggggagctgcctgtggatgccaggttcccccccagagtgcccaagagcttcc
ccttcaacacctctgtggtgtacaagaagaccctgtttgtggagttcactgaccacctgttcaacattgccaagcccaggcccccct
ggatgggcctgctgggccccaccatccaggctgaggtgtatgacactgtggtgatcaccctgaagaacatggccagccaccct
gtgagcctgcatgctgtgggggtgagctactggaaggcctctgagggggctgagtatgatgaccagaccagccagagggaga
aggaggatgacaaggtgttccctgggggcagccacacctatgtgtggcaggtgctgaaggagaatggccccatggcctctgac
cccctgtgcctgacctacagctacctgagccatgtggacctggtgaaggacctgaactctggcctgattggggccctgctggtgtg
cagggagggcagcctggccaaggagaagacccagaccctgcacaagttcatcctgctgtttgctgtgtttgatgagggcaaga
gctggcactctgaaaccaagaacagcctgatgcaggacagggatgctgcctctgccagggcctggcccaagatgcacactgt
gaatggctatgtgaacaggagcctgcctggcctgattggctgccacaggaagtctgtgtactggcatgtgattggcatgggcacc
acccctgaggtgcacagcatcttcctggagggccacaccttcctggtcaggaaccacaggcaggccagcctggagatcagcc
ccatcaccttcctgactgcccagaccctgctgatggacctgggccagttcctgctgttctgccacatcagcagccaccagcatgat
ggcatggaggcctatgtgaaggtggacagctgccctgaggagccccagctgcagatgaagaacaatgaggaggctgagga
ctatgatgatgacctgactgactctgagatggatgtggtgaggtttgatgatgacaacagccccagcttcatccagatcaggtctgt
ggccaagaagcaccccaagacctgggtgcactacattgctgctgaggaggaggactgggactatgcccccctggtgctggcc
cctgatgacaggagctacaagagccagtacctgaacaatggcccccagaggattggcaggaagtacaagaaggtcaggttc
atggcctacactgatgaaaccttcaagaccagggaggccatccagcatgagtctggcatcctgggccccctgctgtatggggag
gtgggggacaccctgctgatcatcttcaagaaccaggccagcaggccctacaacatctacccccatggcatcactgatgtgagg
cccctgtacagcaggaggctgcccaagggggtgaagcacctgaaggacttccccatcctgcctggggagatcttcaagtacaa
gtggactgtgactgtggaggatggccccaccaagtctgaccccaggtgcctgaccagatactacagcagctttgtgaacatgga
gagggacctggcctctggcctgattggccccctgctgatctgctacaaggagtctgtggaccagcagggcaaccagatcatgtct
gacaagaggaatgtgatcctgttctctgtgtttgatgagaacaggagctggtacctgactgagaacatccagaggttcctgcccaa
ccctgctggggtgcagctggaggaccctgagttccaggccagcaacatcatgcacagcatcaatggctatgtgtttgacagcctg
cagctgtctgtgtgcctgcatgaggtggcctactggtacatcctgagcattggggcccagactgacttcctgtctgtgttcttctctggc
tacaccttcaagcacaagatggtgtatgaggacaccctgaccctgttccccttctctggggagactgtgttcatgagcatggagaa
ccctggcctgtggattctgggctgccacaactctgacttcaggaacaggggcatgactgccctgctgaaagtctccagctgtgac
aagaacactggggactactatgaggacagctatgaggacatctctgcctacctgctgagcaagaacaatgccattgagcccag
gagcttcagccagaaccccccagtgctgaagaggcaccagagggagatcaccaggaccaccctgcagtctgaccaggagg
agattgactatgatgacaccatctctgtggagatgaagaaggaggactttgacatctacgacgaggacgagaaccagagcccc
aggagcttccagaagaagaccaggcactacttcattgctgctgtggagaggctgtgggactatggcatgagcagcagccccca
tgtgctgaggaacagggcccagtctggctctgtgccccagttcaagaaggtggtgttccaggagttcactgatggcagcttcaccc
agcccctgtacagaggggagctgaatgagcacctgggcctgctgggcccctacatcagggctgaggtggaggacaacatcat
ggtgaccttcaggaaccaggccagcaggccctacagcttctacagcagcctgatcagctatgaggaggaccagaggcaggg
ggctgagcccaggaagaactttgtgaagcccaatgaaaccaagacctacttctggaaggtgcagcaccacatggcccccacc
aaggatgagtttgactgcaaggcctgggcctacttctctgatgtggacctggagaaggatgtgcactctggcctgattggccccct
gctggtgtgccacaccaacaccctgaaccctgcccatggcaggcaggtgactgtgcaggagtttgccctgttcttcaccatctttga
tgaaaccaagagctggtacttcactgagaacatggagaggaactgcagggccccctgcaacatccagatggaggaccccac
cttcaaggagaactacaggttccatgccatcaatggctacatcatggacaccctgcctggcctggtgatggcccaggaccagag
gatcaggtggtacctgctgagcatgggcagcaatgagaacatccacagcatccacttctctggccatgtgttcactgtgaggaag
aaggaggagtacaagatggccctgtacaacctgtaccctggggtgtttgagactgtggagatgctgcccagcaaggctggcatc
tggagggtggagtgcctgattggggagcacctgcatgctggcatgagcaccctgttcctggtgtacagcaacaagtgccagacc
cccctgggcatggcctctggccacatcagggacttccagatcactgcctctggccagtatggccagtgggcccccaagctggcc
aggctgcactactctggcagcatcaatgcctggagcaccaaggagcccttcagctggatcaaggtggacctgctggcccccatg
atcatccatggcatcaagacccagggggccaggcagaagttcagcagcctgtacatcagccagttcatcatcatgtacagcctg
gatggcaagaagtggcagacctacaggggcaacagcactggcaccctgatggtgttctttggcaatgtggacagctctggcatc
aagcacaacatcttcaacccccccatcattgccagatacatcaggctgcaccccacccactacagcatcaggagcaccctgag
gatggagctgatgggctgtgacctgaacagctgcagcatgcccctgggcatggagagcaaggccatctctgatgcccagatca
ctgccagcagctacttcaccaacatgtttgccacctggagccccagcaaggccaggctgcacctgcagggcaggagcaatgc
ctggaggccccaggtcaacaaccccaaggagtggctgcaggtggacttccagaagaccatgaaggtgactggggtgaccac
ccagggggtgaagagcctgctgaccagcatgtatgtgaaggagttcctgatcagcagcagccaggatggccaccagtggacc
ctgttcttccagaatggcaaggtgaaggtgttccagggcaaccaggacagcttcacccctgtggtgaacagcctggaccccccc
ctgctgaccagatacctgaggattcacccccagagctgggtgcaccagattgccctgaggatggaggtgctgggctgtgaggcc
caggacctgtactga (SEQ ID NO: 125)
F8-SQ
atgcagattgagctgagcacctgcttcttcctgtgcctgctgaggttctgcttctctgccaccaggagatactacctgggggctgtgg
agctgagctgggactacatgcagtctgacctgggggagctgcctgtggatgccaggttcccccccagagtgcccaagagcttcc
ccttcaacacctctgtggtgtacaagaagaccctgtttgtggagttcactgaccacctgttcaacattgccaagcccaggcccccct
ggatgggcctgctgggccccaccatccaggctgaggtgtatgacactgtggtgatcaccctgaagaacatggccagccaccct
gtgagcctgcatgctgtgggggtgagctactggaaggcctctgagggggctgagtatgatgaccagaccagccagagggaga
aggaggatgacaaggtgttccctgggggcagccacacctatgtgtggcaggtgctgaaggagaatggccccatggcctctgac
cccctgtgcctgacctacagctacctgagccatgtggacctggtgaaggacctgaactctggcctgattggggccctgctggtgtg
cagggagggcagcctggccaaggagaagacccagaccctgcacaagttcatcctgctgtttgctgtgtttgatgagggcaaga
gctggcactctgaaaccaagaacagcctgatgcaggacagggatgctgcctctgccagggcctggcccaagatgcacactgt
gaatggctatgtgaacaggagcctgcctggcctgattggctgccacaggaagtctgtgtactggcatgtgattggcatgggcacc
acccctgaggtgcacagcatcttcctggagggccacaccttcctggtcaggaaccacaggcaggccagcctggagatcagcc
ccatcaccttcctgactgcccagaccctgctgatggacctgggccagttcctgctgttctgccacatcagcagccaccagcatgat
ggcatggaggcctatgtgaaggtggacagctgccctgaggagccccagctgaggatgaagaacaatgaggaggctgagga
ctatgatgatgacctgactgactctgagatggatgtggtgaggtttgatgatgacaacagccccagcttcatccagatcaggtctgt
ggccaagaagcaccccaagacctgggtgcactacattgctgctgaggaggaggactgggactatgcccccctggtgctggcc
cctgatgacaggagctacaagagccagtacctgaacaatggcccccagaggattggcaggaagtacaagaaggtcaggttc
atggcctacactgatgaaaccttcaagaccagggaggccatccagcatgagtctggcatcctgggccccctgctgtatggggag
gtgggggacaccctgctgatcatcttcaagaaccaggccagcaggccctacaacatctacccccatggcatcactgatgtgagg
cccctgtacagcaggaggctgcccaagggggtgaagcacctgaaggacttccccatcctgcctggggagatcttcaagtacaa
gtggactgtgactgtggaggatggccccaccaagtctgaccccaggtgcctgaccagatactacagcagctttgtgaacatgga
gagggacctggcctctggcctgattggccccctgctgatctgctacaaggagtctgtggaccagaggggcaaccagatcatgtct
gacaagaggaatgtgatcctgttctctgtgtttgatgagaacaggagctggtacctgactgagaacatccagaggttcctgcccaa
ccctgctggggtgcagctggaggaccctgagttccaggccagcaacatcatgcacagcatcaatggctatgtgtttgacagcctg
cagctgtctgtgtgcctgcatgaggtggcctactggtacatcctgagcattggggcccagactgacttcctgtctgtgttcttctctggc
tacaccttcaagcacaagatggtgtatgaggacaccctgaccctgttccccttctctggggagactgtgttcatgagcatggagaa
ccctggcctgtggattctgggctgccacaactctgacttcaggaacaggggcatgactgccctgctgaaagtctccagctgtgac
aagaacactggggactactatgaggacagctatgaggacatctctgcctacctgctgagcaagaacaatgccattgagcccag
gagcttcagccagaaccccccagtgctgaagaggcaccagagggagatcaccaggaccaccctgcagtctgaccaggagg
agattgactatgatgacaccatctctgtggagatgaagaaggaggactttgacatctacgacgaggacgagaaccagagcccc
aggagcttccagaagaagaccaggcactacttcattgctgctgtggagaggctgtgggactatggcatgagcagcagccccca
tgtgctgaggaacagggcccagtctggctctgtgccccagttcaagaaggtggtgttccaggagttcactgatggcagcttcaccc
agcccctgtacagaggggagctgaatgagcacctgggcctgctgggcccctacatcagggctgaggtggaggacaacatcat
ggtgaccttcaggaaccaggccagcaggccctacagcttctacagcagcctgatcagctatgaggaggaccagaggcaggg
ggctgagcccaggaagaactttgtgaagcccaatgaaaccaagacctacttctggaaggtgcagcaccacatggcccccacc
aaggatgagtttgactgcaaggcctgggcctacttctctgatgtggacctggagaaggatgtgcactctggcctgattggccccct
gctggtgtgccacaccaacaccctgaaccctgcccatggcaggcaggtgactgtgcaggagtttgccctgttcttcaccatctttga
tgaaaccaagagctggtacttcactgagaacatggagaggaactgcagggccccctgcaacatccagatggaggaccccac
cttcaaggagaactacaggttccatgccatcaatggctacatcatggacaccctgcctggcctggtgatggcccaggaccagag
gatcaggtggtacctgctgagcatgggcagcaatgagaacatccacagcatccacttctctggccatgtgttcactgtgaggaag
aaggaggagtacaagatggccctgtacaacctgtaccctggggtgtttgagactgtggagatgctgcccagcaaggctggcatc
tggagggtggagtgcctgattggggagcacctgcatgctggcatgagcaccctgttcctggtgtacagcaacaagtgccagacc
cccctgggcatggcctctggccacatcagggacttccagatcactgcctctggccagtatggccagtgggcccccaagctggcc
aggctgcactactctggcagcatcaatgcctggagcaccaaggagcccttcagctggatcaaggtggacctgctggcccccatg
atcatccatggcatcaagacccagggggccaggcagaagttcagcagcctgtacatcagccagttcatcatcatgtacagcctg
gatggcaagaagtggcagacctacaggggcaacagcactggcaccctgatggtgttctttggcaatgtggacagctctggcatc
aagcacaacatcttcaacccccccatcattgccagatacatcaggctgcaccccacccactacagcatcaggagcaccctgag
gatggagctgatgggctgtgacctgaacagctgcagcatgcccctgggcatggagagcaaggccatctctgatgcccagatca
ctgccagcagctacttcaccaacatgtttgccacctggagccccagcaaggccaggctgcacctgcagggcaggagcaatgc
ctggaggccccaggtcaacaaccccaaggagtggctgcaggtggacttccagaagaccatgaaggtgactggggtgaccac
ccagggggtgaagagcctgctgaccagcatgtatgtgaaggagttcctgatcagcagcagccaggatggccaccagtggacc
ctgttcttccagaatggcaaggtgaaggtgttccagggcaaccaggacagcttcacccctgtggtgaacagcctggaccccccc
ctgctgaccagatacctgaggattcacccccagagctgggtgcaccagattgccctgaggatggaggtgctgggctgtgaggcc
caggacctgtactga (SEQ ID NO: 126)
FVIII-QQ01
atgcagattgagctgagcacctgcttcttcctgtgcctgctgagattctgcttctctgccaccagaagatactacctgggtgcagtgg
agctgagctgggactacatgcagagtgacctgggtgagctgcctgttgatgccagattcccccccagagtgcccaagagcttcc
ccttcaacacctctgttgtgtacaagaagaccctgtttgttgagttcactgaccacctgttcaacattgccaagcccagacccccctg
gatgggcctgctgggccccaccatccaggcagaggtgtatgatactgtggtgatcaccctgaagaacatggccagccacccag
ttagcctgcatgcagtgggggtcagctactggaaggcctcagaaggggctgagtatgatgatcagaccagccagagagagaa
ggaggatgacaaggtgttcccagggggtagccacacctatgtatggcaggtgctgaaggagaatggccccatggcctctgacc
ccctgtgcctgacctacagctacctgagccatgtggacctggtgaaggacctgaacagtggactgattggtgccctgctggtgtgc
agagagggcagcctggccaaggagaagacccagaccctgcacaagttcatcctgctgtttgctgtgtttgatgaaggcaagag
ctggcacagtgagaccaagaacagcctgatgcaggacagagatgctgcatcagctagagcctggcccaagatgcacactgtc
aatggttatgtaaacagaagcctgcctggcctgattggctgccacagaaagtcagtatactggcatgttataggcatgggcacca
ccccagaggtgcacagcatcttcctggagggccacaccttcctggtgagaaaccacagacaggccagcctggagatcagccc
catcaccttcctgacagcccagaccctgctgatggacctgggccagttcctgctgttctgccacatcagcagccaccagcatgatg
ggatggaggcctatgtaaaggtggacagctgcccagaggagccccagctgcagatgaagaacaatgaagaggctgaagac
tatgatgatgatctgactgattcagagatggatgttgtgagatttgatgatgacaacagccccagcttcatccagatcagatcagtg
gccaagaagcaccccaagacctgggtgcactacatagctgcagaggaggaggactgggactatgcccccctggtgctggcc
ccagatgatagaagctacaagagccagtacctgaacaatggcccccagagaatagggagaaagtacaagaaggtgagatt
catggcctacacagatgagaccttcaagaccagagaggccatccagcatgagtcaggtatcctgggccccctgctgtatgggg
aggtgggtgacaccctgctgatcatcttcaagaaccaggccagcagaccctacaacatctacccccatgggatcacagatgtca
gacccctgtacagcagaagactgcccaagggagtcaagcacctgaaggacttccccatcctgcctggagaaatcttcaagtac
aagtggacagtgactgtggaggatggtcccaccaagagtgaccccagatgcctgaccagatactacagcagctttgtcaacat
ggagagagacctggccagtggcctgataggccccctgctgatctgctacaaggagtctgtggaccagcagggcaaccagatc
atgtctgacaagagaaatgttatcctgttcagtgtatttgatgagaacagaagctggtacctgactgagaacatccagagattcctg
cccaacccagctggggtccagctggaggaccctgagttccaggccagcaacatcatgcacagcatcaatggctatgtgtttgac
agcctgcagctgtctgtctgcctgcatgaagtggcctactggtacatcctgagcataggagcccagacagacttcctgagtgtattc
ttctctggctacaccttcaagcacaagatggtgtatgaggacaccctgaccctgttccccttctctggagagactgtattcatgagca
tggagaaccctggcctgtggatcctgggctgccacaactctgacttcagaaacagaggcatgactgccctgctgaaggtgagca
gctgtgataagaacacaggggactactatgaggacagctatgaggacatctcagcctacctgctgagcaagaacaatgccatt
gagcccagaagcttcagccagaacccccctgtgctgaagagacaccagagagagatcaccagaaccaccctgcagagtga
ccaggaggagattgactatgatgacaccatctctgttgagatgaagaaggaggactttgacatctatgatgaggatgagaacca
gagccccagaagcttccagaagaagaccagacactacttcattgcagctgtggagagactgtgggactatggcatgagcagc
agcccccatgtgctgagaaacagagcccagtcaggctctgtgccccagttcaagaaggtggtgttccaggagttcacagatgg
gagcttcacccagcccctgtacagaggtgaactgaatgagcacctgggcctgctgggcccctacatcagagctgaagtggagg
acaacatcatggtgaccttcagaaaccaggccagcagaccctacagcttctacagcagcctgatcagctatgaagaggacca
gagacagggtgctgagcccagaaagaactttgtgaagcccaatgaaaccaagacctacttctggaaggtgcagcaccacatg
gcccccaccaaggatgaatttgactgcaaggcctgggcctacttctcagatgtagacctggagaaggatgttcactcaggcctga
ttgggcccctgctggtgtgccacaccaacaccctgaacccagcccatggtagacaggtgacagtgcaggagtttgccctgttcttc
accatctttgatgagaccaagagctggtacttcactgagaacatggagagaaactgcagagccccctgcaacatccagatgga
ggaccccaccttcaaggagaactacagattccatgccatcaatgggtacatcatggacaccctgccaggcctggtgatggccca
ggaccagagaatcagatggtacctgctgagcatgggcagcaatgagaacatccacagcatccacttcagtggtcatgtcttcac
agtgagaaagaaggaggagtacaagatggccctgtacaacctgtacccaggggtgtttgaaactgtagagatgctgcccagc
aaggcaggcatctggagagtggagtgcctgataggagaacacctgcatgcaggaatgagcaccctgttcctggtgtacagcaa
caagtgccagacccccctgggcatggcctctgggcacatcagagacttccagatcactgcctctggccagtatgggcagtgggc
ccccaagctggccagactgcactactcaggtagcatcaatgcctggagcaccaaggagcccttcagctggatcaaggtggacc
tgctggcccccatgatcatccatggtatcaagacccagggtgccagacagaagttcagcagcctgtacatcagccagttcatcat
catgtacagcctggatggcaagaagtggcagacctacagaggcaacagcactggcaccctgatggtgttctttggcaatgtgga
cagctctggcatcaagcacaacatcttcaacccccccatcatagccagatacatcagactgcaccccacccactacagcatca
gaagcaccctgagaatggagctgatgggctgtgatctgaacagctgcagcatgcccctgggcatggagagcaaggccatctct
gatgctcagatcactgcaagcagctacttcaccaacatgtttgccacctggagccccagcaaggccagactgcacctgcaggg
cagaagcaatgcctggagaccccaggtgaacaaccccaaggagtggctgcaggtggacttccagaagaccatgaaggtga
caggtgtcaccacccagggggtgaagagcctgctgaccagcatgtatgtgaaggagttcctgatcagcagcagccaggatgg
ccaccagtggaccctgttcttccagaatggcaaggtgaaggtgttccagggcaaccaggacagcttcacccctgtggtgaacag
cctggacccccccctgctgaccagatacctgagaatccacccccagagctgggtgcaccagattgccctgagaatggaggtgc
tgggctgtgaggcccaggacctgtactga (SEQ ID NO: 61)
FVIII-QQ02
atgcagattgagctgagcacctgcttcttcctgtgcctgctgagattctgcttcagtgccaccagaagatactacctgggagcagtg
gagctgagctgggactacatgcagagtgatctgggagagctgccagtagatgccagattcccccccagagtgcccaagagctt
ccccttcaacacctcagtggtgtacaagaagaccctgtttgttgagttcactgaccacctgttcaacattgcaaagcccagacccc
cctggatgggcctgctgggccccaccatccaggcagaagtgtatgacacagtagtgatcaccctgaagaacatggccagcca
ccctgtcagcctgcatgcagttggtgtaagctactggaaggcctctgagggtgctgaatatgatgaccagaccagccagagaga
gaaggaggatgataaggtgttcccagggggcagccacacctatgtttggcaggtgctgaaggagaatgggcccatggcctctg
atcccctgtgcctgacctacagctacctgagccatgtggacctggtgaaggacctgaactctgggctgataggagccctgctggt
gtgcagagagggcagcctggccaaggagaagacccagaccctgcacaagttcatcctgctgtttgctgtgtttgatgaaggcaa
gagctggcacagtgaaaccaagaacagcctgatgcaggacagagatgcagctagtgccagagcctggcccaagatgcaca
cagtaaatggttatgtgaacagaagcctgcctggcctgataggctgccacagaaagtctgtgtactggcatgttataggtatgggc
accacccctgaggtgcacagcatcttcctggagggccacaccttcctggtgagaaaccacagacaggccagcctggagatca
gccccatcaccttcctgactgcccagaccctgctgatggacctgggccagttcctgctgttctgccacatcagcagccaccagcat
gatggaatggaggcctatgtcaaggtggacagctgcccagaggagccccagctgcagatgaagaacaatgaggaggcaga
ggactatgatgatgacctgactgacagtgaaatggatgtggtcagatttgatgatgataacagccccagcttcatccagatcagat
cagtggccaagaagcaccccaagacctgggtgcactacattgcagctgaggaggaggactgggactatgcacccctggtgct
ggccccagatgatagaagctacaagagccagtacctgaacaatggaccccagagaattggcagaaagtacaagaaggtga
gattcatggcctacactgatgagaccttcaagaccagagaggccatccagcatgaatcaggcatcctgggccccctgctgtatg
gggaggtgggagacaccctgctgatcatcttcaagaaccaggccagcagaccctacaacatctacccccatggaatcacaga
tgttagacccctgtacagcagaagactgcccaagggggtgaagcacctgaaggacttccccatcctgcctggggagatcttcaa
gtacaagtggacagttacagtagaggatggccccaccaagagtgaccccagatgcctgaccagatactacagcagctttgtaa
acatggagagagacctggcctctggcctgataggccccctgctgatctgctacaaggagtcagttgaccagcagggcaaccag
atcatgagtgataagagaaatgtcatcctgttcagtgtgtttgatgagaacagaagctggtacctgacagagaacatccagagatt
cctgcccaaccctgctggagtgcagctggaggacccagaattccaggccagcaacatcatgcacagcatcaatggttatgtcttt
gatagcctgcagctgtcagtttgcctgcatgaggtggcctactggtacatcctgagcattggggcccagactgacttcctgagtgtat
tcttcagtggctacaccttcaagcacaagatggtgtatgaggacaccctgaccctgttccccttctctggagagactgtgttcatgag
catggagaaccctgggctgtggatcctgggctgccacaactcagacttcagaaacagaggcatgacagcactgctgaaggtg
agcagctgtgataagaacactggtgactactatgaggacagctatgaagacatctcagcctacctgctgagcaagaacaatgc
catagagcccagaagcttcagccagaaccccccagttctgaagagacaccagagagagatcaccagaaccaccctgcaga
gtgaccaggaggagatagattatgatgataccatctcagttgagatgaagaaggaggactttgatatctatgatgaggatgagaa
ccagagccccagaagcttccagaagaagaccagacactacttcatagcagctgtggagagactgtgggactatggcatgagc
agcagcccccatgttctgagaaacagagcccagagtggatcagtaccccagttcaagaaggtggtgttccaggagttcactgat
gggagcttcacccagcccctgtacagaggtgaactgaatgagcacctgggcctgctgggcccctacatcagagctgaagtgga
ggacaacatcatggtgaccttcagaaaccaggccagcagaccctacagcttctacagcagcctgatcagctatgaggaggac
cagagacagggagctgagcccagaaagaactttgtcaagcccaatgaaaccaagacctacttctggaaggtgcagcaccac
atggcccccaccaaggatgagtttgactgcaaggcctgggcctacttcagtgatgtagacctggagaaggatgtccactcaggc
ctgattggccccctgctggtgtgccacaccaacaccctgaacccagcacatgggagacaggtgactgtacaggagtttgctctgt
tcttcaccatctttgatgagaccaagagctggtacttcacagaaaacatggagagaaactgcagagccccctgcaacatccaga
tggaggaccccaccttcaaggagaactacagattccatgccatcaatggttacatcatggacaccctgcctggcctggtgatggc
ccaggaccagagaatcagatggtacctgctgagcatgggcagcaatgaaaacatccacagcatccacttctcagggcatgtgt
tcactgtgagaaagaaggaggagtacaagatggccctgtacaacctgtacccaggtgtgtttgaaactgtggagatgctgccca
gcaaggcaggcatctggagagtggagtgcctgataggggagcacctgcatgcagggatgagcaccctgttcctggtgtacagc
aacaagtgccagacccccctgggcatggcctcaggccacatcagagacttccagatcacagcttctggacagtatggccagtg
ggcccccaagctggccagactgcactacagtggcagcatcaatgcctggagcaccaaggagcccttcagctggatcaaggtg
gacctgctggcccccatgatcatccatggcatcaagacccagggagccagacagaagttcagcagcctgtacatcagccagtt
catcatcatgtacagcctggatggcaagaagtggcagacctacagaggcaacagcacaggcaccctgatggtgttctttggga
atgtggacagcagtggaatcaagcacaacatcttcaacccccccatcattgcaagatacatcagactgcaccccacccactac
agcatcagaagcaccctgagaatggagctgatgggctgtgacctgaacagctgcagcatgcccctgggcatggagagcaag
gccatctctgatgcccagatcacagctagcagctacttcaccaacatgtttgccacctggagccccagcaaggccagactgcac
ctgcagggcagaagcaatgcctggagaccccaggtgaacaaccccaaggagtggctgcaggtggacttccagaagaccat
gaaggtgactggtgttaccacccagggggtcaagagcctgctgaccagcatgtatgtgaaggagttcctgatcagcagcagcc
aggatggccaccagtggaccctgttcttccagaatggcaaggtgaaggtgttccagggcaaccaggacagcttcacccctgtgg
tgaacagcctggacccccccctgctgaccagatacctgagaatccacccccagagctgggtgcaccagatagcactgagaat
ggaggtgctgggctgtgaggcccaggacctgtactga (SEQ ID NO: 62)
FVIII-QQ03
atgcaaattgagttgtccacctgcttcttcttgtgcttgttgaggttctgcttcagtgccaccaggaggtactacttgggggctgtggagt
tgtcctgggactacatgcagtcagacttgggggagttgcctgtggatgccaggttcccccccagggtccccaagtccttccccttca
acaccagtgtggtctacaagaagaccttgtttgtggagttcactgaccacttgttcaacattgccaagcccaggcccccctggatg
ggcttgttgggccccaccatccaggctgaggtctatgacacagtggtcatcaccttgaagaacatggcctcccaccctgtctccttg
catgcagtgggggtctcctactggaaggccagtgagggggctgagtatgatgaccagacctcccagagggagaaggaggat
gacaaggtcttccctgggggctcccacacctatgtctggcaggtcttgaaggagaatggccccatggcctctgaccccttgtgcttg
acctactcctacttgtcccatgtggacttggtcaaggacttgaactcaggcttgattggggccttgttggtctgcagggagggctcctt
ggccaaggagaagacccagaccttgcacaagttcatcttgttgtttgcagtctttgatgagggcaagtcctggcacagtgagacc
aagaactccttgatgcaggacagggatgctgccagtgccagggcctggcccaagatgcacactgtcaatggctatgtcaacag
gtccttgcctggcttgattggctgccacaggaagtcagtctactggcatgtcattggcatgggcaccacccctgaggtccactccat
cttcttggagggccacaccttcttggtcaggaaccacaggcaggcctccttggagatctcccccatcaccttcttgacagcccaga
ccttgttgatggacttgggccagttcttgttgttctgccacatctcctcccaccagcatgatggcatggaggcctatgtcaaggtggac
tcctgccctgaggagccccagttgcagatgaagaacaatgaggaggcagaggactatgatgatgacttgactgactctgagat
ggatgtggtcaggtttgatgatgacaactccccctccttcatccagatcaggagtgtggccaagaagcaccccaagacctgggtc
cactacatagcagcagaggaggaggactgggactatgcccccttggtcttggcccctgatgacaggtcctacaagtcccagtac
ttgaacaatggcccccagaggattggcaggaagtacaagaaggtcaggttcatggcctacacagatgagaccttcaagacca
gggaggccatccagcatgagtcaggcatcttgggccccttgttgtatggggaggtgggggacaccttgttgatcatcttcaagaac
caggcctccaggccctacaacatctacccccatggcatcactgatgtcaggcccttgtactccaggaggttgcccaagggggtc
aagcacttgaaggacttccccatcttgccaggggagatcttcaagtacaagtggacagtcactgtggaggatggccccaccaa
gtcagaccccaggtgcttgaccaggtactactcctcctttgtcaacatggagagggacttggccagtggcttgattggccccttgttg
atctgctacaaggagtctgtggaccagcagggcaaccagatcatgtcagacaagaggaatgtcatcttgttcagtgtctttgatga
gaacaggtcctggtacttgacagagaacatccagaggttcttgcccaacccagctggggtccagttggaggaccctgagttcca
ggcctccaacatcatgcactccatcaatggctatgtctttgactccttgcagttgtcagtctgcttgcatgaggtggcctactggtacat
cttgtccattggggcccagactgacttcttgtctgtcttcttcagtggctacaccttcaagcacaagatggtctatgaggacaccttga
ccttgttccccttcagtggggagactgtcttcatgtccatggagaacccaggcttgtggatcttgggctgccacaactcagacttcag
gaacaggggcatgacagccttgttgaaggtctcctcctgtgacaagaacacaggggactactatgaggactcctatgaggacat
ctcagcctacttgttgtccaagaacaatgccatagagcccaggtccttctcccagaacccccctgtcttgaagaggcaccagagg
gagatcaccaggaccaccttgcagtcagaccaggaggagatagactatgatgacaccatctcagtggagatgaagaaggag
gactttgacatctatgatgaggatgagaaccagtcccccaggtccttccagaagaagaccaggcactacttcattgcagcagtg
gagaggttgtgggactatggcatgtcctcctccccccatgtcttgaggaacagggcccagtcaggcagtgtcccccagttcaaga
aggtggtcttccaggagttcactgatggctccttcacccagcccttgtacaggggggagttgaatgagcacttgggcttgttgggcc
cctacatcagggctgaggtggaggacaacatcatggtcaccttcaggaaccaggcctccaggccctactccttctactcctccttg
atctcctatgaggaggaccagaggcagggggcagagcccaggaagaactttgtcaagcccaatgagaccaagacctacttct
ggaaggtccagcaccacatggcccccaccaaggatgagtttgactgcaaggcctgggcctacttctctgatgtggacttggaga
aggatgtccactctggcttgattggccccttgttggtctgccacaccaacaccttgaaccctgcccatggcaggcaggtcactgtcc
aggagtttgccttgttcttcaccatctttgatgagaccaagtcctggtacttcacagagaacatggagaggaactgcagggccccct
gcaacatccagatggaggaccccaccttcaaggagaactacaggttccatgccatcaatggctacatcatggacaccttgcca
ggcttggtcatggcccaggaccagaggatcaggtggtacttgttgtccatgggctccaatgagaacatccactccatccacttcag
tggccatgtcttcactgtcaggaagaaggaggagtacaagatggccttgtacaacttgtacccaggggtctttgagacagtggag
atgttgccctccaaggcaggcatctggagggtggagtgcttgattggggagcacttgcatgctggcatgtccaccttgttcttggtct
actccaacaagtgccagacccccttgggcatggcctctggccacatcagggacttccagatcactgcctcaggccagtatggcc
agtgggcccccaagttggccaggttgcactacagtggctccatcaatgcctggtccaccaaggagcccttctcctggatcaaggt
ggacttgttggcccccatgatcatccatggcatcaagacccagggggccaggcagaagttctcctccttgtacatctcccagttcat
catcatgtactccttggatggcaagaagtggcagacctacaggggcaactccacaggcaccttgatggtcttctttggcaatgtgg
actcctctggcatcaagcacaacatcttcaacccccccatcattgccaggtacatcaggttgcaccccacccactactccatcag
gtccaccttgaggatggagttgatgggctgtgacttgaactcctgctccatgcccttgggcatggagtccaaggccatctcagatgc
ccagatcactgcctcctcctacttcaccaacatgtttgccacctggtccccctccaaggccaggttgcacttgcagggcaggtcca
atgcctggaggccccaggtcaacaaccccaaggagtggttgcaggtggacttccagaagaccatgaaggtcacaggggtca
ccacccagggggtcaagtccttgttgacctccatgtatgtcaaggagttcttgatctcctcctcccaggatggccaccagtggacctt
gttcttccagaatggcaaggtcaaggtcttccagggcaaccaggactccttcacccctgtggtcaactccttggacccccccttgtt
gaccaggtacttgaggatccacccccagtcctgggtccaccagattgccttgaggatggaggtcttgggctgtgaggcccagga
cttgtactga (SEQ ID NO: 63)
FVIII-QQ04
atgcagatagagctgagcacctgcttcttcctgtgcctgctgagattctgcttctcagccaccagaagatactacctgggggctgta
gagctgagctgggactacatgcagtcagacctgggtgagctgccagtggatgccagattcccccccagagtgcccaagagctt
ccccttcaacacctctgtggtgtacaagaagaccctgtttgtggagttcactgatcacctgttcaacatagctaagcccagaccccc
ctggatgggcctgctgggccccaccatccaggctgaggtgtatgatacagtggtgatcaccctgaagaacatggccagccacc
ctgtgagcctgcatgcagtgggtgtgagctactggaaggcctctgagggtgctgaatatgatgaccagaccagccagagagag
aaggaggatgataaggtgttccctgggggcagccacacctatgtgtggcaggtgctgaaggagaatggacccatggccagtg
accccctgtgcctgacctacagctacctgagccatgtagacctggtgaaggacctgaactctggactgattggggcactgctggt
gtgcagagagggcagcctggccaaggagaagacccagaccctgcacaagttcatcctgctgtttgctgtgtttgatgagggcaa
gagctggcactctgaaaccaagaacagcctgatgcaggacagagatgctgcctcagcaagagcctggcccaagatgcacac
tgtgaatggctatgttaacagaagcctgcctgggctgattgggtgccacagaaagagtgtgtactggcatgtaataggaatgggc
accaccccagaggtgcacagcatcttcctggagggccacaccttcctggtgagaaaccacagacaggccagcctggagatca
gccccatcaccttcctgactgctcagaccctgctgatggacctgggccagttcctgctgttctgccacatcagcagccaccagcat
gatggaatggaggcctatgtaaaggtggacagctgccctgaggagccccagctgcagatgaagaacaatgaggaggcaga
ggactatgatgatgacctgactgacagtgagatggatgtggtgagatttgatgatgataacagccccagcttcatccagatcagat
ctgtagccaagaagcaccccaagacctgggtgcactacatagctgcagaagaggaggactgggactatgcccccctggtgct
ggcccctgatgatagaagctacaagagccagtacctgaacaatggtccccagagaatagggagaaagtacaagaaggtga
gattcatggcctacactgatgagaccttcaagaccagagaggccatccagcatgagtcaggcatcctgggccccctgctgtatg
gagaagtgggtgacaccctgctgatcatcttcaagaaccaggccagcagaccctacaacatctacccccatggcatcactgatg
tcagacccctgtacagcagaagactgcccaagggtgtgaagcacctgaaggacttccccatcctgccaggtgagatcttcaagt
acaagtggacagtgactgtagaggatgggcccaccaagtcagaccccagatgcctgaccagatactacagcagctttgtgaa
catggagagagacctggccagtggcctgattggccccctgctgatctgctacaaggagtcagttgaccagcagggcaaccaga
tcatgtctgacaagagaaatgtcatcctgttcagtgtgtttgatgagaacagaagctggtacctgactgagaacatccagagattc
ctgcccaacccagcaggagttcagctggaggaccctgagttccaggccagcaacatcatgcacagcatcaatggctatgtttttg
acagcctgcagctgtcagtgtgcctgcatgaagtggcctactggtacatcctgagcattggtgcccagacagatttcctgagtgtctt
cttctcaggctacaccttcaagcacaagatggtgtatgaggacaccctgaccctgttccccttctctggggaaacagtgttcatgag
catggagaaccctggtctgtggatcctgggctgccacaactcagacttcagaaacagaggcatgacagcactgctgaaggtga
gcagctgtgacaagaacacaggtgactactatgaagacagctatgaggacatctcagcctacctgctgagcaagaacaatgc
cattgagcccagaagcttcagccagaacccccctgtgctgaagagacaccagagagagatcaccagaaccaccctgcagtc
agaccaggaggagattgactatgatgacaccatcagtgtagaaatgaagaaggaggactttgacatctatgatgaggatgaaa
accagagccccagaagcttccagaagaagaccagacactacttcatagctgcagttgagagactgtgggactatggaatgag
cagcagcccccatgtgctgagaaacagagcccagtctggctcagttccccagttcaagaaggtggtgttccaggagttcacaga
tgggagcttcacccagcccctgtacagaggggagctgaatgagcacctgggcctgctgggcccctacatcagagcagaggtg
gaggacaacatcatggtgaccttcagaaaccaggccagcagaccctacagcttctacagcagcctgatcagctatgaagagg
accagagacagggagcagaacccagaaagaactttgttaagcccaatgaaaccaagacctacttctggaaggtgcagcacc
acatggcccccaccaaggatgaatttgactgcaaggcctgggcctacttctctgatgttgacctggagaaggatgtgcactcagg
cctgataggccccctgctggtgtgccacaccaacaccctgaacccagctcatgggagacaggtgactgttcaggagtttgccctg
ttcttcaccatctttgatgagaccaagagctggtacttcacagagaacatggagagaaactgcagagccccctgcaacatccag
atggaggaccccaccttcaaggagaactacagattccatgccatcaatggttacatcatggacaccctgcctgggctggtgatgg
cccaggaccagagaatcagatggtacctgctgagcatgggcagcaatgaaaacatccacagcatccacttctctggccatgtgt
tcactgtcagaaagaaggaggagtacaagatggccctgtacaacctgtaccctggagtatttgagactgtggagatgctgccca
gcaaggcaggtatctggagagtggagtgcctgattggggagcacctgcatgcaggtatgagcaccctgttcctggtgtacagca
acaagtgccagacccccctgggcatggcctctggacacatcagagacttccagatcactgccagtggccagtatggccagtgg
gcccccaagctggccagactgcactactcagggagcatcaatgcctggagcaccaaggagcccttcagctggatcaaggtgg
acctgctggcccccatgatcatccatggtatcaagacccagggtgccagacagaagttcagcagcctgtacatcagccagttcat
catcatgtacagcctggatggcaagaagtggcagacctacagaggcaacagcacaggcaccctgatggtgttctttggcaatgt
agacagctctggcatcaagcacaacatcttcaacccccccatcatagctagatacatcagactgcaccccacccactacagcat
cagaagcaccctgagaatggagctgatgggctgtgacctgaacagctgcagcatgcccctgggcatggagagcaaggccat
ctcagatgctcagatcactgccagcagctacttcaccaacatgtttgccacctggagccccagcaaggccagactgcacctgca
gggcagaagcaatgcctggagaccccaggtgaacaaccccaaggagtggctgcaggtggacttccagaagaccatgaagg
tgacaggagtgaccacccagggggtgaagagcctgctgaccagcatgtatgtgaaggagttcctgatcagcagcagccagga
tggacaccagtggaccctgttcttccagaatggaaaggtgaaggtgttccagggcaaccaggacagcttcacccctgtggtgaa
cagcctggacccccccctgctgaccagatacctgagaatccacccccagagctgggtgcaccagattgccctgagaatggag
gtgctgggctgtgaggcccaggacctgtactga (SEQ ID NO: 64)
FVIII-QQ05
atgcagattgagctgagcacctgcttcttcctgtgcctgctgagattctgcttctctgctaccagaagatactacctgggagctgtgga
gctgagctgggactacatgcagtcagacctgggtgaactgcctgtggatgccagattcccccccagagtgcccaagagcttccc
cttcaacaccagtgttgtctacaagaagaccctgtttgtggagttcacagaccacctgttcaacattgccaagcccagacccccct
ggatgggcctgctgggccccaccatccaggctgaagtgtatgatacagtggtgatcaccctgaagaacatggccagccaccca
gtgagcctgcatgcagtgggtgtgagctactggaaggcctctgaaggtgcagagtatgatgaccagaccagccagagagaga
aggaggatgacaaggtgttccctgggggcagccacacctatgtgtggcaggtgctgaaggagaatggccccatggccagtgat
cccctgtgcctgacctacagctacctgagccatgttgacctggtgaaggacctgaactcaggtctgattggagccctgctggtgtg
cagagagggcagcctggccaaggagaagacccagaccctgcacaagttcatcctgctgtttgctgtctttgatgaaggcaaga
gctggcacagtgagaccaagaacagcctgatgcaggacagagatgcagcctctgccagagcctggcccaagatgcacaca
gtaaatggctatgtgaacagaagcctgccagggctgattggctgccacagaaagtctgtttactggcatgtgattgggatgggca
ccacccctgaagtgcacagcatcttcctggagggccacaccttcctggtgagaaaccacagacaggccagcctggagatcag
ccccatcaccttcctgacagcacagaccctgctgatggacctgggccagttcctgctgttctgccacatcagcagccaccagcat
gatgggatggaggcctatgtgaaggtggacagctgcccagaagagccccagctgcagatgaagaacaatgaggaggcaga
ggactatgatgatgacctgactgactctgagatggatgttgtgagatttgatgatgacaacagccccagcttcatccagatcagaa
gtgttgccaagaagcaccccaagacctgggtgcactacattgcagctgaggaggaggactgggactatgcccccctggtgctg
gccccagatgacagaagctacaagagccagtacctgaacaatggcccccagagaattggtagaaagtacaagaaggtgag
attcatggcctacacagatgagaccttcaagaccagagaggccatccagcatgagtcaggaatcctgggccccctgctgtatgg
ggaggtgggggataccctgctgatcatcttcaagaaccaggccagcagaccctacaacatctacccccatggcatcacagatg
tgagacccctgtacagcagaagactgcccaagggagtgaagcacctgaaggacttccccatcctgcctggtgagatcttcaagt
acaagtggacagttactgtggaagatggtcccaccaagtcagaccccagatgcctgaccagatactacagcagctttgtgaac
atggagagagacctggcctctgggctgattgggcccctgctgatctgctacaaggagagtgtggaccagcagggcaaccagat
catgtctgataagagaaatgtgatcctgttctctgtatttgatgaaaacagaagctggtacctgactgagaacatccagagattcct
gcccaacccagctggagtccagctggaggacccagagttccaggccagcaacatcatgcacagcatcaatggatatgtgtttg
acagcctgcagctgtctgtatgcctgcatgaggtggcctactggtacatcctgagcattggggcccagactgacttcctgtctgtgtt
cttctcaggatacaccttcaagcacaagatggtgtatgaggacaccctgaccctgttccccttctcaggggaaactgtgttcatgag
catggagaaccctggcctgtggatcctgggctgccacaactctgacttcagaaacagaggcatgactgccctgctgaaggtgag
cagctgtgacaagaacactggggattactatgaggacagctatgaggacatctcagcctacctgctgagcaagaacaatgcca
ttgaacccagaagcttcagccagaacccccctgtcctgaagagacaccagagagagatcaccagaaccaccctgcagtctga
tcaggaggagattgactatgatgataccatctcagtggagatgaagaaggaggactttgacatctatgatgaagatgaaaacca
gagccccagaagcttccagaagaagaccagacactacttcattgctgctgtggagagactgtgggactatgggatgagcagca
gcccccatgtgctgagaaacagagcccagtcaggcagtgtgccccagttcaagaaggtggtgttccaggagttcacagatgga
agcttcacccagcccctgtacagaggtgagctgaatgaacacctgggcctgctgggcccctacatcagagctgaagtggagga
caacatcatggtgaccttcagaaaccaggccagcagaccctacagcttctacagcagcctgatcagctatgaggaggaccag
agacagggagcagagcccagaaagaactttgtcaagcccaatgaaaccaagacctacttctggaaggtgcagcaccacatg
gcccccaccaaggatgaatttgactgcaaggcctgggcctacttctctgatgtagacctggagaaggatgtgcactctgggctga
ttggccccctgctggtgtgccacaccaacaccctgaacccagcccatggcagacaggtgactgtgcaggagtttgccctgttcttc
accatctttgatgaaaccaagagctggtacttcactgagaacatggagagaaactgcagagccccctgcaacatccagatgga
ggaccccaccttcaaggagaactacagattccatgctatcaatggatacatcatggacaccctgcctgggctggtgatggccca
ggaccagagaatcagatggtacctgctgagcatgggcagcaatgagaacatccacagcatccacttctcaggacatgtcttcac
agtaagaaagaaggaggagtacaagatggccctgtacaacctgtacccaggggtctttgaaactgtagagatgctgcccagc
aaggcaggcatctggagagtggagtgcctgattggagagcacctgcatgctggtatgagcaccctgttcctggtgtacagcaac
aagtgccagacccccctgggcatggcctctggccacatcagagacttccagatcacagccagtggccagtatgggcagtgggc
ccccaagctggccagactgcactactctggcagcatcaatgcctggagcaccaaggagcccttcagctggatcaaggtggacc
tgctggcccccatgatcatccatgggatcaagacccagggtgccagacagaagttcagcagcctgtacatcagccagttcatca
tcatgtacagcctggatggcaagaagtggcagacctacagaggcaacagcacaggtaccctgatggtgttctttggcaatgtgg
acagctcaggcatcaagcacaacatcttcaacccccccatcattgccagatacatcagactgcaccccacccactacagcatc
agaagcaccctgagaatggagctgatgggctgtgacctgaacagctgcagcatgcccctgggcatggagagcaaggccatct
ctgatgcccagatcacagctagcagctacttcaccaacatgtttgccacctggagccccagcaaggccagactgcacctgcag
ggcagaagcaatgcttggagaccccaggtgaacaaccccaaggagtggctgcaggtggacttccagaagaccatgaaggtg
acaggtgtgaccacccagggggttaagagcctgctgaccagcatgtatgtgaaggagttcctgatcagcagcagccaggatgg
ccaccagtggaccctgttcttccagaatggcaaggtgaaggtgttccagggcaaccaggacagcttcacccctgttgtgaacag
cctggacccccccctgctgaccagatacctgagaatccacccccagagctgggtgcaccagattgccctgagaatggaggtgc
tgggctgtgaggcccaggacctgtactga (SEQ ID NO: 65)
FVIII-QQ06
atgcagatagaactgagcacctgcttcttcctgtgcctgctgagattctgcttctcagccaccagaagatactacctgggggcagtt
gagctgagctgggactacatgcagagtgatctgggggagctgccagtggatgcaagattcccccccagagtgcccaagagctt
ccccttcaacaccagtgtggtctacaagaagaccctgtttgttgagttcacagaccacctgttcaacattgctaagcccagacccc
cctggatgggcctgctgggccccaccatccaggcagaggtgtatgatacagttgtgatcaccctgaagaacatggccagccac
ccagttagcctgcatgcagttggagtgagctactggaaggcctcagagggagctgagtatgatgaccagaccagccagagag
agaaggaggatgataaggtgttcccaggggggagccacacctatgtgtggcaggtgctgaaggagaatggacccatggcca
gtgaccccctgtgcctgacctacagctacctgagccatgtggacctggtgaaggacctgaactcaggactgataggtgctctgct
ggtgtgcagagagggcagcctggccaaggagaagacccagaccctgcacaagttcatcctgctgtttgcagtttttgatgaagg
caagagctggcactcagagaccaagaacagcctgatgcaggacagagatgcagcttctgctagagcctggcccaagatgca
cactgtcaatggctatgtaaacagaagcctgcctggcctgattggctgccacagaaagtctgtctactggcatgttataggtatggg
caccacccctgaggtgcacagcatcttcctggagggccacaccttcctggtgagaaaccacagacaggccagcctggagatc
agccccatcaccttcctgactgcccagaccctgctgatggacctgggccagttcctgctgttctgccacatcagcagccaccagc
atgatggcatggaggcctatgtcaaggtggacagctgcccagaagagccccagctgcagatgaagaacaatgaggaggctg
aggactatgatgatgacctgactgacagtgagatggatgtagtgagatttgatgatgacaacagccccagcttcatccagatcag
aagtgtagccaagaagcaccccaagacctgggtgcactacattgctgcagaggaggaggactgggactatgcccccctggtg
ctggccccagatgacagaagctacaagagccagtacctgaacaatggtccccagagaattggcagaaagtacaagaaggtg
agattcatggcctacacagatgagaccttcaagaccagagaggccatccagcatgagtcaggcatcctgggccccctgctgtat
ggagaggtgggggacaccctgctgatcatcttcaagaaccaggccagcagaccctacaacatctacccccatggaatcacag
atgtgagacccctgtacagcagaagactgcccaagggggtgaagcacctgaaggacttccccatcctgcctggagagatcttc
aagtacaagtggactgtgacagtagaggatggacccaccaagtcagaccccagatgcctgaccagatactacagcagctttgt
aaacatggagagagacctggccagtggcctgataggccccctgctgatctgctacaaggagtcagtggaccagcagggcaa
ccagatcatgtcagacaagagaaatgtgatcctgttctctgtatttgatgagaacagaagctggtacctgactgaaaacatccag
agattcctgcccaaccctgctggagtacagctggaggaccctgagttccaggccagcaacatcatgcacagcatcaatggttat
gtgtttgacagcctgcagctgtctgtgtgcctgcatgaagtggcctactggtacatcctgagcataggtgcccagactgacttcctgt
ctgtgttcttcagtggctacaccttcaagcacaagatggtgtatgaagacaccctgaccctgttccccttctctggggagacagtatt
catgagcatggagaacccaggactgtggatcctgggctgccacaacagtgacttcagaaacagaggcatgactgctctgctga
aggtgagcagctgtgataagaacacaggtgactactatgaggacagctatgaagacatcagtgcttacctgctgagcaagaac
aatgctatagaacccagaagcttcagccagaaccccccagttctgaagagacaccagagagagatcaccagaaccaccctg
cagtcagaccaggaggagatagactatgatgataccatctcagtagagatgaagaaggaggactttgacatctatgatgagga
tgaaaaccagagccccagaagcttccagaagaagaccagacactacttcatagctgctgtagagagactgtgggactatgga
atgagcagcagcccccatgtgctgagaaacagagcccagtctgggagtgtgccccagttcaagaaggtggtgttccaggagtt
cacagatgggagcttcacccagcccctgtacagaggggagctgaatgaacacctgggcctgctgggcccctacatcagagca
gaggtggaggacaacatcatggtgaccttcagaaaccaggccagcagaccctacagcttctacagcagcctgatcagctatga
ggaggaccagagacagggagctgagcccagaaagaactttgttaagcccaatgaaaccaagacctacttctggaaggtgca
gcaccacatggcccccaccaaggatgagtttgactgcaaggcctgggcctacttctctgatgttgacctggagaaggatgtgcac
tctggactgattgggcccctgctggtgtgccacaccaacaccctgaaccctgcccatgggagacaggtgacagtacaggagttt
gctctgttcttcaccatctttgatgagaccaagagctggtacttcacagaaaacatggagagaaactgcagagccccctgcaaca
tccagatggaggaccccaccttcaaggagaactacagattccatgctatcaatggctacatcatggacaccctgcctggcctggt
gatggcccaggaccagagaatcagatggtacctgctgagcatgggcagcaatgaaaacatccacagcatccacttctctggtc
atgttttcacagttagaaagaaggaggagtacaagatggccctgtacaacctgtaccctggggtgtttgagactgtggagatgctg
cccagcaaggctggaatctggagagtggagtgcctgattggagaacacctgcatgcagggatgagcaccctgttcctggtgtac
agcaacaagtgccagacccccctgggcatggccagtggtcacatcagagacttccagatcacagcctcaggccagtatggac
agtgggcccccaagctggccagactgcactacagtggtagcatcaatgcctggagcaccaaggagcccttcagctggatcaa
ggtggacctgctggcccccatgatcatccatggaatcaagacccagggggccagacagaagttcagcagcctgtacatcagc
cagttcatcatcatgtacagcctggatggcaagaagtggcagacctacagaggcaacagcactggtaccctgatggtgttctttg
gcaatgtggacagctctgggatcaagcacaacatcttcaacccccccatcatagccagatacatcagactgcaccccacccact
acagcatcagaagcaccctgagaatggagctgatgggctgtgacctgaacagctgcagcatgcccctgggcatggagagca
aggccatcagtgatgctcagatcacagcaagcagctacttcaccaacatgtttgccacctggagccccagcaaggccagactg
cacctgcagggcagaagcaatgcttggagaccccaggtgaacaaccccaaggagtggctgcaggtggacttccagaagacc
atgaaggtgactggggtgaccacccagggggtgaagagcctgctgaccagcatgtatgtcaaggagttcctgatcagcagcag
ccaggatgggcaccagtggaccctgttcttccagaatgggaaggtgaaggtgttccagggcaaccaggacagcttcaccccag
tagtgaacagcctggacccccccctgctgaccagatacctgagaatccacccccagagctgggtgcaccagatagccctgag
aatggaggtgctgggctgtgaggcccaggacctgtactga (SEQ ID NO: 66)
FVIII-QQ07
atgcagatagagctgagcacctgcttcttcctgtgcctgctgagattctgcttctcagccaccagaagatactacctgggtgcagtg
gagctgagctgggactacatgcagagtgacctgggggaactgcctgtggatgccagattcccccccagagtgcccaagagctt
ccccttcaacacctctgttgtttacaagaagaccctgtttgtagagttcactgaccacctgttcaacatagccaagcccagaccccc
ctggatgggcctgctgggccccaccatccaggcagaggtgtatgatacagtggtgatcaccctgaagaacatggccagccacc
ctgttagcctgcatgcagtgggggtgagctactggaaggccagtgagggagcagagtatgatgatcagaccagccagagaga
gaaggaggatgacaaggtgttccctgggggcagccacacctatgtatggcaggtgctgaaggagaatggccccatggcctca
gatcccctgtgcctgacctacagctacctgagccatgtggacctggtgaaggacctgaacagtggtctgattggggccctgctggt
gtgcagagagggcagcctggccaaggagaagacccagaccctgcacaagttcatcctgctgtttgctgtctttgatgagggcaa
gagctggcactcagagaccaagaacagcctgatgcaggacagagatgctgcctctgccagagcctggcccaagatgcacac
tgtgaatgggtatgtgaacagaagcctgccaggtctgataggctgccacagaaagtcagtatactggcatgtgattggcatgggc
accaccccagaggtgcacagcatcttcctggagggccacaccttcctggtgagaaaccacagacaggccagcctggagatca
gccccatcaccttcctgactgcccagaccctgctgatggacctgggccagttcctgctgttctgccacatcagcagccaccagcat
gatggcatggaggcctatgttaaggtggacagctgcccagaggagccccagctgcagatgaagaacaatgaagaggctgag
gactatgatgatgatctgacagactcagaaatggatgtagtcagatttgatgatgataacagccccagcttcatccagatcagatc
tgtagccaagaagcaccccaagacctgggtgcactacatagcagcagaggaggaggactgggactatgcacccctggtgct
ggcccctgatgacagaagctacaagagccagtacctgaacaatggcccccagagaataggcagaaagtacaagaaggtga
gattcatggcctacactgatgagaccttcaagaccagagaggccatccagcatgagtctggaatcctgggccccctgctgtatgg
tgaggtgggtgacaccctgctgatcatcttcaagaaccaggccagcagaccctacaacatctacccccatggcatcactgatgtg
agacccctgtacagcagaagactgcccaagggggtgaagcacctgaaggacttccccatcctgccaggtgagatcttcaagta
caagtggactgtgacagtggaggatggccccaccaagagtgaccccagatgcctgaccagatactacagcagctttgtcaaca
tggagagagacctggccagtgggctgatagggcccctgctgatctgctacaaggagtcagtggaccagcagggcaaccagat
catgagtgataagagaaatgtgatcctgttctcagtttttgatgaaaacagaagctggtacctgacagaaaacatccagagattcc
tgcccaacccagcaggggtacagctggaggaccctgaattccaggccagcaacatcatgcacagcatcaatggctatgtatttg
acagcctgcagctgtctgtttgcctgcatgaggtggcctactggtacatcctgagcataggagcccagacagacttcctgagtgtgt
tcttcagtggctacaccttcaagcacaagatggtgtatgaggacaccctgaccctgttccccttctcaggggagactgttttcatgag
catggagaaccctggactgtggatcctgggctgccacaactctgacttcagaaacagaggcatgactgccctgctgaaggtga
gcagctgtgataagaacactggagactactatgaggacagctatgaggacatctcagcctacctgctgagcaagaacaatgcc
attgaacccagaagcttcagccagaacccccctgtactgaagagacaccagagagagatcaccagaaccaccctgcagagt
gaccaggaggagatagactatgatgataccatctctgtggagatgaagaaggaggactttgatatctatgatgaggatgagaac
cagagccccagaagcttccagaagaagaccagacactacttcatagcagcagtggagagactgtgggactatggcatgagc
agcagcccccatgtgctgagaaacagagcccagtcaggcagtgttccccagttcaagaaggtggtgttccaggagttcactgat
ggtagcttcacccagcccctgtacagaggagagctgaatgagcacctgggcctgctgggcccctacatcagagctgaggtgga
ggacaacatcatggtgaccttcagaaaccaggccagcagaccctacagcttctacagcagcctgatcagctatgaggaggac
cagagacagggagcagagcccagaaagaactttgttaagcccaatgaaaccaagacctacttctggaaggtgcagcaccac
atggcccccaccaaggatgagtttgattgcaaggcctgggcctacttcagtgatgtagacctggagaaggatgtgcactcaggc
ctgattggacccctgctggtgtgccacaccaacaccctgaaccctgcacatggaagacaggtgacagtgcaggagtttgccctg
ttcttcaccatctttgatgagaccaagagctggtacttcacagagaacatggagagaaactgcagagccccctgcaacatccag
atggaggaccccaccttcaaggagaactacagattccatgctatcaatggctacatcatggacaccctgcctggcctggtgatgg
cccaggaccagagaatcagatggtacctgctgagcatgggcagcaatgagaacatccacagcatccacttcagtgggcatgt
gttcacagtgagaaagaaggaggagtacaagatggccctgtacaacctgtaccctggagtgtttgagactgtggagatgctgcc
cagcaaggcaggcatctggagagtggagtgcctgataggagaacacctgcatgctggcatgagcaccctgttcctggtgtaca
gcaacaagtgccagacccccctgggcatggccagtggacacatcagagacttccagatcacagcctctggacagtatggaca
gtgggcccccaagctggccagactgcactactcaggaagcatcaatgcatggagcaccaaggagcccttcagctggatcaag
gtggacctgctggcccccatgatcatccatggcatcaagacccagggggcaagacagaagttcagcagcctgtacatcagcc
agttcatcatcatgtacagcctggatggcaagaagtggcagacctacagaggcaacagcactggaaccctgatggtgttctttgg
gaatgtggacagctcagggatcaagcacaacatcttcaacccccccatcattgctagatacatcagactgcaccccacccacta
cagcatcagaagcaccctgagaatggagctgatgggctgtgatctgaacagctgcagcatgcccctgggcatggagagcaag
gccatctcagatgcccagatcactgccagcagctacttcaccaacatgtttgccacctggagccccagcaaggccagactgcac
ctgcagggcagaagcaatgcttggagaccccaggtgaacaaccccaaggagtggctgcaggtggacttccagaagaccatg
aaggtgactggagtaaccacccagggggtgaagagcctgctgaccagcatgtatgtgaaggagttcctgatcagcagcagcc
aggatggtcaccagtggaccctgttcttccagaatggcaaggtgaaggtgttccagggcaaccaggacagcttcacccctgttgt
gaacagcctggacccccccctgctgaccagatacctgagaatccacccccagagctgggtgcaccagattgccctgagaatg
gaggtgctgggctgtgaggcccaggacctgtactga (SEQ ID NO: 67)
FVIII-QQ08
atgcagatagagctgagcacctgcttcttcctgtgcctgctgagattctgcttctctgccaccagaagatactacctgggtgcagtgg
agctgagctgggactacatgcagtcagacctgggagagctgccagtggatgctagattcccccccagagtgcccaagagcttc
cccttcaacacctcagtggtgtacaagaagaccctgtttgtagagttcactgaccacctgttcaacattgccaagcccagaccccc
ctggatgggcctgctgggccccaccatccaggctgaggtgtatgatacagtggtaatcaccctgaagaacatggccagccacc
cagtgagcctgcatgctgttggagttagctactggaaggcctctgagggtgctgagtatgatgatcagaccagccagagagaga
aggaggatgataaggtgttcccagggggaagccacacctatgtatggcaggtgctgaaggagaatgggcccatggccagtga
ccccctgtgcctgacctacagctacctgagccatgtggatctggtgaaggacctgaactcaggtctgataggggccctgctggtgt
gcagagagggcagcctggccaaggagaagacccagaccctgcacaagttcatcctgctgtttgctgtatttgatgagggcaag
agctggcacagtgaaaccaagaacagcctgatgcaggacagagatgctgcctctgccagagcctggcccaagatgcacaca
gtgaatggctatgtgaacagaagcctgcctggtctgatagggtgccacagaaagtcagtgtactggcatgtcattggcatgggca
ccaccccagaggtgcacagcatcttcctggagggccacaccttcctggtgagaaaccacagacaggccagcctggagatcag
ccccatcaccttcctgacagctcagaccctgctgatggacctgggccagttcctgctgttctgccacatcagcagccaccagcatg
atggcatggaggcctatgttaaggtggacagctgcccagaggagccccagctgcagatgaagaacaatgaggaggcagag
gactatgatgatgatctgacagacagtgagatggatgtggtgagatttgatgatgacaacagccccagcttcatccagatcagat
ctgtggccaagaagcaccccaagacctgggtgcactacattgcagctgaagaggaggactgggactatgcccccctggtgctg
gccccagatgatagaagctacaagagccagtacctgaacaatggcccccagagaattggaagaaagtacaagaaggtgag
attcatggcctacactgatgaaaccttcaagaccagagaggccatccagcatgagtcaggcatcctgggccccctgctgtatggt
gaggtgggtgataccctgctgatcatcttcaagaaccaggccagcagaccctacaacatctacccccatggcatcactgatgta
agacccctgtacagcagaagactgcccaagggtgtaaagcacctgaaggacttccccatcctgcctggggaaatcttcaagta
caagtggactgtgactgtagaggatggtcccaccaagagtgaccccagatgcctgaccagatactacagcagctttgtgaacat
ggagagagacctggcctctggtctgataggtcccctgctgatctgctacaaggagtcagtagaccagcagggcaaccagatca
tgtctgacaagagaaatgtgatcctgttcagtgtgtttgatgagaacagaagctggtacctgacagaaaacatccagagattcctg
cccaacccagctggagtgcagctggaggaccctgagttccaggccagcaacatcatgcacagcatcaatgggtatgtctttgac
agcctgcagctgagtgtgtgcctgcatgaggtggcctactggtacatcctgagcattggtgctcagacagatttcctgtctgtgttcttc
agtggttacaccttcaagcacaagatggtgtatgaagacaccctgaccctgttccccttcagtggagagacagtattcatgagcat
ggagaaccctggcctgtggatcctgggctgccacaacagtgatttcagaaacagaggcatgactgcactgctgaaggtgagca
gctgtgacaagaacactggggattactatgaagacagctatgaggacatcagtgcctacctgctgagcaagaacaatgctattg
agcccagaagcttcagccagaaccccccagtcctgaagagacaccagagagagatcaccagaaccaccctgcagagtgac
caggaggagatagactatgatgacaccatctcagtggagatgaagaaggaggactttgacatctatgatgaggatgagaacc
agagccccagaagcttccagaagaagaccagacactacttcattgcagcagtagagagactgtgggactatgggatgagcag
cagcccccatgtactgagaaacagagcccagtctggatctgtgccccagttcaagaaggtggtgttccaggagttcactgatggc
agcttcacccagcccctgtacagaggagagctgaatgagcacctgggcctgctgggcccctacatcagagcagaggtggagg
acaacatcatggtgaccttcagaaaccaggccagcagaccctacagcttctacagcagcctgatcagctatgaggaggacca
gagacagggtgctgagcccagaaagaactttgttaagcccaatgagaccaagacctacttctggaaggtgcagcaccacatg
gcccccaccaaggatgaatttgattgcaaggcctgggcctacttcagtgatgtagacctggagaaggatgtgcacagtggactg
attgggcccctgctggtgtgccacaccaacaccctgaacccagcccatggcagacaggtgactgtgcaggagtttgctctgttctt
caccatctttgatgaaaccaagagctggtacttcacagagaacatggagagaaactgcagagccccctgcaacatccagatg
gaggaccccaccttcaaggagaactacagattccatgctatcaatggctacatcatggacaccctgccaggtctggtgatggcc
caggaccagagaatcagatggtacctgctgagcatgggcagcaatgagaacatccacagcatccacttctctggccatgtcttc
actgtgagaaagaaggaggagtacaagatggccctgtacaacctgtacccaggtgtctttgagactgtagagatgctgcccagc
aaggctggcatctggagagtggagtgcctgattggagagcacctgcatgcaggaatgagcaccctgttcctggtgtacagcaac
aagtgccagacccccctgggcatggccagtggccacatcagagacttccagatcacagcctctgggcagtatggacagtggg
cccccaagctggccagactgcactactcaggaagcatcaatgcctggagcaccaaggagcccttcagctggatcaaggtgga
cctgctggcccccatgatcatccatgggatcaagacccagggggccagacagaagttcagcagcctgtacatcagccagttcat
catcatgtacagcctggatggtaagaagtggcagacctacagaggcaacagcacaggcaccctgatggtgttctttggaaatgt
agacagctctggcatcaagcacaacatcttcaacccccccatcatagccagatacatcagactgcaccccacccactacagca
tcagaagcaccctgagaatggagctgatgggctgtgacctgaacagctgcagcatgcccctgggcatggagagcaaggccat
ctctgatgctcagatcactgccagcagctacttcaccaacatgtttgcaacctggagccccagcaaggccagactgcacctgca
gggcagaagcaatgcctggagaccccaggtgaacaaccccaaggagtggctgcaggtggacttccagaagaccatgaagg
tgactggtgtaaccacccagggagtgaagagcctgctgaccagcatgtatgtcaaggagttcctgatcagcagcagccaggat
ggtcaccagtggaccctgttcttccagaatggaaaggtgaaggtgttccagggcaaccaggacagcttcaccccagttgtgaac
agcctggacccccccctgctgaccagatacctgagaatccacccccagagctgggtgcaccagattgccctgagaatggaggt
gctgggctgtgaggcccaggacctgtactga (SEQ ID NO: 68)
FVIII-QQ09
atgcagatagagctgagcacctgcttcttcctgtgcctgctgagattctgcttcagtgcaaccagaagatactacctgggtgcagtg
gagctgagctgggactacatgcagtctgatctgggggagctgccagttgatgccagattcccccccagagtgcccaagagcttc
cccttcaacaccagtgttgtttacaagaagaccctgtttgtggaattcactgaccacctgttcaacatagccaagcccagaccccc
ctggatgggcctgctgggccccaccatccaggcagaggtgtatgacacagtagtgatcaccctgaagaacatggccagccac
cctgtgagcctgcatgcagtaggggtaagctactggaaggcctctgaaggtgctgaatatgatgaccagaccagccagagaga
gaaggaggatgacaaggtgttcccaggtggaagccacacctatgtctggcaggtgctgaaggagaatggccccatggccagt
gaccccctgtgcctgacctacagctacctgagccatgtggacctggtgaaggacctgaactcaggtctgattggagcactgctgg
tgtgcagagagggcagcctggccaaggagaagacccagaccctgcacaagttcatcctgctgtttgcagtgtttgatgaaggca
agagctggcacagtgagaccaagaacagcctgatgcaggacagagatgcagccagtgccagagcctggcccaagatgca
cactgtgaatggttatgtcaacagaagcctgccagggctgataggctgccacagaaagagtgtttactggcatgtaattgggatg
ggcaccaccccagaggtgcacagcatcttcctggagggccacaccttcctggtgagaaaccacagacaggccagcctggag
atcagccccatcaccttcctgacagctcagaccctgctgatggacctgggccagttcctgctgttctgccacatcagcagccacca
gcatgatggcatggaggcctatgtaaaggtggacagctgccctgaggagccccagctgcagatgaagaacaatgaggaggc
agaggactatgatgatgacctgactgacagtgagatggatgtggtgagatttgatgatgacaacagccccagcttcatccagatc
agaagtgttgctaagaagcaccccaagacctgggtgcactacattgcagcagaggaggaggactgggactatgctcccctggt
gctggcccctgatgacagaagctacaagagccagtacctgaacaatggcccccagagaattggcagaaagtacaagaaggt
gagattcatggcctacactgatgagaccttcaagaccagagaggccatccagcatgaaagtggcatcctgggccccctgctgta
tggagaggtgggagacaccctgctgatcatcttcaagaaccaggccagcagaccctacaacatctacccccatggtatcacag
atgtgagacccctgtacagcagaagactgcccaagggggtgaagcacctgaaggacttccccatcctgccaggtgaaatcttc
aagtacaagtggacagtcacagtggaggatggccccaccaagagtgaccccagatgcctgaccagatactacagcagctttg
tgaacatggagagagacctggcctctggcctgataggccccctgctgatctgctacaaggagtcagtggaccagcagggcaac
cagatcatgagtgataagagaaatgtgatcctgttctcagtatttgatgagaacagaagctggtacctgactgaaaacatccaga
gattcctgcccaaccctgcaggggtgcagctggaggaccctgaattccaggccagcaacatcatgcacagcatcaatggctat
gtatttgatagcctgcagctgagtgtgtgcctgcatgaggtggcctactggtacatcctgagcataggggcccagacagacttcct
gtctgtgttcttctcaggttacaccttcaagcacaagatggtgtatgaagacaccctgaccctgttccccttcagtggggaaacagta
ttcatgagcatggagaaccctgggctgtggatcctgggctgccacaactcagatttcagaaacagaggcatgactgccctgctg
aaggtgagcagctgtgacaagaacactggagactactatgaggacagctatgaggacatcagtgcctacctgctgagcaaga
acaatgctatagagcccagaagcttcagccagaaccccccagtcctgaagagacaccagagagagatcaccagaaccacc
ctgcagtcagatcaggaggagatagactatgatgacaccatcagtgtggagatgaagaaggaggactttgacatctatgatga
ggatgagaaccagagccccagaagcttccagaagaagaccagacactacttcatagctgctgtagagagactgtgggactat
ggcatgagcagcagcccccatgtcctgagaaacagagcccagtctggctctgttccccagttcaagaaggtggtgttccaggag
ttcacagatggcagcttcacccagcccctgtacagaggggagctgaatgaacacctgggcctgctgggcccctacatcagagct
gaagtggaggacaacatcatggtgaccttcagaaaccaggccagcagaccctacagcttctacagcagcctgatcagctatga
ggaggaccagagacagggtgcagaacccagaaagaactttgttaagcccaatgagaccaagacctacttctggaaggtgca
gcaccacatggcccccaccaaggatgaatttgactgcaaggcctgggcctacttctcagatgttgacctggagaaggatgtcca
ctcaggactgatagggcccctgctggtgtgccacaccaacaccctgaacccagcccatggcagacaggtgactgtgcaggagt
ttgctctgttcttcaccatctttgatgagaccaagagctggtacttcacagagaacatggagagaaactgcagagccccctgcaac
atccagatggaggaccccaccttcaaggagaactacagattccatgctatcaatggatacatcatggacaccctgccaggcctg
gtgatggcccaggaccagagaatcagatggtacctgctgagcatgggcagcaatgagaacatccacagcatccacttcagtg
gtcatgtgttcacagtaagaaagaaggaggagtacaagatggccctgtacaacctgtacccaggggtgtttgaaactgtggaaa
tgctgcccagcaaggcagggatctggagagtggagtgcctgattggagaacacctgcatgcaggcatgagcaccctgttcctg
gtgtacagcaacaagtgccagacccccctgggcatggccagtgggcacatcagagacttccagatcacagcctctgggcagt
atggccagtgggcccccaagctggccagactgcactactcaggcagcatcaatgcctggagcaccaaggagcccttcagctg
gatcaaggtggacctgctggcccccatgatcatccatggcatcaagacccagggagcaagacagaagttcagcagcctgtac
atcagccagttcatcatcatgtacagcctggatgggaagaagtggcagacctacagaggcaacagcactgggaccctgatggt
gttctttggcaatgtagacagctctggcatcaagcacaacatcttcaacccccccatcattgcaagatacatcagactgcacccca
cccactacagcatcagaagcaccctgagaatggagctgatgggctgtgacctgaacagctgcagcatgcccctgggcatgga
gagcaaggccatcagtgatgcccagatcactgctagcagctacttcaccaacatgtttgccacctggagccccagcaaggcca
gactgcacctgcagggcagaagcaatgcatggagaccccaggtgaacaaccccaaggagtggctgcaggtggacttccag
aagaccatgaaggtgacaggggttaccacccagggtgtgaagagcctgctgaccagcatgtatgtgaaggagttcctgatcag
cagcagccaggatggccaccagtggaccctgttcttccagaatgggaaggtgaaggtgttccagggcaaccaggacagcttc
acccctgtggtgaacagcctggacccccccctgctgaccagatacctgagaatccacccccagagctgggtgcaccagattgc
cctgagaatggaggtgctgggctgtgaggcccaggacctgtactga (SEQ ID NO: 69)
FVIII-QQ10
atgcagatagaactgagcacctgcttcttcctgtgcctgctgagattctgcttctctgccaccagaagatactacctgggggctgtgg
agctgagctgggactacatgcagagtgacctgggagagctgccagttgatgcaagattcccccccagagtgcccaagagcttc
cccttcaacacctctgtggtgtacaagaagaccctgtttgtggagttcactgatcacctgttcaacatagccaagcccagaccccc
ctggatgggcctgctgggccccaccatccaggctgaagtgtatgacactgtagtgatcaccctgaagaacatggccagccaccc
tgtgagcctgcatgctgtgggagtgagctactggaaggcctcagagggagcagagtatgatgaccagaccagccagagaga
gaaggaggatgacaaggtgttcccaggaggcagccacacctatgtttggcaggtgctgaaggagaatgggcccatggcctca
gaccccctgtgcctgacctacagctacctgagccatgtagacctggtgaaggacctgaactctggcctgataggagccctgctgg
tgtgcagagagggcagcctggccaaggagaagacccagaccctgcacaagttcatcctgctgtttgcagtgtttgatgagggca
agagctggcactcagagaccaagaacagcctgatgcaggacagagatgcagccagtgctagagcctggcccaagatgcac
acagtgaatgggtatgttaacagaagcctgccaggcctgataggatgccacagaaagtctgtgtactggcatgtgattggtatgg
gcaccaccccagaggtgcacagcatcttcctggagggccacaccttcctggtgagaaaccacagacaggccagcctggagat
cagccccatcaccttcctgacagcccagaccctgctgatggacctgggccagttcctgctgttctgccacatcagcagccaccag
catgatggcatggaggcctatgttaaggtggacagctgcccagaggagccccagctgcagatgaagaacaatgaagaggca
gaggactatgatgatgacctgacagattcagagatggatgtggtgagatttgatgatgacaacagccccagcttcatccagatca
gaagtgtggccaagaagcaccccaagacctgggtgcactacattgctgctgaagaggaggactgggactatgcccccctggt
gctggccccagatgacagaagctacaagagccagtacctgaacaatgggccccagagaattggaagaaagtacaagaagg
tgagattcatggcctacacagatgaaaccttcaagaccagagaggccatccagcatgagagtgggatcctgggccccctgctgt
atggggaggtgggtgataccctgctgatcatcttcaagaaccaggccagcagaccctacaacatctacccccatggcatcaca
gatgtgagacccctgtacagcagaagactgcccaagggagttaagcacctgaaggacttccccatcctgccaggagagatctt
caagtacaagtggacagtcacagtggaggatgggcccaccaagtctgaccccagatgcctgaccagatactacagcagctttg
taaacatggagagagacctggcctcaggtctgataggccccctgctgatctgctacaaggagagtgttgatcagcagggcaac
cagatcatgtctgacaagagaaatgtgatcctgttctcagtgtttgatgaaaacagaagctggtacctgactgagaacatccaga
gattcctgcccaacccagctggggtgcagctggaggaccctgagttccaggccagcaacatcatgcacagcatcaatggatat
gtgtttgacagcctgcagctgtcagtgtgcctgcatgaggtggcctactggtacatcctgagcataggagctcagacagacttcctg
agtgtattcttctcagggtacaccttcaagcacaagatggtgtatgaggacaccctgaccctgttccccttctcaggtgagacagtgt
tcatgagcatggagaacccagggctgtggatcctgggctgccacaactctgatttcagaaacagaggcatgactgccctgctga
aggtgagcagctgtgacaagaacacaggggactactatgaggacagctatgaggacatctctgcctacctgctgagcaagaa
caatgctattgagcccagaagcttcagccagaacccccctgtactgaagagacaccagagagagatcaccagaaccaccctg
cagagtgatcaggaggagatagattatgatgacaccatctcagtggagatgaagaaggaggactttgatatctatgatgaggat
gaaaaccagagccccagaagcttccagaagaagaccagacactacttcattgcagctgtagagagactgtgggactatggtat
gagcagcagcccccatgtcctgagaaacagagcccagtctggatcagtcccccagttcaagaaggtggtgttccaggagttca
cagatggtagcttcacccagcccctgtacagaggagagctgaatgagcacctgggcctgctgggcccctacatcagagcaga
ggtggaggacaacatcatggtgaccttcagaaaccaggccagcagaccctacagcttctacagcagcctgatcagctatgaag
aggaccagagacagggagcagagcccagaaagaactttgtgaagcccaatgagaccaagacctacttctggaaggtgcag
caccacatggcccccaccaaggatgagtttgattgcaaggcctgggcctacttcagtgatgttgacctggagaaggatgttcaca
gtggcctgataggtcccctgctggtgtgccacaccaacaccctgaacccagcccatggcagacaggtgactgtccaggagtttg
ccctgttcttcaccatctttgatgagaccaagagctggtacttcactgaaaacatggagagaaactgcagagccccctgcaacat
ccagatggaggaccccaccttcaaggagaactacagattccatgccatcaatggctacatcatggacaccctgcctggactggt
gatggcccaggaccagagaatcagatggtacctgctgagcatgggcagcaatgagaacatccacagcatccacttctcaggc
catgtattcactgtgagaaagaaggaggagtacaagatggccctgtacaacctgtacccaggtgtgtttgagacagtggagatg
ctgcccagcaaggcaggtatctggagagtggagtgcctgataggagaacacctgcatgctggcatgagcaccctgttcctggtg
tacagcaacaagtgccagacccccctgggcatggccagtggccacatcagagacttccagatcacagcctcaggccagtatg
ggcagtgggcccccaagctggccagactgcactactctggcagcatcaatgcatggagcaccaaggagcccttcagctggatc
aaggtggacctgctggcccccatgatcatccatggaatcaagacccagggtgctagacagaagttcagcagcctgtacatcag
ccagttcatcatcatgtacagcctggatggcaagaagtggcagacctacagaggcaacagcactggcaccctgatggtgttcttt
ggcaatgttgacagctctggcatcaagcacaacatcttcaacccccccatcatagcaagatacatcagactgcaccccacccac
tacagcatcagaagcaccctgagaatggagctgatgggctgtgatctgaacagctgcagcatgcccctgggcatggagagca
aggccatcagtgatgctcagatcactgccagcagctacttcaccaacatgtttgcaacctggagccccagcaaggccagactgc
acctgcagggcagaagcaatgcctggagaccccaggtgaacaaccccaaggagtggctgcaggtggacttccagaagacc
atgaaggtgacaggtgttaccacccagggtgtcaagagcctgctgaccagcatgtatgtaaaggagttcctgatcagcagcagc
caggatggccaccagtggaccctgttcttccagaatggaaaggtgaaggtgttccagggcaaccaggacagcttcacccctgtg
gtgaacagcctggacccccccctgctgaccagatacctgagaatccacccccagagctgggtgcaccagatagccctgagaa
tggaggtgctgggctgtgaagcccaggacctgtactga (seq id no: 70)
FVIII-QQ11
atgcagatagagctgagcacctgcttcttcctgtgcctgctgagattctgcttctctgccaccagaagatactacctgggtgctgttga
gctgagctgggactacatgcagtctgatctgggtgagctgcctgttgatgccagattcccccccagagtgcccaagagcttcccctt
caacacctcagttgtgtacaagaagaccctgtttgttgagttcacagaccacctgttcaacattgctaagcccagacccccctggat
gggcctgctgggccccaccatccaggcagaggtgtatgatacagttgtgatcaccctgaagaacatggccagccacccagtga
gcctgcatgcagtaggtgtgagctactggaaggccagtgagggggctgaatatgatgatcagaccagccagagagagaagg
aggatgacaaggtgttcccaggtggtagccacacctatgtatggcaggtgctgaaggagaatggccccatggccagtgacccc
ctgtgcctgacctacagctacctgagccatgtggacctggtgaaggacctgaactctggactgattggagcactgctggtgtgca
gagagggcagcctggccaaggagaagacccagaccctgcacaagttcatcctgctgtttgcagtgtttgatgagggcaagagc
tggcactcagagaccaagaacagcctgatgcaggacagagatgcagcttctgccagagcctggcccaagatgcacactgtca
atggctatgtgaacagaagcctgccaggcctgattggttgccacagaaagagtgtgtactggcatgttatagggatgggcacca
cccctgaggtgcacagcatcttcctggagggccacaccttcctggtgagaaaccacagacaggccagcctggagatcagccc
catcaccttcctgacagcacagaccctgctgatggacctgggccagttcctgctgttctgccacatcagcagccaccagcatgat
ggcatggaggcctatgttaaggtggacagctgccctgaggagccccagctgcagatgaagaacaatgaggaggctgaggac
tatgatgatgacctgacagactcagagatggatgtggtgagatttgatgatgataacagccccagcttcatccagatcagatcagt
ggccaagaagcaccccaagacctgggtgcactacatagctgctgaagaggaggactgggactatgcccccctggtgctggcc
ccagatgacagaagctacaagagccagtacctgaacaatgggccccagagaattggcagaaagtacaagaaggtgagatt
catggcctacactgatgagaccttcaagaccagagaggccatccagcatgagtctggaatcctgggccccctgctgtatggaga
ggtgggtgacaccctgctgatcatcttcaagaaccaggccagcagaccctacaacatctacccccatggcatcactgatgttaga
cccctgtacagcagaagactgcccaagggtgtgaagcacctgaaggacttccccatcctgcctggggagatcttcaagtacaa
gtggacagtcactgtagaggatggccccaccaagagtgaccccagatgcctgaccagatactacagcagctttgtgaacatgg
agagagacctggcctctggactgattggacccctgctgatctgctacaaggagagtgtggaccagcagggcaaccagatcatg
tcagataagagaaatgtcatcctgttctcagtatttgatgagaacagaagctggtacctgactgagaacatccagagattcctgcc
caacccagctggggtgcagctggaggaccctgaattccaggccagcaacatcatgcacagcatcaatggctatgtatttgatag
cctgcagctgtctgtgtgcctgcatgaggtggcctactggtacatcctgagcattggggcccagactgatttcctgtctgtcttcttctct
gggtacaccttcaagcacaagatggtgtatgaggacaccctgaccctgttccccttctcaggagagacagtgttcatgagcatgg
agaacccaggcctgtggatcctgggctgccacaactcagacttcagaaacagaggcatgactgccctgctgaaggtgagcag
ctgtgacaagaacacaggtgactactatgaggacagctatgaagacatctctgcctacctgctgagcaagaacaatgctataga
acccagaagcttcagccagaacccccctgtgctgaagagacaccagagagagatcaccagaaccaccctgcagtcagatca
ggaggagatagattatgatgataccatctcagtagaaatgaagaaggaggactttgacatctatgatgaagatgaaaaccaga
gccccagaagcttccagaagaagaccagacactacttcatagctgctgtagagagactgtgggactatggcatgagcagcag
cccccatgtactgagaaacagagcccagagtggctcagtgccccagttcaagaaggtggtgttccaggagttcactgatggca
gcttcacccagcccctgtacagaggtgaactgaatgagcacctgggcctgctgggcccctacatcagagcagaggtggagga
caacatcatggtgaccttcagaaaccaggccagcagaccctacagcttctacagcagcctgatcagctatgaagaggaccag
agacagggtgctgagcccagaaagaactttgtgaagcccaatgagaccaagacctacttctggaaggtgcagcaccacatgg
cccccaccaaggatgagtttgactgcaaggcctgggcctacttcagtgatgtagacctggagaaggatgtgcacagtgggctga
taggtcccctgctggtgtgccacaccaacaccctgaacccagcacatggtagacaggtgacagttcaggagtttgccctgttcttc
accatctttgatgaaaccaagagctggtacttcactgagaacatggagagaaactgcagagccccctgcaacatccagatgga
ggaccccaccttcaaggagaactacagattccatgccatcaatggctacatcatggacaccctgccaggcctggtgatggccca
ggaccagagaatcagatggtacctgctgagcatgggcagcaatgagaacatccacagcatccacttctcaggacatgtgttca
ctgtgagaaagaaggaggagtacaagatggccctgtacaacctgtacccaggggtatttgaaacagtagagatgctgcccag
caaggcaggcatctggagagtggagtgcctgattggagaacacctgcatgcaggtatgagcaccctgttcctggtgtacagcaa
caagtgccagacccccctgggcatggcctcaggccacatcagagacttccagatcacagcttctggccagtatgggcagtggg
cccccaagctggccagactgcactactcaggtagcatcaatgcttggagcaccaaggagcccttcagctggatcaaggtggac
ctgctggcccccatgatcatccatgggatcaagacccagggagccagacagaagttcagcagcctgtacatcagccagttcatc
atcatgtacagcctggatggtaagaagtggcagacctacagaggcaacagcactggcaccctgatggtgttctttggaaatgttg
atagctcagggatcaagcacaacatcttcaacccccccatcatagccagatacatcagactgcaccccacccactacagcatc
agaagcaccctgagaatggagctgatgggctgtgatctgaacagctgcagcatgcccctgggcatggagagcaaggccatct
ctgatgcacagatcacagcaagcagctacttcaccaacatgtttgccacctggagccccagcaaggccagactgcacctgcag
ggcagaagcaatgcttggagaccccaggtgaacaaccccaaggagtggctgcaggtggacttccagaagaccatgaaggtg
acaggggtgaccacccagggggtgaagagcctgctgaccagcatgtatgtcaaggagttcctgatcagcagcagccaggatg
gccaccagtggaccctgttcttccagaatgggaaggtgaaggtgttccagggcaaccaggacagcttcaccccagtagtgaac
agcctggacccccccctgctgaccagatacctgagaatccacccccagagctgggtgcaccagattgctctgagaatggaggt
gctgggctgtgaggcccaggacctgtactga (SEQ ID NO: 71)
FVIII-QQ12
atgcagatagaactgagcacctgcttcttcctgtgcctgctgagattctgcttctctgccaccagaagatactacctgggggctgtgg
agctgagctgggactacatgcagtctgacctgggagaactgcctgttgatgctagattcccccccagagtgcccaagagcttccc
cttcaacacctcagtggtgtacaagaagaccctgtttgtagagttcactgaccacctgttcaacattgcaaagcccagacccccct
ggatgggcctgctgggccccaccatccaggctgaggtgtatgatactgttgtgatcaccctgaagaacatggccagccaccctgt
gagcctgcatgctgtgggagtcagctactggaaggcctcagagggggctgagtatgatgaccagaccagccagagagagaa
ggaggatgacaaggtgttccctggtgggagccacacctatgtgtggcaggtgctgaaggagaatggacccatggcctcagacc
ccctgtgcctgacctacagctacctgagccatgtagacctggtgaaggacctgaacagtggtctgataggtgcactgctggtgtgc
agagagggcagcctggccaaggagaagacccagaccctgcacaagttcatcctgctgtttgcagtgtttgatgagggcaagag
ctggcactctgagaccaagaacagcctgatgcaggacagagatgctgcctcagccagagcctggcccaagatgcacactgtt
aatgggtatgtgaacagaagcctgccaggcctgattggctgccacagaaagagtgtgtactggcatgtcataggaatgggcac
cacccctgaggtgcacagcatcttcctggagggccacaccttcctggtgagaaaccacagacaggccagcctggagatcagc
cccatcaccttcctgactgcacagaccctgctgatggacctgggccagttcctgctgttctgccacatcagcagccaccagcatga
tgggatggaggcctatgtaaaggtggacagctgccctgaagagccccagctgcagatgaagaacaatgaggaggcagaag
actatgatgatgatctgacagacagtgaaatggatgtggtgagatttgatgatgacaacagccccagcttcatccagatcagaag
tgtggccaagaagcaccccaagacctgggtgcactacattgctgcagaggaggaggactgggactatgcccccctggtgctgg
ccccagatgacagaagctacaagagccagtacctgaacaatgggccccagagaatagggagaaagtacaagaaggtgag
attcatggcctacacagatgagaccttcaagaccagagaggccatccagcatgagagtggtatcctgggccccctgctgtatgg
agaagtgggggataccctgctgatcatcttcaagaaccaggccagcagaccctacaacatctacccccatggcatcacagatg
tgagacccctgtacagcagaagactgcccaagggtgtaaagcacctgaaggacttccccatcctgccaggggaaatcttcaag
tacaagtggactgtgactgtggaggatggacccaccaagagtgatcccagatgcctgaccagatactacagcagctttgttaac
atggagagagacctggcctcaggactgattggccccctgctgatctgctacaaggagagtgtggaccagcagggcaaccaga
tcatgagtgacaagagaaatgtcatcctgttcagtgtctttgatgaaaacagaagctggtacctgacagaaaacatccagagatt
cctgcccaaccctgcaggtgtgcagctggaggacccagagttccaggccagcaacatcatgcacagcatcaatgggtatgtgtt
tgatagcctgcagctgagtgtatgcctgcatgaggtggcctactggtacatcctgagcataggtgctcagactgacttcctgtcagt
attcttctcaggctacaccttcaagcacaagatggtgtatgaggacaccctgaccctgttccccttcagtggggaaacagtgttcat
gagcatggagaaccctgggctgtggatcctgggctgccacaacagtgacttcagaaacagaggcatgacagccctgctgaag
gtgagcagctgtgacaagaacacaggagactactatgaagacagctatgaagacatcagtgcttacctgctgagcaagaaca
atgcaatagagcccagaagcttcagccagaaccccccagtgctgaagagacaccagagagagatcaccagaaccaccctg
cagtcagaccaggaggagattgactatgatgacaccatctctgtggagatgaagaaggaggactttgacatctatgatgaggat
gagaaccagagccccagaagcttccagaagaagaccagacactacttcatagcagcagtggagagactgtgggactatggg
atgagcagcagcccccatgtcctgagaaacagagcccagtcaggaagtgtcccccagttcaagaaggtggtgttccaggagtt
cactgatgggagcttcacccagcccctgtacagaggagagctgaatgaacacctgggcctgctgggcccctacatcagagca
gaggtggaggacaacatcatggtgaccttcagaaaccaggccagcagaccctacagcttctacagcagcctgatcagctatga
ggaggaccagagacagggagctgaacccagaaagaactttgtgaagcccaatgaaaccaagacctacttctggaaggtgca
gcaccacatggcccccaccaaggatgagtttgactgcaaggcctgggcctacttctctgatgttgatctggagaaggatgtacact
ctgggctgattggccccctgctggtgtgccacaccaacaccctgaacccagcccatggcagacaggtgactgtgcaggagtttg
ccctgttcttcaccatctttgatgagaccaagagctggtacttcactgaaaacatggagagaaactgcagagccccctgcaacat
ccagatggaggaccccaccttcaaggagaactacagattccatgctatcaatggctacatcatggacaccctgcctggactggt
gatggcccaggaccagagaatcagatggtacctgctgagcatgggcagcaatgaaaacatccacagcatccacttctcaggc
catgtattcactgtgagaaagaaggaggagtacaagatggccctgtacaacctgtacccaggtgtgtttgaaacagtggaaatg
ctgcccagcaaggctggcatctggagagtggagtgcctgattggtgagcacctgcatgctggaatgagcaccctgttcctggtgt
acagcaacaagtgccagacccccctgggcatggcctcagggcacatcagagacttccagatcactgcctctggccagtatgga
cagtgggcccccaagctggccagactgcactacagtggcagcatcaatgcctggagcaccaaggagcccttcagctggatca
aggtggacctgctggcccccatgatcatccatggcatcaagacccagggtgccagacagaagttcagcagcctgtacatcagc
cagttcatcatcatgtacagcctggatggcaagaagtggcagacctacagaggcaacagcacaggtaccctgatggtgttctttg
gcaatgtggacagctctggtatcaagcacaacatcttcaacccccccatcatagccagatacatcagactgcaccccacccact
acagcatcagaagcaccctgagaatggagctgatgggctgtgatctgaacagctgcagcatgcccctgggcatggagagcaa
ggccatctctgatgctcagatcacagctagcagctacttcaccaacatgtttgccacctggagccccagcaaggccagactgca
cctgcagggcagaagcaatgcatggagaccccaggtgaacaaccccaaggagtggctgcaggtggacttccagaagacca
tgaaggtgacaggagtgaccacccagggagtgaagagcctgctgaccagcatgtatgtgaaggagttcctgatcagcagcag
ccaggatggccaccagtggaccctgttcttccagaatgggaaggtgaaggtgttccagggcaaccaggacagcttcaccccag
tagtgaacagcctggacccccccctgctgaccagatacctgagaatccacccccagagctgggtgcaccagatagctctgaga
atggaggtgctgggctgtgaagcccaggacctgtactga (SEQ ID NO: 72)
FVIII-QQ13
atgcagatagaactgagcacctgcttcttcctgtgcctgctgagattctgcttcagtgccaccagaagatactacctgggtgctgttg
agctgagctgggactacatgcagtctgacctgggtgaactgccagtagatgcaagattcccccccagagtgcccaagagcttcc
ccttcaacacctctgtagtctacaagaagaccctgtttgtggagttcacagatcacctgttcaacattgctaagcccagacccccct
ggatgggcctgctgggccccaccatccaggcagaggtgtatgacactgtagtgatcaccctgaagaacatggccagccaccc
agtgagcctgcatgctgtgggtgtgagctactggaaggccagtgaaggagcagagtatgatgatcagaccagccagagagag
aaggaggatgacaaggtgttcccaggtggaagccacacctatgtctggcaggtgctgaaggagaatgggcccatggcctcag
atcccctgtgcctgacctacagctacctgagccatgttgacctggtgaaggacctgaacagtggactgataggtgctctgctggtgt
gcagagagggcagcctggccaaggagaagacccagaccctgcacaagttcatcctgctgtttgcagtgtttgatgaaggcaag
agctggcacagtgagaccaagaacagcctgatgcaggacagagatgcagcatcagccagagcctggcccaagatgcacac
agtgaatggctatgtgaacagaagcctgccaggcctgataggatgccacagaaagtctgtatactggcatgtcattggtatgggc
accacccctgaagtgcacagcatcttcctggagggccacaccttcctggtgagaaaccacagacaggccagcctggagatca
gccccatcaccttcctgacagcccagaccctgctgatggacctgggccagttcctgctgttctgccacatcagcagccaccagca
tgatgggatggaggcctatgtcaaggtggacagctgcccagaggagccccagctgcagatgaagaacaatgaggaggctga
ggactatgatgatgacctgactgactcagagatggatgtggtgagatttgatgatgataacagccccagcttcatccagatcagat
ctgtggccaagaagcaccccaagacctgggtgcactacatagctgcagaagaggaggactgggactatgcccccctggtgct
ggccccagatgatagaagctacaagagccagtacctgaacaatggcccccagagaattgggagaaagtacaagaaggtga
gattcatggcctacacagatgaaaccttcaagaccagagaggccatccagcatgagtctggcatcctgggccccctgctgtatg
gggaagtgggagacaccctgctgatcatcttcaagaaccaggccagcagaccctacaacatctacccccatggtatcacagat
gtgagacccctgtacagcagaagactgcccaagggggtgaagcacctgaaggacttccccatcctgcctggggagatcttcaa
gtacaagtggacagtaactgtggaggatggccccaccaagtctgaccccagatgcctgaccagatactacagcagctttgtga
acatggagagagacctggccagtggcctgattggccccctgctgatctgctacaaggagagtgttgaccagcagggcaacca
gatcatgagtgataagagaaatgttatcctgttcagtgtgtttgatgaaaacagaagctggtacctgacagagaacatccagaga
ttcctgcccaacccagctggtgtccagctggaggaccctgagttccaggccagcaacatcatgcacagcatcaatggctatgtatt
tgacagcctgcagctgagtgtgtgcctgcatgaagtggcctactggtacatcctgagcataggtgctcagacagacttcctgagtg
tcttcttcagtggctacaccttcaagcacaagatggtgtatgaagacaccctgaccctgttccccttcagtggagagacagtgttcat
gagcatggagaacccaggactgtggatcctgggctgccacaacagtgacttcagaaacagaggcatgacagccctgctgaa
ggtgagcagctgtgacaagaacactggggactactatgaggacagctatgaggacatctctgcctacctgctgagcaagaaca
atgcaatagagcccagaagcttcagccagaacccccctgtgctgaagagacaccagagagagatcaccagaaccaccctg
cagtcagatcaggaggagatagactatgatgataccatctctgtggagatgaagaaggaggactttgatatctatgatgaggatg
agaaccagagccccagaagcttccagaagaagaccagacactacttcatagctgcagtggagagactgtgggactatggcat
gagcagcagcccccatgtactgagaaacagagcccagtcaggatctgtcccccagttcaagaaggtggtgttccaggagttca
ctgatgggagcttcacccagcccctgtacagaggtgaactgaatgaacacctgggcctgctgggcccctacatcagagcagag
gtggaggacaacatcatggtgaccttcagaaaccaggccagcagaccctacagcttctacagcagcctgatcagctatgaaga
ggaccagagacagggagctgagcccagaaagaactttgtgaagcccaatgaaaccaagacctacttctggaaggtgcagca
ccacatggcccccaccaaggatgaatttgactgcaaggcctgggcctacttctcagatgtagacctggagaaggatgtgcactc
aggactgattggccccctgctggtgtgccacaccaacaccctgaacccagcacatggcagacaggtgacagttcaggagtttg
cactgttcttcaccatctttgatgagaccaagagctggtacttcacagagaacatggagagaaactgcagagccccctgcaacat
ccagatggaggaccccaccttcaaggagaactacagattccatgctatcaatggctacatcatggacaccctgccagggctggt
gatggcccaggaccagagaatcagatggtacctgctgagcatgggcagcaatgaaaacatccacagcatccacttcagtggc
catgtgttcacagttagaaagaaggaggagtacaagatggccctgtacaacctgtaccctggtgtgtttgaaacagtggagatgc
tgcccagcaaggctggcatctggagagtggagtgcctgattggggaacacctgcatgcaggaatgagcaccctgttcctggtgt
acagcaacaagtgccagacccccctgggcatggcctcaggccacatcagagacttccagatcactgccagtgggcagtatggt
cagtgggcccccaagctggccagactgcactactctggcagcatcaatgcctggagcaccaaggagcccttcagctggatcaa
ggtggacctgctggcccccatgatcatccatggcatcaagacccagggggccagacagaagttcagcagcctgtacatcagcc
agttcatcatcatgtacagcctggatggcaagaagtggcagacctacagaggcaacagcactggaaccctgatggtgttctttgg
caatgtagacagctcaggcatcaagcacaacatcttcaacccccccatcattgccagatacatcagactgcaccccacccacta
cagcatcagaagcaccctgagaatggagctgatgggctgtgacctgaacagctgcagcatgcccctgggcatggagagcaa
ggccatctctgatgcccagatcactgccagcagctacttcaccaacatgtttgccacctggagccccagcaaggccagactgca
cctgcagggcagaagcaatgcttggagaccccaggtgaacaaccccaaggagtggctgcaggtggacttccagaagaccat
gaaggtgactggtgtgaccacccagggtgtgaagagcctgctgaccagcatgtatgtcaaggagttcctgatcagcagcagcc
aggatggtcaccagtggaccctgttcttccagaatggcaaggtgaaggtgttccagggcaaccaggacagcttcaccccagtg
gtgaacagcctggacccccccctgctgaccagatacctgagaatccacccccagagctgggtgcaccagatagccctgagaa
tggaggtgctgggctgtgaggcccaggacctgtactga (SEQ ID NO: 73)
FVIII-QQ14
atgcagattgaactgagcacctgcttcttcctgtgcctgctgagattctgcttcagtgccaccagaagatactacctgggggcagtg
gagctgagctgggactacatgcagagtgacctgggggaactgcctgttgatgccagattcccccccagagtgcccaagagcttc
cccttcaacacctcagttgtgtacaagaagaccctgtttgtggagttcactgaccacctgttcaacattgctaagcccagacccccc
tggatgggcctgctgggccccaccatccaggctgaggtgtatgacactgtggtgatcaccctgaagaacatggccagccaccct
gtgagcctgcatgcagtgggggtgagctactggaaggcctcagaaggggcagagtatgatgatcagaccagccagagagag
aaggaggatgacaaggtgttccctggtggtagccacacctatgtgtggcaggtgctgaaggagaatggccccatggcctcaga
ccccctgtgcctgacctacagctacctgagccatgtggacctggtgaaggacctgaacagtggcctgattggagctctgctggtgt
gcagagagggcagcctggccaaggagaagacccagaccctgcacaagttcatcctgctgtttgctgtgtttgatgagggcaag
agctggcactcagagaccaagaacagcctgatgcaggacagagatgctgccagtgcaagagcctggcccaagatgcacact
gtcaatggatatgtgaacagaagcctgccaggactgataggctgccacagaaagtctgtatactggcatgttataggtatgggca
ccaccccagaggtgcacagcatcttcctggagggccacaccttcctggtgagaaaccacagacaggccagcctggagatcag
ccccatcaccttcctgactgcccagaccctgctgatggacctgggccagttcctgctgttctgccacatcagcagccaccagcatg
atggtatggaggcctatgtgaaggtggacagctgccctgaggagccccagctgcagatgaagaacaatgaggaggcagagg
actatgatgatgacctgacagacagtgagatggatgtggtgagatttgatgatgacaacagccccagcttcatccagatcagaa
gtgtggccaagaagcaccccaagacctgggtgcactacattgctgctgaagaggaggactgggactatgcacccctggtgctg
gccccagatgatagaagctacaagagccagtacctgaacaatggaccccagagaattggaagaaagtacaagaaggtgag
attcatggcctacactgatgagaccttcaagaccagagaggccatccagcatgaatcagggatcctgggccccctgctgtatgg
agaggtgggagacaccctgctgatcatcttcaagaaccaggccagcagaccctacaacatctacccccatggtatcactgatgt
aagacccctgtacagcagaagactgcccaagggagtgaagcacctgaaggacttccccatcctgcctggggaaatcttcaagt
acaagtggacagtcacagttgaagatggacccaccaagagtgatcccagatgcctgaccagatactacagcagctttgtgaac
atggagagagacctggcctcaggcctgataggccccctgctgatctgctacaaggagtctgttgaccagcagggcaaccagat
catgagtgacaagagaaatgtgatcctgttctcagtgtttgatgagaacagaagctggtacctgactgaaaacatccagagattc
ctgcccaacccagcaggggtgcagctggaggacccagagttccaggccagcaacatcatgcacagcatcaatggctatgtctt
tgatagcctgcagctgtctgtatgcctgcatgaggtggcctactggtacatcctgagcataggagcccagactgatttcctgagtgtt
ttcttcagtggctacaccttcaagcacaagatggtgtatgaggacaccctgaccctgttccccttctctggtgagactgtgttcatgag
catggagaaccctggtctgtggatcctgggctgccacaactcagatttcagaaacagaggcatgacagccctgctgaaggtga
gcagctgtgacaagaacactggggactactatgaagacagctatgaagacatctctgcttacctgctgagcaagaacaatgcc
attgagcccagaagcttcagccagaacccccctgtgctgaagagacaccagagagagatcaccagaaccaccctgcagtca
gaccaggaggagatagactatgatgataccatctctgtggagatgaagaaggaggactttgatatctatgatgaggatgaaaac
cagagccccagaagcttccagaagaagaccagacactacttcatagcagctgtagagagactgtgggactatggcatgagca
gcagcccccatgtactgagaaacagagcccagagtggaagtgtgccccagttcaagaaggtggtgttccaggagttcacagat
ggcagcttcacccagcccctgtacagaggagaactgaatgagcacctgggcctgctgggcccctacatcagagctgaggtgg
aggacaacatcatggtgaccttcagaaaccaggccagcagaccctacagcttctacagcagcctgatcagctatgaggagga
ccagagacagggagctgagcccagaaagaactttgtcaagcccaatgagaccaagacctacttctggaaggtgcagcacca
catggcccccaccaaggatgaatttgactgcaaggcctgggcctacttctcagatgtggacctggagaaggatgtgcactcagg
tctgattggccccctgctggtgtgccacaccaacaccctgaacccagcccatggtagacaggtgactgtgcaggagtttgctctgtt
cttcaccatctttgatgagaccaagagctggtacttcacagagaacatggagagaaactgcagagccccctgcaacatccagat
ggaggaccccaccttcaaggagaactacagattccatgccatcaatgggtacatcatggacaccctgccaggtctggtgatggc
ccaggaccagagaatcagatggtacctgctgagcatgggcagcaatgaaaacatccacagcatccacttcagtggacatgtgt
tcacagttagaaagaaggaggagtacaagatggccctgtacaacctgtaccctggagtgtttgaaactgtggagatgctgccca
gcaaggcaggcatctggagagtggagtgcctgataggggagcacctgcatgctggtatgagcaccctgttcctggtgtacagca
acaagtgccagacccccctgggcatggcctcaggccacatcagagacttccagatcactgctagtggccagtatggtcagtgg
gcccccaagctggccagactgcactactctggaagcatcaatgcctggagcaccaaggagcccttcagctggatcaaggtgg
acctgctggcccccatgatcatccatgggatcaagacccagggggccagacagaagttcagcagcctgtacatcagccagttc
atcatcatgtacagcctggatggaaagaagtggcagacctacagaggcaacagcacaggcaccctgatggtgttctttggcaat
gtagacagctcaggcatcaagcacaacatcttcaacccccccatcatagcaagatacatcagactgcaccccacccactacag
catcagaagcaccctgagaatggagctgatgggctgtgacctgaacagctgcagcatgcccctgggcatggagagcaaggc
catctcagatgcccagatcacagccagcagctacttcaccaacatgtttgccacctggagccccagcaaggccagactgcacct
gcagggcagaagcaatgcctggagaccccaggtgaacaaccccaaggagtggctgcaggtggacttccagaagaccatga
aggtgactggtgtgaccacccagggtgttaagagcctgctgaccagcatgtatgtgaaggagttcctgatcagcagcagccagg
atgggcaccagtggaccctgttcttccagaatggaaaggtgaaggtgttccagggcaaccaggacagcttcacccctgttgtga
acagcctggacccccccctgctgaccagatacctgagaatccacccccagagctgggtgcaccagatagcactgagaatgga
ggtgctgggctgtgaggcccaggacctgtactga (SEQ ID NO: 74)
FVIII-QQ15
atgcagattgaactgagcacctgcttcttcctgtgcctgctgagattctgcttcagtgctaccagaagatactacctgggggcagtgg
agctgagctgggactacatgcagtctgacctgggagagctgccagtagatgccagattcccccccagagtgcccaagagcttc
cccttcaacacctctgtggtgtacaagaagaccctgtttgtggagttcacagaccacctgttcaacatagctaagcccagaccccc
ctggatgggcctgctgggccccaccatccaggcagaggtgtatgacactgttgtgatcaccctgaagaacatggccagccaccc
agttagcctgcatgctgttggtgttagctactggaaggcctctgagggagcagagtatgatgaccagaccagccagagagaga
aggaggatgacaaggtgttcccaggggggagccacacctatgtctggcaggtgctgaaggagaatggccccatggcctcaga
ccccctgtgcctgacctacagctacctgagccatgtggacctggtgaaggacctgaactctggcctgattggtgcactgctggtgt
gcagagagggcagcctggccaaggagaagacccagaccctgcacaagttcatcctgctgtttgctgtctttgatgagggcaag
agctggcactcagaaaccaagaacagcctgatgcaggacagagatgcagcttctgctagagcctggcccaagatgcacactg
tgaatggctatgtcaacagaagcctgcctggcctgataggctgccacagaaagtctgtgtactggcatgtgattgggatgggcac
caccccagaggtgcacagcatcttcctggagggccacaccttcctggtgagaaaccacagacaggccagcctggagatcagc
cccatcaccttcctgactgcccagaccctgctgatggacctgggccagttcctgctgttctgccacatcagcagccaccagcatga
tggtatggaggcctatgtgaaggtggacagctgcccagaggagccccagctgcagatgaagaacaatgaagaggcagagg
actatgatgatgacctgacagactcagaaatggatgtggtgagatttgatgatgataacagccccagcttcatccagatcagatct
gttgccaagaagcaccccaagacctgggtgcactacattgcagctgaggaggaggactgggactatgctcccctggtgctggc
cccagatgacagaagctacaagagccagtacctgaacaatggaccccagagaatagggagaaagtacaagaaggtgaga
ttcatggcctacacagatgagaccttcaagaccagagaggccatccagcatgaatcagggatcctgggccccctgctgtatggg
gaggtgggtgacaccctgctgatcatcttcaagaaccaggccagcagaccctacaacatctacccccatggaatcacagatgtc
agacccctgtacagcagaagactgcccaagggagtaaagcacctgaaggacttccccatcctgccaggtgagatcttcaagta
caagtggacagtaacagtggaggatggtcccaccaagtctgaccccagatgcctgaccagatactacagcagctttgttaacat
ggagagagacctggccagtggcctgataggccccctgctgatctgctacaaggagtctgtagaccagcagggcaaccagatc
atgtcagataagagaaatgtaatcctgttcagtgtgtttgatgagaacagaagctggtacctgacagagaacatccagagattcct
gcccaaccctgcaggtgtgcagctggaggaccctgaattccaggccagcaacatcatgcacagcatcaatggatatgtttttgac
agcctgcagctgtcagtttgcctgcatgaggtggcctactggtacatcctgagcattggggcccagacagatttcctgtctgtgttctt
cagtgggtacaccttcaagcacaagatggtgtatgaggacaccctgaccctgttccccttctcaggtgaaactgtgttcatgagcat
ggagaacccagggctgtggatcctgggctgccacaactcagacttcagaaacagaggcatgactgctctgctgaaggtgagc
agctgtgacaagaacacaggtgactactatgaggacagctatgaagacatctcagcttacctgctgagcaagaacaatgccat
agagcccagaagcttcagccagaaccccccagttctgaagagacaccagagagagatcaccagaaccaccctgcagagtg
accaggaggagattgactatgatgacaccatctctgtagagatgaagaaggaggactttgacatctatgatgaggatgagaacc
agagccccagaagcttccagaagaagaccagacactacttcatagctgcagtggagagactgtgggactatggcatgagcag
cagcccccatgtgctgagaaacagagcccagagtggctcagtgccccagttcaagaaggtggtgttccaggagttcacagatg
gcagcttcacccagcccctgtacagaggggagctgaatgagcacctgggcctgctgggcccctacatcagagctgaggtgga
ggacaacatcatggtgaccttcagaaaccaggccagcagaccctacagcttctacagcagcctgatcagctatgaggaggac
cagagacagggggcagagcccagaaagaactttgtaaagcccaatgaaaccaagacctacttctggaaggtgcagcacca
catggcccccaccaaggatgaatttgactgcaaggcctgggcctacttcagtgatgtggacctggagaaggatgtgcactcagg
cctgattggtcccctgctggtgtgccacaccaacaccctgaacccagcccatggcagacaggtgactgtacaggagtttgccctg
ttcttcaccatctttgatgagaccaagagctggtacttcacagagaacatggagagaaactgcagagccccctgcaacatccag
atggaggaccccaccttcaaggagaactacagattccatgctatcaatgggtacatcatggacaccctgccaggcctggtgatg
gcccaggaccagagaatcagatggtacctgctgagcatgggcagcaatgagaacatccacagcatccacttcagtggccatgt
gttcacagtgagaaagaaggaggagtacaagatggccctgtacaacctgtaccctggagtgtttgaaactgtggagatgctgcc
cagcaaggcaggtatctggagagtggagtgcctgattggtgagcacctgcatgctgggatgagcaccctgttcctggtgtacagc
aacaagtgccagacccccctgggcatggcctcagggcacatcagagacttccagatcactgccagtgggcagtatgggcagt
gggcccccaagctggccagactgcactacagtggaagcatcaatgcatggagcaccaaggagcccttcagctggatcaaggt
ggacctgctggcccccatgatcatccatgggatcaagacccagggggccagacagaagttcagcagcctgtacatcagccag
ttcatcatcatgtacagcctggatggcaagaagtggcagacctacagaggcaacagcacaggtaccctgatggtgttctttggaa
atgtggacagctcaggtatcaagcacaacatcttcaacccccccatcatagcaagatacatcagactgcaccccacccactac
agcatcagaagcaccctgagaatggagctgatgggctgtgacctgaacagctgcagcatgcccctgggcatggagagcaag
gccatctcagatgctcagatcactgccagcagctacttcaccaacatgtttgccacctggagccccagcaaggccagactgcac
ctgcagggcagaagcaatgcctggagaccccaggtgaacaaccccaaggagtggctgcaggtggacttccagaagaccat
gaaggtgacaggggttaccacccagggagtgaagagcctgctgaccagcatgtatgtaaaggagttcctgatcagcagcagc
caggatggtcaccagtggaccctgttcttccagaatggaaaggtgaaggtgttccagggcaaccaggacagcttcaccccagt
ggtgaacagcctggacccccccctgctgaccagatacctgagaatccacccccagagctgggtgcaccagatagccctgaga
atggaggtgctgggctgtgaagcccaggacctgtactga (SEQ ID NO: 75)
FVIII-QQ16
atgcaaatagaactctccacctgcttctttctgtgccttttgaggttctgcttttctgcaactagaagatactacctgggtgcagtagaac
tgtcatgggactatatgcaatcagatctaggagaactgccagtggatgccagatttcctccaagggtgcccaaatcatttccattca
acacaagtgttgtgtacaaaaagaccctgtttgtggaattcacagatcaccttttcaacatagccaagcctagaccaccttggatg
ggtctgttgggtcctaccatccaggcagaagtttatgatactgtggtcattacacttaagaacatggctagccatccagtcagtcttc
atgctgttggggtatcctactggaaagcttcagagggagcagaatatgatgaccagacaagtcaaagggagaaagaagatga
caaagtcttccctggtggaagccatacctatgtctggcaggtcctgaaagagaatgggccaatggccagtgacccactgtgcctt
acctactcttatcttagtcatgtggacctggtaaaagacttgaatagtggcctcattggagcactactggtatgtagagaagggagc
ttggctaaggaaaagacacagactttgcacaaatttatactactttttgctgtatttgatgaggggaaaagttggcacagtgaaaca
aagaactccttgatgcaggatagggatgcagcaagtgctagggcttggccaaaaatgcacacagttaatgggtatgttaacaga
tcactgcctgggctgattggttgccacagaaaatctgtctattggcatgtgattggaatgggcacaactcctgaagtgcactcaatat
tccttgaaggtcacacatttcttgtgagaaaccatagacaggcttccttggaaatcagccccataaccttccttacagctcaaacac
tcttgatggaccttggacagtttctactgttttgtcatatctcttcccaccaacatgatggaatggaagcctatgtcaaagtagacagct
gtcctgaggaaccacaactacagatgaaaaataatgaagaagcagaagactatgatgatgatcttactgattcagaaatggat
gtggtcaggtttgatgatgataactctccttcctttatccaaattagatctgtagcaaagaagcatcctaaaacttgggtacattacatt
gctgcagaagaggaggactgggactatgcacccttggtcttggcaccagatgatagaagttataaaagtcaatatttgaacaatg
gcccccagagaattggtaggaagtacaaaaaagtcagatttatggcatacacagatgaaacatttaagacaagagaagctatt
cagcatgagtcagggatcttgggacctttactttatggtgaagttggagatacactgttgattatatttaagaatcaagcaagcagac
catataacatctaccctcatggtatcacagatgtaagacccttgtattcaagaagattacccaaaggtgtaaaacatttgaaggatt
ttccaattctgccaggggagatattcaaatataaatggacagtgacagtagaggatgggccaactaaatcagatcctaggtgcct
gaccaggtattactctagttttgttaatatggagagagatctagctagtggactcattggacctctcctcatctgctacaaagaatcag
ttgatcaacagggaaaccagataatgtcagataagaggaatgtcatcctgttttctgtatttgatgaaaacaggagctggtacctca
cagaaaatatacaaagatttctccccaatcctgctggagtgcagcttgaagatccagaattccaagcttcaaacatcatgcacag
catcaatggctatgtatttgattccttgcagttgtcagtgtgtttgcatgaggtggcatactggtacattctatctattggagctcagactg
acttcctttctgtgttcttctcaggatatacattcaaacacaaaatggtctatgaagacacactcaccctattcccattctcaggggaa
acagtcttcatgtcaatggaaaacccaggtctatggattttggggtgccacaactcagactttagaaacagaggcatgacagcttt
actgaaggttagtagttgtgataagaacactggggattattatgaggactcttatgaagatatttctgcatacttgctgagtaaaaac
aatgcaattgaaccaagaagcttctctcaaaaccccccagtcttgaaaagacatcaaagagaaataaccagaactactcttca
gtctgatcaagaggaaattgactatgatgataccatatcagttgaaatgaagaaggaagattttgacatttatgatgaagatgaaa
atcagagccccaggagctttcaaaagaaaacaagacactattttattgcagcagtggagaggctctgggattatggtatgagtag
ctccccacatgtactaaggaacagggcccagagtggctctgtcccacagttcaagaaagtagttttccaggaatttacagatggc
tcctttactcagcctttatacaggggagaactaaatgaacatttgggactcctagggccatatataagagcagaggttgaagataa
tatcatggtaactttcaggaatcaggcctctaggccctattccttctattctagccttatttcttatgaggaagatcagaggcaaggag
cagagcctaggaaaaactttgttaagcctaatgaaaccaaaacatacttttggaaagtgcaacatcatatggcccccaccaaag
atgaatttgactgcaaagcctgggcatatttctctgatgtggacttagaaaaagatgttcactcaggcctgataggaccacttctggt
ctgccacactaacacactgaacccagcccatgggagacaagtgactgtacaggaatttgcactgtttttcaccatctttgatgaaa
caaaaagctggtacttcactgaaaatatggaaagaaactgcagggctccctgcaatatccagatggaagatccaacatttaaa
gagaattatagattccatgctatcaatggctacataatggatacactaccaggcttagtaatggctcaggatcaaaggattagatg
gtatctgctcagcatgggcagtaatgagaacatccattctattcatttctcaggacatgtattcactgtaagaaaaaaagaggagta
taaaatggcactgtacaatctctatccaggtgtttttgagacagtagaaatgttaccttcaaaagcaggaatttggagagtggaatg
ccttattggtgagcatctacatgctggaatgagcacactttttctggtgtacagcaataagtgtcagacccctctgggaatggcaagt
ggacacattagagactttcagattacagcttcaggacaatatggacagtgggccccaaagctggccagacttcattattcagggt
caatcaatgcatggagcaccaaggagccttttagctggatcaaggtggatctgttggcaccaatgattattcatggcatcaagact
cagggagccagacagaagttctcaagcctctacatctctcagtttatcatcatgtattcccttgatgggaagaagtggcagacttat
agaggaaatagtacaggaaccttaatggtcttctttgggaatgtggattcaagtgggataaaacacaatatttttaaccctccaatta
ttgctaggtacatcaggttgcaccccactcattatagcattaggagcactcttagaatggagttgatgggctgtgatttaaatagttgc
agtatgccattgggaatggagagtaaagcaatatcagatgcacagattactgcatcatcttactttactaatatgtttgcaacttggtc
tccatcaaaagcaagacttcacctccaagggaggtctaatgcttggagacctcaggtgaataatccaaaagagtggctgcaagt
ggacttccagaagacaatgaaagtcacaggtgtgaccactcagggtgtaaaatctctgcttaccagcatgtatgttaaggagttcc
tcatctccagcagtcaagatggacatcagtggactctcttttttcagaatggtaaagtaaaggtttttcagggaaatcaagactccttc
acaccagtggttaacagtctagacccaccattactgactaggtaccttagaattcacccacagagttgggtgcaccagattgccct
gaggatggaggttctgggctgtgaagcacaggacctctactga (SEQ ID NO: 76)
FVIII-QQ17
atgcaaatagagctctccacctgcttctttctgtgccttttgaggttctgcttttctgccacaaggagatactacctgggtgcagtggaa
ctgtcatgggactatatgcaaagtgacctaggagagctgccagtggatgcaaggtttcctcctagagtgcccaaaagttttccattc
aacacatcagttgtgtacaaaaagacactgtttgtagaattcacagatcaccttttcaacatagcaaagcccaggccaccatgga
tgggtctgctaggtcctaccatccaggcagaggtttatgatactgtggtcattacacttaagaacatggcttcacatcctgtcagtcttc
atgctgtgggtgtatcctactggaaagcttcagagggagcagaatatgatgaccagactagtcaaagagagaaagaagatgat
aaagtcttcccaggtggatcccatacctatgtctggcaggtcctgaaagagaatggtcctatggccagtgatcctctgtgccttactt
acagttatcttagtcatgttgacctggtaaaagacttgaattcaggactcattggagcactattggtatgtagagaagggtctttagct
aaggaaaagacacagaccttgcacaaatttatactactttttgcagtatttgatgaagggaaaagttggcacagtgaaacaaag
aactccttgatgcaggatagggatgcagcatctgctagggcctggcccaaaatgcacacagtcaatgggtatgtgaacaggtct
ctgcctggtctgattggatgccacagaaaatcagtatattggcatgtgataggaatgggcacaactcctgaggtgcactcaatattc
ttagaaggtcacacatttcttgtaagaaaccatagacaggcatccttggaaatctctcccataacattccttacagctcaaaccctct
tgatggaccttggacagtttctactgttttgtcatatctcttcccaccaacatgatgggatggaagcatatgtcaaagtagacagctgt
ccagaggaaccacaactacagatgaaaaataatgaagaagcagaagactatgatgatgatcttactgattcagaaatggatgt
agtcaggtttgatgatgataactctccttcctttatccaaattaggtctgttgccaagaagcatcctaaaacttgggtacattacattgct
gcagaagaggaggactgggactatgcaccattagtactagcacctgatgacagaagttataaaagtcaatatttgaacaatgga
ccccagagaattgggaggaagtacaaaaaagtcagatttatggcatacactgatgagacatttaagactagagaagctattcag
catgaatcaggaatcttgggacctttactttatggggaagtaggagacacactgttgattatatttaagaatcaagcaagcagacc
atataacatctacccacatggaatcacagatgttagacccttgtattcaaggaggttaccaaaaggtgtaaaacatttgaaggattt
tcctattctgccaggtgaaatattcaaatataaatggactgtgactgttgaagatggaccaactaaatcagaccccaggtgcctga
ccaggtattactctagttttgttaatatggagagagatctagcttcaggactcataggccctctcctcatctgctacaaagaatcagta
gatcaacagggaaaccagataatgtcagacaagaggaatgtaatcctgttttctgtatttgatgaaaacagaagctggtacctca
cagagaatatacaaaggtttctccccaatcctgcaggagtgcagcttgaggatcctgagttccaagcttccaacatcatgcacag
catcaatgggtatgtgtttgattctttgcagttgtctgtttgtttgcatgaagtggcatactggtacattctatctattggtgctcagactg
acttcctttctgtattcttctcaggctatactttcaaacacaaaatggtctatgaagacaccctcaccctattcccattctctggggaaactg
tgttcatgtccatggaaaaccctggactatggattctgggctgccacaactctgactttagaaacagaggcatgactgctttactga
aggtttcaagttgtgacaagaacactggtgattattatgaagactcctatgaagatattagtgcctacttgctgagtaaaaacaatgc
aatagaaccaaggagcttctctcaaaaccccccagtcttgaaaaggcatcaaagagaaataaccagaactactcttcagtcag
atcaagaggaaattgactatgatgataccataagtgttgaaatgaagaaggaagattttgacatttatgatgaggatgaaaatca
gagccccaggagctttcaaaagaaaacaagacactattttatagctgctgtagagagactctgggattatgggatgagttcatcc
cctcatgtactaaggaacagggcccagtcaggttcagtcccccagttcaagaaagttgtcttccaggaatttacagatgggtccttt
actcagcctttatacaggggagaactaaatgaacatttgggactcctgggaccatatataagagcagaagttgaagataatatca
tggtaactttcaggaatcaggcctctagaccctattccttctattctagccttatttcttatgaagaagatcagaggcaaggagcaga
acctagaaaaaactttgtcaagcctaatgaaactaaaacttacttttggaaagtgcaacatcatatggctcccactaaagatgagt
ttgattgcaaagcctgggcatatttctcagatgttgacctagagaaagatgtacactcaggactgattggacctcttctggtctgcca
cactaacacactgaaccctgctcatggtagacaagtgactgtacaggaatttgccctgtttttcaccatctttgatgagacaaaaag
ctggtacttcacagaaaatatggaaagaaactgcagggctccctgcaatatccagatggaagatcctacctttaaagagaattat
aggttccatgcaatcaatggctacataatggatacactacctgggttagtaatggctcaggatcaaaggattaggtggtatctgctc
agcatgggctccaatgagaacatccattccattcatttctcaggacatgtgttcacagttaggaaaaaagaggagtataaaatgg
cactgtacaatctctatccaggtgtttttgaaacagtagaaatgttacccagtaaagctggaatttggagggtggagtgccttatagg
tgagcatctacatgcaggaatgagcacactttttctggtgtacagcaataagtgtcagacacccctgggaatggcctcaggacac
attagggactttcagattacagccagtggacaatatggtcagtgggccccaaagctggccagacttcattatagtggttcaatcaat
gcttggagcactaaggagcccttttcttggatcaaggtggatctgctggctcccatgattattcatggcatcaagacacagggtgct
aggcagaagttcagcagtctctacatctctcagtttatcatcatgtattcacttgatgggaagaagtggcagacttatagaggaaatt
ccacaggaaccttaatggtcttctttggaaatgtggattcatctggaataaaacacaatatttttaaccctccaattattgctagataca
tcaggttgcacccaactcattatagcattagaagcactcttagaatggagttgatgggctgtgatttaaatagttgctccatgcccttg
ggaatggagagtaaagcaatatcagatgcccagattacagcttcatcatactttaccaatatgtttgcaacatggtctccatcaaaa
gcaagacttcacctccaagggagaagtaatgcctggagacctcaggtgaataatccaaaagagtggctgcaagtggacttcca
gaagactatgaaagtcacaggagtaacaactcagggagttaaatctctgcttaccagcatgtatgtaaaggagttcctcatctcca
gcagtcaagatgggcatcagtggactctcttttttcagaatggaaaagtaaaggtttttcagggaaatcaagactccttcacacca
gtggttaacagtctggaccccccttactgactagataccttaggattcaccctcagagttgggtgcaccagattgcactgagaatg
gaggttctgggctgtgaggcacaggacctctactga (SEQ ID NO: 77)
FVIII-QQ18
atgcaaatagagctctccacctgcttctttctgtgccttttgaggttctgctttagtgcaacaagaagatactacctgggtgcagtaga
actgtcatgggactatatgcaatctgatttaggggagctgcctgtggatgcaagatttccacccagggtgcctaaaagctttcccttc
aacacttcagttgtgtacaaaaagaccctgtttgtagaattcactgatcaccttttcaacattgctaagccaaggccaccctggatgg
gtctgcttggtcctaccatccaggcagaagtttatgatactgtggtcattacacttaagaacatggcttcccatccagtgagtcttcatg
ctgtgggtgtatcctactggaaagcttctgagggagctgaatatgatgaccagaccagtcaaagggagaaagaagatgacaaa
gtcttcccaggtggaagccatacctatgtgtggcaggtcctgaaagagaatggtccaatggcctctgatcctctgtgccttacctact
cctatctttcacatgttgacctggtaaaagacttgaattcaggcctcattggggccctactagtttgtagagaagggtctctggctaag
gaaaagactcagacattgcacaaatttatactactttttgctgtatttgatgaggggaaaagttggcactcagagacaaagaactc
cttgatgcaggatagagatgctgcttcagctagagcatggcctaaaatgcacactgtcaatggatatgtaaacagaagcctgcca
ggtctgattggttgccacaggaaatctgtctattggcatgtgattggcatgggcaccactccagaagtgcactcaatattcttagagg
gtcacacatttcttgtaagaaaccatagacaggcttccttggaaatctcacctataaccttccttacagctcaaaccctcttgatgga
cctgggacagtttctactgttttgtcatatctcttcccaccaacatgatggaatggaagcttatgtcaaagtagacagctgtccagag
gaaccccaactacagatgaaaaataatgaagaagctgaagactatgatgatgatcttactgatagtgaaatggatgtagtcagg
tttgatgatgacaactctccttcctttatccaaattaggtcagttgctaagaagcatcctaaaacttgggtacattacattgcagcaga
agaggaggactgggactatgccccattagtcctagcccctgatgatagaagttataaaagtcaatatttgaacaatggaccaca
gagaattggtaggaagtacaaaaaagtcagatttatggcatacactgatgagacctttaagacaagggaagctattcagcatga
atctggaatcttgggacctttactttatggagaagttggggacacactgttgattatatttaagaatcaagcaagcagaccatataac
atctacccccatggaatcactgatgtcaggcccttgtattcaagaaggttacctaaaggtgtaaaacatttgaaggattttcctattct
gccaggtgaaatattcaaatataaatggacagtgactgtagaagatggcccaactaaaagtgatcccaggtgcctgaccagat
attactctagttttgtcaatatggagagagatctagcttcaggactcattggtcctctcctcatctgctacaaagaatcagtagatcaa
cagggaaaccagataatgagtgataagaggaatgtcatcctgtttagtgtatttgatgaaaacagaagctggtacctcacagaa
aatatacaaagatttctccccaatccagctggggtgcagttggaggatccagaattccaagcaagtaacatcatgcacagcatc
aatgggtatgtatttgactctttgcagttgtcagtatgtttgcatgaagtggcatactggtacattctatcaattggggcccagactgattt
ccttagtgtcttcttctctggttatacattcaaacacaaaatggtctatgaagacaccctcaccctattcccattctcaggggaaactgt
cttcatgagtatggaaaacccaggtctatggattttggggtgccacaactcagactttaggaacagaggcatgactgcattactga
aggtttcaagttgtgataagaacactggtgattattatgaagacagttatgaggatatttcagcatacttgctgagtaaaaacaatgc
aatagaaccaagaagcttctctcaaaacccaccagtcttgaaaagacatcaaagagaaataaccaggactactcttcagtctg
atcaagaggaaattgattatgatgacaccatatcagttgaaatgaagaaggaagattttgatatttatgatgaggatgaaaatcag
agcccaaggagctttcaaaagaaaaccagacactattttattgcagcagtagagagactctgggattatgggatgagttcatccc
cccatgttctaagaaacagggcacagagtggcagtgtcccacagttcaagaaagtagttttccaggaatttacagatggttccttta
ctcagccattatacaggggagaactaaatgaacatttgggactcttagggccatatataagagctgaagttgaagataatatcatg
gtaactttcaggaatcaggcctctagaccatattccttctatagtagccttatttcttatgaggaagatcagaggcaaggagcagag
cctaggaaaaactttgttaagcctaatgaaacaaaaacttacttttggaaagtgcaacatcatatggcacccaccaaagatgagt
ttgactgcaaagcctgggcctatttctctgatgttgacttagaaaaagatgtgcactctgggctgattggaccacttctggtctgccac
actaacacactgaacccagcacatggcagacaagtgactgtccaggaatttgctctgtttttcaccatctttgatgagacaaaaag
ctggtacttcactgaaaatatggaaaggaactgcagggctccctgcaatatccagatggaagatccaacctttaaagagaattat
agattccatgccatcaatgggtacataatggatacactacctggcttagtaatggctcaggatcaaaggattaggtggtatctgctc
agcatgggctccaatgaaaacatccatagcattcatttcagtggacatgttttcactgtaaggaaaaaagaggagtataaaatgg
cactgtacaatctctatccaggagtttttgagacagttgaaatgttaccatctaaagcaggaatttggagggttgaatgccttattggg
gaacatctacatgcaggtatgagcacactttttctggtgtacagcaataagtgtcagactccactgggaatggcatctggccacatt
agagattttcagattactgcatcagggcaatatggacagtgggccccaaagctggccagacttcattatagtggatcaatcaatgc
ttggagcaccaaggagcccttttcttggatcaaggtggatctgcttgcacccatgattattcatgggatcaagacccagggtgcca
gacagaagttcagcagcctctacatctctcagtttatcatcatgtatagccttgatggaaagaagtggcagacttataggggaaat
agcacaggaaccttaatggtcttctttggtaatgttgattcaagtgggataaaacacaatatttttaaccctccaattatagctagata
catcagattgcaccccactcattatagcattaggagcactcttaggatggagttgatgggctgtgatttaaatagttgcagtatgccct
tgggaatggagagtaaagcaatatcagatgctcagattactgcatcaagctactttactaatatgtttgctacatggtctccatcaaa
agctagacttcacctccaagggagatccaatgcttggagacctcaggtgaataatccaaaagagtggctgcaagtggacttcca
gaagactatgaaagtcacaggggtaacaacccagggagtaaaatctctgcttaccagcatgtatgtcaaggagttcctcatctcc
agctcacaagatgggcatcagtggactctcttttttcagaatgggaaagtaaaggtttttcagggaaatcaagactccttcacacca
gtggtgaactctttggacccacctttactgactagataccttaggattcacccacagagttgggtgcaccagatagctctgagaatg
gaggttctgggctgtgaggcccaggacctctactga (SEQ ID NO: 78)
Isoform A human FVIII
mqielstcfflcllrfcfsatrryylgavelswdymqsdlgelpvdarfpprvpksfpfntsvvykktlfveftdhlfniakprppwmgllg
ptiqaevydtvvitlknmashpvslhavgvsywkasegaeyddqtsqrekeddkvfpggshtyvwqvlkengpmasdplclt
ysylshvdlvkdlnsgligallvcregslakektqtlhkfillfavfdegkswhsetknslmqdrdaasarawpkmhtvngyvnrslp
gligchrksvywhvigmgttpevhsifleghtflvrnhrqasleispitfltaqtllmdlgqfllfchisshqhdgmeayvkvdscpeep
qlrmknneeaedydddltdsemdvvrfdddnspsfiqirsvakkhpktwvhyiaaeeedwdyaplvlapddrsyksqylnng
pqrigrkykkvrfmaytdetfktreaiqhesgilgpllygevgdtlliifknqasrpyniyphgitdvrplysrrlpkgvkhlkdfpilpge
ifkykwtvtvedgptksdprcltryyssfvnmerdlasgligpllicykesvdqrgnqimsdkrnvilfsvfdenrswylteniqrflpnp
agvqledpefqasnimhsingyvfdslqlsvclhevaywyilsigaqtdflsvffsgytfkhkmvyedtltlfpfsgetvfmsmenpg
lwilgchnsdfrnrgmtallkvsscdkntgdyyedsyedisayllsknnaieprsfsqnsrhpstrqkqfnattipendiektdpwfa
hrtpmpkiqnvsssdllmllrqsptphglslsdlqeakyetfsddpspgaidsnnslsemthfrpqlhhsgdmvftpesglqlrine
klgttaatelkkldfkvsstsnnlistipsdnlaagtdntsslgppsmpvhydsqldttlfgkksspltesggplslseenndskllesgl
mnsqesswgknvsstesgrlfkgkrahgpalltkdnalfkvsisllktnktsnnsatnrkthidgpsllienspsvwqnilesdtefkk
vtplihdrmlmdknatalrlnhmsnkttssknmemvqqkkegpippdaqnpdmsffkmlflpesarwiqrthgknslnsgqgp
spkqlvslgpeksvegqnflseknkvvvgkgeftkdvglkemvfpssrnlfltnldnlhennthnqekkiqeeiekketliqenvvlp
qihtvtgtknfmknlfllstrqnvegsydgayapvlqdfrslndstnrtkkhtahfskkgeeenleglgnqtkqivekyacttrispnts
qqnfvtqrskralkqfrlpleetelekriivddtstqwsknmkhltpstltqidynekekgaitqsplsdcltrshsipqanrsplpiakvs
sfpsirpiyltrvlfqdnsshlpaasyrkkdsgvqesshflqgakknnlslailtlemtgdqrevgslgtsatnsvtykkventvlpkpdl
pktsgkvellpkvhiyqkdlfptetsngspghldlvegsllqgtegaikwneanrpgkvpflrvatessaktpsklldplawdnhygt
qipkeewksqekspektafkkkdtilslnacesnhaiaainegqnkpeievtwakqgrterlcsqnppvlkrhqreitrttlqsdqe
eidyddtisvemkkedfdiydedenqsprsfqkktrhyfiaaverlwdygmsssphvlrnraqsgsvpqfkkvvfqeftdgsftqp
lyrgelnehlgllgpyiraevednimvtfrnqasrpysfysslisyeedqrqgaeprknfvkpnetktyfwkvqhhmaptkdefdc
kawayfsdvdlekdvhsgligpllvchtntlnpahgrqvtvqefalfftifdetkswyftenmerncrapcniqmedptfkenyrfhai
ngyimdtlpglvmaqdqrirwyllsmgsnenihsihfsghvftvrkkeeykmalynlypgvfetvemlpskagiwrvecligehlh
agmstlflvysnkcqtplgmasghirdfqitasgqygqwapklarlhysgsinawstkepfswikvdllapmiihgiktqgarqkfs
slyisqfiimysldgkkwqtyrgnstgtlmvffgnvdssgikhnifnppiiaryirlhpthysirstlrmelmgcdlnscsmplgmesk
aisdaqitassyftnmfatwspskarlhlqgrsnawrpqvnnpkewlqvdfqktmkvtgvttqgvkslltsmyvkeflisssqdgh
qwtlffqngkvkvfqgnqdsftpvvnsldpplltrylrihpqswvhqialrmevlgceaqdly (SEQ ID NO: 95)
Isoform B Human FVIII
mriqdpgkvffgnvdssgikhnifnppiiaryirlhpthysirstlrmelmgcdlnscsmlgmeskaisdaqitassyftnmfatws
pskarlhlqgrsnawrpqvnnpkewlqvdfqkmkvtgvttqgvkslltsmyvkeflisssqdghqwtlffqngkvkvfqgnqdsft
pvvnsldpplltrylrihpqswvhqialrmevlgceaqdly (SEQ ID NO: 96)

TABLE 16
signal peptides

>201 signal peptide
MEFGLSWVFLVALLKGVQCE (SEQ ID NO: 81)
>wt IL2 signal peptide
MYRMQLLSCIALSLALVTNS (SEQ ID NO: 82)
>mut IL2 signal peptide
MYRMQLLLLIALSLALVTNS (SEQ ID NO: 83)
>A2M signal peptide
MGKNKLLHPSLVLLLLVLLPTDA (SEQ ID NO: 84)
>PZP signal peptide
MRKDRLLHLCLVLLLILLSASDSNS (SEQ ID NO: 85)
>artificial signal peptide
MWWRLWWLLLLLLLLWPMVWA (SEQ ID NO: 86)
>cathpetsin L signal peptide
LLLLAVLCLGT (SEQ ID NO: 87)
>nucleotide of hAAT signal peptide
ATGCCGTCTTCTGTCTCGTGGGGCATCCTCCTGCTGGCAGGCCTGTGCT
GCCTGGTCCCTGTCTCCCTGGCT (SEQ ID NO: 88)
>FIBrat Signal Peptide
MLRGPGPGRLLLLAVLCLGTSVRCTETGKSKR (SEQ ID NO: 89)
>FIBrat Signal Peptide
MLRGPGPGRLLLLAVLCLGTSVRCTETGKSKRLALQIV
(SEQ ID NO: 90)
>FN1 human signal peptide
MLRGPGPGLLLLAVQCLGTAVPSTGA (SEQ ID NO: 91)
>FN1 x. leavis signal peptide
MRRGALTGLLLVLCLSVVLRAAPSATSKKRR (SEQ ID NO: 92)
>preprocathepsin L signal peptide (rat)
MTPLLLLAVLCLGTALA (SEQ ID NO: 93)
>prepro-alpha 2 type collagen signal peptide
(homo sapiens)
MLSFVDTRTLLLLAVTLCLATC (SEQ ID NO: 94)

### Example 2—In Vivo Study 1

### Introduction

The aim was to generate an expression cassette containing a coding sequence (CDS) encoding hFVIII under the control of a liver-specific promoter. Here the potency of liver-specific promoters driving the expression of FVIII were assessed in an in vivo study in mice.

### Methods

2 novel promoters (SP0472 and SP0412+UTR (SP0412_v2)) and a state-of-the-art benchmark promoter (HLP) were placed upstream of a coding sequence encoding a FVIII sequence (hereby indicates as F8-SQ00) and packaged within an AAV8 capsid; their potency was analysed following intravenous injection of the AAV vectors to C57BL/6J mice over the course of 4 weeks. Table 17 summarises the study design.

TABLE 17
Study design

| Vector | Pro- | | Dose | |
|---|---|---|---|---|
| ID | moter | CDS | (vg/kg) | Main readouts |
| Vector 1 | HLP | F8-SQ00 | 1 × 10¹⁰ | |
| Vector 1 | HLP | F8-SQ00 | 1.2 × 10⁹ | Blood collection at weeks 2 |
| Low dose | | | | and 4 for measurement of |
| | | | | human FVIII antigen |
| | | | | levels by ELISA. |
| | | | | Tissue collection at sacrifice |
| | | | | for measurement of liver |
| Vector 17 | 472 | F8-SQ00 | 1 × 10¹⁰ | vector copy number. |
| Vector 24 | 412 + | F8-SQ00 | 1 × 10¹⁰ | |
| | UTR | | | |

### AAV Production

AAVs were produced by triple transfection in high density Pro10 cells. Helper plasmid xx680 and RepCap plasmid GSK2/8 were used. The cells were harvested on day three and lysed by sonication. The lysates were purified by iodixanol gradient and concentrated on amicon filtration units. The produced AAVs were quantified by PCR-based methods as described below and their purity was confirmed by silver stain.

### AAV Titration

AAVs were titrated by ITR droplet digital PCR (ddPCR). To extract the DNA from the AAV preparations, vectors were treated first with DNase and then with proteinase K. Next, the AAV DNA was quantified by ddPCR targeting the ITR region of the vector (Forward primer: 5′-GGAACCCCTAGTGATGGAGTT-3′ (SEQ ID NO: 45); Reverse primer: 5′-CGGCCTCAGTGAGCGA-3′ (SEQ ID NO: 46); Probe: 5′-FAM-CACTCCCTCTCTGCGCGCTCG-BHQ1-3′ (SEQ ID NO: 47) and using an appropriate dilution.

### AAV Administration

AAV vectors were administered to 8- to 12-week-old male C57BL/6JOlaHsd mice (n=5 mice/vector) in a volume of 200 μL of phosphate-buffered saline/0.001% Pluronic via tail vein injection.

### Sample Collection

Blood samples were obtained by venesection of the lateral vein at 2 weeks post AAV injection. At 4 weeks post injections blood was collected via terminal cardiac bleeding. This was performed under anaesthesia using isoflurane. 3.8% sodium citrate was added to sterile, low protein binding 1.5 mL tubes to a 1/10 final volume. Blood was then added, gently mixed, and kept at 4° C. until processing. Samples were immediately centrifuged at 10,000 G for 10 minutes at 4° C. and thereafter plasma was collected and stored at −80° C. for further analysis. After sacrifice at week 4, the following tissues were harvested: liver, heart, kidney, lung, brain, spleen, inguinal lymph nodes, adrenal glands, large intestine, testicles, quadriceps and gallbladder. For tissue collection, each tissue was divided in two, keeping one piece in a tube containing RNAlater® while the other was directly kept in an empty 1.5 ml tube. Both tubes were frozen in liquid nitrogen and stored at −80° C. for further analysis.

### Human Coagulation Factor VIII ELISA

Quantification of hFVIII was based on an ELISA assay using the commercial kit Anti-hFVIII (F8C-EIA, Affinity). The ELISA plate (442404, ThermoFisher) was coated with the capture antibody diluted 1/100 in carbonate buffer and incubated at room temperature for 2 hours. Blocking was not required under the conditions described. The capture antibody was removed by washing three times with wash buffer (PBS tween; 0, 1% v/v). Standards and samples were diluted in green sample diluent supplied by the manufacturer and placed in the appropriate wells. The plate was incubated at room temperature for 2 hours. After incubation, plates were washed 3 times with wash buffer and the pre-diluted detection antibody was added to each well. Plates were incubated for 60 minutes at room temperature. After the incubation time, plates were washed 3 times with wash buffer and TMB (A: 51-2606KC; B: 51-2607KC; BD) was added to develop the plates. Following incubation for 20 minutes at room temperature, the reaction was stopped with H₂SO₄ 1M. Absorbance was measured at a wavelength of 450 nm in a microplate reader.

The hFVIII concentration of the samples was determined by interpolating their optical density (OD) on the curve generated by a simple nonlinear regression analysis (sigmoidal, 4PL, X is concentration) relating the OD of the standard dilutions to their concentration.

### DNA Extraction

Maxwell® RSC Tissue DNA Kit (Promega AS1610) was used for DNA extraction, following manufacturer's instructions. Briefly, a piece of liver (20 mg approximately) was put in a 1.5 ml tube, 80 mL of TE buffer were added and the sample was disrupted and homogenized using a pestle. The sample was added to the 1^{st} position of the cartridge and a plunger was placed on well 8. An empty elution tube was placed into the elution tube position and 100 μl of Elution Buffer were added to the bottom of each elution tube. The tissue DNA method was run and once the extraction process was finished, the elution tube containing DNA was stored at −20° C. until the presence of vector DNA in the sample was analyzed.

### Hepatic Vector Copy Number Quantification (VCN)

VCN was determined by qPCR using the hF8co as target (Gapdh was used for normalization). The concentration of plasmid DNA was obtained by interpolating their Ct value on the standard curve generated by simple linear regression. The primers and probes used for VCN determination are listed below:

TABLE 18
Specific probe and primers for hF8co sequence used for VCN analysis

| ID | Sequence | SEQ ID NO |
|---|---|---|
| hF8co_Probe | CCCCTGGGCATGGAGAGCAAGGCC- | 48 |
| | FAM | |
| hF8co_Forward | GATGGGCTGTGACCTGAACA | 49 |
| hF8co_Reverse | CCAGGTGGCAAACATGTTGG | 50 |
| Mouse | 5HEX/TTGTCATCAACGGGAAGCCCATCA | 51 |
| GAPDH_Probe | | |
| Mouse | ACGGCAAATTCAACGGCAC | 52 |
| GAPDH_Forward | | |
| Mouse | TAGTGGGGTCTCGCTCCTGG | 53 |
| GAPDH_Reverse | | |

### RNA Extraction

The Maxwell® RSC Tissue RNA Kit (#AS1340, Promega) was used for tissue RNA extraction, following manufacturer's instructions. Briefly, a piece of tissue was placed in a 1.5 ml tube, 200 μL of chilled 1-Thioglycerol/Homogenization solution were added and the sample was disrupted and homogenized using the TissueLyser II (Qiagen). 200 μL of Lysis buffer were added to the homogenate and vortexed for 15 seconds. The lysate was transferred to the cartridge, 10 μL of DNase I were added and the RNA was purified following the Maxwell® RSC simplyRNA method. The RNA was eluted in 50 μL of nuclease free water (NFW) and stored at −80° C. until the sample was quantified and analyzed.

In tissues with low RNA extraction yield, such as the muscle, TRIzol (#15596026, ThermoFisher) was used. Briefly, a piece of tissue was homogenized in 500 μL of TRIzol using the TissueLyser. After adding 500 μL of TRIzol, samples were incubated during 5 min at room temperature following a 10 min centrifugation at 4° C. and 12000 g. The supernatant was transferred to a new Eppendorf and incubated during 2 min at room temperature after adding 200 μL of chloroform. The non-coloured aqueous phase was mixed with 500 μL isopropanol, incubated during 10 min at room temperature and centrifuged for 10 min at 4° C. and 12000 g for RNA precipitation. Finally, the pellet was washed with ₇₅% EtOH, dried at room temperature and resuspended in 50 μL NFW, following a final incubation of 2 min at 56° C.

### Reverse Transcription

1 μg RNA was reverse transcribed to its complementary DNA (cDNA) using a High Capacity cDNA Reverse Transcription Kit (#4368813, ThermoFisher) following the instructions indicated by the manufacturer.

### Gene Expression Determination

For gene expression determination, a quantitative polymerase chain reaction (qPCR) was performed using the TaqMan gene expression assay on the Applied Biosystems™ QuantStudio™ 5 system. The primers and probes used were specific to the hFVIII-SQ sequence, common to all the plasmids, and are listed in Table 19.

TABLE 19
Specific probe and primers

| ID | Sequence | SEQ ID NO |
|---|---|---|
| SQ_Probe | CCCCTGGGCATGGAGAGCAAGGCC-FAM | 54 |
| SQ_Forward | GATGGGCTGTGACCTGAACA | 55 |
| SQ_Reverse | CCAGGTGGCAAACATGTTGG | 56 |
| Rplp0_Probe | 5HEX/TGGAGGGTG/ZEN/TCCGCAACGTGGCC | 57 |
| Rplp0_Forward | CAGGTGTTTGACAACGGCAG | 58 |
| Rplp0_Reverse | CCGATCTGCAGACACACACT | 59 |

The mRNA levels of the gene of interest were normalized with Rplp0 mRNA levels as endogenous control obtaining the relative expression by the ΔΔCt method (2^{-ΔΔCt}) using the liver of each animal as reference sample to determine the fold decrease versus the liver. Samples were analyzed in triplicates and for each one no retrotranscribed RNA was used as negative control.

### Statistical Analysis

Analysis was performed using GraphPad Prism version 9.3.1. Data are presented as mean±SD unless otherwise indicated.

### Results

Two novel, liver-specific promoters were generated and placed upstream of a sequence encoding FVIII. The potency of these promoters at driving FVIII expression was benchmarked against HLP, a previously published promoter (McIntosh et al., 2013) and assessed following IV administration of AAV8 vectors. In summary, the vector containing 472 elicited circulating human FVIII levels above the baseline (FIG. 1).

Two different doses of the vector containing the benchmark promoter (HLP) were used, resulting in the same FVIII/VCN ratio (only high dose HLP shown). This was to show that VCN can effectively be used as a normalizer at the doses used. For the in vivo assessment of promoter potency, mice (N=5 per group) were intravenously administered either one of the different vectors. Two and four weeks after administration, citrate plasma samples were collected to analyze for the presence of circulating hFVIII as measured by ELISA (FIG. 1).

### Example 3—Quality Control Post-AAV Administration

### Introduction

To rule out that the differences across promoters as shown in FIG. 1 might be due to technical artifacts such as mis-titering of the viral preparation by qPCR, suboptimal dilutions of the test article or to slightly different injection volumes, following sacrifice at week 4 post-AAV administration, tissues were collected for further analysis.

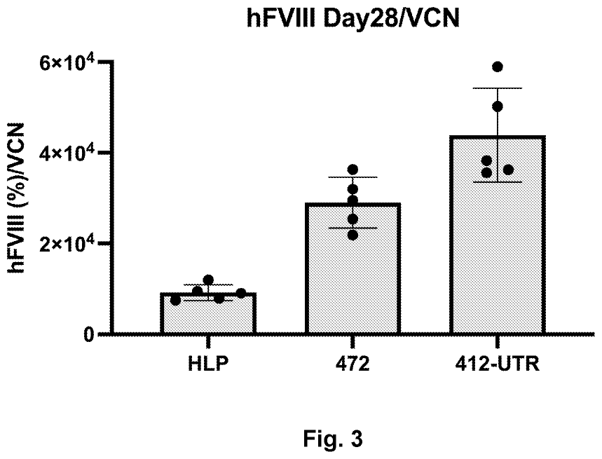

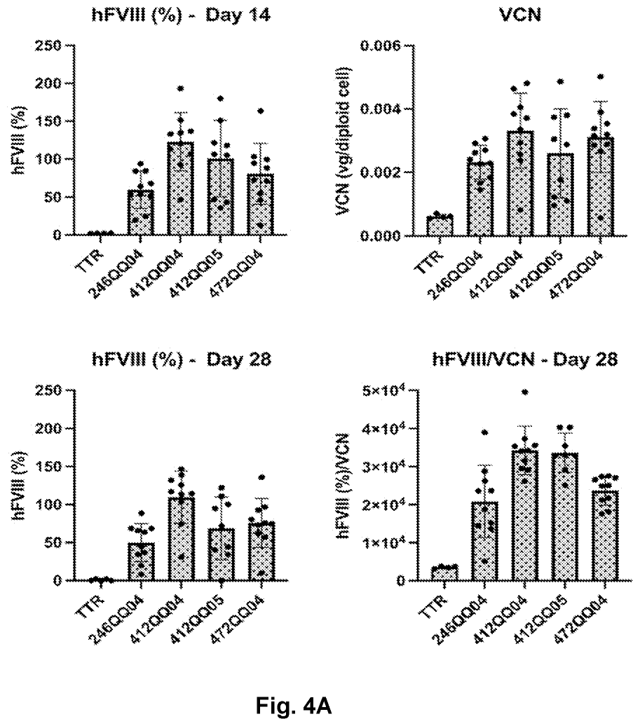

Firstly, vector copy number was assessed in the liver and is shown in FIG. 2. The hepatic vector copy number was then used to normalize the potency of each promoter (FIG. 3).Following normalization to vector copy number in each individual animal, both HLP groups showed the same expression levels regardless of vector dose (only high dose shown), suggesting that at the doses used normalization to VCN is a useful tool to normalize expression to vector load. As shown in FIG. 3 and summarized in Table 20 below, all the promoters tested showed increased potency vs the HLP benchmark.

TABLE 20
VCN-normalized hFVIII expression to the HLP group

| | FVIII Ag/VCN |
|---|---|
| | (relative to HLP) |
| HLP low dose | 1.1 |
| 412 + UTR-hF8co | 4.8 |
| 472-hF8co | 3.2 |

### Example 4—In Vivo Study 2

### Introduction

The aim was to generate an expression cassette containing a coding sequence (CDS) encoding codon optimised hFVIII under the control of liver-specific promoter. Here the potency of liver-specific promoter SP0472 in driving the expression of codon optimised factor VIII was compared with the promoters SP0412, SP0246 and TTR in an in vivo study in mice.

TABLE 21
Study design

| Promoter | CDS |
|---|---|
| TTR | F8-SQ |
| 246 | F8-QQ04 |
| 412 | F8-QQ04 |
| 412 | F8-QQ05 |
| 472 | F8-QQ04 |

### Methods

Methods for production of AAV expressing the constructs described in Table 21 and administration of the AAV were completed as described in Example 2.

AAV vectors described herein in this Example were administered to 8 to 12-week-old male C57BL/6JOlaHsd mice (n=5 mice/vector) in a volume of 200 μL of phosphate-buffered saline/0.001% Pluronic via tail vein injection. Mice were injected with two different doses: one group was injected with 5e9 vg/mouse and 1.68e9 vg/mouse. Mice were sacrificed at 4 weeks.

AAV vectors described herein in this Example were titrated by ITR droplet digital PCR (ddPCR). To extract the DNA from the AAV preparations, vectors were treated first with DNase and then with proteinase K. Next, the AAV DNA was quantified by ddPCR targeting the ITR region of the vector (Forward primer: 5′-GGAACCCCTAGTGATGGAGTT-3′ (SEQ ID NO: 45); Reverse primer: 5′-CGGCCTCAGTGAGCGA-3′ (SEQ ID NO: 46); Probe: 5′-FAM-CACTCCCTCTCTGCGCGCTCG-BHQ1-3′ (SEQ ID NO: 47) and using an appropriate dilution.

Sample collections were performed as described in Example 2. Additionally, all diagnostic assays performed, e.g., Human coagulation factor VIII ELISA, DNA extraction, Hepatic vector copy number quantification (VCN) were performed as described in Example 2. The primers and probes used for VCN determination are listed in Table 22.

Statistical analysis was performed using GraphPad Prism version 9.3.1. Data are presented as mean±SD unless otherwise indicated.

### Results

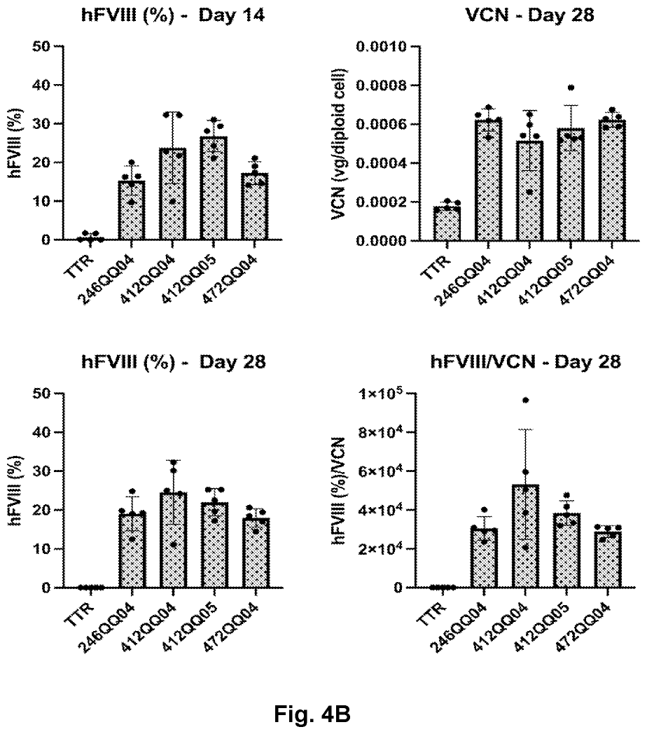

Liver-specific promoters SP0246 (SEQ ID NO: 124), SP0412 (SEQ ID NO: 123), and SP0472 (SEQ ID NO: 26) were placed upstream of a nucleic acid sequence encoding codon-optimized FVIII F8-QQ04 (SEQ ID NO: 64) or F8-QQ05 (SEQ ID NO: 65). The potency of these promoters at driving codon-optimized FVIII expression was benchmarked against TTR as described above and assessed following IV administration of AAV8 vectors. In summary, all candidates vectors elicited circulating human FVIII levels above the baseline (see, e.g., FIGS. 4A and 4B).

Two different doses of the vectors were used with results for 5e9 vg/mouse shown in FIG. 4A and 1.68e9 vg/mouse shown in FIG. 4B. The results show similar FVIII/VCN ratios. For an in vivo assessment of vector potency, mice (N=5 per group) were intravenously administered either one of the different vectors. Two and four weeks post-administration, citrate plasma samples were collected to analyse for the presence of circulating hFVIII as measured by ELISA (FIGS. 4A and 4B).

Following sacrifice at week 4 post-AAV administration, vector copy number was assessed in the liver and is shown in FIGS. 4A and 4B.

To rule out that some of the apparent differences across vectors might be due to technical artifacts such as mis-titering of the viral preparation by ddPCR, suboptimal dilutions of the test article or to slightly different injection volumes, we used the hepatic vector copy number to normalize the potency of each promoter (FIGS. 4A and 4B).

TABLE 22
Specific probe and primers for hF8co sequence used for VCN analysis

| ID | Sequence | SEQ ID NO. |
|---|---|---|
| hF8co_Probe | CCCCTGGGCATGGAGAGCAAGGCC- | SEQ ID NO: 48 |
| | FAM | |
| hF8co_Forward | GATGGGCTGTGACCTGAACA | SEQ ID NO: 49 |
| hF8co_Reverse | CCAGGTGGCAAACATGTTGG | SEQ ID NO: 50 |
| Mouse | 5HEX/TTGTCATCAACGGGAAGCCCATCA | SEQ ID NO: 51 |
| GAPDH_Probe | | |
| Mouse | ACGGCAAATTCAACGGCAC | SEQ ID NO: 52 |
| GAPDH_Forward | | |
| Mouse | TAGTGGGGTCTCGCTCCTGG | SEQ ID NO: 53 |
| GAPDH_Reverse | | |
| hF8SQ_Probe | GTTTGAAACTGTGGAGATGCTGCCCT- | SEQ ID NO: 120 |
| | FAM | |
| hF8SQ_Forward | CCACTTCTCTGGGCATGTGT | SEQ ID NO: 121 |
| hF8SQ_Reverse | CCTCCAGATGCCAGCCTTAG | SEQ ID NO: 122 |

### Example 5—Hepato-Specificity of the Promoters

### Introduction

To assess the liver specificity of the promoters as described herein, the expression of FVIII mRNA in the liver and 11 non-hepatic tissues obtained at week 4 after sacrifice was assessed.

### Methods

PCR-based expression analysis was conducted to measure vector-derived FVIII mRNA relative to the expression of an endogenous reference gene.

### Results

As expected, the liver was the organ with the highest levels of FVIII mRNA followed by the gall bladder, with levels approximately 100-fold lower than the liver depending on the promoter. Very minimal levels of FVIII mRNA were observed in the adrenal glands with SP0412+UTR, with levels approximately 1000-fold lower than in the liver (FIG. 5). All other tissues had FVIII mRNA levels <10-5-fold compared to the liver. Importantly, vector-derived expression in brain and testis was over a million times lower vs liver (FIG. 5). These data suggest that all the promoter assessed in the study are fundamentally hepato-specific.

### Summary

In summary, the preclinical studies presented here showed that we have developed liver-specific promoters, some of which are substantially more potent at driving FVIII expression in mice that a benchmark promoter currently used in clinical trials for the treatment of haemophilia A. In particular, synthetic promoter SP0472 is substantially more potent at driving FVIII expression than control. Similarly, when normalised, SP0412+UTR is also substantially more potent than control at driving FVIII expression in the liver.

### Example 6—In Vitro Study

Synthetic promoters to be tested are cloned upstream of the luciferase reporter gene followed by SV40 late PolyA signal into a vector with a backbone having properties essentially identical to pUC19. DNA preparations are transfected into either Huh7 (a hepato-cellular carcinoma cell line), HeLa (an immortal cell line derived from cervical cancer) or HEK293 (human embryonic kidney cells) to assess transcriptional activity. Huh-7 cells are sourced from JCRB Cell Bank (JCRB0403), HeLa and HEK293 are sourced from ECACC cell bank. All cell lines are grown and maintained according to the cell banks' recommendations.

Transfections are performed in 48 well plates in triplicate using FuGene HD Transfection Reagent (Promega #E2311) at a DNA:FuGene HD ratio of 1:1.1. Luciferase activity is measured 24 hours after transfection. Cells are washed with phosphate buffered saline (PBS), lysed in 100 μl Passive Lysis Buffer (Promega #E194A) and stored at −80° C. overnight. Luciferase activity is quantified using the Luciferase Reporter 1000 assay system (Promega #E4550) following manufacturer's guidelines in 10 μl of lysate using 96 well flat bottom solid white Microplate FluoroNunc plates (ThermoFisher #236105) and luminescence quantified in a FLUOstar Omega plate reader (BMG Labtech) machine.

Synthetic promoters which have high activity in Huh7 cells (e.g. higher than a reference promoter such as LP1, CMV-IE, TBG) but low activity in HeLa and/or HEK293 cells (e.g. lower than a reference promoter such as LP1, CMV-IE, TBG) may be of particular interest.

### Example 7

### Introduction

In an effort to improve in vivo expression of therapeutic human Factor (FVIII) delivered by an AAV vector, codon optimization of a nucleic acid encoding a functional variant of native human FVIII was pursued. The functional variant was a B-domain deleted APC resistant variant (FVIII-R355Q/R581Q) that has demonstrated approximately 5-fold increased procoagulant function relative to wild type B-domain deleted in FVIII injury models in hemophilia A (HA) mice (Wilhelm et al., 2021). This polypeptide is referred to as BDD-FVIII-QQ, or FVIII-QQ for simplicity. The amino acid sequence of the polypeptide is set forth in SEQ ID NO: 60. A series of nucleic acids encoding FVIII-QQ was generated from the codon-optimization process. The codon optimization process involved removal of all CpG's, minimization of alternative open reading frames, maximization of sequence diversity. 18 coding sequences encoding FVIII-QQ (also referred to herein as the human FVIII polypeptide) and assessed for potency with in vivo assays. These nucleic acids were then tested for expression of the encoded protein, for their potential use in the setting of AAV-mediated gene therapy.

### Materials and Methods

Test articles. The nucleic acid sequence that encodes a B domain deleted variant of FVIII was adapted by modifying two codons (355 and 581) to encode Q rather than R (R355Q/R581Q) so that the nucleic acid sequence now encoded the B domain deleted FVIII-QQ variant This nucleic acid sequence (referred to as FVIII-QQ00) served as a benchmark for the codon optimization process. Eighteen coding sequences encoding FVIII-QQ were generated using a proprietary pipeline. These sequences are referred to consecutively from FVIII-QQ01 to QQ18. The nucleotide sequences are shown in Table 15 and correspond to sequences set forth in SEQ ID NOS: 61-78, respectively. Nucleic acids with the respective sequences and the baseline sequence were synthesized by GeneWiz and placed into an AAV based expression plasmid under the control of a liver-specific promoter (Mcintosh et al, 2013), thereby generating a total of 19 different plasmids. These plasmids were identical except for the inserted FVIII-QQ encoding nucleic acids.

Plasmids. AAV based plasmids were generated from the 18 new sequences and the benchmark sequence. The nucleic acid sequences were operatively linked to a liver specific promoter (Mcintosh et al.), SEQ ID NO: 127, and a polyA signal (Levitt et al.). SEQ ID NO: 128 provides the plasmid sequence containing an exemplary codon-optimized sequence.

Hydrodynamic tail vein injection of Plasmid DNA (HTVi administration). 8-week-old B6N-Tyrc-Brd/BrdCrCrl male mice (N=5 per group) were used in hydrodynamic injection experiments. Before plasmid administration, animals were weighted to determine the exact volume for the injection. The volume administered was proportional to the weight of the animal, adjusting it to 100 ml/kg. E.g., for a 20 g mouse the injected volume was 2 ml. The dose of plasmid administered was 2 mg/kg, adjusting it to a final concentration of 20 μg/ml of the solution. For maximal transduction efficacy, the reagent was injected as rapidly as possible (5-7 seconds). Twenty-four hours after administration, citrate plasma samples were collected to analyse for the presence of circulating hFVIII as measured by ELISA.

Sample collection. For HTVi, blood was collected retro-orbitally 24 hours post plasmid administration. 3.8% sodium citrate was added to sterile, low protein binding 1.5 mL tubes to a 1/10 final volume. Blood was then added, gently mixed, and kept at 4° C. until processing. Samples were immediately centrifuged at 10,000 G for 10 minutes at 4° C. and thereafter plasma was collected and stored at −80° C. for further analysis. For tissue collection, the liver was cut into 2 small pieces of ~10 mg and the remaining liver collected in a separate tube. Samples were snap frozen in liquid nitrogen and stored at −80° C. for further analysis.

Human coagulation factor VIII ELISA. Quantification of hFVIII was based on an ELISA assay using the commercial kit Anti-hFVIII (F8C-EIA, Affinity Biologicals). The ELISA plate (442404, Thermo Fisher Scientific) was coated with the capture antibody diluted 1/100 in carbonate buffer and incubated at room temperature for 2 hours. Blocking was not required under the conditions described. The capture antibody was removed by washing three times with wash buffer (PBS tween; 0.1% v/v). Standards and samples were diluted in green sample diluent supplied by the manufacturer and placed in the appropriate wells. The plate was incubated at room temperature for 2 hours. After incubation, plates were washed 3 times with wash buffer and the pre-diluted detection antibody was added to each well. Plates were incubated for 60 minutes at room temperature. After the incubation time, plates were washed 3 times with wash buffer and TMB (A: 51-2606KC; B: 51-2607KC; BD) was added to develop the plates. Following incubation for 20 minutes at room temperature, the reaction was stopped with H₂SO₄ IM. Absorbance was measured at a wavelength of 450 nm in a microplate reader.

The hFVIII concentration of the samples was determined by interpolating their optical density (OD) on the curve generated by a simple nonlinear regression analysis (sigmoidal, 4PL, X is concentration) relating the OD of the standard dilutions to their concentration.

TABLE 14
Specific probe and primers for HLP to determine
vector copy number

| ID | Sequence |
|---|---|
| Probe_HLP | Gggggcgactcagatcccagcc-FAM (SEQ |
| | ID NO: 129) |
| Forward_HLP | Aggacgctgtggtttctgag |
| | (SEQ ID NO: 130) |
| Reverse_HLP | Aggtcaccccagttatcgga (SEQ ID NO: |
| | 131) |

Statistical analysis. Analysis was performed using GraphPad Prism version 9.3.1. Data are presented as mean±SD unless otherwise indicated.

### Results

Eighteen modified coding sequences encoding FVIII-QQ were generated using a proprietary pipeline. These sequences, referred to consecutively as FVIII-QQ01 to QQ18, were each inserted into an AAV based expression plasmid under the control of a liver specific promoter. The respective plasmids were then used to analyze the sequences for expression in vivo systems.

### In Vivo Analysis of Expression of FVIII-QQ-Hydrodynamic Tail Vein Injection of Plasmids

Mice (N=5 per group) were administered the different FVIII-QQ plasmids or an empty plasmid via hydrodynamic tail vein injection. Twenty-four hours after administration, citrate plasma samples were collected to analyze for the presence of circulating hFVIII as measured by ELISA.

Preliminary analysis of the results indicated unexpected variability in detected levels within each plasmid group. This prompted us to further examine the mice for appropriate delivery of the plasmid. Most of the hFVIII expressing plasmids resulted in plasma hVIII levels above background, although QQ16 and QQ18 resulted in minimally detectable levels.

The level of gate expression has been shown to decrease significantly with increasing injection time (Liu, Song, & Liu, 1999). Thus, it was hypothesized that the intra-group variability observed for the circulating hFVIII levels could be due to some animals having received a suboptimal hydrodynamic tail vein injection. To ensure the mice had been successfully dosed, DNA was isolated from livers following terminal sacrifice, and the amount of hFVIII plasmid in the livers was quantified by qPCR. The results showed that some of the mice had levels of plasmid DNA in the liver that suggested mis-injection. These mice had levels similar to that of the vehicle-injected animals and orders of magnitude lower than anticipated. The mice with a plasmid copy number <10000/liver sample were considered as having been mis-injected and were excluded from subsequent hFVIII analysis.

Results are presented in FIG. 6. The animals injected with the empty plasmid (vehicle) showed no evidence of human FVIII in the circulation. Most of the mice that received the plasmids exhibited hFVIII levels above background, although when comparing circulating hFVIII levels to the benchmark QQ00 plasmid group, no statistically significant differences were observed for groups QQ01 through QQ15 (FIG. 6). Of note, cassettes QQ16, QQ17, and QQ18 elicited extremely low levels of hFVIII which cannot be attributed to failed plasmid injection. For all of the animals that received a successful administration of the plasmid, circulating hFVIII levels above background were observed.

In summary, except for mice in groups QQ16, QQ17, and QQ18, all animals that were successfully administered FVIII-encoding plasmids via hydrodynamic tail vein injection had circulating levels of human FVIII above background levels.

### Example 8

The suitability of the synthetic promoters of the present invention in a gene therapy context to drive expression of different genes of interest is confirmed as shown below. In particular, the suitability of the synthetic promoters of the present invention to drive expression of genes encoding a secreted protein are tested.

### Pre-Clinical Animal Studies for FVIII, FIX and GAA

The synthetic liver-specific promoters of the present invention are tested in combination with transgenes FVIII, FIX and GAA in an expression cassette administered to animals via hydrodynamic tail vein injection and then in a gene therapy context in a preclinical animal model using AAV serotypes AAV XL32.1 and AAV8. AAV3 is also being tested.

### Materials and Methods

### In Vivo Analysis of Expression of FVIII, FIX and GAA-Hydrodynamic Tail Vein Injection of Plasmids

Expression cassettes constructs are generated comprising nucleic acid sequences encoding FVIII, FIX or GAA operatively linked to a synthetic liver-specific promoter (e.g., SP0472 or SP0412 v2) and a polyA signal. These cassettes in the form of plasmid DNA are injected via hydrodynamic tail vein injection, and the circulating concentration of FVIII or FIX, or enzymatic activity of GAA is determined in plasma samples 24 hours after injection. Mice (N=5 per group) are administered a plasmid as detailed in Table 15 or a negative control plasmid via hydrodynamic tail vein injection. Plasma samples are collected at twenty-four hours after administration to analyse for the presence of circulating human FVIII or FIX as measured by ELISA or the specific GAA activity.

TABLE 15

| Test Plasmids | Promoter | Transgene |
|---|---|---|
| #1 | SP0412 v2 | FIX |
| #2 | SP0412 v2 | GAA |
| #3 | SP0412 v2 | FVIII |
| #4 | SP0472 | FIX |
| #5 | SP0472 | GAA |
| #6 | SP0472 | FVIII |
| #7 | SP0382 | FVIII |
| #8 | SP0382 | FIX |
| #9 | SP0382 | GAA |
| #10 | SP0383 | FIX |
| #11 | SP0383 | GAA |
| #12 | SP0383 | FVIII |
| #13 | SP0471 | FIX |
| #14 | SP0471 | GAA |
| #15 | SP0471 | FVIII |
| #16 | SP0473 | FIX |
| #17 | SP0473 | GAA |
| #18 | SP0473 | FVIII |
| #19 | SP0474 | FIX |
| #20 | SP0474 | GAA |
| #21 | SP0474 | FVIII |
| #22 | SP0475 | FIX |
| #23 | SP0475 | GAA |
| #24 | SP0475 | FVIII |
| #25 | SP0476 | FIX |
| #26 | SP0476 | GAA |
| #27 | SP0476 | FVIII |
| #28 | SP0477 | FIX |
| #29 | SP0477 | GAA |
| #30 | SP0477 | FVIII |
| #31 | SP0478 | FIX |
| #32 | SP0478 | GAA |
| #33 | SP0478 | FVIII |
| #34 | SP0479 | FIX |
| #35 | SP0479 | GAA |
| #36 | SP0479 | FVIII |
| #37 | SP0480 | FIX |
| #38 | SP0480 | GAA |
| #39 | SP0480 | FVIII |
| #40 | SP0481 | FIX |
| #41 | SP0481 | GAA |
| #42 | SP0481 | FVIII |

### In Vivo Analysis of Expression of FVIII, FIX and GAA-Injection of AAV Particles

Promising candidates from the hydrodynamic tail vein experiment showing increased expression of circulating FVIII, FIX or GAA activity are assessed in vivo when packaged in AAV particles AAV8 and AAVXL32.1. The candidates are also tested with AAV3 capsids.

AAV production. The selected candidates selected from Table 15 are then packaged in different capsids (AAV XL32.1, AAV3 and AAV8). The AAV vectors are produced by triple transfection in high density Pro10 cells, helper plasmid xx680 and repcap plasmid GSK2/8 are used. The cells are harvested on day three and lysed by sonication.

The cells are harvested on day three and lysed by sonication. C57/Bl6 mice are administered the different AAV8 or AAVXL32.1-FVIII, FIX or GAA vectors via tail vein injection. C57/Bl6 mice are also administered AAV3-FVIII, FIX or GAA vectors via tail vein injection. Circulating FVIII or FIX antigen levels or GAA activity are measured two-(day 14) and four-weeks (day 28) following vector administration.

These studies indicate that synthetic liver specific promoters of the present invention can drive expression of different genes in a gene therapy context with a variety of different AAV plasmids. It is contemplated herein that SP0412v2 (SP0412+UTR) can provide more expression of a therapeutic transgene e.g. FVIII, FIX and GAA in liver compared to promoter SP0412.

### Human FVIII Clinical Trial

To assess the synthetic liver-specific promoters of the present invention in the human gene therapy context, a Haemophilia A clinical trial is performed using FVIII replacement therapy where the FVIII expressing gene therapy is administered in the event of a breakthrough bleeding episode.

20 adult patients with haemophilia A (aged >18 years) are enrolled in four dose cohorts per AAV serotype; the lowest-dose cohort are receiving 5×10¹¹ vector genomes/kg (vg/kg) and the highest-dose cohort are receiving 2×10¹² vg/kg of an capsid derived from AAV8 or AAVXL32.1 with a liver-specific promoter of the present invention (e.g., SP0472 or SP0412 v2) and codon-optimized FVIII cDNA encoding FVIII. AAV3 derived capsids with a liver-specific promoter and codon-optimized FVIII cDNA encoding FVIII of the present invention are also tested. Glucocorticoids are administered in cases of suspected immune response.

The primary clinical outcome is changes from baseline in FVIII activity levels after a single outpatient dose of a composition containing a capsid as described above. Circulating human FVIII levels in plasma are measured using a chromogenix FVIII assay.

The circulating levels of FVIII are assessed at a 1-year, 2-year and 5-year interval after first administration of the gene therapy.

These results show that administration of the composition comprising the capsid derived from AAV8 or AAVXL32.1 with a liver-specific promoter of the present invention (e.g. SP0472 or SP0412 v2) and codon-optimized FVIII cDNA encoding FVIII result in a sustained elevated level of FVIII at the 1-year, 2-year and 5-year time point following treatment. Similar results will be observed for AAV3 derived capsids. These results support the suitability of the promoters described herein for use in gene therapy.

### Human FIX Clinical Trial

To assess the synthetic liver-specific promoters of the present invention in the human gene therapy context a Haemophilia B clinical trial are performed using FIX replacement therapy.

20 adult patients with haemophilia B (aged >18 years) with baseline FIX coagulation activity (FIX: C) of less than 2 IU/dL, no FIX inhibitor, and low titre of neutralising antibodies (≤1:4) against vector capsid are enrolled in four dose cohorts per AAV serotype. The study participants are intravenously infused with 2×10¹¹, 1×10¹², 3×10¹² or 5×10¹² vg/kg of a composition containing a capsid derived from AAV8 or AAVXL32.1 with a liver-specific promoter of the present invention (e.g. SP0472 or SP0412 v2) and codon-optimized FIX cDNA encoding FIX after 1 week of prophylactic prednisone treatment (1 mg/kg per day). AAV3 derived capsids with a liver-specific promoter and codon-optimized FIX cDNA encoding FIX of the present invention are also tested.

The primary clinical outcome are changes in FIX levels after a single dose of the composition containing a capsid derived from AAV8 or AAVXL32.1 with a liver-specific promoter of the present invention (e.g. SP0472 or SP0412 v2) and codon-optimized FIX cDNA encoding FIX. AAV3 derived capsids with a liver-specific promoter and codon-optimized FIX cDNA encoding FIX of the present invention are also tested. The output reading is measured by circulating levels of FIX in the blood using a human FIX ELISA.

The circulating levels of FIX are assessed at a 1-year interval after first administration of the gene therapy.

These results show that administration of the gene therapy capsid derived from AAV8 or AAVXL32.1 with a liver-specific promoter of the present invention (e.g. SP0472 or SP0412 v2) and codon-optimized FIX cDNA encoding FIX result in a sustained elevated level of FIX at the 1-year time point following treatment. Similar results will be observed for AAV3 derived capsids. These results support the suitability of the promoters described herein for use in gene therapy.

A clinical trial is being performed using a similar clinical protocol as described in Human FIX Clinical Trial above. Patients enrolled in the GAA study are Pompe patients and GAA enzyme activity is measured at a 1-year interval after first administration of the gene therapy. These results support the suitability of the promoters described herein for use in gene therapy.

### REFERENCES

Ward N J, Buckley S M, Waddington S N, Vandendriessche T, Chuah M K, Nathwani A C, Mcintosh J, Tuddenham E G, Kinnon C, Thrasher A J, McVey J H. Codon optimization of human factor VIII cDNAs leads to high-level expression. Blood. 2011 Jan. 20; 117(3):798-807. doi: 10.1182/blood-2010-05-282707. Epub 2010 Nov. 1. PMID: 21041718.

1. A synthetic liver-specific promoter comprising a combination of one or more cis-regulatory elements (CREs) or functional variants of any thereof operably linked to a promoter element (or a functional variant of any thereof) selected from the group consisting of:
CRE0051, CRE0042, CRE0059 and 5′UTR;
CRE0051, CRE0058 and CRE0070;
CRE0051, CRE0058 and CRE0071;
CRE0042 and CRE0099;
CRE0042 and CRE0073;
CRE0051 and CRE0099;
CRE0051 and CRE0073;
CRE0051, CRE0058 and CRE0054;
CRE0094 and CRE0052;
CRE0056, CRE0094 and CRE0052;
CRE0056, CRE0094 and CRE0059;
CRE0048, CRE0056 and CRE0054;
CRE0048, CRE0056 and CRE0052; and
CRE0048, CRE0056 and CRE0059.

2. The synthetic liver-specific promoter of claim 1 which comprises a promoter selected from the group consisting of: SP0412_v2, SP0382, SP0383, SP0471, SP0472, SP0473, SP0474, SP0475, SP0476, SP0477, SP0478, SP0479, SP0480, and SP0481, or a functional variant of any thereof, wherein the functional variant of any of said promoters comprises a sequence that is at least 70% identical to a reference synthetic liver-specific promoter, more preferably at least 80%, 90%, 95%, 96%, 97%, 98% or 99% identical to the reference synthetic liver-specific promoter.

3. The synthetic liver-specific promoter of claim 1 or 2 which comprises a sequence selected from the group consisting of SEQ ID NO: 22 to SEQ ID NO: 35, or a functional variant of any thereof, wherein the functional variant of any of said promoters comprises a sequence that is at least 70% identical to a reference synthetic liver-specific promoter sequence, more preferably at least 80%, 90%, 95%, 96%, 97%, 98% or 99% identical to the reference synthetic liver-specific promoter sequence.

4. The synthetic liver-specific promoter of any preceding claim which has a length of 350 or fewer nucleotides, 340, 330, 320, 310, 300, 290, 280, 270, 260, 250, 240, 230, 220 or fewer nucleotides.

5. A synthetic liver-specific promoter comprising one of the following promoter elements: CRE0059, CRE0070, CRE0071, CRE0054, CRE0073, CRE0099 and CRE0052 or a functional variant thereof.

6. The synthetic liver-specific promoter according to claim 5 comprising one of the following promoter elements: CRE0070, CRE0071, CRE0099 and CRE0054 or a functional variant thereof.

7. The synthetic liver-specific promoter of claim 5 or 6 which has a length of 350 or fewer nucleotides, 340, 330, 320, 310, 300, 290, 280, 270, 260, 250, 240, 230, 220 or fewer nucleotides.

8. A promoter element comprising any one of CRE0059, CRE0070, CRE0071, CRE0054, CRE0073, CRE0099 and CRE0052 or a functional variant thereof.

9. The promoter element according to claim 8 comprising any one of CRE0070, CRE0071, CRE0099 and CRE0054 or a functional variant thereof.

10. The promoter element of claim 8 or 9 wherein the promoter element has a length of 200 or fewer nucleotides, 190, 180, 170, 160, 150, 140, 130, 120, 110, 100, 90, 80 or fewer nucleotides.

11. A liver-specific CRE selected from the group consisting of: CRE0051, CRE0042, CRE0058, CRE0048, CRE0094 and CRE0056, or a functional variant of any thereof, wherein the functional variant of any of said CREs comprises a sequence that is at least 70% identical to a reference synthetic liver-specific CRE, more preferably at least 80%, 90%, 95%, 96%, 97%, 98% or 99% identical to the reference synthetic liver-specific CRE.

12. The liver-specific CRE according to claim 11, wherein the CRE comprises CRE0094 or a functional variant thereof.

13. A synthetic liver-specific cis-regulatory module (CRM) or a synthetic liver-specific promoter comprising one or more of the liver-specific CREs according to claim 11, or functional variants thereof.

14. The synthetic liver-specific CRM according to claim 13 which comprises one of the following combinations of CREs, or functional variants thereof:
CRE0051 and CRE0042;
CRE0051 and CRE0058;
CRE0056 and CRE0094; and
CRE0048 and CRE0056.

15. The synthetic liver-specific CRM according to claim 14 which comprises one of the following combinations of CREs, or functional variants thereof:
CRE0048 and CRE0056; or
CRE0056 and CRE0094.

16. The synthetic liver-specific CRM according to claim 14 wherein the CRM is selected from the group consisting of: CRM_SP0412_v2, CRM_SP0382 and SP0383, CRM_SP0471, CRM_SP0473, CRM_SP0475, CRM_SP0477 and SP0478, CRM_SP0479 and CRM_SP0480 and SP0481, or a functional variant of any thereof, wherein the functional variant of any of said CRMs comprises a sequence that is at least 70% identical to a reference synthetic liver-specific CRM, more preferably at least 80%, 90%, 95%, 96%, 97%, 98% or 99% identical to the reference synthetic liver-specific CRM.

17. The synthetic liver-specific CRM according to claim 15 wherein the CRM is selected from the group consisting of: CRM_SP0477 and SP0478, CRM_SP0479, and CRM_SP0480 and SP0481, or a functional variant of any thereof, wherein the functional variant of any of said CRMs comprises a sequence that is at least 70% identical to a reference synthetic liver-specific CRM, more preferably at least 80%, 90%, 95%, 96%, 97%, 98% or 99% identical to the reference synthetic liver-specific CRM.

18. A synthetic liver-specific promoter comprising at least one liver-specific CRE according to claim 11 operably linked to a promoter element.

19. The synthetic liver-specific promoter according to claim 18 wherein the promoter element is selected from the group consisting of: CRE0054, CRE0099, CRE0070 and CRE0071, or a functional variant thereof.

20. An expression cassette comprising a synthetic liver-specific promoter according to any one of claims 1-7, 18-19 or a promoter element according to any one of claims 8-10, a CRE according to any one of claims 11-12, or a CRM according to any one of claims 13-17, operably linked to a sequence encoding an expression product, optionally wherein the sequence encoding the expression product is codon optimised.

21. A vector comprising a synthetic liver-specific promoter according to any one of claims 1-7, 18-19, a promoter element according to any one of claims 8-10, a CRE according to any one of claims 11-12, or a CRM according to any one of claims 13-17, or an expression cassette according to claim 20.

22. The vector of claim 21 which is a viral vector.

23. The vector of claim 22 wherein the viral vector is an AAV vector.

24. A virion comprising a vector according to any one of claims 22-23.

25. A pharmaceutical composition comprising a synthetic liver-specific promoter according to any one of claims 1-7, 18-19, a promoter element according to any one of claims 8-10, a CRE according to any one of claims 11-12, or a CRM according to any one of claims 13-17, or an expression cassette according to claim 20, a vector according to any one of claims 22-23, or a virion according to claim 24.

26. A synthetic liver-specific promoter according to any one of claims 1-7, 18-19, a promoter element according to any one of claims 8-10, a CRE according to any one of claims 11-12, or a CRM according to any one of claims 13-17, or an expression cassette according to claim 20, a vector according to any one of claims 22-23, a virion according to claim 24, or a pharmaceutical composition according to claim 25 for use in therapy.

27. The composition for use according to claim 26 wherein the use is for gene therapy, suitably wherein the gene therapy involves expression of a therapeutic expression product in the liver.

28. The composition for use according to any one of claims 26-27 wherein the use is for gene therapy for Haemophilia A or Haemophilia B.

29. An expression cassette according to claim 20, a vector according to any one of claims 22-23, a virion according to claim 24, or a pharmaceutical composition according to claim 25 for use in gene therapy for Haemophilia A or Haemophilia B, wherein the expression product is Factor VIII or Factor IX.

30. The composition for use according to claim 29 wherein the use is for gene therapy for Haemophilia A, wherein the expression product is Factor VIII.

31. The composition for use according to claim 30, wherein the expression product is a codon optimised Factor VIII.

32. A cell comprising a synthetic liver-specific promoter according to any one of 1-7, 18-19, a promoter element according to any one of claims 8-10, a CRE according to any one of claims 11-12, or a CRM according to any one of claims 13-17, or an expression cassette according to claim 20, a vector according to any one of claims 22-23, or a virion according to claim 24.

33. The cell of claim 32 wherein the cell is a liver cell, optionally a human liver cell.

34. A synthetic liver-specific promoter according to any one of 1-7, 18-19, a promoter element according to any one of claims 8-10, a CRE according to any one of claims 11-12, or a CRM according to any one of claims 13-17, or an expression cassette according to claim 20, a vector according to any one of claims 22-23, a virion according to claim 24, or a pharmaceutical composition according to claim 25 for use in the manufacture of a pharmaceutical composition for the treatment of a medical condition or disease.

35. A method for producing an expression product, the method comprising providing a synthetic liver-specific expression cassette according to claim 20 in a liver cell and expressing the gene present in the synthetic liver-specific expression cassette.

36. A method of expressing a therapeutic transgene in a liver cell, the method comprising introducing into the liver cell a synthetic liver-specific expression cassette according to claim 20, a vector according to any one of claims 21-23, or a virion according to claim 24.

37. A method of therapy of a subject, preferably a human, in need thereof, the method comprising:
administering to the subject an expression cassette according to claim 20, a vector according to any one of claims 22-23, a virion according to claim 24, or a pharmaceutical composition according to claim 25, which comprises a sequence encoding a therapeutic product operably linked to the synthetic liver-specific promoter; and
expressing a therapeutic amount of the therapeutic product in the liver of said subject.

38. The method according to claim 37 wherein the therapeutic product is FVIII and the subject has Haemophilia A.

39. The method according to any one of claims 37-38 comprising administering a vector according to any one of claims 22-23, a virion according to claim 24, or a pharmaceutical composition according to claim 25.


SEQUENCE LISTING (131 sequences)

SEQ ID NO: 1 - DNA, 100 bp, synthetic construct
  source 1..100: mol_type = other DNA; note = CRE0051
aggttaatttttaaaaagcagtcaaaagtccaagtggcccttggcagcatttactctctctgtttgctctggttaataatctcaggagcacaaacattcc

SEQ ID NO: 2 - DNA, 80 bp, synthetic construct
  source 1..80: mol_type = other DNA; note = CRE0042
ccctgttcaaacatgtcctaatactctgtctctgcaagggtcatcagtagttttccatcttactcaacatcctcccagtg

SEQ ID NO: 3 - DNA, 40 bp, synthetic construct
  source 1..40: mol_type = other DNA; note = CRE0058
ggcccgggaggcgccctttggaccttttgcaatcctggcg

SEQ ID NO: 4 - DNA, 92 bp, synthetic construct
  source 1..92: mol_type = other DNA; note = CRE0048
tgctctctgacaaagatacggtgggtcccactgatgaactgtgctgccacagtaaatgtagccactatgcctatctccattctgaagatgtg

SEQ ID NO: 5 - DNA, 171 bp, synthetic construct
  source 1..171: mol_type = other DNA; note = CRE0094
cctgaagtccaaaggtagaggaaccgagtgtttaaaaattactgtggctgtggagtcaacatgatgtaaaaaaacaaacatttggataacaccaagaagccagatatggttgaaatgttgactggttgacaaaaataatttgggttgcttaatggtgcacaaaggtaatgc

SEQ ID NO: 6 - DNA, 79 bp, synthetic construct
  source 1..79: mol_type = other DNA; note = CRE0056
ccgcccccactgaacccttgacccctgccctgcagcccccgcagcttgctgtttgcccactctatttgcccagccccag

SEQ ID NO: 7 - DNA, 95 bp, synthetic construct
  source 1..95: mol_type = other DNA; note = CRE0059
agtcatatgtttgctcactgaaggttactagttaacaggcatcccttaaacaggatataaaaggacttcagcaggactgctcgaaacatcccact

SEQ ID NO: 8 - DNA, 89 bp, synthetic construct
  source 1..89: mol_type = other DNA; note = CRE0070
tctagagggtatataatgggggccactagtgccagcagcagcctgaccacatctcatcctcaatcaccaccaagcctggaataactgca

SEQ ID NO: 9 - DNA, 74 bp, synthetic construct
  source 1..74: mol_type = other DNA; note = CRE0071
tctagagggtatataatgggggccagccgctggcgtttccgttgcttggatcagtctaggtgcagctgccggat

SEQ ID NO: 10 - DNA, 136 bp, synthetic construct
  source 1..136: mol_type = other DNA; note = CRE0054
agatcccagccagtggacttagcccctgtttgctcctccgataactggggtgaccttggttaatattcaccagcagcctcccccgttgcccctctggatccactgcttaaatacggacgaggacagggccctgtct

SEQ ID NO: 11 - DNA, 186 bp, synthetic construct
  source 1..186: mol_type = other DNA; note = CRE0073
gggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 12 - DNA, 76 bp, synthetic construct
  source 1..76: mol_type = other DNA; note = CRE0052
gggcatataaaacaggggcaaggcacagactcatagcagagcaatcaccaccaagcctggaataactgcagccacc

SEQ ID NO: 13 - DNA, 43 bp, synthetic construct
  source 1..43: mol_type = other DNA; note = 5 UTR
caggtggattcttgggcatttgcttaagcttggtaccgccacc

SEQ ID NO: 14 - DNA, 180 bp, synthetic construct
  source 1..180: mol_type = other DNA; note = CRM_SP0412_v2
aggttaatttttaaaaagcagtcaaaagtccaagtggcccttggcagcatttactctctctgtttgctctggttaataatctcaggagcacaaacattccccctgttcaaacatgtcctaatactctgtctctgcaagggtcatcagtagttttccatcttactcaacatcctcccagtg

SEQ ID NO: 15 - DNA, 140 bp, synthetic construct
  source 1..140: mol_type = other DNA; note = CRM_SP0382 and SP0383
aggttaatttttaaaaagcagtcaaaagtccaagtggcccttggcagcatttactctctctgtttgctctggttaataatctcaggagcacaaacattccggcccgggaggcgccctttggaccttttgcaatcctggcg

SEQ ID NO: 16 - DNA, 90 bp, synthetic construct
  source 1..90: mol_type = other DNA; note = CRM_SP0471
ccctgttcaaacatgtcctaatactctgtctctgcaagggtcatcagtagttttccatcttactcaacatcctcccagtgcgggcgactc

SEQ ID NO: 17 - DNA, 110 bp, synthetic construct
  source 1..110: mol_type = other DNA; note = CRM_SP0473
aggttaatttttaaaaagcagtcaaaagtccaagtggcccttggcagcatttactctctctgtttgctctggttaataatctcaggagcacaaacattcccgggcgactc

SEQ ID NO: 18 - DNA, 146 bp, synthetic construct
  source 1..146: mol_type = other DNA; note = CRM_SP0475
aggttaatttttaaaaagcagtcaaaagtccaagtggcccttggcagcatttactctctctgtttgctctggttaataatctcaggagcacaaacattcctgtaccggcccgggaggcgccctttggaccttttgcaatcctggcg

SEQ ID NO: 19 - DNA, 250 bp, synthetic construct
  source 1..250: mol_type = other DNA; note = CRM_SP0477 and SP0478
ccgcccccactgaacccttgacccctgccctgcagcccccgcagcttgctgtttgcccactctatttgcccagccccagcctgaagtccaaaggtagaggaaccgagtgtttaaaaattactgtggctgtggagtcaacatgatgtaaaaaaacaaacatttggataacaccaagaagccagatatggttgaaatgttgactggttgacaaaaataatttgggttgcttaatggtgcacaaaggtaatgc

SEQ ID NO: 20 - DNA, 180 bp, synthetic construct
  source 1..180: mol_type = other DNA; note = CRM_SP0479
tgctctctgacaaagatacggtgggtcccactgatgaactgtgctgccacagtaaatgtagccactatgcctatctccattctgaagatgtgccgcccccactgaacccttgacccctgccctgcagcccccgcagcttgctgtttgcccactctatttgcccagccccaggggcgactc

SEQ ID NO: 21 - DNA, 171 bp, synthetic construct
  source 1..171: mol_type = other DNA; note = CRM_SP0480 and SP0481
tgctctctgacaaagatacggtgggtcccactgatgaactgtgctgccacagtaaatgtagccactatgcctatctccattctgaagatgtgccgcccccactgaacccttgacccctgccctgcagcccccgcagcttgctgtttgcccactctatttgcccagccccag

SEQ ID NO: 22 - DNA, 318 bp, synthetic construct
  source 1..318: mol_type = other DNA; note = SP0412_v2
aggttaatttttaaaaagcagtcaaaagtccaagtggcccttggcagcatttactctctctgtttgctctggttaataatctcaggagcacaaacattccccctgttcaaacatgtcctaatactctgtctctgcaagggtcatcagtagttttccatcttactcaacatcctcccagtgagtcatatgtttgctcactgaaggttactagttaacaggcatcccttaaacaggatataaaaggacttcagcaggactgctcgaaacatcccactcaggtggattcttgggcatttgcttaagcttggtaccgccacc

SEQ ID NO: 23 - DNA, 229 bp, synthetic construct
  source 1..229: mol_type = other DNA; note = SP0382
aggttaatttttaaaaagcagtcaaaagtccaagtggcccttggcagcatttactctctctgtttgctctggttaataatctcaggagcacaaacattccggcccgggaggcgccctttggaccttttgcaatcctggcgtctagagggtatataatgggggccactagtgccagcagcagcctgaccacatctcatcctcaatcaccaccaagcctggaataactgca

SEQ ID NO: 24 - DNA, 214 bp, synthetic construct
  source 1..214: mol_type = other DNA; note = SP0383
aggttaatttttaaaaagcagtcaaaagtccaagtggcccttggcagcatttactctctctgtttgctctggttaataatctcaggagcacaaacattccggcccgggaggcgccctttggaccttttgcaatcctggcgtctagagggtatataatgggggccagccgctggcgtttccgttgcttggatcagtctaggtgcagctgccggat

SEQ ID NO: 25 - DNA, 226 bp, synthetic construct
  source 1..226: mol_type = other DNA; note = SP0471
ccctgttcaaacatgtcctaatactctgtctctgcaagggtcatcagtagttttccatcttactcaacatcctcccagtgcgggcgactcagatcccagccagtggacttagcccctgtttgctcctccgataactggggtgaccttggttaatattcaccagcagcctcccccgttgcccctctggatccactgcttaaatacggacgaggacagggccctgtct

SEQ ID NO: 26 - DNA, 268 bp, synthetic construct
  source 1..268: mol_type = other DNA; note = SP0472
ccctgttcaaacatgtcctaatactctgtctctgcaagggtcatcagtagttttccatcttactcaacatcctcccagtgcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcg

SEQ ID NO: 27 - DNA, 246 bp, synthetic construct
  source 1..246: mol_type = other DNA; note = SP0473
aggttaatttttaaaaagcagtcaaaagtccaagtggcccttggcagcatttactctctctgtttgctctggttaataatctcaggagcacaaacattcccgggcgactcagatcccagccagtggacttagcccctgtttgctcctccgataactggggtgaccttggttaatattcaccagcagcctcccccgttgcccctctggatccactgcttaaatacggacgaggacagggccctgtct

SEQ ID NO: 28 - DNA, 288 bp, synthetic construct
  source 1..288: mol_type = other DNA; note = SP0474
aggttaatttttaaaaagcagtcaaaagtccaagtggcccttggcagcatttactctctctgtttgctctggttaataatctcaggagcacaaacattcccgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcg

SEQ ID NO: 29 - DNA, 290 bp, synthetic construct
  source 1..290: mol_type = other DNA; note = SP0475
aggttaatttttaaaaagcagtcaaaagtccaagtggcccttggcagcatttactctctctgtttgctctggttaataatctcaggagcacaaacattcctgtaccggcccgggaggcgccctttggaccttttgcaatcctggcgggcgactcagatcccagccagtggacttagcccctgtttgctcctccgataactggggtgaccttggttaatattcaccagcagcctcccccgttgcccctctggatccactgcttaaatacggacgaggacagggccctgtct

SEQ ID NO: 30 - DNA, 247 bp, synthetic construct
  source 1..247: mol_type = other DNA; note = SP0476
cctgaagtccaaaggtagaggaaccgagtgtttaaaaattactgtggctgtggagtcaacatgatgtaaaaaaacaaacatttggataacaccaagaagccagatatggttgaaatgttgactggttgacaaaaataatttgggttgcttaatggtgcacaaaggtaatgcgggcatataaaacaggggcaaggcacagactcatagcagagcaatcaccaccaagcctggaataactgcagccacc

SEQ ID NO: 31 - DNA, 326 bp, synthetic construct
  source 1..326: mol_type = other DNA; note = SP0477
ccgcccccactgaacccttgacccctgccctgcagcccccgcagcttgctgtttgcccactctatttgcccagccccagcctgaagtccaaaggtagaggaaccgagtgtttaaaaattactgtggctgtggagtcaacatgatgtaaaaaaacaaacatttggataacaccaagaagccagatatggttgaaatgttgactggttgacaaaaataatttgggttgcttaatggtgcacaaaggtaatgcgggcatataaaacaggggcaaggcacagactcatagcagagcaatcaccaccaagcctggaataactgcagccacc

SEQ ID NO: 32 - DNA, 345 bp, synthetic construct
  source 1..345: mol_type = other DNA; note = SP0478
ccgcccccactgaacccttgacccctgccctgcagcccccgcagcttgctgtttgcccactctatttgcccagccccagcctgaagtccaaaggtagaggaaccgagtgtttaaaaattactgtggctgtggagtcaacatgatgtaaaaaaacaaacatttggataacaccaagaagccagatatggttgaaatgttgactggttgacaaaaataatttgggttgcttaatggtgcacaaaggtaatgcagtcatatgtttgctcactgaaggttactagttaacaggcatcccttaaacaggatataaaaggacttcagcaggactgctcgaaacatcccact

SEQ ID NO: 33 - DNA, 316 bp, synthetic construct
  source 1..316: mol_type = other DNA; note = SP0479
tgctctctgacaaagatacggtgggtcccactgatgaactgtgctgccacagtaaatgtagccactatgcctatctccattctgaagatgtgccgcccccactgaacccttgacccctgccctgcagcccccgcagcttgctgtttgcccactctatttgcccagccccaggggcgactcagatcccagccagtggacttagcccctgtttgctcctccgataactggggtgaccttggttaatattcaccagcagcctcccccgttgcccctctggatccactgcttaaatacggacgaggacagggccctgtct

SEQ ID NO: 34 - DNA, 247 bp, synthetic construct
  source 1..247: mol_type = other DNA; note = SP0480
tgctctctgacaaagatacggtgggtcccactgatgaactgtgctgccacagtaaatgtagccactatgcctatctccattctgaagatgtgccgcccccactgaacccttgacccctgccctgcagcccccgcagcttgctgtttgcccactctatttgcccagccccaggggcatataaaacaggggcaaggcacagactcatagcagagcaatcaccaccaagcctggaataactgcagccacc

SEQ ID NO: 35 - DNA, 266 bp, synthetic construct
  source 1..266: mol_type = other DNA; note = SP0481
tgctctctgacaaagatacggtgggtcccactgatgaactgtgctgccacagtaaatgtagccactatgcctatctccattctgaagatgtgccgcccccactgaacccttgacccctgccctgcagcccccgcagcttgctgtttgcccactctatttgcccagccccagagtcatatgtttgctcactgaaggttactagttaacaggcatcccttaaacaggatataaaaggacttcagcaggactgctcgaaacatcccact

SEQ ID NO: 36 - DNA, 130 bp, synthetic construct
  source 1..130: mol_type = other DNA; note = CRE0099
cgggcgactcagatcccagccagtggacttagcccctgtttgctcctccgataactggggtgaccttggttaatattcaccagcagcctcccccgttgcccctctggatccactgcttaaatacggacga

SEQ ID NO: 37 - DNA, 51 bp, synthetic construct
  source 1..51: mol_type = other DNA; note = CRE0078 (V2)
ggcgccctttggaccttttgcaatcctggagcaaacagcaaacactgtacc

SEQ ID NO: 38 - DNA, 58 bp, synthetic construct
  source 1..58: mol_type = other DNA; note = portion of the SEPRINA1 promoter
ggacttagcccctgtttgctcctccgataactggggtgaccttggttaatattcacca

SEQ ID NO: 39 - DNA, 51 bp, synthetic construct
  source 1..51: mol_type = other DNA; note = portion of the SEPRINA1 promoter
gcccctgtttgctcctccgataactggggtgaccttggttaatattcacca

SEQ ID NO: 40 - DNA, 179 bp, synthetic construct
  source 1..179: mol_type = other DNA; note = HBB2
tactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaatt

SEQ ID NO: 41 - DNA, 818 bp, synthetic construct
  source 1..818: mol_type = other DNA; note = UBC intron
acttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatgcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagac

SEQ ID NO: 42 - DNA, 173 bp, synthetic construct
  source 1..173: mol_type = other DNA; note = CMV-IE intron
caagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcag

SEQ ID NO: 43 - DNA, 92 bp, synthetic construct
  source 1..92: mol_type = other DNA; note = Exon and splice donor_ CRE0059
tccagcactgcctgcggtgaaggaaccagcagcctacatttcgctgcgtccaaagcattgcacgaaaaagagtttgggataggtaagatgct

SEQ ID NO: 44 - DNA, 144 bp, synthetic construct
  source 1..144: mol_type = other DNA; note = Exon and splice donor_CRE0052.1
atcttggtgtccgtgatcgcagacctcaggaagccttctacgtcctcttccccatctggttccatcttcaggaagctgtgggcattaaactcctttgggtagctgtgattggagactggctcaacctcgtctttaagtggtaag

SEQ ID NO: 45 - DNA, 21 bp, synthetic construct
  source 1..21: mol_type = other DNA; note = Forward primer
ggaacccctagtgatggagtt

SEQ ID NO: 46 - DNA, 16 bp, synthetic construct
  source 1..16: mol_type = other DNA; note = Reverse primer
cggcctcagtgagcga

SEQ ID NO: 47 - DNA, 21 bp, synthetic construct
  source 1..21: mol_type = other DNA; note = probe
  misc_feature 1: note = FAM
  misc_feature 21: note = BHQ1
cactccctctctgcgcgctcg

SEQ ID NO: 48 - DNA, 24 bp, synthetic construct
  source 1..24: mol_type = other DNA; note = hF8co_Probe
  misc_feature 24: note = FAM
cccctgggcatggagagcaaggcc

SEQ ID NO: 49 - DNA, 20 bp, synthetic construct
  source 1..20: mol_type = other DNA; note = hF8co_Forward
gatgggctgtgacctgaaca

SEQ ID NO: 50 - DNA, 20 bp, synthetic construct
  source 1..20: mol_type = other DNA; note = hF8co_Reverse
ccaggtggcaaacatgttgg

SEQ ID NO: 51 - DNA, 24 bp, synthetic construct
  source 1..24: mol_type = other DNA; note = Mouse GAPDH_Probe
  misc_feature 1: note = 5HEX
ttgtcatcaacgggaagcccatca

SEQ ID NO: 52 - DNA, 19 bp, synthetic construct
  source 1..19: mol_type = other DNA; note = Mouse GAPDH_Forward
acggcaaattcaacggcac

SEQ ID NO: 53 - DNA, 20 bp, synthetic construct
  source 1..20: mol_type = other DNA; note = Mouse GAPDH_Reverse
tagtggggtctcgctcctgg

SEQ ID NO: 54 - DNA, 24 bp, synthetic construct
  source 1..24: mol_type = other DNA; note = SQ_Probe
  misc_feature 24: note = FAM
cccctgggcatggagagcaaggcc

SEQ ID NO: 55 - DNA, 20 bp, synthetic construct
  source 1..20: mol_type = other DNA; note = SQ_Forward
gatgggctgtgacctgaaca

SEQ ID NO: 56 - DNA, 20 bp, synthetic construct
  source 1..20: mol_type = other DNA; note = SQ_Reverse
ccaggtggcaaacatgttgg

SEQ ID NO: 57 - DNA, 23 bp, synthetic construct
  source 1..23: mol_type = other DNA; note = Rplp0_Probe
  misc_feature 1: note = 5HEX
  misc_feature 9..10: note = ZEN
tggagggtgtccgcaacgtggcc

SEQ ID NO: 58 - DNA, 20 bp, synthetic construct
  source 1..20: mol_type = other DNA; note = Rplp0_Forward
caggtgtttgacaacggcag

SEQ ID NO: 59 - DNA, 20 bp, synthetic construct
  source 1..20: mol_type = other DNA; note = Rplp0_Reverse
ccgatctgcagacacacact

SEQ ID NO: 60 - protein, 1457 aa, Homo sapiens
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLQMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQQGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 61 - DNA, 4374 bp, synthetic construct
  source 1..4374: mol_type = other DNA; note = FVIII-QQ01
atgcagattgagctgagcacctgcttcttcctgtgcctgctgagattctgcttctctgccaccagaagatactacctgggtgcagtggagctgagctgggactacatgcagagtgacctgggtgagctgcctgttgatgccagattcccccccagagtgcccaagagcttccccttcaacacctctgttgtgtacaagaagaccctgtttgttgagttcactgaccacctgttcaacattgccaagcccagacccccctggatgggcctgctgggccccaccatccaggcagaggtgtatgatactgtggtgatcaccctgaagaacatggccagccacccagttagcctgcatgcagtgggggtcagctactggaaggcctcagaaggggctgagtatgatgatcagaccagccagagagagaaggaggatgacaaggtgttcccagggggtagccacacctatgtatggcaggtgctgaaggagaatggccccatggcctctgaccccctgtgcctgacctacagctacctgagccatgtggacctggtgaaggacctgaacagtggactgattggtgccctgctggtgtgcagagagggcagcctggccaaggagaagacccagaccctgcacaagttcatcctgctgtttgctgtgtttgatgaaggcaagagctggcacagtgagaccaagaacagcctgatgcaggacagagatgctgcatcagctagagcctggcccaagatgcacactgtcaatggttatgtaaacagaagcctgcctggcctgattggctgccacagaaagtcagtatactggcatgttataggcatgggcaccaccccagaggtgcacagcatcttcctggagggccacaccttcctggtgagaaaccacagacaggccagcctggagatcagccccatcaccttcctgacagcccagaccctgctgatggacctgggccagttcctgctgttctgccacatcagcagccaccagcatgatgggatggaggcctatgtaaaggtggacagctgcccagaggagccccagctgcagatgaagaacaatgaagaggctgaagactatgatgatgatctgactgattcagagatggatgttgtgagatttgatgatgacaacagccccagcttcatccagatcagatcagtggccaagaagcaccccaagacctgggtgcactacatagctgcagaggaggaggactgggactatgcccccctggtgctggccccagatgatagaagctacaagagccagtacctgaacaatggcccccagagaatagggagaaagtacaagaaggtgagattcatggcctacacagatgagaccttcaagaccagagaggccatccagcatgagtcaggtatcctgggccccctgctgtatggggaggtgggtgacaccctgctgatcatcttcaagaaccaggccagcagaccctacaacatctacccccatgggatcacagatgtcagacccctgtacagcagaagactgcccaagggagtcaagcacctgaaggacttccccatcctgcctggagaaatcttcaagtacaagtggacagtgactgtggaggatggtcccaccaagagtgaccccagatgcctgaccagatactacagcagctttgtcaacatggagagagacctggccagtggcctgataggccccctgctgatctgctacaaggagtctgtggaccagcagggcaaccagatcatgtctgacaagagaaatgttatcctgttcagtgtatttgatgagaacagaagctggtacctgactgagaacatccagagattcctgcccaacccagctggggtccagctggaggaccctgagttccaggccagcaacatcatgcacagcatcaatggctatgtgtttgacagcctgcagctgtctgtctgcctgcatgaagtggcctactggtacatcctgagcataggagcccagacagacttcctgagtgtattcttctctggctacaccttcaagcacaagatggtgtatgaggacaccctgaccctgttccccttctctggagagactgtattcatgagcatggagaaccctggcctgtggatcctgggctgccacaactctgacttcagaaacagaggcatgactgccctgctgaaggtgagcagctgtgataagaacacaggggactactatgaggacagctatgaggacatctcagcctacctgctgagcaagaacaatgccattgagcccagaagcttcagccagaacccccctgtgctgaagagacaccagagagagatcaccagaaccaccctgcagagtgaccaggaggagattgactatgatgacaccatctctgttgagatgaagaaggaggactttgacatctatgatgaggatgagaaccagagccccagaagcttccagaagaagaccagacactacttcattgcagctgtggagagactgtgggactatggcatgagcagcagcccccatgtgctgagaaacagagcccagtcaggctctgtgccccagttcaagaaggtggtgttccaggagttcacagatgggagcttcacccagcccctgtacagaggtgaactgaatgagcacctgggcctgctgggcccctacatcagagctgaagtggaggacaacatcatggtgaccttcagaaaccaggccagcagaccctacagcttctacagcagcctgatcagctatgaagaggaccagagacagggtgctgagcccagaaagaactttgtgaagcccaatgaaaccaagacctacttctggaaggtgcagcaccacatggcccccaccaaggatgaatttgactgcaaggcctgggcctacttctcagatgtagacctggagaaggatgttcactcaggcctgattgggcccctgctggtgtgccacaccaacaccctgaacccagcccatggtagacaggtgacagtgcaggagtttgccctgttcttcaccatctttgatgagaccaagagctggtacttcactgagaacatggagagaaactgcagagccccctgcaacatccagatggaggaccccaccttcaaggagaactacagattccatgccatcaatgggtacatcatggacaccctgccaggcctggtgatggcccaggaccagagaatcagatggtacctgctgagcatgggcagcaatgagaacatccacagcatccacttcagtggtcatgtcttcacagtgagaaagaaggaggagtacaagatggccctgtacaacctgtacccaggggtgtttgaaactgtagagatgctgcccagcaaggcaggcatctggagagtggagtgcctgataggagaacacctgcatgcaggaatgagcaccctgttcctggtgtacagcaacaagtgccagacccccctgggcatggcctctgggcacatcagagacttccagatcactgcctctggccagtatgggcagtgggcccccaagctggccagactgcactactcaggtagcatcaatgcctggagcaccaaggagcccttcagctggatcaaggtggacctgctggcccccatgatcatccatggtatcaagacccagggtgccagacagaagttcagcagcctgtacatcagccagttcatcatcatgtacagcctggatggcaagaagtggcagacctacagaggcaacagcactggcaccctgatggtgttctttggcaatgtggacagctctggcatcaagcacaacatcttcaacccccccatcatagccagatacatcagactgcaccccacccactacagcatcagaagcaccctgagaatggagctgatgggctgtgatctgaacagctgcagcatgcccctgggcatggagagcaaggccatctctgatgctcagatcactgcaagcagctacttcaccaacatgtttgccacctggagccccagcaaggccagactgcacctgcagggcagaagcaatgcctggagaccccaggtgaacaaccccaaggagtggctgcaggtggacttccagaagaccatgaaggtgacaggtgtcaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgagaatccacccccagagctgggtgcaccagattgccctgagaatggaggtgctgggctgtgaggcccaggacctgtactga

SEQ ID NO: 62 - DNA, 4374 bp, synthetic construct
  source 1..4374: mol_type = other DNA; note = FVIII-QQ02
atgcagattgagctgagcacctgcttcttcctgtgcctgctgagattctgcttcagtgccaccagaagatactacctgggagcagtggagctgagctgggactacatgcagagtgatctgggagagctgccagtagatgccagattcccccccagagtgcccaagagcttccccttcaacacctcagtggtgtacaagaagaccctgtttgttgagttcactgaccacctgttcaacattgcaaagcccagacccccctggatgggcctgctgggccccaccatccaggcagaagtgtatgacacagtagtgatcaccctgaagaacatggccagccaccctgtcagcctgcatgcagttggtgtaagctactggaaggcctctgagggtgctgaatatgatgaccagaccagccagagagagaaggaggatgataaggtgttcccagggggcagccacacctatgtttggcaggtgctgaaggagaatgggcccatggcctctgatcccctgtgcctgacctacagctacctgagccatgtggacctggtgaaggacctgaactctgggctgataggagccctgctggtgtgcagagagggcagcctggccaaggagaagacccagaccctgcacaagttcatcctgctgtttgctgtgtttgatgaaggcaagagctggcacagtgaaaccaagaacagcctgatgcaggacagagatgcagctagtgccagagcctggcccaagatgcacacagtaaatggttatgtgaacagaagcctgcctggcctgataggctgccacagaaagtctgtgtactggcatgttataggtatgggcaccacccctgaggtgcacagcatcttcctggagggccacaccttcctggtgagaaaccacagacaggccagcctggagatcagccccatcaccttcctgactgcccagaccctgctgatggacctgggccagttcctgctgttctgccacatcagcagccaccagcatgatggaatggaggcctatgtcaaggtggacagctgcccagaggagccccagctgcagatgaagaacaatgaggaggcagaggactatgatgatgacctgactgacagtgaaatggatgtggtcagatttgatgatgataacagccccagcttcatccagatcagatcagtggccaagaagcaccccaagacctgggtgcactacattgcagctgaggaggaggactgggactatgcacccctggtgctggccccagatgatagaagctacaagagccagtacctgaacaatggaccccagagaattggcagaaagtacaagaaggtgagattcatggcctacactgatgagaccttcaagaccagagaggccatccagcatgaatcaggcatcctgggccccctgctgtatggggaggtgggagacaccctgctgatcatcttcaagaaccaggccagcagaccctacaacatctacccccatggaatcacagatgttagacccctgtacagcagaagactgcccaagggggtgaagcacctgaaggacttccccatcctgcctggggagatcttcaagtacaagtggacagttacagtagaggatggccccaccaagagtgaccccagatgcctgaccagatactacagcagctttgtaaacatggagagagacctggcctctggcctgataggccccctgctgatctgctacaaggagtcagttgaccagcagggcaaccagatcatgagtgataagagaaatgtcatcctgttcagtgtgtttgatgagaacagaagctggtacctgacagagaacatccagagattcctgcccaaccctgctggagtgcagctggaggacccagaattccaggccagcaacatcatgcacagcatcaatggttatgtctttgatagcctgcagctgtcagtttgcctgcatgaggtggcctactggtacatcctgagcattggggcccagactgacttcctgagtgtattcttcagtggctacaccttcaagcacaagatggtgtatgaggacaccctgaccctgttccccttctctggagagactgtgttcatgagcatggagaaccctgggctgtggatcctgggctgccacaactcagacttcagaaacagaggcatgacagcactgctgaaggtgagcagctgtgataagaacactggtgactactatgaggacagctatgaagacatctcagcctacctgctgagcaagaacaatgccatagagcccagaagcttcagccagaaccccccagttctgaagagacaccagagagagatcaccagaaccaccctgcagagtgaccaggaggagatagattatgatgataccatctcagttgagatgaagaaggaggactttgatatctatgatgaggatgagaaccagagccccagaagcttccagaagaagaccagacactacttcatagcagctgtggagagactgtgggactatggcatgagcagcagcccccatgttctgagaaacagagcccagagtggatcagtaccccagttcaagaaggtggtgttccaggagttcactgatgggagcttcacccagcccctgtacagaggtgaactgaatgagcacctgggcctgctgggcccctacatcagagctgaagtggaggacaacatcatggtgaccttcagaaaccaggccagcagaccctacagcttctacagcagcctgatcagctatgaggaggaccagagacagggagctgagcccagaaagaactttgtcaagcccaatgaaaccaagacctacttctggaaggtgcagcaccacatggcccccaccaaggatgagtttgactgcaaggcctgggcctacttcagtgatgtagacctggagaaggatgtccactcaggcctgattggccccctgctggtgtgccacaccaacaccctgaacccagcacatgggagacaggtgactgtacaggagtttgctctgttcttcaccatctttgatgagaccaagagctggtacttcacagaaaacatggagagaaactgcagagccccctgcaacatccagatggaggaccccaccttcaaggagaactacagattccatgccatcaatggttacatcatggacaccctgcctggcctggtgatggcccaggaccagagaatcagatggtacctgctgagcatgggcagcaatgaaaacatccacagcatccacttctcagggcatgtgttcactgtgagaaagaaggaggagtacaagatggccctgtacaacctgtacccaggtgtgtttgaaactgtggagatgctgcccagcaaggcaggcatctggagagtggagtgcctgataggggagcacctgcatgcagggatgagcaccctgttcctggtgtacagcaacaagtgccagacccccctgggcatggcctcaggccacatcagagacttccagatcacagcttctggacagtatggccagtgggcccccaagctggccagactgcactacagtggcagcatcaatgcctggagcaccaaggagcccttcagctggatcaaggtggacctgctggcccccatgatcatccatggcatcaagacccagggagccagacagaagttcagcagcctgtacatcagccagttcatcatcatgtacagcctggatggcaagaagtggcagacctacagaggcaacagcacaggcaccctgatggtgttctttgggaatgtggacagcagtggaatcaagcacaacatcttcaacccccccatcattgcaagatacatcagactgcaccccacccactacagcatcagaagcaccctgagaatggagctgatgggctgtgacctgaacagctgcagcatgcccctgggcatggagagcaaggccatctctgatgcccagatcacagctagcagctacttcaccaacatgtttgccacctggagccccagcaaggccagactgcacctgcagggcagaagcaatgcctggagaccccaggtgaacaaccccaaggagtggctgcaggtggacttccagaagaccatgaaggtgactggtgttaccacccagggggtcaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgagaatccacccccagagctgggtgcaccagatagcactgagaatggaggtgctgggctgtgaggcccaggacctgtactga

SEQ ID NO: 63 - DNA, 4374 bp, synthetic construct
  source 1..4374: mol_type = other DNA; note = FVIII-QQ03
atgcaaattgagttgtccacctgcttcttcttgtgcttgttgaggttctgcttcagtgccaccaggaggtactacttgggggctgtggagttgtcctgggactacatgcagtcagacttgggggagttgcctgtggatgccaggttcccccccagggtccccaagtccttccccttcaacaccagtgtggtctacaagaagaccttgtttgtggagttcactgaccacttgttcaacattgccaagcccaggcccccctggatgggcttgttgggccccaccatccaggctgaggtctatgacacagtggtcatcaccttgaagaacatggcctcccaccctgtctccttgcatgcagtgggggtctcctactggaaggccagtgagggggctgagtatgatgaccagacctcccagagggagaaggaggatgacaaggtcttccctgggggctcccacacctatgtctggcaggtcttgaaggagaatggccccatggcctctgaccccttgtgcttgacctactcctacttgtcccatgtggacttggtcaaggacttgaactcaggcttgattggggccttgttggtctgcagggagggctccttggccaaggagaagacccagaccttgcacaagttcatcttgttgtttgcagtctttgatgagggcaagtcctggcacagtgagaccaagaactccttgatgcaggacagggatgctgccagtgccagggcctggcccaagatgcacactgtcaatggctatgtcaacaggtccttgcctggcttgattggctgccacaggaagtcagtctactggcatgtcattggcatgggcaccacccctgaggtccactccatcttcttggagggccacaccttcttggtcaggaaccacaggcaggcctccttggagatctcccccatcaccttcttgacagcccagaccttgttgatggacttgggccagttcttgttgttctgccacatctcctcccaccagcatgatggcatggaggcctatgtcaaggtggactcctgccctgaggagccccagttgcagatgaagaacaatgaggaggcagaggactatgatgatgacttgactgactctgagatggatgtggtcaggtttgatgatgacaactccccctccttcatccagatcaggagtgtggccaagaagcaccccaagacctgggtccactacatagcagcagaggaggaggactgggactatgcccccttggtcttggcccctgatgacaggtcctacaagtcccagtacttgaacaatggcccccagaggattggcaggaagtacaagaaggtcaggttcatggcctacacagatgagaccttcaagaccagggaggccatccagcatgagtcaggcatcttgggccccttgttgtatggggaggtgggggacaccttgttgatcatcttcaagaaccaggcctccaggccctacaacatctacccccatggcatcactgatgtcaggcccttgtactccaggaggttgcccaagggggtcaagcacttgaaggacttccccatcttgccaggggagatcttcaagtacaagtggacagtcactgtggaggatggccccaccaagtcagaccccaggtgcttgaccaggtactactcctcctttgtcaacatggagagggacttggccagtggcttgattggccccttgttgatctgctacaaggagtctgtggaccagcagggcaaccagatcatgtcagacaagaggaatgtcatcttgttcagtgtctttgatgagaacaggtcctggtacttgacagagaacatccagaggttcttgcccaacccagctggggtccagttggaggaccctgagttccaggcctccaacatcatgcactccatcaatggctatgtctttgactccttgcagttgtcagtctgcttgcatgaggtggcctactggtacatcttgtccattggggcccagactgacttcttgtctgtcttcttcagtggctacaccttcaagcacaagatggtctatgaggacaccttgaccttgttccccttcagtggggagactgtcttcatgtccatggagaacccaggcttgtggatcttgggctgccacaactcagacttcaggaacaggggcatgacagccttgttgaaggtctcctcctgtgacaagaacacaggggactactatgaggactcctatgaggacatctcagcctacttgttgtccaagaacaatgccatagagcccaggtccttctcccagaacccccctgtcttgaagaggcaccagagggagatcaccaggaccaccttgcagtcagaccaggaggagatagactatgatgacaccatctcagtggagatgaagaaggaggactttgacatctatgatgaggatgagaaccagtcccccaggtccttccagaagaagaccaggcactacttcattgcagcagtggagaggttgtgggactatggcatgtcctcctccccccatgtcttgaggaacagggcccagtcaggcagtgtcccccagttcaagaaggtggtcttccaggagttcactgatggctccttcacccagcccttgtacaggggggagttgaatgagcacttgggcttgttgggcccctacatcagggctgaggtggaggacaacatcatggtcaccttcaggaaccaggcctccaggccctactccttctactcctccttgatctcctatgaggaggaccagaggcagggggcagagcccaggaagaactttgtcaagcccaatgagaccaagacctacttctggaaggtccagcaccacatggcccccaccaaggatgagtttgactgcaaggcctgggcctacttctctgatgtggacttggagaaggatgtccactctggcttgattggccccttgttggtctgccacaccaacaccttgaaccctgcccatggcaggcaggtcactgtccaggagtttgccttgttcttcaccatctttgatgagaccaagtcctggtacttcacagagaacatggagaggaactgcagggccccctgcaacatccagatggaggaccccaccttcaaggagaactacaggttccatgccatcaatggctacatcatggacaccttgccaggcttggtcatggcccaggaccagaggatcaggtggtacttgttgtccatgggctccaatgagaacatccactccatccacttcagtggccatgtcttcactgtcaggaagaaggaggagtacaagatggccttgtacaacttgtacccaggggtctttgagacagtggagatgttgccctccaaggcaggcatctggagggtggagtgcttgattggggagcacttgcatgctggcatgtccaccttgttcttggtctactccaacaagtgccagacccccttgggcatggcctctggccacatcagggacttccagatcactgcctcaggccagtatggccagtgggcccccaagttggccaggttgcactacagtggctccatcaatgcctggtccaccaaggagcccttctcctggatcaaggtggacttgttggcccccatgatcatccatggcatcaagacccagggggccaggcagaagttctcctccttgtacatctcccagttcatcatcatgtactccttggatggcaagaagtggcagacctacaggggcaactccacaggcaccttgatggtcttctttggcaatgtggactcctctggcatcaagcacaacatcttcaacccccccatcattgccaggtacatcaggttgcaccccacccactactccatcaggtccaccttgaggatggagttgatgggctgtgacttgaactcctgctccatgcccttgggcatggagtccaaggccatctcagatgcccagatcactgcctcctcctacttcaccaacatgtttgccacctggtccccctccaaggccaggttgcacttgcagggcaggtccaatgcctggaggccccaggtcaacaaccccaaggagtggttgcaggtggacttccagaagaccatgaaggtcacaggggtcaccacccagggggtcaagtccttgttgacctccatgtatgtcaaggagttcttgatctcctcctcccaggatggccaccagtggaccttgttcttccagaatggcaaggtcaaggtcttccagggcaaccaggactccttcacccctgtggtcaactccttggacccccccttgttgaccaggtacttgaggatccacccccagtcctgggtccaccagattgccttgaggatggaggtcttgggctgtgaggcccaggacttgtactga

SEQ ID NO: 64 - DNA, 4374 bp, synthetic construct
  source 1..4374: mol_type = other DNA; note = FVIII-QQ04
atgcagatagagctgagcacctgcttcttcctgtgcctgctgagattctgcttctcagccaccagaagatactacctgggggctgtagagctgagctgggactacatgcagtcagacctgggtgagctgccagtggatgccagattcccccccagagtgcccaagagcttccccttcaacacctctgtggtgtacaagaagaccctgtttgtggagttcactgatcacctgttcaacatagctaagcccagacccccctggatgggcctgctgggccccaccatccaggctgaggtgtatgatacagtggtgatcaccctgaagaacatggccagccaccctgtgagcctgcatgcagtgggtgtgagctactggaaggcctctgagggtgctgaatatgatgaccagaccagccagagagagaaggaggatgataaggtgttccctgggggcagccacacctatgtgtggcaggtgctgaaggagaatggacccatggccagtgaccccctgtgcctgacctacagctacctgagccatgtagacctggtgaaggacctgaactctggactgattggggcactgctggtgtgcagagagggcagcctggccaaggagaagacccagaccctgcacaagttcatcctgctgtttgctgtgtttgatgagggcaagagctggcactctgaaaccaagaacagcctgatgcaggacagagatgctgcctcagcaagagcctggcccaagatgcacactgtgaatggctatgttaacagaagcctgcctgggctgattgggtgccacagaaagagtgtgtactggcatgtaataggaatgggcaccaccccagaggtgcacagcatcttcctggagggccacaccttcctggtgagaaaccacagacaggccagcctggagatcagccccatcaccttcctgactgctcagaccctgctgatggacctgggccagttcctgctgttctgccacatcagcagccaccagcatgatggaatggaggcctatgtaaaggtggacagctgccctgaggagccccagctgcagatgaagaacaatgaggaggcagaggactatgatgatgacctgactgacagtgagatggatgtggtgagatttgatgatgataacagccccagcttcatccagatcagatctgtagccaagaagcaccccaagacctgggtgcactacatagctgcagaagaggaggactgggactatgcccccctggtgctggcccctgatgatagaagctacaagagccagtacctgaacaatggtccccagagaatagggagaaagtacaagaaggtgagattcatggcctacactgatgagaccttcaagaccagagaggccatccagcatgagtcaggcatcctgggccccctgctgtatggagaagtgggtgacaccctgctgatcatcttcaagaaccaggccagcagaccctacaacatctacccccatggcatcactgatgtcagacccctgtacagcagaagactgcccaagggtgtgaagcacctgaaggacttccccatcctgccaggtgagatcttcaagtacaagtggacagtgactgtagaggatgggcccaccaagtcagaccccagatgcctgaccagatactacagcagctttgtgaacatggagagagacctggccagtggcctgattggccccctgctgatctgctacaaggagtcagttgaccagcagggcaaccagatcatgtctgacaagagaaatgtcatcctgttcagtgtgtttgatgagaacagaagctggtacctgactgagaacatccagagattcctgcccaacccagcaggagttcagctggaggaccctgagttccaggccagcaacatcatgcacagcatcaatggctatgtttttgacagcctgcagctgtcagtgtgcctgcatgaagtggcctactggtacatcctgagcattggtgcccagacagatttcctgagtgtcttcttctcaggctacaccttcaagcacaagatggtgtatgaggacaccctgaccctgttccccttctctggggaaacagtgttcatgagcatggagaaccctggtctgtggatcctgggctgccacaactcagacttcagaaacagaggcatgacagcactgctgaaggtgagcagctgtgacaagaacacaggtgactactatgaagacagctatgaggacatctcagcctacctgctgagcaagaacaatgccattgagcccagaagcttcagccagaacccccctgtgctgaagagacaccagagagagatcaccagaaccaccctgcagtcagaccaggaggagattgactatgatgacaccatcagtgtagaaatgaagaaggaggactttgacatctatgatgaggatgaaaaccagagccccagaagcttccagaagaagaccagacactacttcatagctgcagttgagagactgtgggactatggaatgagcagcagcccccatgtgctgagaaacagagcccagtctggctcagttccccagttcaagaaggtggtgttccaggagttcacagatgggagcttcacccagcccctgtacagaggggagctgaatgagcacctgggcctgctgggcccctacatcagagcagaggtggaggacaacatcatggtgaccttcagaaaccaggccagcagaccctacagcttctacagcagcctgatcagctatgaagaggaccagagacagggagcagaacccagaaagaactttgttaagcccaatgaaaccaagacctacttctggaaggtgcagcaccacatggcccccaccaaggatgaatttgactgcaaggcctgggcctacttctctgatgttgacctggagaaggatgtgcactcaggcctgataggccccctgctggtgtgccacaccaacaccctgaacccagctcatgggagacaggtgactgttcaggagtttgccctgttcttcaccatctttgatgagaccaagagctggtacttcacagagaacatggagagaaactgcagagccccctgcaacatccagatggaggaccccaccttcaaggagaactacagattccatgccatcaatggttacatcatggacaccctgcctgggctggtgatggcccaggaccagagaatcagatggtacctgctgagcatgggcagcaatgaaaacatccacagcatccacttctctggccatgtgttcactgtcagaaagaaggaggagtacaagatggccctgtacaacctgtaccctggagtatttgagactgtggagatgctgcccagcaaggcaggtatctggagagtggagtgcctgattggggagcacctgcatgcaggtatgagcaccctgttcctggtgtacagcaacaagtgccagacccccctgggcatggcctctggacacatcagagacttccagatcactgccagtggccagtatggccagtgggcccccaagctggccagactgcactactcagggagcatcaatgcctggagcaccaaggagcccttcagctggatcaaggtggacctgctggcccccatgatcatccatggtatcaagacccagggtgccagacagaagttcagcagcctgtacatcagccagttcatcatcatgtacagcctggatggcaagaagtggcagacctacagaggcaacagcacaggcaccctgatggtgttctttggcaatgtagacagctctggcatcaagcacaacatcttcaacccccccatcatagctagatacatcagactgcaccccacccactacagcatcagaagcaccctgagaatggagctgatgggctgtgacctgaacagctgcagcatgcccctgggcatggagagcaaggccatctcagatgctcagatcactgccagcagctacttcaccaacatgtttgccacctggagccccagcaaggccagactgcacctgcagggcagaagcaatgcctggagaccccaggtgaacaaccccaaggagtggctgcaggtggacttccagaagaccatgaaggtgacaggagtgaccacccagggggtgaagagcctgctgaccagcatgtatgtgaaggagttcctgatcagcagcagccaggatggacaccagtggaccctgttcttccagaatggaaaggtgaaggtgttccagggcaaccaggacagcttcacccctgtggtgaacagcctggacccccccctgctgaccagatacctgagaatccacccccagagctgggtgcaccagattgccctgagaatggaggtgctgggctgtgaggcccaggacctgtactga

SEQ ID NO: 65 - DNA, 4374 bp, synthetic construct
  source 1..4374: mol_type = other DNA; note = FVIII-QQ05
atgcagattgagctgagcacctgcttcttcctgtgcctgctgagattctgcttctctgctaccagaagatactacctgggagctgtggagctgagctgggactacatgcagtcagacctgggtgaactgcctgtggatgccagattcccccccagagtgcccaagagcttccccttcaacaccagtgttgtctacaagaagaccctgtttgtggagttcacagaccacctgttcaacattgccaagcccagacccccctggatgggcctgctgggccccaccatccaggctgaagtgtatgatacagtggtgatcaccctgaagaacatggccagccacccagtgagcctgcatgcagtgggtgtgagctactggaaggcctctgaaggtgcagagtatgatgaccagaccagccagagagagaaggaggatgacaaggtgttccctgggggcagccacacctatgtgtggcaggtgctgaaggagaatggccccatggccagtgatcccctgtgcctgacctacagctacctgagccatgttgacctggtgaaggacctgaactcaggtctgattggagccctgctggtgtgcagagagggcagcctggccaaggagaagacccagaccctgcacaagttcatcctgctgtttgctgtctttgatgaaggcaagagctggcacagtgagaccaagaacagcctgatgcaggacagagatgcagcctctgccagagcctggcccaagatgcacacagtaaatggctatgtgaacagaagcctgccagggctgattggctgccacagaaagtctgtttactggcatgtgattgggatgggcaccacccctgaagtgcacagcatcttcctggagggccacaccttcctggtgagaaaccacagacaggccagcctggagatcagccccatcaccttcctgacagcacagaccctgctgatggacctgggccagttcctgctgttctgccacatcagcagccaccagcatgatgggatggaggcctatgtgaaggtggacagctgcccagaagagccccagctgcagatgaagaacaatgaggaggcagaggactatgatgatgacctgactgactctgagatggatgttgtgagatttgatgatgacaacagccccagcttcatccagatcagaagtgttgccaagaagcaccccaagacctgggtgcactacattgcagctgaggaggaggactgggactatgcccccctggtgctggccccagatgacagaagctacaagagccagtacctgaacaatggcccccagagaattggtagaaagtacaagaaggtgagattcatggcctacacagatgagaccttcaagaccagagaggccatccagcatgagtcaggaatcctgggccccctgctgtatggggaggtgggggataccctgctgatcatcttcaagaaccaggccagcagaccctacaacatctacccccatggcatcacagatgtgagacccctgtacagcagaagactgcccaagggagtgaagcacctgaaggacttccccatcctgcctggtgagatcttcaagtacaagtggacagttactgtggaagatggtcccaccaagtcagaccccagatgcctgaccagatactacagcagctttgtgaacatggagagagacctggcctctgggctgattgggcccctgctgatctgctacaaggagagtgtggaccagcagggcaaccagatcatgtctgataagagaaatgtgatcctgttctctgtatttgatgaaaacagaagctggtacctgactgagaacatccagagattcctgcccaacccagctggagtccagctggaggacccagagttccaggccagcaacatcatgcacagcatcaatggatatgtgtttgacagcctgcagctgtctgtatgcctgcatgaggtggcctactggtacatcctgagcattggggcccagactgacttcctgtctgtgttcttctcaggatacaccttcaagcacaagatggtgtatgaggacaccctgaccctgttccccttctcaggggaaactgtgttcatgagcatggagaaccctggcctgtggatcctgggctgccacaactctgacttcagaaacagaggcatgactgccctgctgaaggtgagcagctgtgacaagaacactggggattactatgaggacagctatgaggacatctcagcctacctgctgagcaagaacaatgccattgaacccagaagcttcagccagaacccccctgtcctgaagagacaccagagagagatcaccagaaccaccctgcagtctgatcaggaggagattgactatgatgataccatctcagtggagatgaagaaggaggactttgacatctatgatgaagatgaaaaccagagccccagaagcttccagaagaagaccagacactacttcattgctgctgtggagagactgtgggactatgggatgagcagcagcccccatgtgctgagaaacagagcccagtcaggcagtgtgccccagttcaagaaggtggtgttccaggagttcacagatggaagcttcacccagcccctgtacagaggtgagctgaatgaacacctgggcctgctgggcccctacatcagagctgaagtggaggacaacatcatggtgaccttcagaaaccaggccagcagaccctacagcttctacagcagcctgatcagctatgaggaggaccagagacagggagcagagcccagaaagaactttgtcaagcccaatgaaaccaagacctacttctggaaggtgcagcaccacatggcccccaccaaggatgaatttgactgcaaggcctgggcctacttctctgatgtagacctggagaaggatgtgcactctgggctgattggccccctgctggtgtgccacaccaacaccctgaacccagcccatggcagacaggtgactgtgcaggagtttgccctgttcttcaccatctttgatgaaaccaagagctggtacttcactgagaacatggagagaaactgcagagccccctgcaacatccagatggaggaccccaccttcaaggagaactacagattccatgctatcaatggatacatcatggacaccctgcctgggctggtgatggcccaggaccagagaatcagatggtacctgctgagcatgggcagcaatgagaacatccacagcatccacttctcaggacatgtcttcacagtaagaaagaaggaggagtacaagatggccctgtacaacctgtacccaggggtctttgaaactgtagagatgctgcccagcaaggcaggcatctggagagtggagtgcctgattggagagcacctgcatgctggtatgagcaccctgttcctggtgtacagcaacaagtgccagacccccctgggcatggcctctggccacatcagagacttccagatcacagccagtggccagtatgggcagtgggcccccaagctggccagactgcactactctggcagcatcaatgcctggagcaccaaggagcccttcagctggatcaaggtggacctgctggcccccatgatcatccatgggatcaagacccagggtgccagacagaagttcagcagcctgtacatcagccagttcatcatcatgtacagcctggatggcaagaagtggcagacctacagaggcaacagcacaggtaccctgatggtgttctttggcaatgtggacagctcaggcatcaagcacaacatcttcaacccccccatcattgccagatacatcagactgcaccccacccactacagcatcagaagcaccctgagaatggagctgatgggctgtgacctgaacagctgcagcatgcccctgggcatggagagcaaggccatctctgatgcccagatcacagctagcagctacttcaccaacatgtttgccacctggagccccagcaaggccagactgcacctgcagggcagaagcaatgcttggagaccccaggtgaacaaccccaaggagtggctgcaggtggacttccagaagaccatgaaggtgacaggtgtgaccacccagggggttaagagcctgctgaccagcatgtatgtgaaggagttcctgatcagcagcagccaggatggccaccagtggaccctgttcttccagaatggcaaggtgaaggtgttccagggcaaccaggacagcttcacccctgttgtgaacagcctggacccccccctgctgaccagatacctgagaatccacccccagagctgggtgcaccagattgccctgagaatggaggtgctgggctgtgaggcccaggacctgtactga

SEQ ID NO: 66 - DNA, 4374 bp, synthetic construct
  source 1..4374: mol_type = other DNA; note = FVIII-QQ06
atgcagatagaactgagcacctgcttcttcctgtgcctgctgagattctgcttctcagccaccagaagatactacctgggggcagttgagctgagctgggactacatgcagagtgatctgggggagctgccagtggatgcaagattcccccccagagtgcccaagagcttccccttcaacaccagtgtggtctacaagaagaccctgtttgttgagttcacagaccacctgttcaacattgctaagcccagacccccctggatgggcctgctgggccccaccatccaggcagaggtgtatgatacagttgtgatcaccctgaagaacatggccagccacccagttagcctgcatgcagttggagtgagctactggaaggcctcagagggagctgagtatgatgaccagaccagccagagagagaaggaggatgataaggtgttcccaggggggagccacacctatgtgtggcaggtgctgaaggagaatggacccatggccagtgaccccctgtgcctgacctacagctacctgagccatgtggacctggtgaaggacctgaactcaggactgataggtgctctgctggtgtgcagagagggcagcctggccaaggagaagacccagaccctgcacaagttcatcctgctgtttgcagtttttgatgaaggcaagagctggcactcagagaccaagaacagcctgatgcaggacagagatgcagcttctgctagagcctggcccaagatgcacactgtcaatggctatgtaaacagaagcctgcctggcctgattggctgccacagaaagtctgtctactggcatgttataggtatgggcaccacccctgaggtgcacagcatcttcctggagggccacaccttcctggtgagaaaccacagacaggccagcctggagatcagccccatcaccttcctgactgcccagaccctgctgatggacctgggccagttcctgctgttctgccacatcagcagccaccagcatgatggcatggaggcctatgtcaaggtggacagctgcccagaagagccccagctgcagatgaagaacaatgaggaggctgaggactatgatgatgacctgactgacagtgagatggatgtagtgagatttgatgatgacaacagccccagcttcatccagatcagaagtgtagccaagaagcaccccaagacctgggtgcactacattgctgcagaggaggaggactgggactatgcccccctggtgctggccccagatgacagaagctacaagagccagtacctgaacaatggtccccagagaattggcagaaagtacaagaaggtgagattcatggcctacacagatgagaccttcaagaccagagaggccatccagcatgagtcaggcatcctgggccccctgctgtatggagaggtgggggacaccctgctgatcatcttcaagaaccaggccagcagaccctacaacatctacccccatggaatcacagatgtgagacccctgtacagcagaagactgcccaagggggtgaagcacctgaaggacttccccatcctgcctggagagatcttcaagtacaagtggactgtgacagtagaggatggacccaccaagtcagaccccagatgcctgaccagatactacagcagctttgtaaacatggagagagacctggccagtggcctgataggccccctgctgatctgctacaaggagtcagtggaccagcagggcaaccagatcatgtcagacaagagaaatgtgatcctgttctctgtatttgatgagaacagaagctggtacctgactgaaaacatccagagattcctgcccaaccctgctggagtacagctggaggaccctgagttccaggccagcaacatcatgcacagcatcaatggttatgtgtttgacagcctgcagctgtctgtgtgcctgcatgaagtggcctactggtacatcctgagcataggtgcccagactgacttcctgtctgtgttcttcagtggctacaccttcaagcacaagatggtgtatgaagacaccctgaccctgttccccttctctggggagacagtattcatgagcatggagaacccaggactgtggatcctgggctgccacaacagtgacttcagaaacagaggcatgactgctctgctgaaggtgagcagctgtgataagaacacaggtgactactatgaggacagctatgaagacatcagtgcttacctgctgagcaagaacaatgctatagaacccagaagcttcagccagaaccccccagttctgaagagacaccagagagagatcaccagaaccaccctgcagtcagaccaggaggagatagactatgatgataccatctcagtagagatgaagaaggaggactttgacatctatgatgaggatgaaaaccagagccccagaagcttccagaagaagaccagacactacttcatagctgctgtagagagactgtgggactatggaatgagcagcagcccccatgtgctgagaaacagagcccagtctgggagtgtgccccagttcaagaaggtggtgttccaggagttcacagatgggagcttcacccagcccctgtacagaggggagctgaatgaacacctgggcctgctgggcccctacatcagagcagaggtggaggacaacatcatggtgaccttcagaaaccaggccagcagaccctacagcttctacagcagcctgatcagctatgaggaggaccagagacagggagctgagcccagaaagaactttgttaagcccaatgaaaccaagacctacttctggaaggtgcagcaccacatggcccccaccaaggatgagtttgactgcaaggcctgggcctacttctctgatgttgacctggagaaggatgtgcactctggactgattgggcccctgctggtgtgccacaccaacaccctgaaccctgcccatgggagacaggtgacagtacaggagtttgctctgttcttcaccatctttgatgagaccaagagctggtacttcacagaaaacatggagagaaactgcagagccccctgcaacatccagatggaggaccccaccttcaaggagaactacagattccatgctatcaatggctacatcatggacaccctgcctggcctggtgatggcccaggaccagagaatcagatggtacctgctgagcatgggcagcaatgaaaacatccacagcatccacttctctggtcatgttttcacagttagaaagaaggaggagtacaagatggccctgtacaacctgtaccctggggtgtttgagactgtggagatgctgcccagcaaggctggaatctggagagtggagtgcctgattggagaacacctgcatgcagggatgagcaccctgttcctggtgtacagcaacaagtgccagacccccctgggcatggccagtggtcacatcagagacttccagatcacagcctcaggccagtatggacagtgggcccccaagctggccagactgcactacagtggtagcatcaatgcctggagcaccaaggagcccttcagctggatcaaggtggacctgctggcccccatgatcatccatggaatcaagacccagggggccagacagaagttcagcagcctgtacatcagccagttcatcatcatgtacagcctggatggcaagaagtggcagacctacagaggcaacagcactggtaccctgatggtgttctttggcaatgtggacagctctgggatcaagcacaacatcttcaacccccccatcatagccagatacatcagactgcaccccacccactacagcatcagaagcaccctgagaatggagctgatgggctgtgacctgaacagctgcagcatgcccctgggcatggagagcaaggccatcagtgatgctcagatcacagcaagcagctacttcaccaacatgtttgccacctggagccccagcaaggccagactgcacctgcagggcagaagcaatgcttggagaccccaggtgaacaaccccaaggagtggctgcaggtggacttccagaagaccatgaaggtgactggggtgaccacccagggggtgaagagcctgctgaccagcatgtatgtcaaggagttcctgatcagcagcagccaggatgggcaccagtggaccctgttcttccagaatgggaaggtgaaggtgttccagggcaaccaggacagcttcaccccagtagtgaacagcctggacccccccctgctgaccagatacctgagaatccacccccagagctgggtgcaccagatagccctgagaatggaggtgctgggctgtgaggcccaggacctgtactga

SEQ ID NO: 67 - DNA, 4374 bp, synthetic construct
  source 1..4374: mol_type = other DNA; note = FVIII-QQ07
atgcagatagagctgagcacctgcttcttcctgtgcctgctgagattctgcttctcagccaccagaagatactacctgggtgcagtggagctgagctgggactacatgcagagtgacctgggggaactgcctgtggatgccagattcccccccagagtgcccaagagcttccccttcaacacctctgttgtttacaagaagaccctgtttgtagagttcactgaccacctgttcaacatagccaagcccagacccccctggatgggcctgctgggccccaccatccaggcagaggtgtatgatacagtggtgatcaccctgaagaacatggccagccaccctgttagcctgcatgcagtgggggtgagctactggaaggccagtgagggagcagagtatgatgatcagaccagccagagagagaaggaggatgacaaggtgttccctgggggcagccacacctatgtatggcaggtgctgaaggagaatggccccatggcctcagatcccctgtgcctgacctacagctacctgagccatgtggacctggtgaaggacctgaacagtggtctgattggggccctgctggtgtgcagagagggcagcctggccaaggagaagacccagaccctgcacaagttcatcctgctgtttgctgtctttgatgagggcaagagctggcactcagagaccaagaacagcctgatgcaggacagagatgctgcctctgccagagcctggcccaagatgcacactgtgaatgggtatgtgaacagaagcctgccaggtctgataggctgccacagaaagtcagtatactggcatgtgattggcatgggcaccaccccagaggtgcacagcatcttcctggagggccacaccttcctggtgagaaaccacagacaggccagcctggagatcagccccatcaccttcctgactgcccagaccctgctgatggacctgggccagttcctgctgttctgccacatcagcagccaccagcatgatggcatggaggcctatgttaaggtggacagctgcccagaggagccccagctgcagatgaagaacaatgaagaggctgaggactatgatgatgatctgacagactcagaaatggatgtagtcagatttgatgatgataacagccccagcttcatccagatcagatctgtagccaagaagcaccccaagacctgggtgcactacatagcagcagaggaggaggactgggactatgcacccctggtgctggcccctgatgacagaagctacaagagccagtacctgaacaatggcccccagagaataggcagaaagtacaagaaggtgagattcatggcctacactgatgagaccttcaagaccagagaggccatccagcatgagtctggaatcctgggccccctgctgtatggtgaggtgggtgacaccctgctgatcatcttcaagaaccaggccagcagaccctacaacatctacccccatggcatcactgatgtgagacccctgtacagcagaagactgcccaagggggtgaagcacctgaaggacttccccatcctgccaggtgagatcttcaagtacaagtggactgtgacagtggaggatggccccaccaagagtgaccccagatgcctgaccagatactacagcagctttgtcaacatggagagagacctggccagtgggctgatagggcccctgctgatctgctacaaggagtcagtggaccagcagggcaaccagatcatgagtgataagagaaatgtgatcctgttctcagtttttgatgaaaacagaagctggtacctgacagaaaacatccagagattcctgcccaacccagcaggggtacagctggaggaccctgaattccaggccagcaacatcatgcacagcatcaatggctatgtatttgacagcctgcagctgtctgtttgcctgcatgaggtggcctactggtacatcctgagcataggagcccagacagacttcctgagtgtgttcttcagtggctacaccttcaagcacaagatggtgtatgaggacaccctgaccctgttccccttctcaggggagactgttttcatgagcatggagaaccctggactgtggatcctgggctgccacaactctgacttcagaaacagaggcatgactgccctgctgaaggtgagcagctgtgataagaacactggagactactatgaggacagctatgaggacatctcagcctacctgctgagcaagaacaatgccattgaacccagaagcttcagccagaacccccctgtactgaagagacaccagagagagatcaccagaaccaccctgcagagtgaccaggaggagatagactatgatgataccatctctgtggagatgaagaaggaggactttgatatctatgatgaggatgagaaccagagccccagaagcttccagaagaagaccagacactacttcatagcagcagtggagagactgtgggactatggcatgagcagcagcccccatgtgctgagaaacagagcccagtcaggcagtgttccccagttcaagaaggtggtgttccaggagttcactgatggtagcttcacccagcccctgtacagaggagagctgaatgagcacctgggcctgctgggcccctacatcagagctgaggtggaggacaacatcatggtgaccttcagaaaccaggccagcagaccctacagcttctacagcagcctgatcagctatgaggaggaccagagacagggagcagagcccagaaagaactttgttaagcccaatgaaaccaagacctacttctggaaggtgcagcaccacatggcccccaccaaggatgagtttgattgcaaggcctgggcctacttcagtgatgtagacctggagaaggatgtgcactcaggcctgattggacccctgctggtgtgccacaccaacaccctgaaccctgcacatggaagacaggtgacagtgcaggagtttgccctgttcttcaccatctttgatgagaccaagagctggtacttcacagagaacatggagagaaactgcagagccccctgcaacatccagatggaggaccccaccttcaaggagaactacagattccatgctatcaatggctacatcatggacaccctgcctggcctggtgatggcccaggaccagagaatcagatggtacctgctgagcatgggcagcaatgagaacatccacagcatccacttcagtgggcatgtgttcacagtgagaaagaaggaggagtacaagatggccctgtacaacctgtaccctggagtgtttgagactgtggagatgctgcccagcaaggcaggcatctggagagtggagtgcctgataggagaacacctgcatgctggcatgagcaccctgttcctggtgtacagcaacaagtgccagacccccctgggcatggccagtggacacatcagagacttccagatcacagcctctggacagtatggacagtgggcccccaagctggccagactgcactactcaggaagcatcaatgcatggagcaccaaggagcccttcagctggatcaaggtggacctgctggcccccatgatcatccatggcatcaagacccagggggcaagacagaagttcagcagcctgtacatcagccagttcatcatcatgtacagcctggatggcaagaagtggcagacctacagaggcaacagcactggaaccctgatggtgttctttgggaatgtggacagctcagggatcaagcacaacatcttcaacccccccatcattgctagatacatcagactgcaccccacccactacagcatcagaagcaccctgagaatggagctgatgggctgtgatctgaacagctgcagcatgcccctgggcatggagagcaaggccatctcagatgcccagatcactgccagcagctacttcaccaacatgtttgccacctggagccccagcaaggccagactgcacctgcagggcagaagcaatgcttggagaccccaggtgaacaaccccaaggagtggctgcaggtggacttccagaagaccatgaaggtgactggagtaaccacccagggggtgaagagcctgctgaccagcatgtatgtgaaggagttcctgatcagcagcagccaggatggtcaccagtggaccctgttcttccagaatggcaaggtgaaggtgttccagggcaaccaggacagcttcacccctgttgtgaacagcctggacccccccctgctgaccagatacctgagaatccacccccagagctgggtgcaccagattgccctgagaatggaggtgctgggctgtgaggcccaggacctgtactga

SEQ ID NO: 68 - DNA, 4374 bp, synthetic construct
  source 1..4374: mol_type = other DNA; note = FVIII-QQ08
atgcagatagagctgagcacctgcttcttcctgtgcctgctgagattctgcttctctgccaccagaagatactacctgggtgcagtggagctgagctgggactacatgcagtcagacctgggagagctgccagtggatgctagattcccccccagagtgcccaagagcttccccttcaacacctcagtggtgtacaagaagaccctgtttgtagagttcactgaccacctgttcaacattgccaagcccagacccccctggatgggcctgctgggccccaccatccaggctgaggtgtatgatacagtggtaatcaccctgaagaacatggccagccacccagtgagcctgcatgctgttggagttagctactggaaggcctctgagggtgctgagtatgatgatcagaccagccagagagagaaggaggatgataaggtgttcccagggggaagccacacctatgtatggcaggtgctgaaggagaatgggcccatggccagtgaccccctgtgcctgacctacagctacctgagccatgtggatctggtgaaggacctgaactcaggtctgataggggccctgctggtgtgcagagagggcagcctggccaaggagaagacccagaccctgcacaagttcatcctgctgtttgctgtatttgatgagggcaagagctggcacagtgaaaccaagaacagcctgatgcaggacagagatgctgcctctgccagagcctggcccaagatgcacacagtgaatggctatgtgaacagaagcctgcctggtctgatagggtgccacagaaagtcagtgtactggcatgtcattggcatgggcaccaccccagaggtgcacagcatcttcctggagggccacaccttcctggtgagaaaccacagacaggccagcctggagatcagccccatcaccttcctgacagctcagaccctgctgatggacctgggccagttcctgctgttctgccacatcagcagccaccagcatgatggcatggaggcctatgttaaggtggacagctgcccagaggagccccagctgcagatgaagaacaatgaggaggcagaggactatgatgatgatctgacagacagtgagatggatgtggtgagatttgatgatgacaacagccccagcttcatccagatcagatctgtggccaagaagcaccccaagacctgggtgcactacattgcagctgaagaggaggactgggactatgcccccctggtgctggccccagatgatagaagctacaagagccagtacctgaacaatggcccccagagaattggaagaaagtacaagaaggtgagattcatggcctacactgatgaaaccttcaagaccagagaggccatccagcatgagtcaggcatcctgggccccctgctgtatggtgaggtgggtgataccctgctgatcatcttcaagaaccaggccagcagaccctacaacatctacccccatggcatcactgatgtaagacccctgtacagcagaagactgcccaagggtgtaaagcacctgaaggacttccccatcctgcctggggaaatcttcaagtacaagtggactgtgactgtagaggatggtcccaccaagagtgaccccagatgcctgaccagatactacagcagctttgtgaacatggagagagacctggcctctggtctgataggtcccctgctgatctgctacaaggagtcagtagaccagcagggcaaccagatcatgtctgacaagagaaatgtgatcctgttcagtgtgtttgatgagaacagaagctggtacctgacagaaaacatccagagattcctgcccaacccagctggagtgcagctggaggaccctgagttccaggccagcaacatcatgcacagcatcaatgggtatgtctttgacagcctgcagctgagtgtgtgcctgcatgaggtggcctactggtacatcctgagcattggtgctcagacagatttcctgtctgtgttcttcagtggttacaccttcaagcacaagatggtgtatgaagacaccctgaccctgttccccttcagtggagagacagtattcatgagcatggagaaccctggcctgtggatcctgggctgccacaacagtgatttcagaaacagaggcatgactgcactgctgaaggtgagcagctgtgacaagaacactggggattactatgaagacagctatgaggacatcagtgcctacctgctgagcaagaacaatgctattgagcccagaagcttcagccagaaccccccagtcctgaagagacaccagagagagatcaccagaaccaccctgcagagtgaccaggaggagatagactatgatgacaccatctcagtggagatgaagaaggaggactttgacatctatgatgaggatgagaaccagagccccagaagcttccagaagaagaccagacactacttcattgcagcagtagagagactgtgggactatgggatgagcagcagcccccatgtactgagaaacagagcccagtctggatctgtgccccagttcaagaaggtggtgttccaggagttcactgatggcagcttcacccagcccctgtacagaggagagctgaatgagcacctgggcctgctgggcccctacatcagagcagaggtggaggacaacatcatggtgaccttcagaaaccaggccagcagaccctacagcttctacagcagcctgatcagctatgaggaggaccagagacagggtgctgagcccagaaagaactttgttaagcccaatgagaccaagacctacttctggaaggtgcagcaccacatggcccccaccaaggatgaatttgattgcaaggcctgggcctacttcagtgatgtagacctggagaaggatgtgcacagtggactgattgggcccctgctggtgtgccacaccaacaccctgaacccagcccatggcagacaggtgactgtgcaggagtttgctctgttcttcaccatctttgatgaaaccaagagctggtacttcacagagaacatggagagaaactgcagagccccctgcaacatccagatggaggaccccaccttcaaggagaactacagattccatgctatcaatggctacatcatggacaccctgccaggtctggtgatggcccaggaccagagaatcagatggtacctgctgagcatgggcagcaatgagaacatccacagcatccacttctctggccatgtcttcactgtgagaaagaaggaggagtacaagatggccctgtacaacctgtacccaggtgtctttgagactgtagagatgctgcccagcaaggctggcatctggagagtggagtgcctgattggagagcacctgcatgcaggaatgagcaccctgttcctggtgtacagcaacaagtgccagacccccctgggcatggccagtggccacatcagagacttccagatcacagcctctgggcagtatggacagtgggcccccaagctggccagactgcactactcaggaagcatcaatgcctggagcaccaaggagcccttcagctggatcaaggtggacctgctggcccccatgatcatccatgggatcaagacccagggggccagacagaagttcagcagcctgtacatcagccagttcatcatcatgtacagcctggatggtaagaagtggcagacctacagaggcaacagcacaggcaccctgatggtgttctttggaaatgtagacagctctggcatcaagcacaacatcttcaacccccccatcatagccagatacatcagactgcaccccacccactacagcatcagaagcaccctgagaatggagctgatgggctgtgacctgaacagctgcagcatgcccctgggcatggagagcaaggccatctctgatgctcagatcactgccagcagctacttcaccaacatgtttgcaacctggagccccagcaaggccagactgcacctgcagggcagaagcaatgcctggagaccccaggtgaacaaccccaaggagtggctgcaggtggacttccagaagaccatgaaggtgactggtgtaaccacccagggagtgaagagcctgctgaccagcatgtatgtcaaggagttcctgatcagcagcagccaggatggtcaccagtggaccctgttcttccagaatggaaaggtgaaggtgttccagggcaaccaggacagcttcaccccagttgtgaacagcctggacccccccctgctgaccagatacctgagaatccacccccagagctgggtgcaccagattgccctgagaatggaggtgctgggctgtgaggcccaggacctgtactga

SEQ ID NO: 69 - DNA, 4374 bp, synthetic construct
  source 1..4374: mol_type = other DNA; note = FVIII-QQ09
atgcagatagagctgagcacctgcttcttcctgtgcctgctgagattctgcttcagtgcaaccagaagatactacctgggtgcagtggagctgagctgggactacatgcagtctgatctgggggagctgccagttgatgccagattcccccccagagtgcccaagagcttccccttcaacaccagtgttgtttacaagaagaccctgtttgtggaattcactgaccacctgttcaacatagccaagcccagacccccctggatgggcctgctgggccccaccatccaggcagaggtgtatgacacagtagtgatcaccctgaagaacatggccagccaccctgtgagcctgcatgcagtaggggtaagctactggaaggcctctgaaggtgctgaatatgatgaccagaccagccagagagagaaggaggatgacaaggtgttcccaggtggaagccacacctatgtctggcaggtgctgaaggagaatggccccatggccagtgaccccctgtgcctgacctacagctacctgagccatgtggacctggtgaaggacctgaactcaggtctgattggagcactgctggtgtgcagagagggcagcctggccaaggagaagacccagaccctgcacaagttcatcctgctgtttgcagtgtttgatgaaggcaagagctggcacagtgagaccaagaacagcctgatgcaggacagagatgcagccagtgccagagcctggcccaagatgcacactgtgaatggttatgtcaacagaagcctgccagggctgataggctgccacagaaagagtgtttactggcatgtaattgggatgggcaccaccccagaggtgcacagcatcttcctggagggccacaccttcctggtgagaaaccacagacaggccagcctggagatcagccccatcaccttcctgacagctcagaccctgctgatggacctgggccagttcctgctgttctgccacatcagcagccaccagcatgatggcatggaggcctatgtaaaggtggacagctgccctgaggagccccagctgcagatgaagaacaatgaggaggcagaggactatgatgatgacctgactgacagtgagatggatgtggtgagatttgatgatgacaacagccccagcttcatccagatcagaagtgttgctaagaagcaccccaagacctgggtgcactacattgcagcagaggaggaggactgggactatgctcccctggtgctggcccctgatgacagaagctacaagagccagtacctgaacaatggcccccagagaattggcagaaagtacaagaaggtgagattcatggcctacactgatgagaccttcaagaccagagaggccatccagcatgaaagtggcatcctgggccccctgctgtatggagaggtgggagacaccctgctgatcatcttcaagaaccaggccagcagaccctacaacatctacccccatggtatcacagatgtgagacccctgtacagcagaagactgcccaagggggtgaagcacctgaaggacttccccatcctgccaggtgaaatcttcaagtacaagtggacagtcacagtggaggatggccccaccaagagtgaccccagatgcctgaccagatactacagcagctttgtgaacatggagagagacctggcctctggcctgataggccccctgctgatctgctacaaggagtcagtggaccagcagggcaaccagatcatgagtgataagagaaatgtgatcctgttctcagtatttgatgagaacagaagctggtacctgactgaaaacatccagagattcctgcccaaccctgcaggggtgcagctggaggaccctgaattccaggccagcaacatcatgcacagcatcaatggctatgtatttgatagcctgcagctgagtgtgtgcctgcatgaggtggcctactggtacatcctgagcataggggcccagacagacttcctgtctgtgttcttctcaggttacaccttcaagcacaagatggtgtatgaagacaccctgaccctgttccccttcagtggggaaacagtattcatgagcatggagaaccctgggctgtggatcctgggctgccacaactcagatttcagaaacagaggcatgactgccctgctgaaggtgagcagctgtgacaagaacactggagactactatgaggacagctatgaggacatcagtgcctacctgctgagcaagaacaatgctatagagcccagaagcttcagccagaaccccccagtcctgaagagacaccagagagagatcaccagaaccaccctgcagtcagatcaggaggagatagactatgatgacaccatcagtgtggagatgaagaaggaggactttgacatctatgatgaggatgagaaccagagccccagaagcttccagaagaagaccagacactacttcatagctgctgtagagagactgtgggactatggcatgagcagcagcccccatgtcctgagaaacagagcccagtctggctctgttccccagttcaagaaggtggtgttccaggagttcacagatggcagcttcacccagcccctgtacagaggggagctgaatgaacacctgggcctgctgggcccctacatcagagctgaagtggaggacaacatcatggtgaccttcagaaaccaggccagcagaccctacagcttctacagcagcctgatcagctatgaggaggaccagagacagggtgcagaacccagaaagaactttgttaagcccaatgagaccaagacctacttctggaaggtgcagcaccacatggcccccaccaaggatgaatttgactgcaaggcctgggcctacttctcagatgttgacctggagaaggatgtccactcaggactgatagggcccctgctggtgtgccacaccaacaccctgaacccagcccatggcagacaggtgactgtgcaggagtttgctctgttcttcaccatctttgatgagaccaagagctggtacttcacagagaacatggagagaaactgcagagccccctgcaacatccagatggaggaccccaccttcaaggagaactacagattccatgctatcaatggatacatcatggacaccctgccaggcctggtgatggcccaggaccagagaatcagatggtacctgctgagcatgggcagcaatgagaacatccacagcatccacttcagtggtcatgtgttcacagtaagaaagaaggaggagtacaagatggccctgtacaacctgtacccaggggtgtttgaaactgtggaaatgctgcccagcaaggcagggatctggagagtggagtgcctgattggagaacacctgcatgcaggcatgagcaccctgttcctggtgtacagcaacaagtgccagacccccctgggcatggccagtgggcacatcagagacttccagatcacagcctctgggcagtatggccagtgggcccccaagctggccagactgcactactcaggcagcatcaatgcctggagcaccaaggagcccttcagctggatcaaggtggacctgctggcccccatgatcatccatggcatcaagacccagggagcaagacagaagttcagcagcctgtacatcagccagttcatcatcatgtacagcctggatgggaagaagtggcagacctacagaggcaacagcactgggaccctgatggtgttctttggcaatgtagacagctctggcatcaagcacaacatcttcaacccccccatcattgcaagatacatcagactgcaccccacccactacagcatcagaagcaccctgagaatggagctgatgggctgtgacctgaacagctgcagcatgcccctgggcatggagagcaaggccatcagtgatgcccagatcactgctagcagctacttcaccaacatgtttgccacctggagccccagcaaggccagactgcacctgcagggcagaagcaatgcatggagaccccaggtgaacaaccccaaggagtggctgcaggtggacttccagaagaccatgaaggtgacaggggttaccacccagggtgtgaagagcctgctgaccagcatgtatgtgaaggagttcctgatcagcagcagccaggatggccaccagtggaccctgttcttccagaatgggaaggtgaaggtgttccagggcaaccaggacagcttcacccctgtggtgaacagcctggacccccccctgctgaccagatacctgagaatccacccccagagctgggtgcaccagattgccctgagaatggaggtgctgggctgtgaggcccaggacctgtactga

SEQ ID NO: 70 - DNA, 4374 bp, synthetic construct
  source 1..4374: mol_type = other DNA; note = FVIII-QQ10
atgcagatagaactgagcacctgcttcttcctgtgcctgctgagattctgcttctctgccaccagaagatactacctgggggctgtggagctgagctgggactacatgcagagtgacctgggagagctgccagttgatgcaagattcccccccagagtgcccaagagcttccccttcaacacctctgtggtgtacaagaagaccctgtttgtggagttcactgatcacctgttcaacatagccaagcccagacccccctggatgggcctgctgggccccaccatccaggctgaagtgtatgacactgtagtgatcaccctgaagaacatggccagccaccctgtgagcctgcatgctgtgggagtgagctactggaaggcctcagagggagcagagtatgatgaccagaccagccagagagagaaggaggatgacaaggtgttcccaggaggcagccacacctatgtttggcaggtgctgaaggagaatgggcccatggcctcagaccccctgtgcctgacctacagctacctgagccatgtagacctggtgaaggacctgaactctggcctgataggagccctgctggtgtgcagagagggcagcctggccaaggagaagacccagaccctgcacaagttcatcctgctgtttgcagtgtttgatgagggcaagagctggcactcagagaccaagaacagcctgatgcaggacagagatgcagccagtgctagagcctggcccaagatgcacacagtgaatgggtatgttaacagaagcctgccaggcctgataggatgccacagaaagtctgtgtactggcatgtgattggtatgggcaccaccccagaggtgcacagcatcttcctggagggccacaccttcctggtgagaaaccacagacaggccagcctggagatcagccccatcaccttcctgacagcccagaccctgctgatggacctgggccagttcctgctgttctgccacatcagcagccaccagcatgatggcatggaggcctatgttaaggtggacagctgcccagaggagccccagctgcagatgaagaacaatgaagaggcagaggactatgatgatgacctgacagattcagagatggatgtggtgagatttgatgatgacaacagccccagcttcatccagatcagaagtgtggccaagaagcaccccaagacctgggtgcactacattgctgctgaagaggaggactgggactatgcccccctggtgctggccccagatgacagaagctacaagagccagtacctgaacaatgggccccagagaattggaagaaagtacaagaaggtgagattcatggcctacacagatgaaaccttcaagaccagagaggccatccagcatgagagtgggatcctgggccccctgctgtatggggaggtgggtgataccctgctgatcatcttcaagaaccaggccagcagaccctacaacatctacccccatggcatcacagatgtgagacccctgtacagcagaagactgcccaagggagttaagcacctgaaggacttccccatcctgccaggagagatcttcaagtacaagtggacagtcacagtggaggatgggcccaccaagtctgaccccagatgcctgaccagatactacagcagctttgtaaacatggagagagacctggcctcaggtctgataggccccctgctgatctgctacaaggagagtgttgatcagcagggcaaccagatcatgtctgacaagagaaatgtgatcctgttctcagtgtttgatgaaaacagaagctggtacctgactgagaacatccagagattcctgcccaacccagctggggtgcagctggaggaccctgagttccaggccagcaacatcatgcacagcatcaatggatatgtgtttgacagcctgcagctgtcagtgtgcctgcatgaggtggcctactggtacatcctgagcataggagctcagacagacttcctgagtgtattcttctcagggtacaccttcaagcacaagatggtgtatgaggacaccctgaccctgttccccttctcaggtgagacagtgttcatgagcatggagaacccagggctgtggatcctgggctgccacaactctgatttcagaaacagaggcatgactgccctgctgaaggtgagcagctgtgacaagaacacaggggactactatgaggacagctatgaggacatctctgcctacctgctgagcaagaacaatgctattgagcccagaagcttcagccagaacccccctgtactgaagagacaccagagagagatcaccagaaccaccctgcagagtgatcaggaggagatagattatgatgacaccatctcagtggagatgaagaaggaggactttgatatctatgatgaggatgaaaaccagagccccagaagcttccagaagaagaccagacactacttcattgcagctgtagagagactgtgggactatggtatgagcagcagcccccatgtcctgagaaacagagcccagtctggatcagtcccccagttcaagaaggtggtgttccaggagttcacagatggtagcttcacccagcccctgtacagaggagagctgaatgagcacctgggcctgctgggcccctacatcagagcagaggtggaggacaacatcatggtgaccttcagaaaccaggccagcagaccctacagcttctacagcagcctgatcagctatgaagaggaccagagacagggagcagagcccagaaagaactttgtgaagcccaatgagaccaagacctacttctggaaggtgcagcaccacatggcccccaccaaggatgagtttgattgcaaggcctgggcctacttcagtgatgttgacctggagaaggatgttcacagtggcctgataggtcccctgctggtgtgccacaccaacaccctgaacccagcccatggcagacaggtgactgtccaggagtttgccctgttcttcaccatctttgatgagaccaagagctggtacttcactgaaaacatggagagaaactgcagagccccctgcaacatccagatggaggaccccaccttcaaggagaactacagattccatgccatcaatggctacatcatggacaccctgcctggactggtgatggcccaggaccagagaatcagatggtacctgctgagcatgggcagcaatgagaacatccacagcatccacttctcaggccatgtattcactgtgagaaagaaggaggagtacaagatggccctgtacaacctgtacccaggtgtgtttgagacagtggagatgctgcccagcaaggcaggtatctggagagtggagtgcctgataggagaacacctgcatgctggcatgagcaccctgttcctggtgtacagcaacaagtgccagacccccctgggcatggccagtggccacatcagagacttccagatcacagcctcaggccagtatgggcagtgggcccccaagctggccagactgcactactctggcagcatcaatgcatggagcaccaaggagcccttcagctggatcaaggtggacctgctggcccccatgatcatccatggaatcaagacccagggtgctagacagaagttcagcagcctgtacatcagccagttcatcatcatgtacagcctggatggcaagaagtggcagacctacagaggcaacagcactggcaccctgatggtgttctttggcaatgttgacagctctggcatcaagcacaacatcttcaacccccccatcatagcaagatacatcagactgcaccccacccactacagcatcagaagcaccctgagaatggagctgatgggctgtgatctgaacagctgcagcatgcccctgggcatggagagcaaggccatcagtgatgctcagatcactgccagcagctacttcaccaacatgtttgcaacctggagccccagcaaggccagactgcacctgcagggcagaagcaatgcctggagaccccaggtgaacaaccccaaggagtggctgcaggtggacttccagaagaccatgaaggtgacaggtgttaccacccagggtgtcaagagcctgctgaccagcatgtatgtaaaggagttcctgatcagcagcagccaggatggccaccagtggaccctgttcttccagaatggaaaggtgaaggtgttccagggcaaccaggacagcttcacccctgtggtgaacagcctggacccccccctgctgaccagatacctgagaatccacccccagagctgggtgcaccagatagccctgagaatggaggtgctgggctgtgaagcccaggacctgtactga

SEQ ID NO: 71 - DNA, 4374 bp, synthetic construct
  source 1..4374: mol_type = other DNA; note = FVIII-QQ11
atgcagatagagctgagcacctgcttcttcctgtgcctgctgagattctgcttctctgccaccagaagatactacctgggtgctgttgagctgagctgggactacatgcagtctgatctgggtgagctgcctgttgatgccagattcccccccagagtgcccaagagcttccccttcaacacctcagttgtgtacaagaagaccctgtttgttgagttcacagaccacctgttcaacattgctaagcccagacccccctggatgggcctgctgggccccaccatccaggcagaggtgtatgatacagttgtgatcaccctgaagaacatggccagccacccagtgagcctgcatgcagtaggtgtgagctactggaaggccagtgagggggctgaatatgatgatcagaccagccagagagagaaggaggatgacaaggtgttcccaggtggtagccacacctatgtatggcaggtgctgaaggagaatggccccatggccagtgaccccctgtgcctgacctacagctacctgagccatgtggacctggtgaaggacctgaactctggactgattggagcactgctggtgtgcagagagggcagcctggccaaggagaagacccagaccctgcacaagttcatcctgctgtttgcagtgtttgatgagggcaagagctggcactcagagaccaagaacagcctgatgcaggacagagatgcagcttctgccagagcctggcccaagatgcacactgtcaatggctatgtgaacagaagcctgccaggcctgattggttgccacagaaagagtgtgtactggcatgttatagggatgggcaccacccctgaggtgcacagcatcttcctggagggccacaccttcctggtgagaaaccacagacaggccagcctggagatcagccccatcaccttcctgacagcacagaccctgctgatggacctgggccagttcctgctgttctgccacatcagcagccaccagcatgatggcatggaggcctatgttaaggtggacagctgccctgaggagccccagctgcagatgaagaacaatgaggaggctgaggactatgatgatgacctgacagactcagagatggatgtggtgagatttgatgatgataacagccccagcttcatccagatcagatcagtggccaagaagcaccccaagacctgggtgcactacatagctgctgaagaggaggactgggactatgcccccctggtgctggccccagatgacagaagctacaagagccagtacctgaacaatgggccccagagaattggcagaaagtacaagaaggtgagattcatggcctacactgatgagaccttcaagaccagagaggccatccagcatgagtctggaatcctgggccccctgctgtatggagaggtgggtgacaccctgctgatcatcttcaagaaccaggccagcagaccctacaacatctacccccatggcatcactgatgttagacccctgtacagcagaagactgcccaagggtgtgaagcacctgaaggacttccccatcctgcctggggagatcttcaagtacaagtggacagtcactgtagaggatggccccaccaagagtgaccccagatgcctgaccagatactacagcagctttgtgaacatggagagagacctggcctctggactgattggacccctgctgatctgctacaaggagagtgtggaccagcagggcaaccagatcatgtcagataagagaaatgtcatcctgttctcagtatttgatgagaacagaagctggtacctgactgagaacatccagagattcctgcccaacccagctggggtgcagctggaggaccctgaattccaggccagcaacatcatgcacagcatcaatggctatgtatttgatagcctgcagctgtctgtgtgcctgcatgaggtggcctactggtacatcctgagcattggggcccagactgatttcctgtctgtcttcttctctgggtacaccttcaagcacaagatggtgtatgaggacaccctgaccctgttccccttctcaggagagacagtgttcatgagcatggagaacccaggcctgtggatcctgggctgccacaactcagacttcagaaacagaggcatgactgccctgctgaaggtgagcagctgtgacaagaacacaggtgactactatgaggacagctatgaagacatctctgcctacctgctgagcaagaacaatgctatagaacccagaagcttcagccagaacccccctgtgctgaagagacaccagagagagatcaccagaaccaccctgcagtcagatcaggaggagatagattatgatgataccatctcagtagaaatgaagaaggaggactttgacatctatgatgaagatgaaaaccagagccccagaagcttccagaagaagaccagacactacttcatagctgctgtagagagactgtgggactatggcatgagcagcagcccccatgtactgagaaacagagcccagagtggctcagtgccccagttcaagaaggtggtgttccaggagttcactgatggcagcttcacccagcccctgtacagaggtgaactgaatgagcacctgggcctgctgggcccctacatcagagcagaggtggaggacaacatcatggtgaccttcagaaaccaggccagcagaccctacagcttctacagcagcctgatcagctatgaagaggaccagagacagggtgctgagcccagaaagaactttgtgaagcccaatgagaccaagacctacttctggaaggtgcagcaccacatggcccccaccaaggatgagtttgactgcaaggcctgggcctacttcagtgatgtagacctggagaaggatgtgcacagtgggctgataggtcccctgctggtgtgccacaccaacaccctgaacccagcacatggtagacaggtgacagttcaggagtttgccctgttcttcaccatctttgatgaaaccaagagctggtacttcactgagaacatggagagaaactgcagagccccctgcaacatccagatggaggaccccaccttcaaggagaactacagattccatgccatcaatggctacatcatggacaccctgccaggcctggtgatggcccaggaccagagaatcagatggtacctgctgagcatgggcagcaatgagaacatccacagcatccacttctcaggacatgtgttcactgtgagaaagaaggaggagtacaagatggccctgtacaacctgtacccaggggtatttgaaacagtagagatgctgcccagcaaggcaggcatctggagagtggagtgcctgattggagaacacctgcatgcaggtatgagcaccctgttcctggtgtacagcaacaagtgccagacccccctgggcatggcctcaggccacatcagagacttccagatcacagcttctggccagtatgggcagtgggcccccaagctggccagactgcactactcaggtagcatcaatgcttggagcaccaaggagcccttcagctggatcaaggtggacctgctggcccccatgatcatccatgggatcaagacccagggagccagacagaagttcagcagcctgtacatcagccagttcatcatcatgtacagcctggatggtaagaagtggcagacctacagaggcaacagcactggcaccctgatggtgttctttggaaatgttgatagctcagggatcaagcacaacatcttcaacccccccatcatagccagatacatcagactgcaccccacccactacagcatcagaagcaccctgagaatggagctgatgggctgtgatctgaacagctgcagcatgcccctgggcatggagagcaaggccatctctgatgcacagatcacagcaagcagctacttcaccaacatgtttgccacctggagccccagcaaggccagactgcacctgcagggcagaagcaatgcttggagaccccaggtgaacaaccccaaggagtggctgcaggtggacttccagaagaccatgaaggtgacaggggtgaccacccagggggtgaagagcctgctgaccagcatgtatgtcaaggagttcctgatcagcagcagccaggatggccaccagtggaccctgttcttccagaatgggaaggtgaaggtgttccagggcaaccaggacagcttcaccccagtagtgaacagcctggacccccccctgctgaccagatacctgagaatccacccccagagctgggtgcaccagattgctctgagaatggaggtgctgggctgtgaggcccaggacctgtactga

SEQ ID NO: 72 - DNA, 4374 bp, synthetic construct
  source 1..4374: mol_type = other DNA; note = FVIII-QQ12
atgcagatagaactgagcacctgcttcttcctgtgcctgctgagattctgcttctctgccaccagaagatactacctgggggctgtggagctgagctgggactacatgcagtctgacctgggagaactgcctgttgatgctagattcccccccagagtgcccaagagcttccccttcaacacctcagtggtgtacaagaagaccctgtttgtagagttcactgaccacctgttcaacattgcaaagcccagacccccctggatgggcctgctgggccccaccatccaggctgaggtgtatgatactgttgtgatcaccctgaagaacatggccagccaccctgtgagcctgcatgctgtgggagtcagctactggaaggcctcagagggggctgagtatgatgaccagaccagccagagagagaaggaggatgacaaggtgttccctggtgggagccacacctatgtgtggcaggtgctgaaggagaatggacccatggcctcagaccccctgtgcctgacctacagctacctgagccatgtagacctggtgaaggacctgaacagtggtctgataggtgcactgctggtgtgcagagagggcagcctggccaaggagaagacccagaccctgcacaagttcatcctgctgtttgcagtgtttgatgagggcaagagctggcactctgagaccaagaacagcctgatgcaggacagagatgctgcctcagccagagcctggcccaagatgcacactgttaatgggtatgtgaacagaagcctgccaggcctgattggctgccacagaaagagtgtgtactggcatgtcataggaatgggcaccacccctgaggtgcacagcatcttcctggagggccacaccttcctggtgagaaaccacagacaggccagcctggagatcagccccatcaccttcctgactgcacagaccctgctgatggacctgggccagttcctgctgttctgccacatcagcagccaccagcatgatgggatggaggcctatgtaaaggtggacagctgccctgaagagccccagctgcagatgaagaacaatgaggaggcagaagactatgatgatgatctgacagacagtgaaatggatgtggtgagatttgatgatgacaacagccccagcttcatccagatcagaagtgtggccaagaagcaccccaagacctgggtgcactacattgctgcagaggaggaggactgggactatgcccccctggtgctggccccagatgacagaagctacaagagccagtacctgaacaatgggccccagagaatagggagaaagtacaagaaggtgagattcatggcctacacagatgagaccttcaagaccagagaggccatccagcatgagagtggtatcctgggccccctgctgtatggagaagtgggggataccctgctgatcatcttcaagaaccaggccagcagaccctacaacatctacccccatggcatcacagatgtgagacccctgtacagcagaagactgcccaagggtgtaaagcacctgaaggacttccccatcctgccaggggaaatcttcaagtacaagtggactgtgactgtggaggatggacccaccaagagtgatcccagatgcctgaccagatactacagcagctttgttaacatggagagagacctggcctcaggactgattggccccctgctgatctgctacaaggagagtgtggaccagcagggcaaccagatcatgagtgacaagagaaatgtcatcctgttcagtgtctttgatgaaaacagaagctggtacctgacagaaaacatccagagattcctgcccaaccctgcaggtgtgcagctggaggacccagagttccaggccagcaacatcatgcacagcatcaatgggtatgtgtttgatagcctgcagctgagtgtatgcctgcatgaggtggcctactggtacatcctgagcataggtgctcagactgacttcctgtcagtattcttctcaggctacaccttcaagcacaagatggtgtatgaggacaccctgaccctgttccccttcagtggggaaacagtgttcatgagcatggagaaccctgggctgtggatcctgggctgccacaacagtgacttcagaaacagaggcatgacagccctgctgaaggtgagcagctgtgacaagaacacaggagactactatgaagacagctatgaagacatcagtgcttacctgctgagcaagaacaatgcaatagagcccagaagcttcagccagaaccccccagtgctgaagagacaccagagagagatcaccagaaccaccctgcagtcagaccaggaggagattgactatgatgacaccatctctgtggagatgaagaaggaggactttgacatctatgatgaggatgagaaccagagccccagaagcttccagaagaagaccagacactacttcatagcagcagtggagagactgtgggactatgggatgagcagcagcccccatgtcctgagaaacagagcccagtcaggaagtgtcccccagttcaagaaggtggtgttccaggagttcactgatgggagcttcacccagcccctgtacagaggagagctgaatgaacacctgggcctgctgggcccctacatcagagcagaggtggaggacaacatcatggtgaccttcagaaaccaggccagcagaccctacagcttctacagcagcctgatcagctatgaggaggaccagagacagggagctgaacccagaaagaactttgtgaagcccaatgaaaccaagacctacttctggaaggtgcagcaccacatggcccccaccaaggatgagtttgactgcaaggcctgggcctacttctctgatgttgatctggagaaggatgtacactctgggctgattggccccctgctggtgtgccacaccaacaccctgaacccagcccatggcagacaggtgactgtgcaggagtttgccctgttcttcaccatctttgatgagaccaagagctggtacttcactgaaaacatggagagaaactgcagagccccctgcaacatccagatggaggaccccaccttcaaggagaactacagattccatgctatcaatggctacatcatggacaccctgcctggactggtgatggcccaggaccagagaatcagatggtacctgctgagcatgggcagcaatgaaaacatccacagcatccacttctcaggccatgtattcactgtgagaaagaaggaggagtacaagatggccctgtacaacctgtacccaggtgtgtttgaaacagtggaaatgctgcccagcaaggctggcatctggagagtggagtgcctgattggtgagcacctgcatgctggaatgagcaccctgttcctggtgtacagcaacaagtgccagacccccctgggcatggcctcagggcacatcagagacttccagatcactgcctctggccagtatggacagtgggcccccaagctggccagactgcactacagtggcagcatcaatgcctggagcaccaaggagcccttcagctggatcaaggtggacctgctggcccccatgatcatccatggcatcaagacccagggtgccagacagaagttcagcagcctgtacatcagccagttcatcatcatgtacagcctggatggcaagaagtggcagacctacagaggcaacagcacaggtaccctgatggtgttctttggcaatgtggacagctctggtatcaagcacaacatcttcaacccccccatcatagccagatacatcagactgcaccccacccactacagcatcagaagcaccctgagaatggagctgatgggctgtgatctgaacagctgcagcatgcccctgggcatggagagcaaggccatctctgatgctcagatcacagctagcagctacttcaccaacatgtttgccacctggagccccagcaaggccagactgcacctgcagggcagaagcaatgcatggagaccccaggtgaacaaccccaaggagtggctgcaggtggacttccagaagaccatgaaggtgacaggagtgaccacccagggagtgaagagcctgctgaccagcatgtatgtgaaggagttcctgatcagcagcagccaggatggccaccagtggaccctgttcttccagaatgggaaggtgaaggtgttccagggcaaccaggacagcttcaccccagtagtgaacagcctggacccccccctgctgaccagatacctgagaatccacccccagagctgggtgcaccagatagctctgagaatggaggtgctgggctgtgaagcccaggacctgtactga

SEQ ID NO: 73 - DNA, 4374 bp, synthetic construct
  source 1..4374: mol_type = other DNA; note = FVIII-QQ13
atgcagatagaactgagcacctgcttcttcctgtgcctgctgagattctgcttcagtgccaccagaagatactacctgggtgctgttgagctgagctgggactacatgcagtctgacctgggtgaactgccagtagatgcaagattcccccccagagtgcccaagagcttccccttcaacacctctgtagtctacaagaagaccctgtttgtggagttcacagatcacctgttcaacattgctaagcccagacccccctggatgggcctgctgggccccaccatccaggcagaggtgtatgacactgtagtgatcaccctgaagaacatggccagccacccagtgagcctgcatgctgtgggtgtgagctactggaaggccagtgaaggagcagagtatgatgatcagaccagccagagagagaaggaggatgacaaggtgttcccaggtggaagccacacctatgtctggcaggtgctgaaggagaatgggcccatggcctcagatcccctgtgcctgacctacagctacctgagccatgttgacctggtgaaggacctgaacagtggactgataggtgctctgctggtgtgcagagagggcagcctggccaaggagaagacccagaccctgcacaagttcatcctgctgtttgcagtgtttgatgaaggcaagagctggcacagtgagaccaagaacagcctgatgcaggacagagatgcagcatcagccagagcctggcccaagatgcacacagtgaatggctatgtgaacagaagcctgccaggcctgataggatgccacagaaagtctgtatactggcatgtcattggtatgggcaccacccctgaagtgcacagcatcttcctggagggccacaccttcctggtgagaaaccacagacaggccagcctggagatcagccccatcaccttcctgacagcccagaccctgctgatggacctgggccagttcctgctgttctgccacatcagcagccaccagcatgatgggatggaggcctatgtcaaggtggacagctgcccagaggagccccagctgcagatgaagaacaatgaggaggctgaggactatgatgatgacctgactgactcagagatggatgtggtgagatttgatgatgataacagccccagcttcatccagatcagatctgtggccaagaagcaccccaagacctgggtgcactacatagctgcagaagaggaggactgggactatgcccccctggtgctggccccagatgatagaagctacaagagccagtacctgaacaatggcccccagagaattgggagaaagtacaagaaggtgagattcatggcctacacagatgaaaccttcaagaccagagaggccatccagcatgagtctggcatcctgggccccctgctgtatggggaagtgggagacaccctgctgatcatcttcaagaaccaggccagcagaccctacaacatctacccccatggtatcacagatgtgagacccctgtacagcagaagactgcccaagggggtgaagcacctgaaggacttccccatcctgcctggggagatcttcaagtacaagtggacagtaactgtggaggatggccccaccaagtctgaccccagatgcctgaccagatactacagcagctttgtgaacatggagagagacctggccagtggcctgattggccccctgctgatctgctacaaggagagtgttgaccagcagggcaaccagatcatgagtgataagagaaatgttatcctgttcagtgtgtttgatgaaaacagaagctggtacctgacagagaacatccagagattcctgcccaacccagctggtgtccagctggaggaccctgagttccaggccagcaacatcatgcacagcatcaatggctatgtatttgacagcctgcagctgagtgtgtgcctgcatgaagtggcctactggtacatcctgagcataggtgctcagacagacttcctgagtgtcttcttcagtggctacaccttcaagcacaagatggtgtatgaagacaccctgaccctgttccccttcagtggagagacagtgttcatgagcatggagaacccaggactgtggatcctgggctgccacaacagtgacttcagaaacagaggcatgacagccctgctgaaggtgagcagctgtgacaagaacactggggactactatgaggacagctatgaggacatctctgcctacctgctgagcaagaacaatgcaatagagcccagaagcttcagccagaacccccctgtgctgaagagacaccagagagagatcaccagaaccaccctgcagtcagatcaggaggagatagactatgatgataccatctctgtggagatgaagaaggaggactttgatatctatgatgaggatgagaaccagagccccagaagcttccagaagaagaccagacactacttcatagctgcagtggagagactgtgggactatggcatgagcagcagcccccatgtactgagaaacagagcccagtcaggatctgtcccccagttcaagaaggtggtgttccaggagttcactgatgggagcttcacccagcccctgtacagaggtgaactgaatgaacacctgggcctgctgggcccctacatcagagcagaggtggaggacaacatcatggtgaccttcagaaaccaggccagcagaccctacagcttctacagcagcctgatcagctatgaagaggaccagagacagggagctgagcccagaaagaactttgtgaagcccaatgaaaccaagacctacttctggaaggtgcagcaccacatggcccccaccaaggatgaatttgactgcaaggcctgggcctacttctcagatgtagacctggagaaggatgtgcactcaggactgattggccccctgctggtgtgccacaccaacaccctgaacccagcacatggcagacaggtgacagttcaggagtttgcactgttcttcaccatctttgatgagaccaagagctggtacttcacagagaacatggagagaaactgcagagccccctgcaacatccagatggaggaccccaccttcaaggagaactacagattccatgctatcaatggctacatcatggacaccctgccagggctggtgatggcccaggaccagagaatcagatggtacctgctgagcatgggcagcaatgaaaacatccacagcatccacttcagtggccatgtgttcacagttagaaagaaggaggagtacaagatggccctgtacaacctgtaccctggtgtgtttgaaacagtggagatgctgcccagcaaggctggcatctggagagtggagtgcctgattggggaacacctgcatgcaggaatgagcaccctgttcctggtgtacagcaacaagtgccagacccccctgggcatggcctcaggccacatcagagacttccagatcactgccagtgggcagtatggtcagtgggcccccaagctggccagactgcactactctggcagcatcaatgcctggagcaccaaggagcccttcagctggatcaaggtggacctgctggcccccatgatcatccatggcatcaagacccagggggccagacagaagttcagcagcctgtacatcagccagttcatcatcatgtacagcctggatggcaagaagtggcagacctacagaggcaacagcactggaaccctgatggtgttctttggcaatgtagacagctcaggcatcaagcacaacatcttcaacccccccatcattgccagatacatcagactgcaccccacccactacagcatcagaagcaccctgagaatggagctgatgggctgtgacctgaacagctgcagcatgcccctgggcatggagagcaaggccatctctgatgcccagatcactgccagcagctacttcaccaacatgtttgccacctggagccccagcaaggccagactgcacctgcagggcagaagcaatgcttggagaccccaggtgaacaaccccaaggagtggctgcaggtggacttccagaagaccatgaaggtgactggtgtgaccacccagggtgtgaagagcctgctgaccagcatgtatgtcaaggagttcctgatcagcagcagccaggatggtcaccagtggaccctgttcttccagaatggcaaggtgaaggtgttccagggcaaccaggacagcttcaccccagtggtgaacagcctggacccccccctgctgaccagatacctgagaatccacccccagagctgggtgcaccagatagccctgagaatggaggtgctgggctgtgaggcccaggacctgtactga

SEQ ID NO: 74 - DNA, 4374 bp, synthetic construct
  source 1..4374: mol_type = other DNA; note = FVIII-QQ14
atgcagattgaactgagcacctgcttcttcctgtgcctgctgagattctgcttcagtgccaccagaagatactacctgggggcagtggagctgagctgggactacatgcagagtgacctgggggaactgcctgttgatgccagattcccccccagagtgcccaagagcttccccttcaacacctcagttgtgtacaagaagaccctgtttgtggagttcactgaccacctgttcaacattgctaagcccagacccccctggatgggcctgctgggccccaccatccaggctgaggtgtatgacactgtggtgatcaccctgaagaacatggccagccaccctgtgagcctgcatgcagtgggggtgagctactggaaggcctcagaaggggcagagtatgatgatcagaccagccagagagagaaggaggatgacaaggtgttccctggtggtagccacacctatgtgtggcaggtgctgaaggagaatggccccatggcctcagaccccctgtgcctgacctacagctacctgagccatgtggacctggtgaaggacctgaacagtggcctgattggagctctgctggtgtgcagagagggcagcctggccaaggagaagacccagaccctgcacaagttcatcctgctgtttgctgtgtttgatgagggcaagagctggcactcagagaccaagaacagcctgatgcaggacagagatgctgccagtgcaagagcctggcccaagatgcacactgtcaatggatatgtgaacagaagcctgccaggactgataggctgccacagaaagtctgtatactggcatgttataggtatgggcaccaccccagaggtgcacagcatcttcctggagggccacaccttcctggtgagaaaccacagacaggccagcctggagatcagccccatcaccttcctgactgcccagaccctgctgatggacctgggccagttcctgctgttctgccacatcagcagccaccagcatgatggtatggaggcctatgtgaaggtggacagctgccctgaggagccccagctgcagatgaagaacaatgaggaggcagaggactatgatgatgacctgacagacagtgagatggatgtggtgagatttgatgatgacaacagccccagcttcatccagatcagaagtgtggccaagaagcaccccaagacctgggtgcactacattgctgctgaagaggaggactgggactatgcacccctggtgctggccccagatgatagaagctacaagagccagtacctgaacaatggaccccagagaattggaagaaagtacaagaaggtgagattcatggcctacactgatgagaccttcaagaccagagaggccatccagcatgaatcagggatcctgggccccctgctgtatggagaggtgggagacaccctgctgatcatcttcaagaaccaggccagcagaccctacaacatctacccccatggtatcactgatgtaagacccctgtacagcagaagactgcccaagggagtgaagcacctgaaggacttccccatcctgcctggggaaatcttcaagtacaagtggacagtcacagttgaagatggacccaccaagagtgatcccagatgcctgaccagatactacagcagctttgtgaacatggagagagacctggcctcaggcctgataggccccctgctgatctgctacaaggagtctgttgaccagcagggcaaccagatcatgagtgacaagagaaatgtgatcctgttctcagtgtttgatgagaacagaagctggtacctgactgaaaacatccagagattcctgcccaacccagcaggggtgcagctggaggacccagagttccaggccagcaacatcatgcacagcatcaatggctatgtctttgatagcctgcagctgtctgtatgcctgcatgaggtggcctactggtacatcctgagcataggagcccagactgatttcctgagtgttttcttcagtggctacaccttcaagcacaagatggtgtatgaggacaccctgaccctgttccccttctctggtgagactgtgttcatgagcatggagaaccctggtctgtggatcctgggctgccacaactcagatttcagaaacagaggcatgacagccctgctgaaggtgagcagctgtgacaagaacactggggactactatgaagacagctatgaagacatctctgcttacctgctgagcaagaacaatgccattgagcccagaagcttcagccagaacccccctgtgctgaagagacaccagagagagatcaccagaaccaccctgcagtcagaccaggaggagatagactatgatgataccatctctgtggagatgaagaaggaggactttgatatctatgatgaggatgaaaaccagagccccagaagcttccagaagaagaccagacactacttcatagcagctgtagagagactgtgggactatggcatgagcagcagcccccatgtactgagaaacagagcccagagtggaagtgtgccccagttcaagaaggtggtgttccaggagttcacagatggcagcttcacccagcccctgtacagaggagaactgaatgagcacctgggcctgctgggcccctacatcagagctgaggtggaggacaacatcatggtgaccttcagaaaccaggccagcagaccctacagcttctacagcagcctgatcagctatgaggaggaccagagacagggagctgagcccagaaagaactttgtcaagcccaatgagaccaagacctacttctggaaggtgcagcaccacatggcccccaccaaggatgaatttgactgcaaggcctgggcctacttctcagatgtggacctggagaaggatgtgcactcaggtctgattggccccctgctggtgtgccacaccaacaccctgaacccagcccatggtagacaggtgactgtgcaggagtttgctctgttcttcaccatctttgatgagaccaagagctggtacttcacagagaacatggagagaaactgcagagccccctgcaacatccagatggaggaccccaccttcaaggagaactacagattccatgccatcaatgggtacatcatggacaccctgccaggtctggtgatggcccaggaccagagaatcagatggtacctgctgagcatgggcagcaatgaaaacatccacagcatccacttcagtggacatgtgttcacagttagaaagaaggaggagtacaagatggccctgtacaacctgtaccctggagtgtttgaaactgtggagatgctgcccagcaaggcaggcatctggagagtggagtgcctgataggggagcacctgcatgctggtatgagcaccctgttcctggtgtacagcaacaagtgccagacccccctgggcatggcctcaggccacatcagagacttccagatcactgctagtggccagtatggtcagtgggcccccaagctggccagactgcactactctggaagcatcaatgcctggagcaccaaggagcccttcagctggatcaaggtggacctgctggcccccatgatcatccatgggatcaagacccagggggccagacagaagttcagcagcctgtacatcagccagttcatcatcatgtacagcctggatggaaagaagtggcagacctacagaggcaacagcacaggcaccctgatggtgttctttggcaatgtagacagctcaggcatcaagcacaacatcttcaacccccccatcatagcaagatacatcagactgcaccccacccactacagcatcagaagcaccctgagaatggagctgatgggctgtgacctgaacagctgcagcatgcccctgggcatggagagcaaggccatctcagatgcccagatcacagccagcagctacttcaccaacatgtttgccacctggagccccagcaaggccagactgcacctgcagggcagaagcaatgcctggagaccccaggtgaacaaccccaaggagtggctgcaggtggacttccagaagaccatgaaggtgactggtgtgaccacccagggtgttaagagcctgctgaccagcatgtatgtgaaggagttcctgatcagcagcagccaggatgggcaccagtggaccctgttcttccagaatggaaaggtgaaggtgttccagggcaaccaggacagcttcacccctgttgtgaacagcctggacccccccctgctgaccagatacctgagaatccacccccagagctgggtgcaccagatagcactgagaatggaggtgctgggctgtgaggcccaggacctgtactga

SEQ ID NO: 75 - DNA, 4374 bp, synthetic construct
  source 1..4374: mol_type = other DNA; note = FVIII-QQ15
atgcagattgaactgagcacctgcttcttcctgtgcctgctgagattctgcttcagtgctaccagaagatactacctgggggcagtggagctgagctgggactacatgcagtctgacctgggagagctgccagtagatgccagattcccccccagagtgcccaagagcttccccttcaacacctctgtggtgtacaagaagaccctgtttgtggagttcacagaccacctgttcaacatagctaagcccagacccccctggatgggcctgctgggccccaccatccaggcagaggtgtatgacactgttgtgatcaccctgaagaacatggccagccacccagttagcctgcatgctgttggtgttagctactggaaggcctctgagggagcagagtatgatgaccagaccagccagagagagaaggaggatgacaaggtgttcccaggggggagccacacctatgtctggcaggtgctgaaggagaatggccccatggcctcagaccccctgtgcctgacctacagctacctgagccatgtggacctggtgaaggacctgaactctggcctgattggtgcactgctggtgtgcagagagggcagcctggccaaggagaagacccagaccctgcacaagttcatcctgctgtttgctgtctttgatgagggcaagagctggcactcagaaaccaagaacagcctgatgcaggacagagatgcagcttctgctagagcctggcccaagatgcacactgtgaatggctatgtcaacagaagcctgcctggcctgataggctgccacagaaagtctgtgtactggcatgtgattgggatgggcaccaccccagaggtgcacagcatcttcctggagggccacaccttcctggtgagaaaccacagacaggccagcctggagatcagccccatcaccttcctgactgcccagaccctgctgatggacctgggccagttcctgctgttctgccacatcagcagccaccagcatgatggtatggaggcctatgtgaaggtggacagctgcccagaggagccccagctgcagatgaagaacaatgaagaggcagaggactatgatgatgacctgacagactcagaaatggatgtggtgagatttgatgatgataacagccccagcttcatccagatcagatctgttgccaagaagcaccccaagacctgggtgcactacattgcagctgaggaggaggactgggactatgctcccctggtgctggccccagatgacagaagctacaagagccagtacctgaacaatggaccccagagaatagggagaaagtacaagaaggtgagattcatggcctacacagatgagaccttcaagaccagagaggccatccagcatgaatcagggatcctgggccccctgctgtatggggaggtgggtgacaccctgctgatcatcttcaagaaccaggccagcagaccctacaacatctacccccatggaatcacagatgtcagacccctgtacagcagaagactgcccaagggagtaaagcacctgaaggacttccccatcctgccaggtgagatcttcaagtacaagtggacagtaacagtggaggatggtcccaccaagtctgaccccagatgcctgaccagatactacagcagctttgttaacatggagagagacctggccagtggcctgataggccccctgctgatctgctacaaggagtctgtagaccagcagggcaaccagatcatgtcagataagagaaatgtaatcctgttcagtgtgtttgatgagaacagaagctggtacctgacagagaacatccagagattcctgcccaaccctgcaggtgtgcagctggaggaccctgaattccaggccagcaacatcatgcacagcatcaatggatatgtttttgacagcctgcagctgtcagtttgcctgcatgaggtggcctactggtacatcctgagcattggggcccagacagatttcctgtctgtgttcttcagtgggtacaccttcaagcacaagatggtgtatgaggacaccctgaccctgttccccttctcaggtgaaactgtgttcatgagcatggagaacccagggctgtggatcctgggctgccacaactcagacttcagaaacagaggcatgactgctctgctgaaggtgagcagctgtgacaagaacacaggtgactactatgaggacagctatgaagacatctcagcttacctgctgagcaagaacaatgccatagagcccagaagcttcagccagaaccccccagttctgaagagacaccagagagagatcaccagaaccaccctgcagagtgaccaggaggagattgactatgatgacaccatctctgtagagatgaagaaggaggactttgacatctatgatgaggatgagaaccagagccccagaagcttccagaagaagaccagacactacttcatagctgcagtggagagactgtgggactatggcatgagcagcagcccccatgtgctgagaaacagagcccagagtggctcagtgccccagttcaagaaggtggtgttccaggagttcacagatggcagcttcacccagcccctgtacagaggggagctgaatgagcacctgggcctgctgggcccctacatcagagctgaggtggaggacaacatcatggtgaccttcagaaaccaggccagcagaccctacagcttctacagcagcctgatcagctatgaggaggaccagagacagggggcagagcccagaaagaactttgtaaagcccaatgaaaccaagacctacttctggaaggtgcagcaccacatggcccccaccaaggatgaatttgactgcaaggcctgggcctacttcagtgatgtggacctggagaaggatgtgcactcaggcctgattggtcccctgctggtgtgccacaccaacaccctgaacccagcccatggcagacaggtgactgtacaggagtttgccctgttcttcaccatctttgatgagaccaagagctggtacttcacagagaacatggagagaaactgcagagccccctgcaacatccagatggaggaccccaccttcaaggagaactacagattccatgctatcaatgggtacatcatggacaccctgccaggcctggtgatggcccaggaccagagaatcagatggtacctgctgagcatgggcagcaatgagaacatccacagcatccacttcagtggccatgtgttcacagtgagaaagaaggaggagtacaagatggccctgtacaacctgtaccctggagtgtttgaaactgtggagatgctgcccagcaaggcaggtatctggagagtggagtgcctgattggtgagcacctgcatgctgggatgagcaccctgttcctggtgtacagcaacaagtgccagacccccctgggcatggcctcagggcacatcagagacttccagatcactgccagtgggcagtatgggcagtgggcccccaagctggccagactgcactacagtggaagcatcaatgcatggagcaccaaggagcccttcagctggatcaaggtggacctgctggcccccatgatcatccatgggatcaagacccagggggccagacagaagttcagcagcctgtacatcagccagttcatcatcatgtacagcctggatggcaagaagtggcagacctacagaggcaacagcacaggtaccctgatggtgttctttggaaatgtggacagctcaggtatcaagcacaacatcttcaacccccccatcatagcaagatacatcagactgcaccccacccactacagcatcagaagcaccctgagaatggagctgatgggctgtgacctgaacagctgcagcatgcccctgggcatggagagcaaggccatctcagatgctcagatcactgccagcagctacttcaccaacatgtttgccacctggagccccagcaaggccagactgcacctgcagggcagaagcaatgcctggagaccccaggtgaacaaccccaaggagtggctgcaggtggacttccagaagaccatgaaggtgacaggggttaccacccagggagtgaagagcctgctgaccagcatgtatgtaaaggagttcctgatcagcagcagccaggatggtcaccagtggaccctgttcttccagaatggaaaggtgaaggtgttccagggcaaccaggacagcttcaccccagtggtgaacagcctggacccccccctgctgaccagatacctgagaatccacccccagagctgggtgcaccagatagccctgagaatggaggtgctgggctgtgaagcccaggacctgtactga

SEQ ID NO: 76 - DNA, 4374 bp, synthetic construct
  source 1..4374: mol_type = other DNA; note = FVIII-QQ16
atgcaaatagaactctccacctgcttctttctgtgccttttgaggttctgcttttctgcaactagaagatactacctgggtgcagtagaactgtcatgggactatatgcaatcagatctaggagaactgccagtggatgccagatttcctccaagggtgcccaaatcatttccattcaacacaagtgttgtgtacaaaaagaccctgtttgtggaattcacagatcaccttttcaacatagccaagcctagaccaccttggatgggtctgttgggtcctaccatccaggcagaagtttatgatactgtggtcattacacttaagaacatggctagccatccagtcagtcttcatgctgttggggtatcctactggaaagcttcagagggagcagaatatgatgaccagacaagtcaaagggagaaagaagatgacaaagtcttccctggtggaagccatacctatgtctggcaggtcctgaaagagaatgggccaatggccagtgacccactgtgccttacctactcttatcttagtcatgtggacctggtaaaagacttgaatagtggcctcattggagcactactggtatgtagagaagggagcttggctaaggaaaagacacagactttgcacaaatttatactactttttgctgtatttgatgaggggaaaagttggcacagtgaaacaaagaactccttgatgcaggatagggatgcagcaagtgctagggcttggccaaaaatgcacacagttaatgggtatgttaacagatcactgcctgggctgattggttgccacagaaaatctgtctattggcatgtgattggaatgggcacaactcctgaagtgcactcaatattccttgaaggtcacacatttcttgtgagaaaccatagacaggcttccttggaaatcagccccataaccttccttacagctcaaacactcttgatggaccttggacagtttctactgttttgtcatatctcttcccaccaacatgatggaatggaagcctatgtcaaagtagacagctgtcctgaggaaccacaactacagatgaaaaataatgaagaagcagaagactatgatgatgatcttactgattcagaaatggatgtggtcaggtttgatgatgataactctccttcctttatccaaattagatctgtagcaaagaagcatcctaaaacttgggtacattacattgctgcagaagaggaggactgggactatgcacccttggtcttggcaccagatgatagaagttataaaagtcaatatttgaacaatggcccccagagaattggtaggaagtacaaaaaagtcagatttatggcatacacagatgaaacatttaagacaagagaagctattcagcatgagtcagggatcttgggacctttactttatggtgaagttggagatacactgttgattatatttaagaatcaagcaagcagaccatataacatctaccctcatggtatcacagatgtaagacccttgtattcaagaagattacccaaaggtgtaaaacatttgaaggattttccaattctgccaggggagatattcaaatataaatggacagtgacagtagaggatgggccaactaaatcagatcctaggtgcctgaccaggtattactctagttttgttaatatggagagagatctagctagtggactcattggacctctcctcatctgctacaaagaatcagttgatcaacagggaaaccagataatgtcagataagaggaatgtcatcctgttttctgtatttgatgaaaacaggagctggtacctcacagaaaatatacaaagatttctccccaatcctgctggagtgcagcttgaagatccagaattccaagcttcaaacatcatgcacagcatcaatggctatgtatttgattccttgcagttgtcagtgtgtttgcatgaggtggcatactggtacattctatctattggagctcagactgacttcctttctgtgttcttctcaggatatacattcaaacacaaaatggtctatgaagacacactcaccctattcccattctcaggggaaacagtcttcatgtcaatggaaaacccaggtctatggattttggggtgccacaactcagactttagaaacagaggcatgacagctttactgaaggttagtagttgtgataagaacactggggattattatgaggactcttatgaagatatttctgcatacttgctgagtaaaaacaatgcaattgaaccaagaagcttctctcaaaaccccccagtcttgaaaagacatcaaagagaaataaccagaactactcttcagtctgatcaagaggaaattgactatgatgataccatatcagttgaaatgaagaaggaagattttgacatttatgatgaagatgaaaatcagagccccaggagctttcaaaagaaaacaagacactattttattgcagcagtggagaggctctgggattatggtatgagtagctccccacatgtactaaggaacagggcccagagtggctctgtcccacagttcaagaaagtagttttccaggaatttacagatggctcctttactcagcctttatacaggggagaactaaatgaacatttgggactcctagggccatatataagagcagaggttgaagataatatcatggtaactttcaggaatcaggcctctaggccctattccttctattctagccttatttcttatgaggaagatcagaggcaaggagcagagcctaggaaaaactttgttaagcctaatgaaaccaaaacatacttttggaaagtgcaacatcatatggcccccaccaaagatgaatttgactgcaaagcctgggcatatttctctgatgtggacttagaaaaagatgttcactcaggcctgataggaccacttctggtctgccacactaacacactgaacccagcccatgggagacaagtgactgtacaggaatttgcactgtttttcaccatctttgatgaaacaaaaagctggtacttcactgaaaatatggaaagaaactgcagggctccctgcaatatccagatggaagatccaacatttaaagagaattatagattccatgctatcaatggctacataatggatacactaccaggcttagtaatggctcaggatcaaaggattagatggtatctgctcagcatgggcagtaatgagaacatccattctattcatttctcaggacatgtattcactgtaagaaaaaaagaggagtataaaatggcactgtacaatctctatccaggtgtttttgagacagtagaaatgttaccttcaaaagcaggaatttggagagtggaatgccttattggtgagcatctacatgctggaatgagcacactttttctggtgtacagcaataagtgtcagacccctctgggaatggcaagtggacacattagagactttcagattacagcttcaggacaatatggacagtgggccccaaagctggccagacttcattattcagggtcaatcaatgcatggagcaccaaggagccttttagctggatcaaggtggatctgttggcaccaatgattattcatggcatcaagactcagggagccagacagaagttctcaagcctctacatctctcagtttatcatcatgtattcccttgatgggaagaagtggcagacttatagaggaaatagtacaggaaccttaatggtcttctttgggaatgtggattcaagtgggataaaacacaatatttttaaccctccaattattgctaggtacatcaggttgcaccccactcattatagcattaggagcactcttagaatggagttgatgggctgtgatttaaatagttgcagtatgccattgggaatggagagtaaagcaatatcagatgcacagattactgcatcatcttactttactaatatgtttgcaacttggtctccatcaaaagcaagacttcacctccaagggaggtctaatgcttggagacctcaggtgaataatccaaaagagtggctgcaagtggacttccagaagacaatgaaagtcacaggtgtgaccactcagggtgtaaaatctctgcttaccagcatgtatgttaaggagttcctcatctccagcagtcaagatggacatcagtggactctcttttttcagaatggtaaagtaaaggtttttcagggaaatcaagactccttcacaccagtggttaacagtctagacccaccattactgactaggtaccttagaattcacccacagagttgggtgcaccagattgccctgaggatggaggttctgggctgtgaagcacaggacctctactga

SEQ ID NO: 77 - DNA, 4374 bp, synthetic construct
  source 1..4374: mol_type = other DNA; note = FVIII-QQ17
atgcaaatagagctctccacctgcttctttctgtgccttttgaggttctgcttttctgccacaaggagatactacctgggtgcagtggaactgtcatgggactatatgcaaagtgacctaggagagctgccagtggatgcaaggtttcctcctagagtgcccaaaagttttccattcaacacatcagttgtgtacaaaaagacactgtttgtagaattcacagatcaccttttcaacatagcaaagcccaggccaccatggatgggtctgctaggtcctaccatccaggcagaggtttatgatactgtggtcattacacttaagaacatggcttcacatcctgtcagtcttcatgctgtgggtgtatcctactggaaagcttcagagggagcagaatatgatgaccagactagtcaaagagagaaagaagatgataaagtcttcccaggtggatcccatacctatgtctggcaggtcctgaaagagaatggtcctatggccagtgatcctctgtgccttacttacagttatcttagtcatgttgacctggtaaaagacttgaattcaggactcattggagcactattggtatgtagagaagggtctttagctaaggaaaagacacagaccttgcacaaatttatactactttttgcagtatttgatgaagggaaaagttggcacagtgaaacaaagaactccttgatgcaggatagggatgcagcatctgctagggcctggcccaaaatgcacacagtcaatgggtatgtgaacaggtctctgcctggtctgattggatgccacagaaaatcagtatattggcatgtgataggaatgggcacaactcctgaggtgcactcaatattcttagaaggtcacacatttcttgtaagaaaccatagacaggcatccttggaaatctctcccataacattccttacagctcaaaccctcttgatggaccttggacagtttctactgttttgtcatatctcttcccaccaacatgatgggatggaagcatatgtcaaagtagacagctgtccagaggaaccacaactacagatgaaaaataatgaagaagcagaagactatgatgatgatcttactgattcagaaatggatgtagtcaggtttgatgatgataactctccttcctttatccaaattaggtctgttgccaagaagcatcctaaaacttgggtacattacattgctgcagaagaggaggactgggactatgcaccattagtactagcacctgatgacagaagttataaaagtcaatatttgaacaatggaccccagagaattgggaggaagtacaaaaaagtcagatttatggcatacactgatgagacatttaagactagagaagctattcagcatgaatcaggaatcttgggacctttactttatggggaagtaggagacacactgttgattatatttaagaatcaagcaagcagaccatataacatctacccacatggaatcacagatgttagacccttgtattcaaggaggttaccaaaaggtgtaaaacatttgaaggattttcctattctgccaggtgaaatattcaaatataaatggactgtgactgttgaagatggaccaactaaatcagaccccaggtgcctgaccaggtattactctagttttgttaatatggagagagatctagcttcaggactcataggccctctcctcatctgctacaaagaatcagtagatcaacagggaaaccagataatgtcagacaagaggaatgtaatcctgttttctgtatttgatgaaaacagaagctggtacctcacagagaatatacaaaggtttctccccaatcctgcaggagtgcagcttgaggatcctgagttccaagcttccaacatcatgcacagcatcaatgggtatgtgtttgattctttgcagttgtctgtttgtttgcatgaagtggcatactggtacattctatctattggtgctcagactgacttcctttctgtattcttctcaggctatactttcaaacacaaaatggtctatgaagacaccctcaccctattcccattctctggggaaactgtgttcatgtccatggaaaaccctggactatggattctgggctgccacaactctgactttagaaacagaggcatgactgctttactgaaggtttcaagttgtgacaagaacactggtgattattatgaagactcctatgaagatattagtgcctacttgctgagtaaaaacaatgcaatagaaccaaggagcttctctcaaaaccccccagtcttgaaaaggcatcaaagagaaataaccagaactactcttcagtcagatcaagaggaaattgactatgatgataccataagtgttgaaatgaagaaggaagattttgacatttatgatgaggatgaaaatcagagccccaggagctttcaaaagaaaacaagacactattttatagctgctgtagagagactctgggattatgggatgagttcatcccctcatgtactaaggaacagggcccagtcaggttcagtcccccagttcaagaaagttgtcttccaggaatttacagatgggtcctttactcagcctttatacaggggagaactaaatgaacatttgggactcctgggaccatatataagagcagaagttgaagataatatcatggtaactttcaggaatcaggcctctagaccctattccttctattctagccttatttcttatgaagaagatcagaggcaaggagcagaacctagaaaaaactttgtcaagcctaatgaaactaaaacttacttttggaaagtgcaacatcatatggctcccactaaagatgagtttgattgcaaagcctgggcatatttctcagatgttgacctagagaaagatgtacactcaggactgattggacctcttctggtctgccacactaacacactgaaccctgctcatggtagacaagtgactgtacaggaatttgccctgtttttcaccatctttgatgagacaaaaagctggtacttcacagaaaatatggaaagaaactgcagggctccctgcaatatccagatggaagatcctacctttaaagagaattataggttccatgcaatcaatggctacataatggatacactacctgggttagtaatggctcaggatcaaaggattaggtggtatctgctcagcatgggctccaatgagaacatccattccattcatttctcaggacatgtgttcacagttaggaaaaaagaggagtataaaatggcactgtacaatctctatccaggtgtttttgaaacagtagaaatgttacccagtaaagctggaatttggagggtggagtgccttataggtgagcatctacatgcaggaatgagcacactttttctggtgtacagcaataagtgtcagacacccctgggaatggcctcaggacacattagggactttcagattacagccagtggacaatatggtcagtgggccccaaagctggccagacttcattatagtggttcaatcaatgcttggagcactaaggagcccttttcttggatcaaggtggatctgctggctcccatgattattcatggcatcaagacacagggtgctaggcagaagttcagcagtctctacatctctcagtttatcatcatgtattcacttgatgggaagaagtggcagacttatagaggaaattccacaggaaccttaatggtcttctttggaaatgtggattcatctggaataaaacacaatatttttaaccctccaattattgctagatacatcaggttgcacccaactcattatagcattagaagcactcttagaatggagttgatgggctgtgatttaaatagttgctccatgcccttgggaatggagagtaaagcaatatcagatgcccagattacagcttcatcatactttaccaatatgtttgcaacatggtctccatcaaaagcaagacttcacctccaagggagaagtaatgcctggagacctcaggtgaataatccaaaagagtggctgcaagtggacttccagaagactatgaaagtcacaggagtaacaactcagggagttaaatctctgcttaccagcatgtatgtaaaggagttcctcatctccagcagtcaagatgggcatcagtggactctcttttttcagaatggaaaagtaaaggtttttcagggaaatcaagactccttcacaccagtggttaacagtctggaccctcccttactgactagataccttaggattcaccctcagagttgggtgcaccagattgcactgagaatggaggttctgggctgtgaggcacaggacctctactga

SEQ ID NO: 78 - DNA, 4374 bp, synthetic construct
  source 1..4374: mol_type = other DNA; note = FVIII-QQ18
atgcaaatagagctctccacctgcttctttctgtgccttttgaggttctgctttagtgcaacaagaagatactacctgggtgcagtagaactgtcatgggactatatgcaatctgatttaggggagctgcctgtggatgcaagatttccacccagggtgcctaaaagctttcccttcaacacttcagttgtgtacaaaaagaccctgtttgtagaattcactgatcaccttttcaacattgctaagccaaggccaccctggatgggtctgcttggtcctaccatccaggcagaagtttatgatactgtggtcattacacttaagaacatggcttcccatccagtgagtcttcatgctgtgggtgtatcctactggaaagcttctgagggagctgaatatgatgaccagaccagtcaaagggagaaagaagatgacaaagtcttcccaggtggaagccatacctatgtgtggcaggtcctgaaagagaatggtccaatggcctctgatcctctgtgccttacctactcctatctttcacatgttgacctggtaaaagacttgaattcaggcctcattggggccctactagtttgtagagaagggtctctggctaaggaaaagactcagacattgcacaaatttatactactttttgctgtatttgatgaggggaaaagttggcactcagagacaaagaactccttgatgcaggatagagatgctgcttcagctagagcatggcctaaaatgcacactgtcaatggatatgtaaacagaagcctgccaggtctgattggttgccacaggaaatctgtctattggcatgtgattggcatgggcaccactccagaagtgcactcaatattcttagagggtcacacatttcttgtaagaaaccatagacaggcttccttggaaatctcacctataaccttccttacagctcaaaccctcttgatggacctgggacagtttctactgttttgtcatatctcttcccaccaacatgatggaatggaagcttatgtcaaagtagacagctgtccagaggaaccccaactacagatgaaaaataatgaagaagctgaagactatgatgatgatcttactgatagtgaaatggatgtagtcaggtttgatgatgacaactctccttcctttatccaaattaggtcagttgctaagaagcatcctaaaacttgggtacattacattgcagcagaagaggaggactgggactatgccccattagtcctagcccctgatgatagaagttataaaagtcaatatttgaacaatggaccacagagaattggtaggaagtacaaaaaagtcagatttatggcatacactgatgagacctttaagacaagggaagctattcagcatgaatctggaatcttgggacctttactttatggagaagttggggacacactgttgattatatttaagaatcaagcaagcagaccatataacatctacccccatggaatcactgatgtcaggcccttgtattcaagaaggttacctaaaggtgtaaaacatttgaaggattttcctattctgccaggtgaaatattcaaatataaatggacagtgactgtagaagatggcccaactaaaagtgatcccaggtgcctgaccagatattactctagttttgtcaatatggagagagatctagcttcaggactcattggtcctctcctcatctgctacaaagaatcagtagatcaacagggaaaccagataatgagtgataagaggaatgtcatcctgtttagtgtatttgatgaaaacagaagctggtacctcacagaaaatatacaaagatttctccccaatccagctggggtgcagttggaggatccagaattccaagcaagtaacatcatgcacagcatcaatgggtatgtatttgactctttgcagttgtcagtatgtttgcatgaagtggcatactggtacattctatcaattggggcccagactgatttccttagtgtcttcttctctggttatacattcaaacacaaaatggtctatgaagacaccctcaccctattcccattctcaggggaaactgtcttcatgagtatggaaaacccaggtctatggattttggggtgccacaactcagactttaggaacagaggcatgactgcattactgaaggtttcaagttgtgataagaacactggtgattattatgaagacagttatgaggatatttcagcatacttgctgagtaaaaacaatgcaatagaaccaagaagcttctctcaaaacccaccagtcttgaaaagacatcaaagagaaataaccaggactactcttcagtctgatcaagaggaaattgattatgatgacaccatatcagttgaaatgaagaaggaagattttgatatttatgatgaggatgaaaatcagagcccaaggagctttcaaaagaaaaccagacactattttattgcagcagtagagagactctgggattatgggatgagttcatccccccatgttctaagaaacagggcacagagtggcagtgtcccacagttcaagaaagtagttttccaggaatttacagatggttcctttactcagccattatacaggggagaactaaatgaacatttgggactcttagggccatatataagagctgaagttgaagataatatcatggtaactttcaggaatcaggcctctagaccatattccttctatagtagccttatttcttatgaggaagatcagaggcaaggagcagagcctaggaaaaactttgttaagcctaatgaaacaaaaacttacttttggaaagtgcaacatcatatggcacccaccaaagatgagtttgactgcaaagcctgggcctatttctctgatgttgacttagaaaaagatgtgcactctgggctgattggaccacttctggtctgccacactaacacactgaacccagcacatggcagacaagtgactgtccaggaatttgctctgtttttcaccatctttgatgagacaaaaagctggtacttcactgaaaatatggaaaggaactgcagggctccctgcaatatccagatggaagatccaacctttaaagagaattatagattccatgccatcaatgggtacataatggatacactacctggcttagtaatggctcaggatcaaaggattaggtggtatctgctcagcatgggctccaatgaaaacatccatagcattcatttcagtggacatgttttcactgtaaggaaaaaagaggagtataaaatggcactgtacaatctctatccaggagtttttgagacagttgaaatgttaccatctaaagcaggaatttggagggttgaatgccttattggggaacatctacatgcaggtatgagcacactttttctggtgtacagcaataagtgtcagactccactgggaatggcatctggccacattagagattttcagattactgcatcagggcaatatggacagtgggccccaaagctggccagacttcattatagtggatcaatcaatgcttggagcaccaaggagcccttttcttggatcaaggtggatctgcttgcacccatgattattcatgggatcaagacccagggtgccagacagaagttcagcagcctctacatctctcagtttatcatcatgtatagccttgatggaaagaagtggcagacttataggggaaatagcacaggaaccttaatggtcttctttggtaatgttgattcaagtgggataaaacacaatatttttaaccctccaattatagctagatacatcagattgcaccccactcattatagcattaggagcactcttaggatggagttgatgggctgtgatttaaatagttgcagtatgcccttgggaatggagagtaaagcaatatcagatgctcagattactgcatcaagctactttactaatatgtttgctacatggtctccatcaaaagctagacttcacctccaagggagatccaatgcttggagacctcaggtgaataatccaaaagagtggctgcaagtggacttccagaagactatgaaagtcacaggggtaacaacccagggagtaaaatctctgcttaccagcatgtatgtcaaggagttcctcatctccagctcacaagatgggcatcagtggactctcttttttcagaatgggaaagtaaaggtttttcagggaaatcaagactccttcacaccagtggtgaactctttggacccacctttactgactagataccttaggattcacccacagagttgggtgcaccagatagctctgagaatggaggttctgggctgtgaggcccaggacctctactga

SEQ ID NO: 79 - protein, 14 aa, synthetic construct
  source 1..14: note = linker
SFSQNPPVLKRHQR

SEQ ID NO: 80 - protein, 19 aa, synthetic construct
  source 1..19: note = N-terminal secretory signal sequence
MQIELSTCFFLCLLRFCFS

SEQ ID NO: 81 - protein, 20 aa, synthetic construct
  source 1..20: note = 201 signal peptide
MEFGLSWVFLVALLKGVQCE

SEQ ID NO: 82 - protein, 20 aa, synthetic construct
  source 1..20: note = wt IL2 signal peptide
MYRMQLLSCIALSLALVTNS

SEQ ID NO: 83 - protein, 20 aa, synthetic construct
  source 1..20: note = mut IL2 signal peptide
MYRMQLLLLIALSLALVTNS

SEQ ID NO: 84 - protein, 23 aa, synthetic construct
  source 1..23: note = A2M signal peptide
MGKNKLLHPSLVLLLLVLLPTDA

SEQ ID NO: 85 - protein, 25 aa, synthetic construct
  source 1..25: note = PZP signal peptide
MRKDRLLHLCLVLLLILLSASDSNS

SEQ ID NO: 86 - protein, 21 aa, synthetic construct
  source 1..21: note = artificial signal peptide
MWWRLWWLLLLLLLLWPMVWA

SEQ ID NO: 87 - protein, 11 aa, synthetic construct
  source 1..11: note = cathpetsin L signal peptide
LLLLAVLCLGT

SEQ ID NO: 88 - DNA, 72 bp, synthetic construct
  source 1..72: mol_type = other DNA; note = nucleotide of hAAT signal peptide
atgccgtcttctgtctcgtggggcatcctcctgctggcaggcctgtgctgcctggtccctgtctccctggct

SEQ ID NO: 89 - protein, 32 aa, synthetic construct
  source 1..32: note = FIBrat Signal Peptide
MLRGPGPGRLLLLAVLCLGTSVRCTETGKSKR

SEQ ID NO: 90 - protein, 38 aa, synthetic construct
  source 1..38: note = FIBrat Signal Peptide
MLRGPGPGRLLLLAVLCLGTSVRCTETGKSKRLALQIV

SEQ ID NO: 91 - protein, 26 aa, Homo sapiens
MLRGPGPGLLLLAVQCLGTAVPSTGA

SEQ ID NO: 92 - protein, 31 aa, Xenopus laevis
MRRGALTGLLLVLCLSVVLRAAPSATSKKRR

SEQ ID NO: 93 - protein, 17 aa, Rattus rattus
MTPLLLLAVLCLGTALA

SEQ ID NO: 94 - protein, 22 aa, Homo sapiens
MLSFVDTRTLLLLAVTLCLATC

SEQ ID NO: 95 - protein, 2351 aa, Homo sapiens
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 96 - protein, 214 aa, Homo sapiens
MRIQDPGKVFFGNVDSSGIKHNIFNPPIIARYIRLHPTHYSIRSTLRMELMGCDLNSCSMLGMESKAISDAQITASSYFTNMFATWSPSKARLHLQGRSNAWRPQVNNPKEWLQVDFQKMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 97 - DNA, 33 bp, synthetic construct
  source 1..33: mol_type = other DNA; note = LVR_CRE0080_PROC
aagcaaatatttgtggttatggattaactcgaa

SEQ ID NO: 98 - DNA, 23 bp, synthetic construct
  source 1..23: mol_type = other DNA; note = LVR_CRE0081_APOA1
ctgtttgcccactctatttgccc

SEQ ID NO: 99 - DNA, 29 bp, synthetic construct
  source 1..29: mol_type = other DNA; note = LVR_CRE0061_APOB
ggcgccctttggaccttttgcaatcctgg

SEQ ID NO: 100 - DNA, 16 bp, synthetic construct
  source 1..16: mol_type = other DNA; note = LVR_CRE0082_APOC4
agcaaacagcaaacac

SEQ ID NO: 101 - DNA, 13 bp, synthetic construct
  source 1..13: mol_type = other DNA; note = HNF1
gttaatttttaaa

SEQ ID NO: 102 - DNA, 11 bp, synthetic construct
  source 1..11: mol_type = other DNA; note = HNF4
gtggcccttgg

SEQ ID NO: 103 - skipped

SEQ ID NO: 104 - DNA, 15 bp, synthetic construct
  source 1..15: mol_type = other DNA; note = HNF1
tggttaataatctca

SEQ ID NO: 105 - skipped

SEQ ID NO: 106 - DNA, 11 bp, synthetic construct
  source 1..11: mol_type = other DNA; note = HNF-3
gttcaaacatg

SEQ ID NO: 107 - DNA, 11 bp, synthetic construct
  source 1..11: mol_type = other DNA; note = C/EBP
ctaatactctg

SEQ ID NO: 108 - DNA, 12 bp, synthetic construct
  source 1..12: mol_type = other DNA; note = HNF-4
tgcaagggtcat

SEQ ID NO: 109 - DNA, 10 bp, synthetic construct
  source 1..10: mol_type = other DNA; note = C/EBP
ttactcaaca

SEQ ID NO: 110 - DNA, 13 bp, synthetic construct
  source 1..13: mol_type = other DNA; note = HNF-4
cgccctttggacc

SEQ ID NO: 111 - DNA, 23 bp, synthetic construct
  source 1..23: mol_type = other DNA; note = HNF-4
actgaacccttgacccctgccct

SEQ ID NO: 112 - DNA, 23 bp, synthetic construct
  source 1..23: mol_type = other DNA; note = HNF-3
ctgtttgcccactctatttgccc

SEQ ID NO: 113 - DNA, 22 bp, synthetic construct
  source 1..22: mol_type = other DNA; note = HNF-3b
tgcccactctatttgcccagcc

SEQ ID NO: 114 - DNA, 15 bp, synthetic construct
  source 1..15: mol_type = other DNA; note = HNF4A
aagtccaaaggtaga

SEQ ID NO: 115 - DNA, 12 bp, synthetic construct
  source 1..12: mol_type = other DNA; note = FOXA1
gagtcaacatga

SEQ ID NO: 116 - DNA, 12 bp, synthetic construct
  source 1..12: mol_type = other DNA; note = FOXA1
cagtcaacattt

SEQ ID NO: 117 - DNA, 18 bp, synthetic construct
  source 1..18: mol_type = other DNA; note = c/EBP
gaccttttgcaatcctgg

SEQ ID NO: 118 - DNA, 62 bp, synthetic construct
  source 1..62: mol_type = other DNA; note = CRE0077 (V1)
aagcaaatatttgtggttatggattaactcgaactgtttgcccactctatttgccctgtacc

SEQ ID NO: 119 - DNA, 24 bp, synthetic construct
  source 1..24: mol_type = other DNA; note = 5 UTR (24bp)
caggtggattcttgggcatttgct

SEQ ID NO: 120 - DNA, 26 bp, synthetic construct
  source 1..26: mol_type = other DNA; note = hF8SQ_Probe
  misc_feature 26: note = FAM
gtttgaaactgtggagatgctgccct

SEQ ID NO: 121 - DNA, 20 bp, synthetic construct
  source 1..20: mol_type = other DNA; note = hF8SQ_Forward
ccacttctctgggcatgtgt

SEQ ID NO: 122 - DNA, 20 bp, synthetic construct
  source 1..20: mol_type = other DNA; note = hF8SQ_Reverse
cctccagatgccagccttag

SEQ ID NO: 123 - DNA, 277 bp, synthetic construct
  source 1..277: mol_type = other DNA; note = SP0412
aggttaatttttaaaaagcagtcaaaagtccaagtggcccttggcagcatttactctctctgtttgctctggttaataatctcaggagcacaaacattccccctgttcaaacatgtcctaatactctgtctctgcaagggtcatcagtagttttccatcttactcaacatcctcccagtgagtcatatgtttgctcactgaaggttactagttaacaggcatcccttaaacaggatataaaaggacttcagcaggactgctcgaaacatcccactca

SEQ ID NO: 124 - DNA, 282 bp, synthetic construct
  source 1..282: mol_type = other DNA; note = SP0246
aggttaatttttaaaaagcagtcaaaagtccaagtggcccttggcagcatttactctctctgtttgctctggttaataatctcaggagcacaaacattccggcccgggaggcgccctttggaccttttgcaatcctggcgcactgaacccttgacccctgccctgcagcccccgcagcttgctgtttgcccactctatttgcccagccccaggggcatataaaacaggggcaaggcacagactcatagcagagcaatcaccaccaagcctggaataactgca

SEQ ID NO: 125 - DNA, 4374 bp, synthetic construct
  source 1..4374: mol_type = other DNA; note = F8QQ-00
atgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgcagatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagcagggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaaccccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactga

SEQ ID NO: 126 - DNA, 4374 bp, synthetic construct
  source 1..4374: mol_type = other DNA; note = F8-SQ
atgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaaccccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactga

SEQ ID NO: 127 - DNA, 251 bp, synthetic construct
  source 1..251: mol_type = other DNA; note = liver specific promoter
gtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 128 - DNA, 8218 bp, synthetic construct
  source 1..8218: mol_type = other DNA; note = polyA signal
tctagagctagcatatggatccatcgatttagggataacagggtaattatcagcacacaattgcccattatacgcgcgtataatggactattgtgtgctgatatctgtacacttaagggctagatcttagcttacgtcactagagggtccacgtttagtttttaagatccattgatctcctaaacgctgcaagattcgcaacctggtatacttagcctaggcgctaggtcctagtgcagcgggactttttttctaaagtcgttgagaggaggagtcgtcagaccagatagctttgatgtcctgatcggaaggatcgttggcccccctgcaggcagctgttaattaactgcgcgctcgctcgctcactgaggccgcccgggcaaagcccgggcgtcgggcgacctttggtcgcccggcctcagtgagcgagcgagcgcgcagagagggagtggccaactccatcactaggggttcctgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgcagatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagcagggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaaccccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgaaataaaagatctttattttcattagatctgtgtgttggttttttgtgtgaggaacccctagtgatggagttggccactccctctctgcgcgctcgctcgctcactgaggccgggcgaccaaaggtcgcccgacgcccgggctttgcccgggcggcctcagtgagcgagcgagcgcgcagttaattaaggcgccctaggccgacccttagactctgtactcagttctataaacgagccattggatacgagatccgtagattgataagggacacggaatatccccggacgcaatagacaccggtggacagcttggtatcctgagcacagtcgcgcgtccgaatctagctctactttagaggccccggattctgatggtcgtagaccgcagaaccgattggggggatgagatctactagttatcagcacacaattgcccattatacgcgcgtataatggactattgtgtgctgatatagggataacagggtaattctagagctagcatatggatccatcgatttgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagcgtataatggtctagagctagcatatggatccatcgattccattatacgcctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 129 - DNA, 22 bp, synthetic construct
  source 1..22: mol_type = other DNA; note = Probe_HLP
  misc_feature 22: note = FAM
gggggcgactcagatcccagcc

SEQ ID NO: 130 - DNA, 20 bp, synthetic construct
  source 1..20: mol_type = other DNA; note = Forward_HLP
aggacgctgtggtttctgag

SEQ ID NO: 131 - DNA, 20 bp, synthetic construct
  source 1..20: mol_type = other DNA; note = Reverse_HLP
aggtcaccccagttatcgga